(19) United States
(12) Patent Application Publication
(10) Pub. No.: US 2006/0094880 A9
(43) Pub. Date: May 4, 2006

(54) Synthetic process for trans-aminocyclohexyl ether compounds

(76) Inventors: Anthony G. M. Barrett, London (GB); Lewis S. L. Choi, Burnaby (CA)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/862,157

(22) Filed: Jun. 4, 2004

Related U.S. Application Data
(60) Provisional application No. 60/476,083, filed on Jun. 4, 2003.
(60) Provisional application No. 60/475,884, filed on Jun. 5, 2003.
(60) Provisional application No. 60/475,912, filed on Jun. 5, 2003.
(60) Provisional application No. 60/476,447, filed on Jun. 5, 2003.
(60) Provisional application No. 60/489,659, filed on Jul. 23, 2003.
(60) Provisional application No. 60/516,486, filed on Oct. 31, 2003.

(51) Int. Cl. C07D 211/20; C07D 207/46

(52) U.S. Cl. 546/236; 548/577; 564/339

(57) ABSTRACT

A method of stereoselectively making an aminocyclohexyl ether comprises, for example, reacting 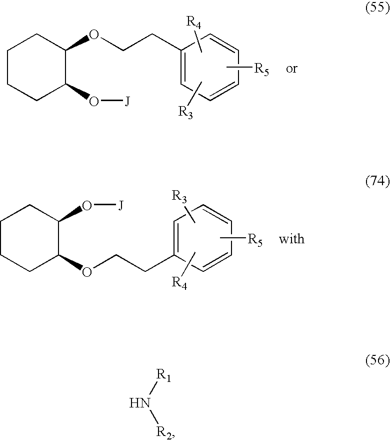
to form the aminocyclohexyl ether having the formula 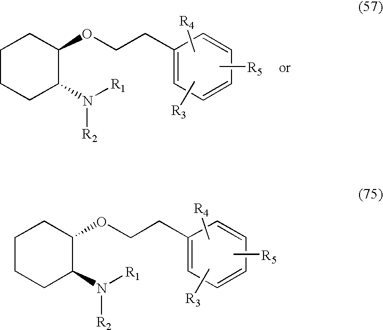 respectively,
wherein independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, and C₇-C₁₂aralkyl; or R₁ and R₂, when taken together with the nitrogen atom to which they are directly attached in formula (57) or (75), form a ring denoted by formula (I): 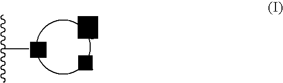
   wherein the ring of formula (I) is formed from the nitrogen as shown as well as three to nine additional ring atoms independently carbon, nitrogen, oxygen, or sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen; and wherein O-J is a leaving group. Methods of making intermediates are also disclosed.

### RELATED PATENT APPLICATIONS

This application claims priority to U.S. Provisional application nos. 60/516,486 filed 31 Oct. 2003; 60/476,083 filed 4 Jun. 2003; 60/475,884 filed 5 Jun. 2003; 60/475,912 filed 5 Jun. 2003; 60/476,447 filed 5 Jun. 2003; and 60/489,659 filed 23 Jul. 2003, each of which is incorporated in its entirety herein by reference.

### TECHNICAL FIELD

The present invention is generally directed toward a method for the preparation of stereoisomerically substantially pure trans-aminocyclohexyl ether compounds such as trans-(1R,2R)-aminocyclohexyl ether compounds and/or trans-(1S,2S)-aminocyclohexyl ether compounds as well as various intermediates and substrates involved. The compounds prepared by methods of the present invention are useful for treating medical conditions or disorders, including for example, cardiac arrhythmia, such as atrial arrhythmia and ventricular arrhythmia.

### BACKGROUND OF THE INVENTION

Arrhythmia is a variation from the normal rhythm of the heart beat and generally represents the end product of abnormal ion-channel structure, number or function. Both atrial arrhythmias and ventricular arrhythmias are known. The major cause of fatalities due to cardiac arrhythmias is the subtype of ventricular arrhythmias known as ventricular fibrillation (VF). Conservative estimates indicate that, in the U.S. alone, each year over one million Americans will have a new or recurrent coronary attack (defined as myocardial infarction or fatal coronary heart disease). About 650,000 of these will be first heart attacks and 450,000 will be recurrent attacks. About one-third of the people experiencing these attacks will die of them. At least 250,000 people a year die of coronary heart disease within 1 hour of the onset of symptoms and before they reach a hospital. These are sudden deaths caused by cardiac arrest, usually resulting from ventricular fibrillation.

Atrial fibrillation (AF) is the most common arrhythmia seen in clinical practice and is a cause of morbidity in many individuals (Pritchett E. L., N. Engl. J. Med. 327(14):1031 Oct. 1, 1992, discussion 1031-2; Kannel and Wolf, Am. Heart J. 123(1):264-7 Jan. 1992). Its prevalence is likely to increase as the population ages and it is estimated that 3-5% of patients over the age of 60 years have AF (Kannel W. B., Abbot R. D., Savage D. D., McNamara P. M., N. Engl. J. Med. 306(17):1018-22, 1982; Wolf P. A., Abbot R. D., Kannel W. B. Stroke. 22(8):983-8, 1991). While AF is rarely fatal, it can impair cardiac function and is a major cause of stroke (Hinton R. C., Kistler J. P., Fallon J. T., Friedlich A. L., Fisher C. M., American Journal of Cardiology 40(4):509-13, 1977; Wolf P. A., Abbot R. D., Kannel W. B., Archives of Internal Medicine 147(9):1561-4, 1987; Wolf P. A., Abbot R. D., Kannel W. B. Stroke. 22(8):983-8, 1991; Cabin H. S., Clubb K. S., Hall C., Perlmutter R. A., Feinstein A. R., American Journal of Cardiology 65(16):1112-6, 1990).

WO99/50225 discloses a class of aminocyclohexylether compounds as useful in the treatment of arrhythmias. Some of the new aminocyclohexylether compounds have been found to be particularly effective in the treatment and/or prevention of AF. However, synthetic methods described in WO099/50225 and elsewhere were non-stereoselective and led to mixture of stereoisomers (see e.g., FIGS. 1-3). As active pharmaceutical compounds, it is often desirable that drug molecules are in stereoisomerically substantially pure form. It may not be feasible or cost effective if the correct stereoisomer has to be isolated from a mixture of stereoisomers after a multi-step synthesis. Therefore, there remains a need in the art to develop method for the preparation of stereoisomerically substantially pure trans-aminocyclohexyl ether compounds.

Although WO 2003/105756 describes a method of stereoselectively preparing a 1,2, di-substituted cycloalkane, the method disclosed therein requires a trans-1R,2R di-substituted cycloalkane. In an alternate embodiment, disclosed is a method that requires reacting a cis-2-substituted cycloalkane with a galactose derivative. The present invention does not have such requirements.

### SUMMARY OF THE INVENTION

In one embodiment, the method of the invention is directed to a method of stereoselectively making an aminocyclohexyl ether comprising
reacting 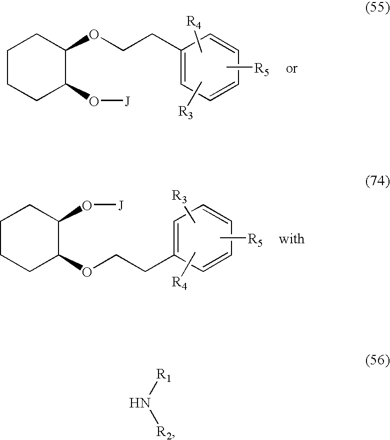
   to form the aminocyclohexyl ether having the formula 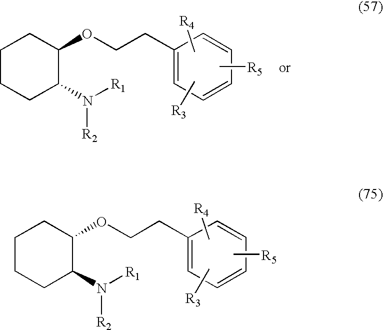
   respectively. This step corresponds to the last step in, for example, FIGS. 5, 45, 85, 104, 121, and 147.

Independently at each occurrence above or in the following intermediates, R₁ and R₂ are independently hydrogen, C₁-C₈ alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, and C₇-C₁₂aralkyl; or R₁ and R₂, when taken together with the nitrogen atom to which they are directly attached in formula (57) or (75), form a ring denoted by formula (I): 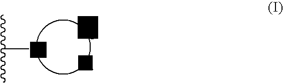
wherein the ring of formula (I) is formed from the nitrogen as shown as well as three to nine additional ring atoms independently carbon, nitrogen, oxygen, or sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl.

Preferably, the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy. R₃, R₄ and R₅ are independently selected from the group consisting of hydrogen, hydroxy and C₁-C₆alkoxy, with the proviso that R₃, R₄ and R₅ cannot all be hydrogen. More preferably, 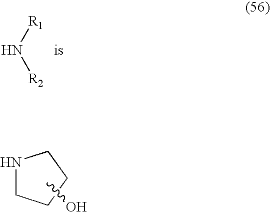
and, even more preferably,

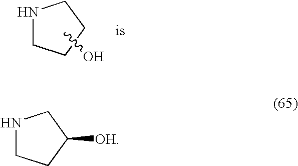

R₃, R₄ and R₅ above or in the following intermediates are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen. Preferably, R₃, R₄ and R₅ are independently selected from the group consisting of hydrogen, hydroxy and C₁-C₆alkoxy, with the proviso that R₃, R₄ and R₅ cannot all be hydrogen, and even more preferably, at least one of R₃, R₄ and R₅ is C₁-C₆alkoxy.

Above and in the following intermediates, O-J is a leaving group. More preferably, O-J is selected from an alkyl sulfonate or an aryl sulfonate. Most preferably, O-J is a mesylate, a benzenesulfonate, a mono- or poly- alkylbenzenesulfonate, a mono- or poly-halobenzenesulfonate, tosylate or nosylate. Even more preferably, O-J is a mesylate, a benzenesulfonate, a tosylate, 2-bromobenzenesulfonate, a 2,6-dichlorobenzenesulfonate or a nosylate.

With regard to the resulting compound, in a preferred embodiment, 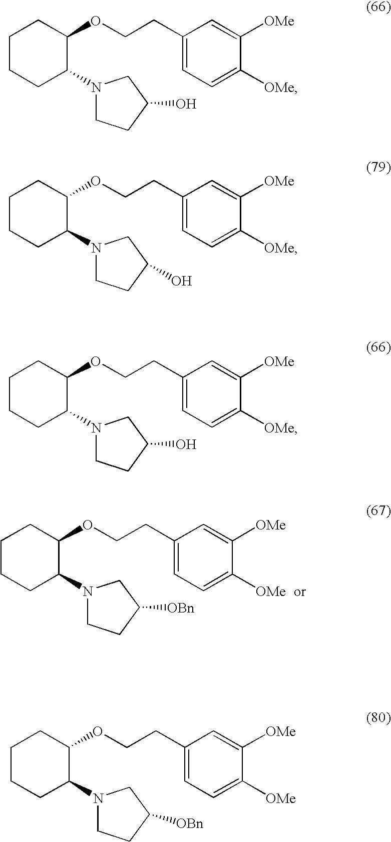
is formed.

In another aspect of the method of the invention, before the reacting step, the method preferably further comprises alkylating 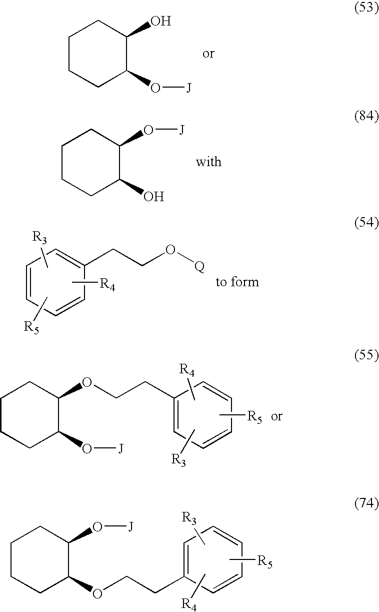
respectively. This step corresponds to the penultimate step in, for example, FIGS. 5 and 85. O-Q is a leaving group that reacts with —OH, for example, in formula (53) or (84), to form the ether of formula (55) or (74), such that the stereochemical configuration of the hydroxyl group is retained in the ether. Preferably, O-Q is trichloroacetimidate.

Optionally, the method may further include protecting 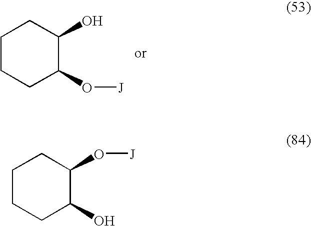
before the alkylating step.

With regard to the intermediates that are formed, preferably,

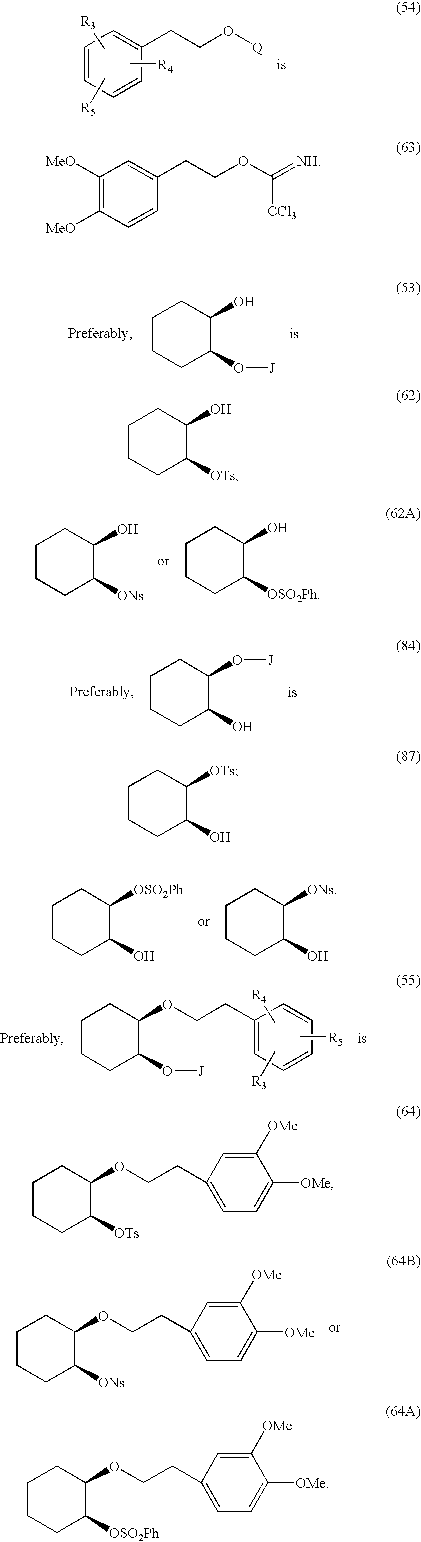

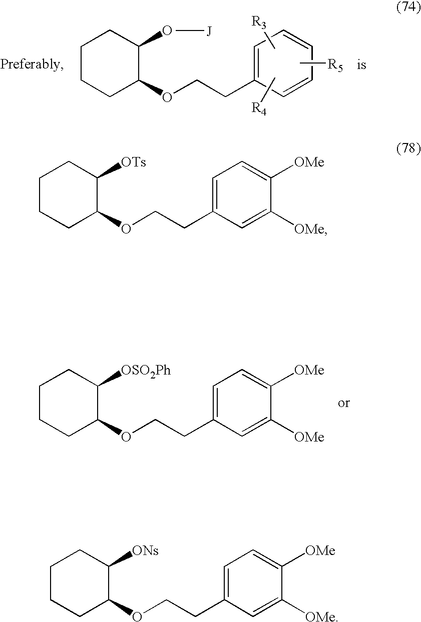

In one aspect, before the alkylating step, the method comprises hydrogenating and hydrogenolyzing 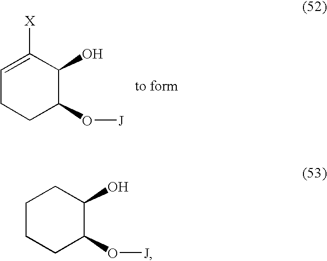
wherein X is a halide. Preferably, 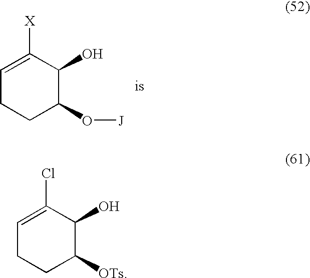
Preferably, before the hydrogenating and hydrogenolyzing step, the method comprises activating 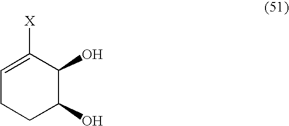
with a hydroxy activating reagent to form 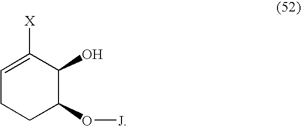
This step corresponds, for example, to an intermediate step in FIG. 5.

In another aspect, before the alkylating step, the method comprises deprotecting 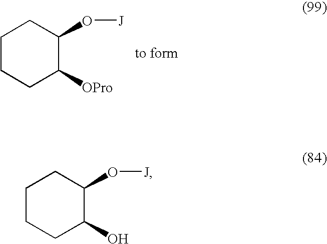
wherein Pro is a protecting group. Preferably, before the deprotecting step, the method comprises activating 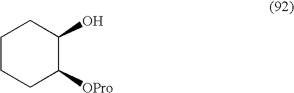
with a hydroxy activating reagent to form 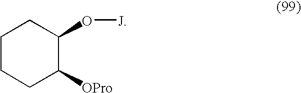
Preferably, the hydroxy activating reagent is tosyl halide, benzenesulfonyl halide or nosyl halide. Preferably, 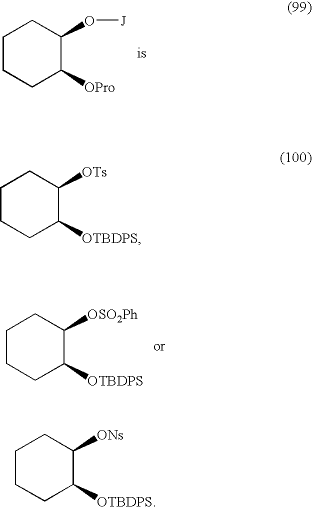
More preferably, before the activating step, the method comprises hydrogenating and hydrogenolyzing 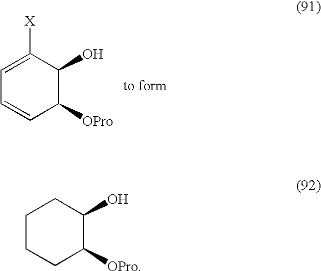
Preferably, 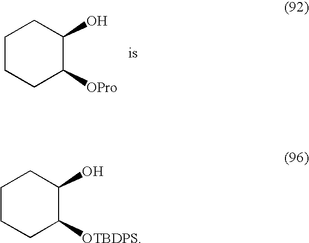
These steps correspond to, for example, portions of the methods of FIGS. 5 and 147.

In one aspect, before the alkylating step, the method preferably comprises removing a functional group G or G₁ from 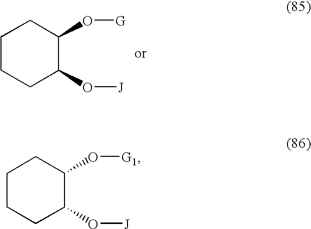
respectively, to form 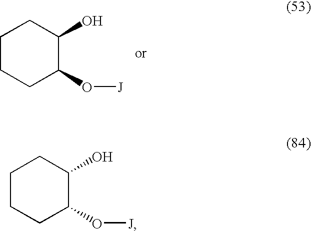
respectively. In another aspect, before the alkylating step, the method preferably comprises separating a racemic mixture of 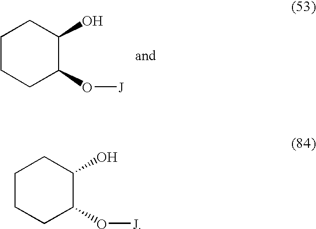
Preferably, the separation step further comprises functionalizing one or both of 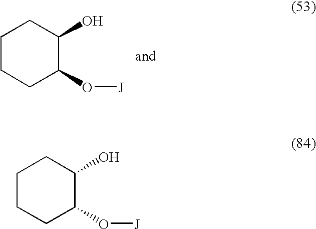
such that the compounds are capable of resolution; performing resolution to separate the compounds; and optionally removing the functional group on the one or both functionalized compounds. These steps correspond to intermediate steps in, for example, FIGS. 85 and 104.

Before the separating step the method preferably further comprises activating 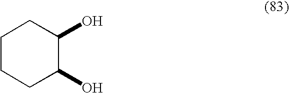
with a hydroxy activating reagent to form the racemic mixture of 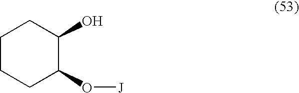
and 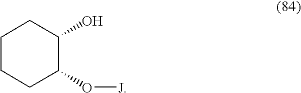
In one aspect, 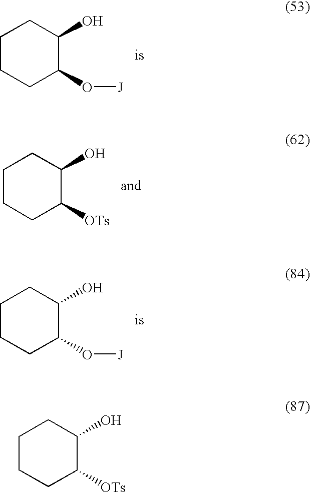
and is enzymatically functionalized with 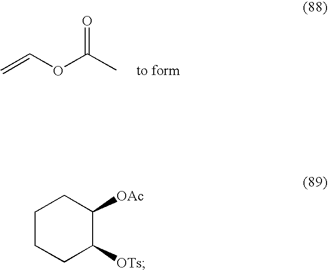
and the method further comprises performing resolution to separate 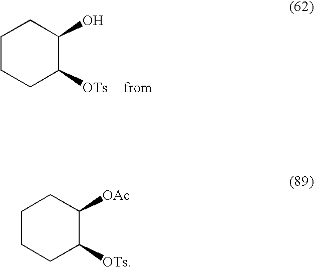
In another aspect, 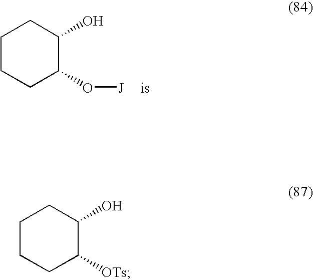
and 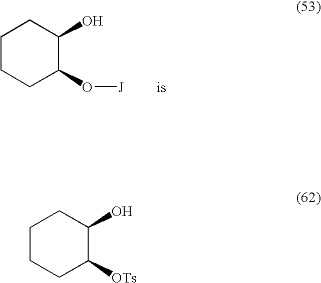
and is functionalized with 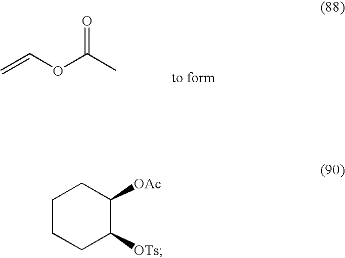
and the method further comprises performing resolution to separate 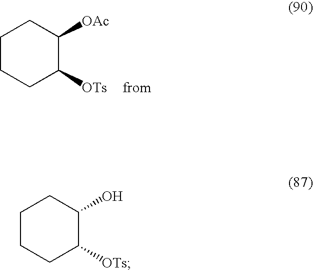
and removing the functional group from 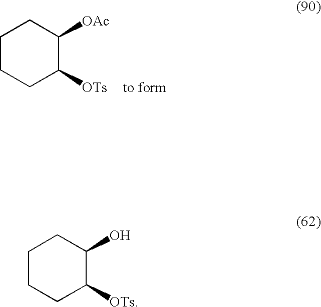
In yet another aspect, the method comprises, before the separating step, activating 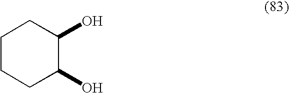
with a hydroxy activating reagent to form the racemic mixture. These steps correspond to, for example, portions of FIGS. 85 and 104.

In another aspect, before the reacting step, the method preferably further comprises activating 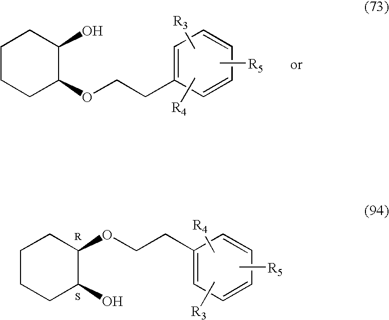
with a hydroxy activating reagent to form 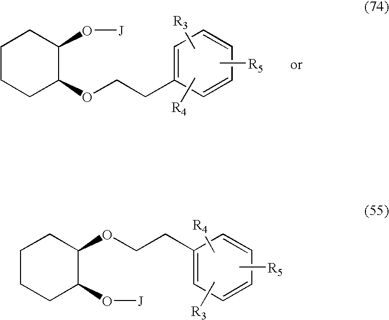
respectively. This step corresponds, for example, to an intermediate step in FIGS. 45 and 121. Preferably, the hydroxy activating reagent is an alkyl sulfonyl halide or an aryl sulfonyl halide. More preferably, the hydroxy activating reagent is tosyl halide, benzenesulfonyl halide or nosyl halide.

With respect to the compound subject to activation, preferably, 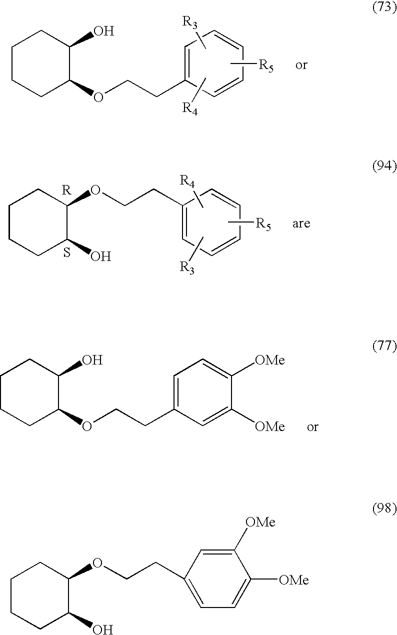
respectively.

With regard to the activated compound, preferably,

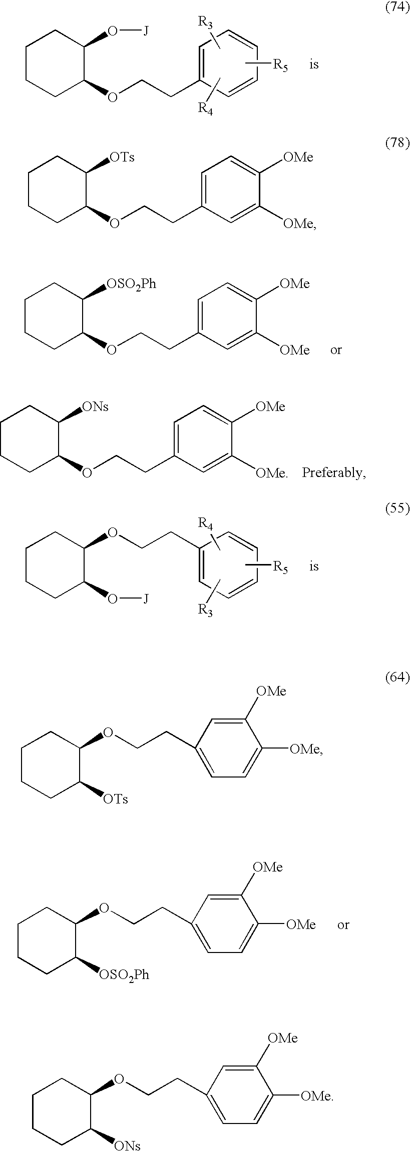

In another aspect, before the activating step, the method preferably further comprises hydrogenating and hydrogenolyzing 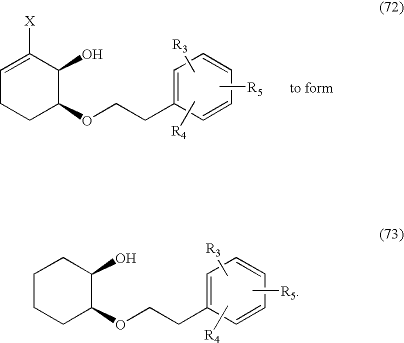
X may be a halide above and in the following intermediates. More preferably, X is a chloride. Preferably, 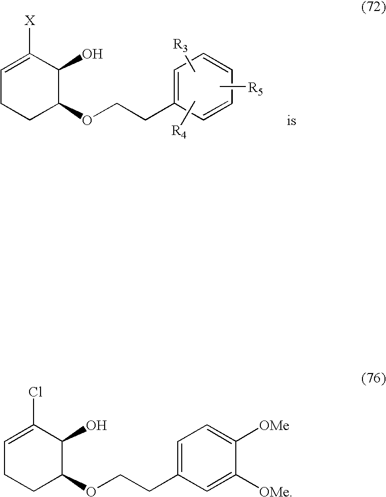
Before the hydrogenating and hydrogenolyzing step, the method preferably further comprises alkylating 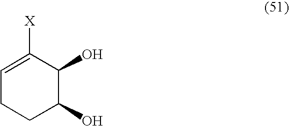
with 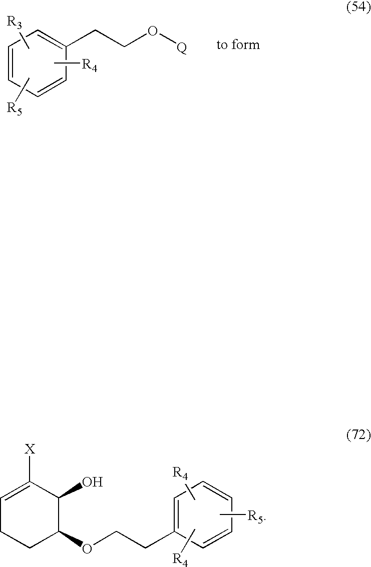
These steps correspond, for example, to intermediate steps in FIG. 45.

In another aspect, before the activating step, the method preferably further comprises deprotecting 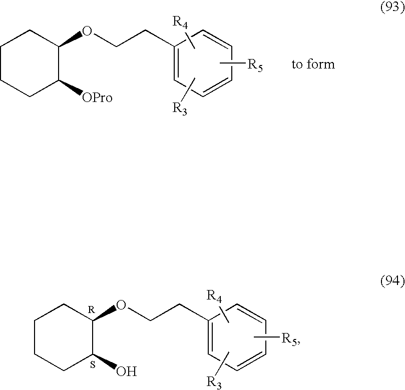
wherein Pro is a protecting group. Preferably, 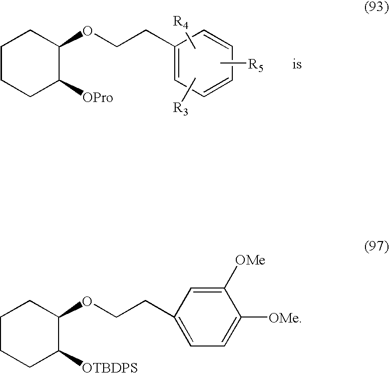
Before the deprotecting step, the method preferably further comprises alkylating 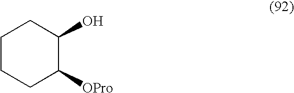
with 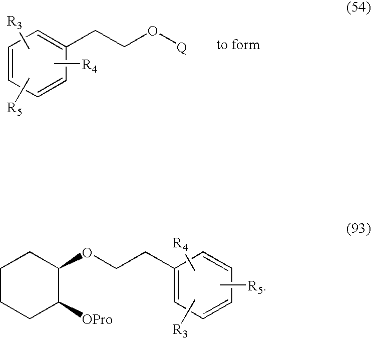
With regard to the protected intermediate, preferably, 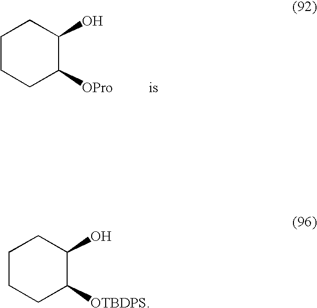
With regard to the compound for use in the alkylating step, preferably, 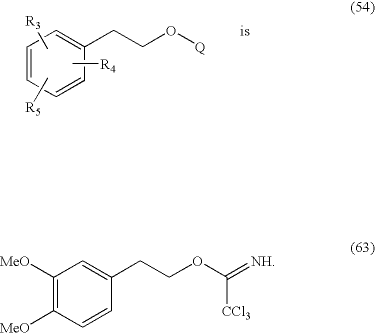
With regard to the alkylated and protected compound, preferably, 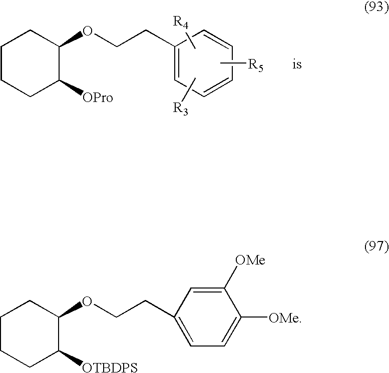
Before the alkylating step, the method preferably further comprises hydrogenating and hydrogenolyzing 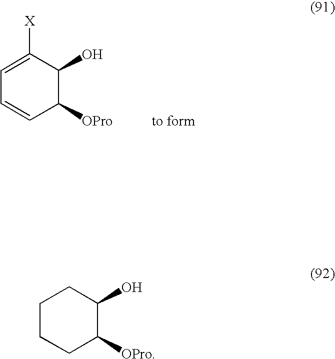
These steps correspond to, for example, intermediate steps in FIG. 121.

In another embodiment, the method of the invention takes advantage of alkylating an intermediate having a cis configuration and is directed to a method of stereoselectively making an aminocyclohexyl ether comprising alkylating 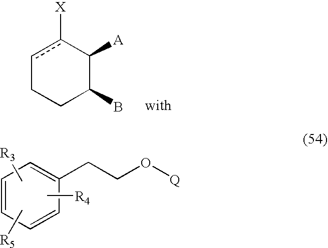
to form a reaction product; and optionally hydrogenating and hydrogenolyzing 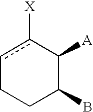
or the reaction product to reduce optional double bond and remove halide if present; reacting the reaction product of the alkylating step with 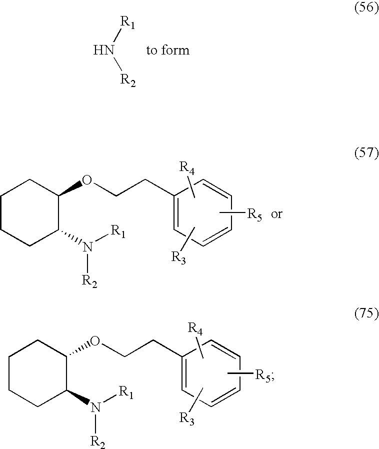
wherein - - - is an optional double bond;
wherein X is H or halide;
wherein A is OH, or a leaving group;
wherein B is OH, a leaving group, or a protecting group;
wherein only one of A and B may be OH;
wherein only one of A and B may be a leaving group; and
R₁, R₂, R₃, R₄, and R₅ and —O-Q are as defined above.

The steps of this embodiment are found, for example, in portions of FIGS. 5, 45, 85, 104, 121, and 147. With regard to the aminocyclohexyl ether, preferably, 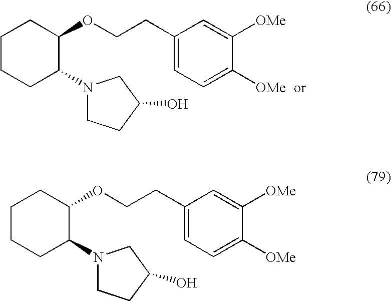
is formed.

In one aspect, 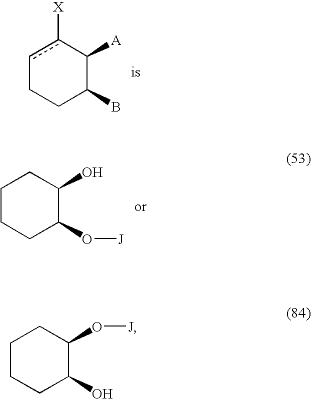
and the generic alkylating step described immediately above further comprises alkylating 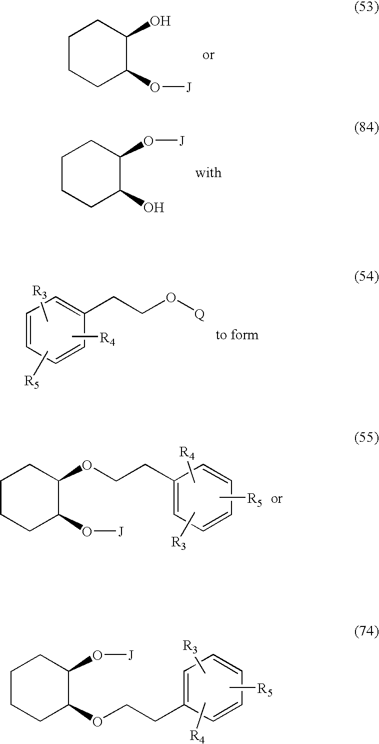
respectively. O-J is defined above. Similarly as described above, O-Q is a leaving group that reacts with —OH in formula (53) or (84) to form the ether of formula (55) or (74), such that the stereochemical configuration of the hydroxyl group is retained in the ether. Optionally, the method further comprises protecting 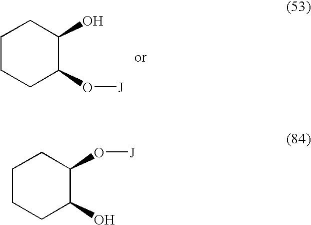
before the alkylating step. These steps represent intermediate steps in, for example, FIGS. 5, 85, 104, and 147.

Before the alkylating step, in one embodiment the method further comprises hydrogenating and hydrogenolyzing 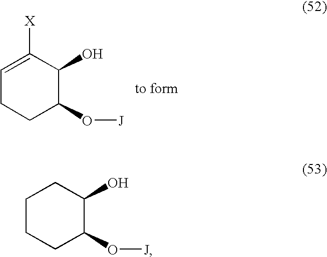
wherein X is a halide. Before the hydrogenating and hydrogenolyzing step, the method preferably further comprises activating 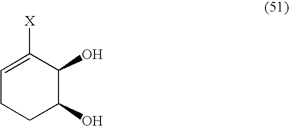
with a hydroxy activating reagent to form 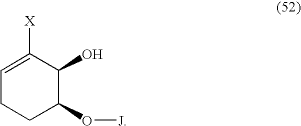
These steps correspond to intermediate steps in, for example, FIG. 5.

In another embodiment, before the alkylating step, the method further comprises hydrogenating and hydrogenolyzing 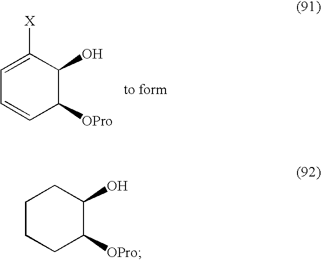
activating 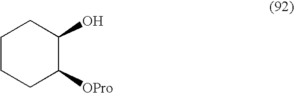
with a hydroxy activating reagent to form 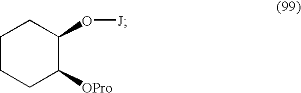
and deprotecting 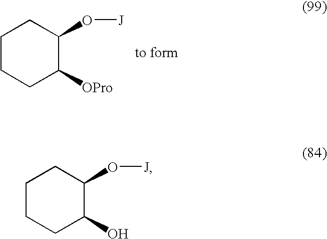
wherein Pro is a protecting group. These steps correspond to intermediate steps in, for example, FIG. 147.

In another embodiment, before the alkylating step, the method further comprises removing a functional group G or G₁ from 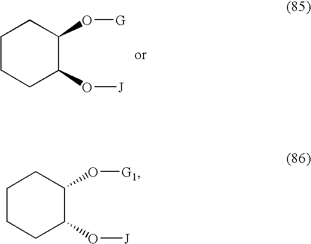
respectively, to form 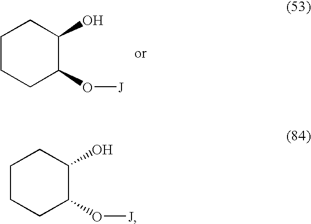
respectively. Preferably before the alkylating step, the method comprises separating a racemic mixture of 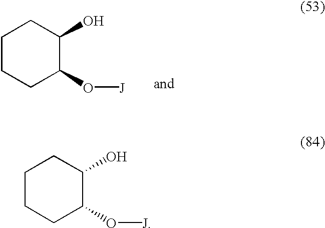
Preferably, the separation step further comprises functionalizing one or both of 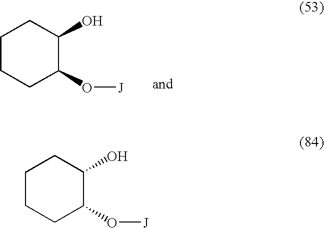
such that the compounds are capable of resolution; performing resolution to separate the compounds; and optionally removing the functional group on the one or both functionalized compounds. Preferably before the separating step the method further comprises activating 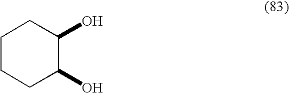
with a hydroxy activating reagent to form the racemic mixture of 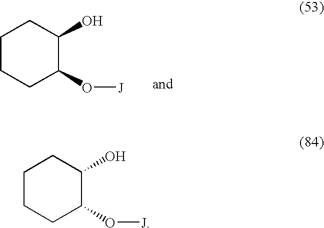
These steps correspond to intermediate steps in, for example, FIGS. 85 and 104.

In another aspect, 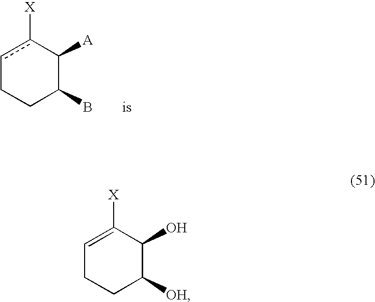
and the generic alkylating step described above further comprises alkylating 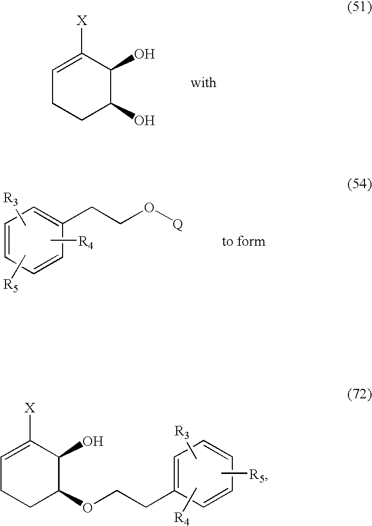
wherein the method further comprises hydrogenating and hydrogenolyzing 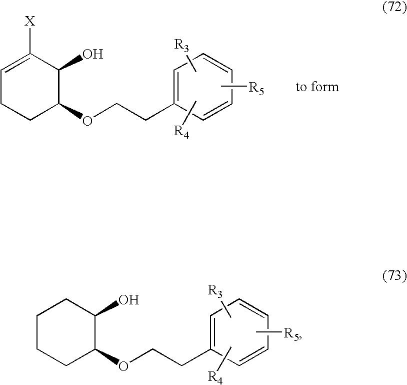
wherein X is a halide; and activating 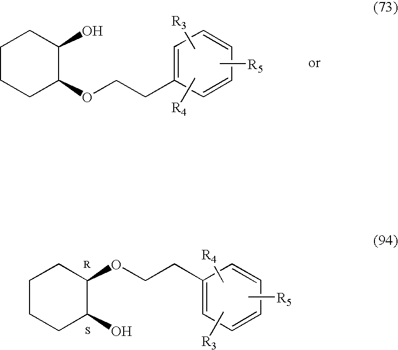
with a hydroxy activating reagent to form 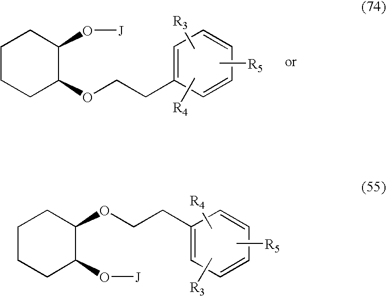
respectively. These steps correspond to intermediate steps in, for example, FIG. 45.

In another aspect, 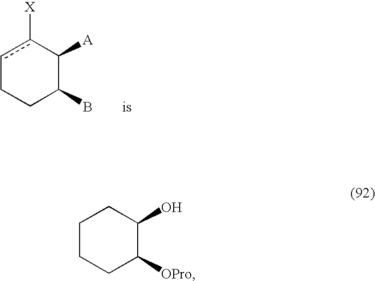
further comprising before the generic alkylating step, hydrogenating and hydrogenolyzing 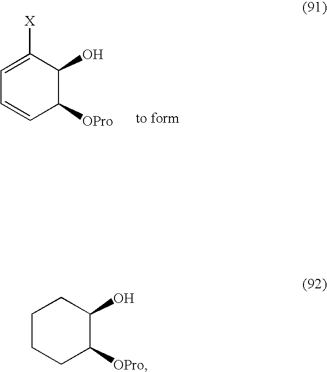
wherein the method further comprises alkylating 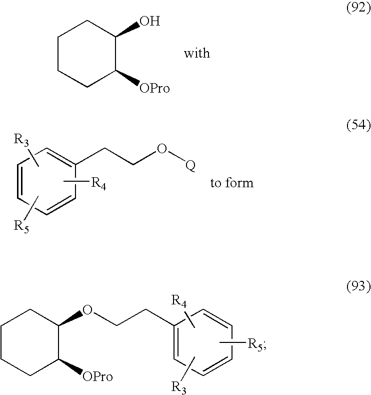
deprotecting 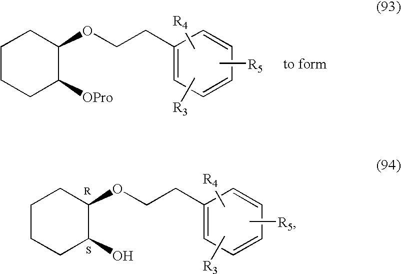
wherein Pro is a protecting group; and activating 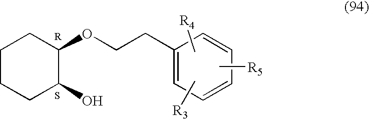
with a hydroxy activating reagent to form 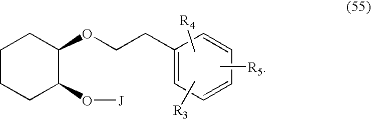
These steps correspond to the intermediate steps in, for example, FIG. 121.

It is also contemplated that individual steps of the methods described above for making intermediates are part of the invention described herein. In one aspect, a method of making intermediates comprises alkylating 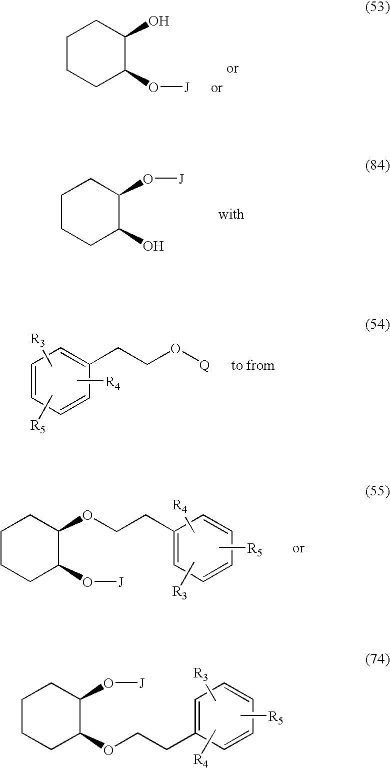
respectively; optionally protecting 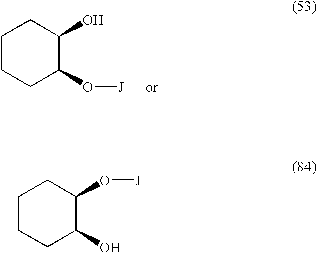
before the reacting step;
wherein O-Q is a leaving group that reacts with —OH in formula (53) or (84) to form the ether of formula (55) or (74), such that the stereochemical configuration of the the hydroxyl group is retained in the ether;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-J is a leaving group.

Another method of making an intermediate comprises activating 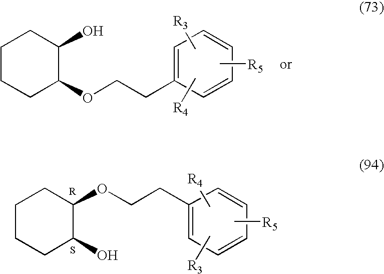
with a hydroxy activating reagent to form 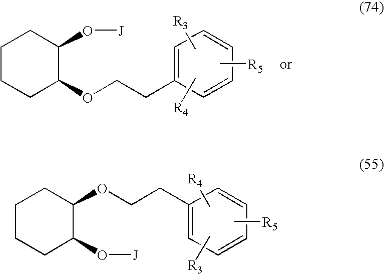
respectively;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-J is a leaving group.

Yet another method of making an intermediate comprises hydrogenating and hydrogenolyzing 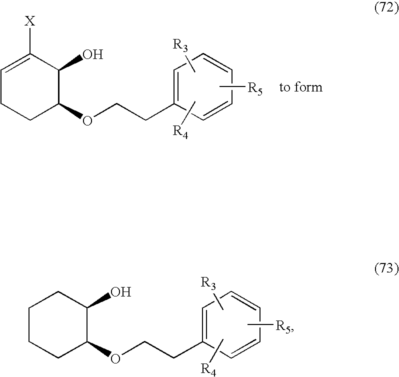
wherein X is a halide;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen.

Still another method for making an intermediate comprises alkylating 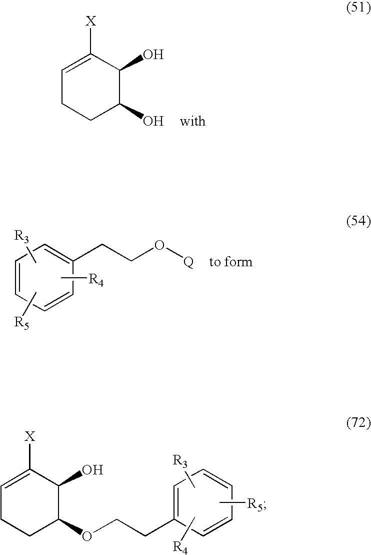
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen;
wherein X is a halide; and
wherein O-Q is a leaving group that reacts with —OH to form the ether, such that the stereochemical configuration of the hydroxyl group is retained in the ether.

Further, another method for making an intermediate comprises alkylating 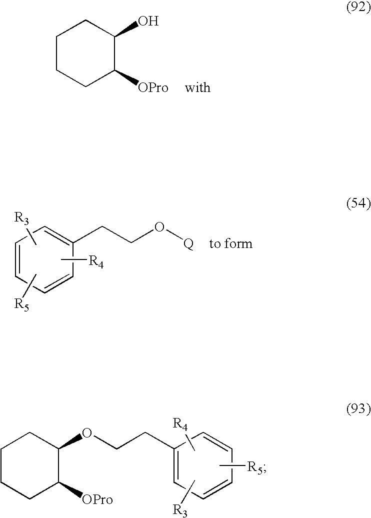
wherein Pro is a protecting group;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-Q is a leaving group that reacts with —OH to form the ether, such that the stereochemical configuration of the hydroxyl group is retained in the ether.

Another method of making an intermediate comprises hydrogenating and hydrogenolyzing 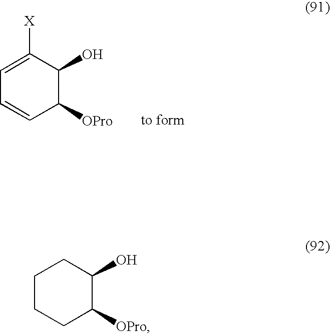
wherein Pro is a protecting group; and wherein X is a halide.

Another method of making an intermediate comprises hydrogenating and hydrogenolyzing 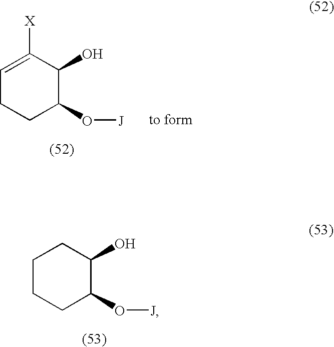
wherein X is a halide; and wherein O-J is a leaving group.

Another method of making an intermediate comprises activating 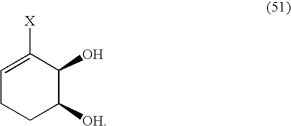
with a hydroxy activating reagent to form 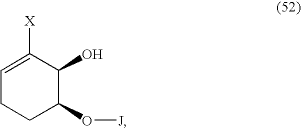
wherein X is a halide; and wherein O-J is a leaving group.

Another method of making an intermediate comprises activating 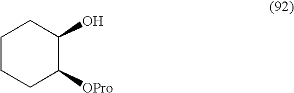
with a hydroxy activating reagent to form 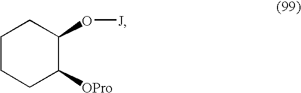
wherein Pro is a protecting group; and wherein O-J is a leaving group.

Another method of making an intermediate comprises hydrogenating and hydrogenolyzing 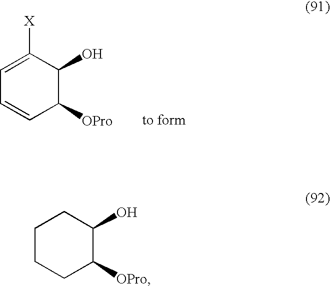
wherein X is a halide and wherein Pro is a protecting group.

Another method of making an intermediate comprises removing a functional group G or G₁ from 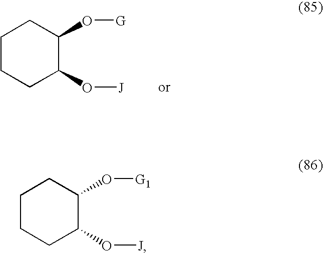
respectively, to form 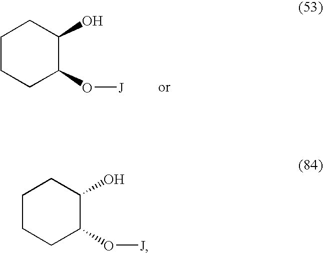
respectively, wherein O-J is a leaving group.

Another method of making an intermediate comprises separating a racemic mixture of 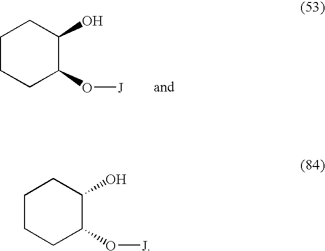
Preferably, the separation step further comprises functionalizing one or both of 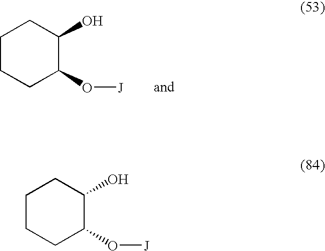
such that the compounds are capable of resolution; performing resolution to separate the compounds; and optionally removing the functional group on the one or both functionalized compounds.

Yet another method of making an intermediate comprises activating 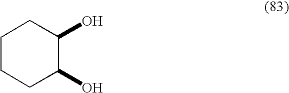
with a hydroxy activating reagent to form the racemic mixture of 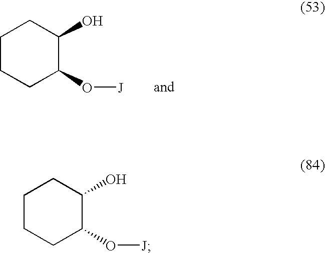
wherein O-J is a leaving group.

### BRIEF DESCRIPTION OF THE DRAWINGS

FIG. 1 illustrates a general synthetic methodology that may be employed to prepare a trans-aminocyclohexyl ether compound.

FIG. 2 illustrates a synthetic methodology that may be employed to prepare the trans-aminocyclohexyl ether compound of formulae (8) and (9).

FIG. 3 illustrates another general synthetic methodology that may be employed to prepare a trans-aminocyclohexyl ether compound.

FIG. 4 illustrates compounds that may be synthesized by the method of the invention as well as major reactants used to arrive at the compounds.

FIG. 5 illustrates a general reaction scheme that may be used as a process for preparing a stereoisomerically substantially pure trans-(1R,2R)-aminocyclohexyl ether compound of formula (57).

FIG. 6 illustrates a reaction scheme that may be used as a process for preparing a stereoisomerically substantially pure trans-(1R,2R)-aminocyclohexyl ether compound of formula (66).

FIG. 6A illustrates a reaction scheme that may be used as a process for preparing a stereoisomerically substantially pure trans-(1R,2R)-aminocyclohexyl ether compound of formula (66).

FIG. 7 illustrates a reaction scheme that may be used as a process for preparing a stereoisomerically substantially pure trans-(1R,2R)-aminocyclohexyl ether compound of formula (66).

FIG. 8 illustrates a reaction scheme that may be used as a process for preparing a stereoisomerically substantially pure trans-(1R,2R)-aminocyclohexyl ether compound of formula (69).

FIG. 9 illustrates a reaction scheme that may be used as a process for preparing a stereoisomerically substantially pure trans-(1R,2R)-aminocyclohexyl ether compound of formula (69).

FIG. 10 illustrates a general reaction scheme that may be used as a process for preparing a stereoisomerically substantially pure trans-(1R,2R)-aminocyclohexyl ether compound of formula (57).

FIG. 11 illustrates a reaction scheme that may be used as a process for preparing a stereoisomerically substantially pure trans-(1R,2R)-aminocyclohexyl ether compound of formula (66).

FIG. 12 illustrates a reaction scheme that may be used as a process for preparing a stereoisomerically substantially pure trans-(1R,2R)-aminocyclohexyl ether compound of formula (66).

FIG. 13 illustrates a reaction scheme that may be used as a process for preparing a stereoisomerically substantially pure trans-(1R,2R)-aminocyclohexyl ether compound of formula (69).

FIG. 14 illustrates a reaction scheme that may be used as a process for preparing a stereoisomerically substantially pure trans-(1R,2R)-aminocyclohexyl ether compound of formula (69).

FIG. 15 illustrates a general reaction scheme that may be used as a process for preparing a stereoisomerically substantially pure trans-(1R,2R)-aminocyclohexyl ether compound of formula (57).

FIG. 16 illustrates a reaction scheme that may be used as a process for preparing a stereoisomerically substantially pure trans-(1R,2R)-aminocyclohexyl ether compound of formula (66).

FIG. 17 illustrates a reaction scheme that may be used as a process for preparing a stereoisomerically substantially pure trans-(1R,2R)-aminocyclohexyl ether compound of formula (66).

FIG. 18 illustrates a reaction scheme that may be used as a process for preparing a stereoisomerically substantially pure trans-(1R,2R)-aminocyclohexyl ether compound of formula (69).

FIG. 19 illustrates a reaction scheme that may be used as a process for preparing a stereoisomerically substantially pure trans-(1R, 2R)-aminocyclohexyl ether compound of formula (69).

FIG. 20 illustrates a general reaction scheme that may be used as a process for preparing a stereoisomerically substantially pure trans-(1R,2R)-aminocyclohexyl ether compound of formula (57).

FIG. 21 illustrates a reaction scheme that may be used as a process for preparing a stereoisomerically substantially pure trans-(1R,2R)-aminocyclohexyl ether compound of formula (66).

FIG. 22 illustrates a reaction scheme that may be used as a process for preparing a stereoisomerically substantially pure trans-(1R,2R)-aminocyclohexyl ether compound of formula (66).

FIG. 23 illustrates a reaction scheme that may be used as a process for preparing a stereoisomerically substantially pure trans-(1R,2R)-aminocyclohexyl ether compound of formula (69).

FIG. 24 illustrates a reaction scheme that may be used as a process for preparing a stereoisomerically substantially pure trans-(1R,2R)-aminocyclohexyl ether compound of formula (69).

FIG. 25 illustrates a general reaction scheme that may be used as a process for preparing a stereoisomerically substantially pure trans-(1R,2R)-aminocyclohexyl ether compound of formula (57).

FIG. 26 illustrates a reaction scheme that may be used as a process for preparing a stereoisomerically substantially pure trans-(1R,2R)-aminocyclohexyl ether compound of formula (66).

FIG. 27 illustrates a reaction scheme that may be used as a process for preparing a stereoisomerically substantially pure trans-(1R,2R)-aminocyclohexyl ether compound of formula FIG. 28 illustrates a reaction scheme that may be used as a process for preparing a stereoisomerically substantially pure trans-(1R,2R)-aminocyclohexyl ether compound of formula (69).

FIG. 28 illustrates a reaction scheme that may be used as a process for preparing a stereoisomerically substantially pure trans-(1R,2R)-aminocyclohexyl ether compound of formula (69).

FIG. 29 illustrates a reaction scheme that may be used as a process for preparing a stereoisomerically substantially pure trans-(1R,2R)-aminocyclohexyl ether compound of formula (69).

FIG. 30 illustrates a general reaction scheme that may be used as a process for preparing a stereoisomerically substantially pure trans-(1R,2R)-aminocyclohexyl ether compound of formula (57).

FIG. 31 illustrates a reaction scheme that may be used as a process for preparing a stereoisomerically substantially pure trans-(1R,2R)-aminocyclohexyl ether compound of formula (66).

FIG. 32 illustrates a reaction scheme that may be used as a process for preparing a stereoisomerically substantially pure trans-(1R,2R)-aminocyclohexyl ether compound of formula (66).

FIG. 33 illustrates a reaction scheme that may be used as a process for preparing a stereoisomerically substantially pure trans-(1R,2R)-aminocyclohexyl ether compound of formula (69).

FIG. 34 illustrates a reaction scheme that may be used as a process for preparing a stereoisomerically substantially pure trans-(1R,2R)-aminocyclohexyl ether compound of formula (69).

FIG. 35 illustrates a general reaction scheme that may be used as a process for preparing a stereoisomerically substantially pure trans-(1R,2R)-aminocyclohexyl ether compound of formula (66).

FIG. 36 illustrates a reaction scheme that may be used as a process for preparing a stereoisomerically substantially pure trans-(1R,2R)-aminocyclohexyl ether compound of formula (69).

FIG. 37 illustrates a reaction scheme that may be used as a process for preparing a stereoisomerically substantially pure compound of formula (55).

FIG. 38 illustrates a reaction scheme that may be used as a process for preparing a stereoisomerically substantially pure compound of formula (64).

FIG. 39 illustrates a reaction scheme that may be used as a process for preparing a stereoisomerically substantially pure compound of formula (67).

FIG. 40 illustrates a reaction scheme that may be used as a process for preparing a stereoisomerically substantially pure compound of formula (71).

FIG. 41 illustrates a reaction scheme that may be used as a process for preparing a stereoisomerically substantially pure compound of formula (53).

FIG. 42 illustrates a reaction scheme that may be used as a process for preparing a stereoisomerically substantially pure compound of formula (62).

FIG. 43 illustrates a reaction scheme that may be used as a process for preparing a stereoisomerically substantially pure compound of formula (52).

FIG. 44 illustrates a reaction scheme that may be used as a process for preparing a stereoisomerically substantially pure compound of formula (61).

FIG. 45 illustrates a general reaction scheme that may be used as a process for preparing a stereoisomerically substantially pure trans-(1S,2S)-aminocyclohexyl ether compound of formula (75).

FIG. 46 illustrates a reaction scheme that may be used as a process for preparing a stereoisomerically substantially pure trans-(1S,2S)-aminocyclohexylether compound of formula (79).

FIG. 47 illustrates a reaction scheme that may be used as a process for preparing a stereoisomerically substantially pure trans-(1S,2S)-aminocyclohexyl ether compound of formula (79).

FIG. 48 illustrates a reaction scheme that may be used as a process for preparing a stereoisomerically substantially pure trans-(1S,2S)-aminocyclohexyl ether compound of formula (81).

FIG. 49 illustrates a reaction scheme that may be used as a process for preparing a stereoisomerically substantially pure trans-(1S,2S)-aminocyclohexyl ether compound of formula (81).

FIG. 50 illustrates a general reaction scheme that may be used as a process for preparing a stereoisomerically substantially pure trans-(1S, 2S)-aminocyclohexyl ether compound of formula (75).

FIG. 51 illustrates a reaction scheme that may be used as a process for preparing a stereoisomerically substantially pure trans-(1S,2S)-aminocyclohexyl ether compound of formula (79).

FIG. 52 illustrates a reaction scheme that may be used as a process for preparing a stereoisomerically substantially pure trans-(1S,2S)-aminocyclohexyl ether compound of formula (79).

FIG. 53 illustrates a reaction scheme that may be used as a process for preparing a stereoisomerically substantially pure trans-(1S,2S)-aminocyclohexyl ether compound of formula (81).

FIG. 54 illustrates a reaction scheme that may be used as a process for preparing a stereoisomerically substantially pure trans-(1S,2S)-aminocyclohexyl ether compound of formula (81).

FIG. 55 illustrates a general reaction scheme that may be used as a process for preparing a stereoisomerically substantially pure trans-(1S,2S)-aminocyclohexyl ether compound of formula (75).

FIG. 56 illustrates a reaction scheme that may be used as a process for preparing a stereoisomerically substantially pure trans-(1S,2S)-aminocyclohexyl ether compound of formula (79).

FIG. 57 illustrates a reaction scheme that may be used as a process for preparing a stereoisomerically substantially pure trans-(1S,2S)-aminocyclohexyl ether compound of formula (79).

FIG. 58 illustrates a reaction scheme that may be used as a process for preparing a stereoisomerically substantially pure trans-(1S,2S)-aminocyclohexyl ether compound of formula (81).

FIG. 59 illustrates a reaction scheme that may be used as a process for preparing a stereoisomerically substantially pure trans-(1S,2S)-aminocyclohexyl ether compound of formula (81).

FIG. 60 illustrates a general reaction scheme that may be used as a process for preparing a stereoisomerically substantially pure trans-(1S,2S)-aminocyclohexyl ether compound of formula (75).

FIG. 61 illustrates a reaction scheme that may be used as a process for preparing a stereoisomerically substantially pure trans-(1S,2S)-aminocyclohexyl ether compound of formula (79).

FIG. 62 illustrates a reaction scheme that may be used as a process for preparing a stereoisomerically substantially pure trans-(1S,2S)-aminocyclohexyl ether compound of formula (79).

FIG. 63 illustrates a reaction scheme that may be used as a process for preparing a stereoisomerically substantially pure trans-(1S,2S)-aminocyclohexyl ether compound of formula (81).

FIG. 64 illustrates a reaction scheme that may be used as a process for preparing a stereoisomerically substantially pure trans-(1S, 2S)-aminocyclohexyl ether compound of formula (81).

FIG. 65 illustrates a general reaction scheme that may be used as a process for preparing a stereoisomerically substantially pure trans-(1S,2S)-aminocyclohexyl ether compound of formula (75).

FIG. 66 illustrates a reaction scheme that may be used as a process for preparing a stereoisomerically substantially pure trans-(1S,2S)-aminocyclohexyl ether compound of formula (79).

FIG. 67 illustrates a reaction scheme that may be used as a process for preparing a stereoisomerically substantially pure trans-(1S,2S)-aminocyclohexyl ether compound of formula (79).

FIG. 68 illustrates a reaction scheme that may be used as a process for preparing a stereoisomerically substantially pure trans-(1S, 2S)-aminocyclohexyl ether compound of formula (81).

FIG. 69 illustrates a reaction scheme that may be used as a process for preparing a stereoisomerically substantially pure trans-(1S, 2S)-aminocyclohexyl ether compound of formula (81).

FIG. 70 illustrates a general reaction scheme that may be used as a process for preparing a stereoisomerically substantially pure trans-(1S, 2S)-aminocyclohexyl ether compound of formula (75).

FIG. 71 illustrates a reaction scheme that may be used as a process for preparing a stereoisomerically substantially pure trans-(1S,2S)-aminocyclohexyl ether compound of formula (79).

FIG. 72 illustrates a reaction scheme that may be used as a process for preparing a stereoisomerically substantially pure trans-(1S,2S)-aminocyclohexyl ether compound of formula (79).

FIG. 73 illustrates a reaction scheme that may be used as a process for preparing a stereoisomerically substantially pure trans-(1S,2S)-aminocyclohexyl ether compound of formula (81).

FIG. 74 illustrates a reaction scheme that may be used as a process for preparing a stereoisomerically substantially pure trans-(1S,2S)-aminocyclohexyl ether compound of formula (81).

FIG. 75 illustrates a general reaction scheme that may be used as a process for preparing a stereoisomerically substantially pure trans-(1S,2S)-aminocyclohexyl ether compound of formula (79).

FIG. 76 illustrates a reaction scheme that may be used as a process for preparing a stereoisomerically substantially pure trans-(1S,2S)-aminocyclohexyl ether compound of formula (81).

FIG. 77 illustrates a reaction scheme that may be used as a process for preparing a stereoisomerically substantially pure compound of formula (74).

FIG. 78 illustrates a reaction scheme that may be used as a process for preparing a stereoisomerically substantially pure compound of formula (78).

FIG. 79 illustrates a reaction scheme that may be used as a process for preparing a stereoisomerically substantially pure compound of formula (80).

FIG. 80 illustrates a reaction scheme that may be used as a process for preparing a stereoisomerically substantially pure compound of formula (82).

FIG. 81 illustrates a reaction scheme that may be used as a process for preparing a stereoisomerically substantially pure compound of formula (73).

FIG. 82 illustrates a reaction scheme that may be used as a process for preparing a stereoisomerically substantially pure compound of formula (77).

FIG. 83 illustrates a reaction scheme that may be used as a process for preparing a stereoisomerically substantially pure compound of formula (72).

FIG. 84 illustrates a reaction scheme that may be used as a process for preparing a stereoisomerically substantially pure compound of formula (76).

FIG. 85 illustrates a general reaction scheme that may be used as a process for preparing a stereoisomerically substantially pure trans-(1R,2R)-aminocyclohexyl ether compound of formula (57).

FIG. 86 illustrates a reaction scheme that may be used as a process for preparing a stereoisomerically substantially pure trans-(1R,2R)-aminocyclohexylether compound of formula (66).

FIG. 87 illustrates a reaction scheme that may be used as a process for preparing a stereoisomerically substantially pure trans-(1R,2R)-aminocyclohexylether compound of formula (66).

FIG. 88 illustrates a reaction scheme that may be used as a process for preparing a stereoisomerically substantially pure trans-(1R,2R)-aminocyclohexylether compound of formula (66).

FIG. 89 illustrates a general reaction scheme that may be used as a process for preparing a stereoisomerically substantially pure trans-(1R,2R)-aminocyclohexyl ether compound of formula (57).

FIG. 90 illustrates a reaction scheme that may be used as a process for preparing a stereoisomerically substantially pure trans-(1R,2R)-aminocyclohexylether compound of formula (66).

FIG. 91 illustrates a reaction scheme that may be used as a process for preparing a stereoisomerically substantially pure trans-(1R,2R)-aminocyclohexylether compound of formula (66).

FIG. 92 illustrates a reaction scheme that may be used as a process for preparing a stereoisomerically substantially pure trans-(1R,2R)-aminocyclohexylether compound of formula (66).

FIG. 93 illustrates a general reaction scheme that may be used as a process for preparing a stereoisomerically substantially pure trans-(1R,2R)-aminocyclohexyl ether compound of formula (57).

FIG. 94 illustrates a reaction scheme that may be used as a process for preparing a stereoisomerically substantially pure trans-(1R,2R)-aminocyclohexylether compound of formula (66).

FIG. 95 illustrates a general reaction scheme that may be used as a process for preparing a stereoisomerically substantially pure compound of formula (55).

FIG. 96 illustrates general a reaction scheme that may be used as a process for preparing a stereoisomerically substantially pure compound of formula (55).

FIG. 97 illustrates a reaction scheme that may be used as a process for preparing a stereoisomerically substantially pure compound of formula (64).

FIG. 98 illustrates a reaction scheme that may be used as a process for preparing a stereoisomerically substantially pure compound of formula (64).

FIG. 99 illustrates a reaction scheme that may be used as a process for preparing a stereoisomerically substantially pure compound of formula (64).

FIG. 100 illustrates a general reaction scheme that may be used as a process for preparing a stereoisomerically substantially pure compound of formula (85) and a stereoisomerically substantially pure compound of formula (86).

FIG. 101 illustrates a reaction scheme that may be used as a process for preparing a stereoisomerically substantially pure compound of formula (62) and a stereoisomerically substantially pure compound of formula (89).

FIG. 102 illustrates a reaction scheme that may be used as a process for preparing a stereoisomerically substantially pure compound of formula (87) and a stereoisomerically substantially pure compound of formula (90).

FIG. 103 illustrates a reaction scheme that may be used as a process for preparing a stereoisomerically substantially pure compound of formula (62) and a stereoisomerically substantially pure compound of formula (87).

FIG. 104 illustrates a general reaction scheme that may be used as a process for preparing a stereoisomerically substantially pure trans-(1S,2S)-aminocyclohexyl ether compound of formula (75).

FIG. 105 illustrates a reaction scheme that may be used as a process for preparing a stereoisomerically substantially pure trans-(1S,2S)-aminocyclohexylether compound of formula (79).

FIG. 106 illustrates a reaction scheme that may be used as a process for preparing a stereoisomerically substantially pure trans-(1S,2S)-aminocyclohexylether compound of formula (79).

FIG. 107 illustrates a reaction scheme that may be used as a process for preparing a stereoisomerically substantially pure trans-(1S,2S)-aminocyclohexylether compound of formula (79).

FIG. 108 illustrates a general reaction scheme that may be used as a process for preparing a stereoisomerically substantially pure trans-(1S,2S)-aminocyclohexyl ether compound of formula (75).

FIG. 109 illustrates a reaction scheme that may be used as a process for preparing a stereoisomerically substantially pure trans-(1S,2S)-aminocyclohexylether compound of formula (79).

FIG. 110 illustrates a reaction scheme that may be used as a process for preparing a stereoisomerically substantially pure trans-(1S,2S)-aminocyclohexylether compound of formula (79).

FIG. 111 illustrates a reaction scheme that may be used as a process for preparing a stereoisomerically substantially pure trans-(1S,2S)-aminocyclohexylether compound of formula (79).

FIG. 112 illustrates a general reaction scheme that may be used as a process for preparing a stereoisomerically substantially pure trans-(1S,2S)-aminocyclohexyl ether compound of formula (75).

FIG. 113 illustrates a general reaction scheme that may be used as a process for preparing a stereoisomerically substantially pure trans-(1S, 2S)-aminocyclohexyl ether compound of formula (75).

FIG. 114 illustrates a reaction scheme that may be used as a process for preparing a stereoisomerically substantially pure trans-(1S,2S)-aminocyclohexylether compound of formula (79).

FIG. 115 illustrates a reaction scheme that may be used as a process for preparing a stereoisomerically substantially pure trans-(1S, 2S)-aminocyclohexylether compound of formula (79).

FIG. 116 illustrates a general reaction scheme that may be used as a process for preparing a stereoisomerically substantially pure compound of formula (74).

FIG. 117 illustrates general a reaction scheme that may be used as a process for preparing a stereoisomerically substantially pure compound of formula (74).

FIG. 118 illustrates a reaction scheme that may be used as a process for preparing a stereoisomerically substantially pure compound of formula (78).

FIG. 119 illustrates a reaction scheme that may be used as a process for preparing a stereoisomerically substantially pure compound of formula (78).

FIG. 120 illustrates a reaction scheme that may be used as a process for preparing a stereoisomerically substantially pure compound of formula (78)

FIG. 121 illustrates a general reaction scheme that may be used as a process for preparing a stereoisomerically substantially pure trans-(1R, 2R)-aminocyclohexyl ether compound of formula (57).

FIG. 122 illustrates a reaction scheme that may be used as a process for preparing a stereoisomerically substantially pure trans-(1R,2R)-aminocyclohexylether compound of formula (66).

FIG. 122A illustrates a reaction scheme that may be used as a process for preparing a stereoisomerically substantially pure trans-(1R,2R)-aminocyclohexylether compound of formula (66).

FIG. 123 illustrates a reaction scheme that may be used as a process for preparing a stereoisomerically substantially pure trans-(1R,2R)-aminocyclohexylether compound of formula (69).

FIG. 124 illustrates a general reaction scheme that may be used as a process for preparing a stereoisomerically substantially pure trans-(1R,2R)-aminocyclohexyl ether compound of formula (57).

FIG. 125 illustrates a reaction scheme that may be used as a process for preparing a stereoisomerically substantially pure trans-(1R,2R)-aminocyclohexylether compound of formula (66).

FIG. 126 illustrates a reaction scheme that may be used as a process for preparing a stereoisomerically substantially pure trans-(1R,2R)-aminocyclohexylether compound of formula (69).

FIG. 127 illustrates a general reaction scheme that may be used as a process for preparing a stereoisomerically substantially pure trans-(1R,2R)-aminocyclohexyl ether compound of formula (57).

FIG. 128 illustrates a reaction scheme that may be used as a process for preparing a stereoisomerically substantially pure trans-(1R,2R)-aminocyclohexylether compound of formula (66).

FIG. 129 illustrates a reaction scheme that may be used as a process for preparing a stereoisomerically substantially pure trans-(1R,2R)-aminocyclohexylether compound of formula (69).

FIG. 130 illustrates a general reaction scheme that may be used as a process for preparing a stereoisomerically substantially pure trans-(1R,2R)-aminocyclohexyl ether compound of formula (57).

FIG. 131 illustrates a reaction scheme that may be used as a process for preparing a stereoisomerically substantially pure trans-(1R,2R)-aminocyclohexylether compound of formula (66).

FIG. 133 illustrates a general reaction scheme that may be used as a process for preparing a stereoisomerically substantially pure trans-(1R,2R)-aminocyclohexyl ether compound of formula (57).

FIG. 134 illustrates a reaction scheme that may be used as a process for preparing a stereoisomerically substantially pure trans-(1R,2R)-aminocyclohexylether compound of formula (66).

FIG. 135 illustrates a reaction scheme that may be used as a process for preparing a stereoisomerically substantially pure trans-(1R,2R)-aminocyclohexylether compound of formula (69).

FIG. 136 illustrates a general reaction scheme that may be used as a process for preparing a stereoisomerically substantially pure trans-(1R,2R)-aminocyclohexyl ether compound of formula (57).

FIG. 137 illustrates a reaction scheme that may be used as a process for preparing a stereoisomerically substantially pure trans-(1R,2R)-aminocyclohexylether compound of formula (66).

FIG. 138 illustrates a reaction scheme that may be used as a process for preparing a stereoisomerically substantially pure trans-(1R,2R)-aminocyclohexylether compound of formula (69).

FIG. 139 illustrates a reaction scheme that may be used as a process for preparing a stereoisomerically substantially pure compound of formula (55).

FIG. 140 illustrates a reaction scheme that may be used as a process for preparing a stereoisomerically substantially pure compound of formula (64).

FIG. 141 illustrates a reaction scheme that may be used as a process for preparing a stereoisomerically substantially pure compound of formula (94).

FIG. 142 illustrates a reaction scheme that may be used as a process for preparing a stereoisomerically substantially pure compound of formula (98).

FIG. 143 illustrates a reaction scheme that may be used as a process for preparing a stereoisomerically substantially pure compound of formula (93).

FIG. 144 illustrates a reaction scheme that may be used as a process for preparing a stereoisomerically substantially pure compound of formula (97).

FIG. 145 illustrates a reaction scheme that may be used as a process for preparing a stereoisomerically substantially pure compound of formula (92).

FIG. 146 illustrates a reaction scheme that may be used as a process for preparing a stereoisomerically substantially pure compound of formula (96).

FIG. 147 illustrates a general reaction scheme that may be used as a process for preparing a stereoisomerically substantially pure trans-(1S,2S)-aminocyclohexyl ether compound of formula (75).

FIG. 148 illustrates a reaction scheme that may be used as a process for preparing a stereoisomerically substantially pure trans-(1S,2S)-aminocyclohexylether compound of formula (79).

FIG. 149 illustrates a reaction scheme that may be used as a process for preparing a stereoisomerically substantially pure trans-(1S,2S)-aminocyclohexylether compound of formula (81).

FIG. 150 illustrates a general reaction scheme that may be used as a process for preparing a stereoisomerically substantially pure trans-(1S,2S)-aminocyclohexyl ether compound of formula (75).

FIG. 151 illustrates a reaction scheme that may be used as a process for preparing a stereoisomerically substantially pure trans-(1S,2S)-aminocyclohexylether compound of formula (79).

FIG. 152 illustrates a reaction scheme that may be used as a process for preparing a stereoisomerically substantially pure trans-(1S,2S)-aminocyclohexylether compound of formula (81).

FIG. 153 illustrates a general reaction scheme that may be used as a process for preparing a stereoisomerically substantially pure trans-(1S,2S)-aminocyclohexyl ether compound of formula (75).

FIG. 154 illustrates a reaction scheme that may be used as a process for preparing a stereoisomerically substantially pure trans-(1S,2S)-aminocyclohexylether compound of formula (79).

FIG. 155 illustrates a reaction scheme that may be used as a process for preparing a stereoisomerically substantially pure trans-(1S,2S)-aminocyclohexylether compound of formula (81).

FIG. 156 illustrates a general reaction scheme that may be used as a process for preparing a stereoisomerically substantially pure trans-(1S,2S)-aminocyclohexyl ether compound of formula (75).

FIG. 157 illustrates a reaction scheme that may be used as a process for preparing a stereoisomerically substantially pure trans-(1S,2S)-aminocyclohexylether compound of formula (79).

FIG. 158 illustrates a reaction scheme that may be used as a process for preparing a stereoisomerically substantially pure trans-(1S,2S)-aminocyclohexylether compound of formula (81).

FIG. 159 illustrates a general reaction scheme that may be used as a process for preparing a stereoisomerically substantially pure trans-(1S, 2S)-aminocyclohexyl ether compound of formula (75).

FIG. 160 illustrates a reaction scheme that may be used as a process for preparing a stereoisomerically substantially pure trans-(1S,2S)-aminocyclohexylether compound of formula (79).

FIG. 161 illustrates a reaction scheme that may be used as a process for preparing a stereoisomerically substantially pure trans-(1S,2S)-aminocyclohexylether compound of formula (81).

FIG. 162 illustrates a reaction scheme that may be used as a process for preparing a stereoisomerically substantially pure compound of formula (74).

FIG. 163 illustrates a reaction scheme that may be used as a process for preparing a stereoisomerically substantially pure compound of formula (78).

FIG. 164 illustrates a reaction scheme that may be used as a process for preparing a stereoisomerically substantially pure compound of formula (84).

FIG. 165 illustrates a reaction scheme that may be used as a process for preparing a stereoisomerically substantially pure compound of formula (62).

FIG. 166 illustrates a reaction scheme that may be used as a process for preparing a stereoisomerically substantially pure compound of formula (99).

FIG. 167 illustrates a reaction scheme that may be used as a process for preparing a stereoisomerically substantially pure compound of formula (100).

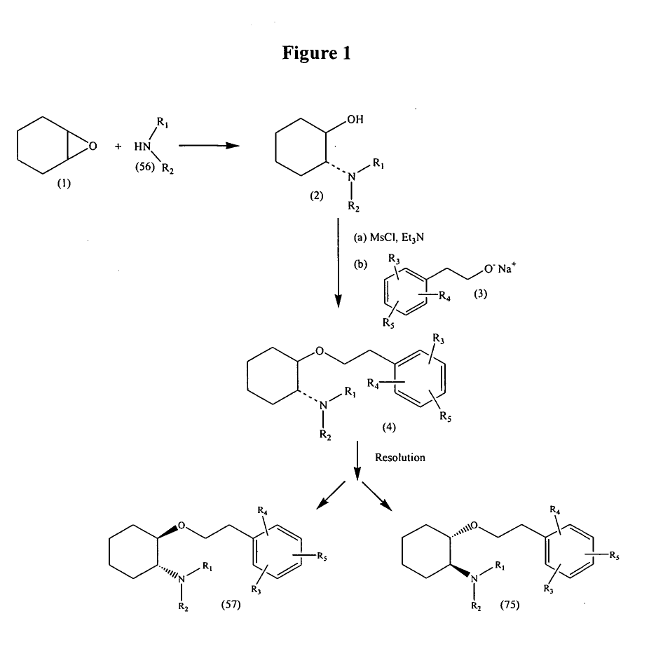

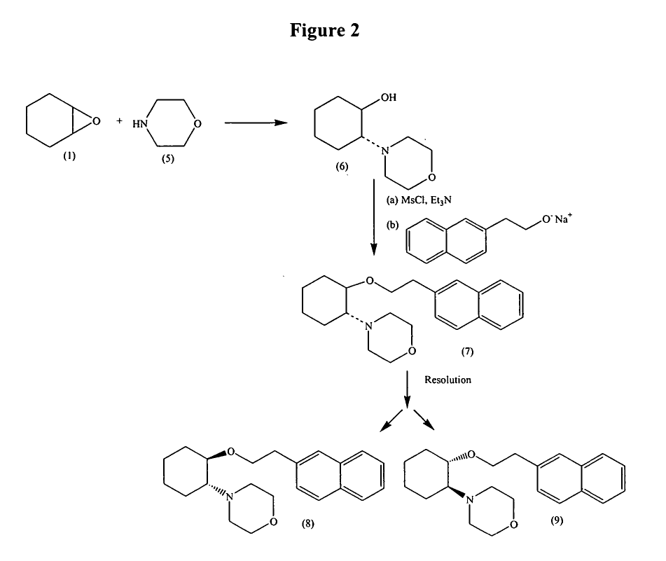

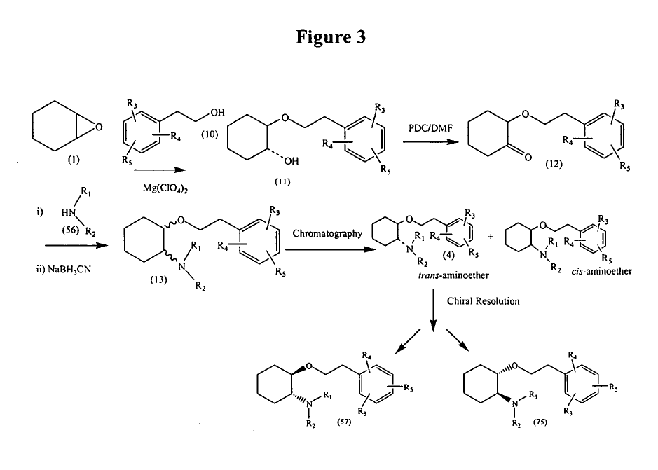

### DETAILED DESCRIPTION OF THE INVENTION

As noted above, the present invention is directed to aminocyclohexyl ether compounds of formula such as (IA), (IB), (IC), (ID), or (IE), methods of manufacture thereof, pharmaceutical compositions containing the aminocyclohexyl ether compounds, and various uses for the compounds and compositions. Such uses include the treatment of arrhythmias, ion channel modulation and other uses as described herein.

An understanding of the present invention may be aided by reference to the following definitions and explanation of conventions used herein:

The aminocyclohexyl ether compounds of the invention have an ether oxygen atom at position 1 of a cyclohexane ring, and an amine nitrogen atom at position 2 of the cyclohexane ring, with other positions numbered, in corresponding order as shown below in structure (A¹):

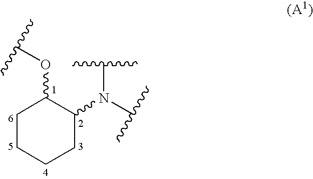

The bonds from the cyclohexane ring to the 1-oxygen and 2-nitrogen atoms in the above formula may be relatively disposed in either a cis or trans relationship. Therefore, the stereochemistry of the amine and ether substituents of the cyclohexane ring is either (R,R)-trans or (S,S)-trans for the transtereoisomers and is either (R,S)-cis or (S,R)-cis for the cis-stereoisomers.

A wavy bond from a substituent to the central cyclohexane ring indicates that that group may be located on either side of the plane of the central ring. When a wavy bond is shown intersecting a ring, this indicates that the indicated substituent group may be attached to any position on the ring capable of bonding to the substituent group and that the substituent group may lie above or below the plane of the ring system to which it is bound.

Following the standard chemical literature description practice and as used in this patent, a full wedge bond means above the ring plane, and a dashed wedge bond means below the ring plane; one full bond and one dashed bond (i.e., -----) means a trans configuration, whereas two full bonds or two dashed bonds means a cis configuration.

In the formulae depicted herein, a bond to a substituent and/or a bond that links a molecular fragment to the remainder of a compound may be shown as intersecting one or more bonds in a ring structure. This indicates that the bond may be attached to any one of the atoms that constitutes the ring structure, so long as a hydrogen atom could otherwise be present at that atom. Where no particular substituent(s) is identified for a particular position in a structure, then hydrogen(s) is present at that position. For example, compounds of the invention containing compounds having the group (B¹): 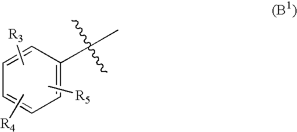
where the group (B¹) is intended to encompass groups wherein any ring atom that could otherwise be substituted with hydrogen, may instead be substituted with either R₃, R₄ or R₅, with the proviso that each of R₃, R₄ and R₅ appears once and only once on the ring. Ring atoms that are not substituted with any of R₃, R₄ or R₅ are substituted with hydrogen. In those instances where the invention specifies that a non-aromatic ring is substituted with one or more functional groups, and those functional groups are shown connected to the non-aromatic ring with bonds that bisect ring bonds, then the functional groups may be present at different atoms of the ring, or on the same atom of the ring, so long as that atom could otherwise be substituted with a hydrogen atom.

The compounds of the present invention contain at least two asymmetric carbon atoms and thus exist as enantiomers and diastereomers. Unless otherwise indicated, the present invention includes all enantiomeric and diastereomeric forms of the aminocyclohexyl ether compounds of the invention. Pure stereoisomers, mixtures of enantiomers and/or diastereomers, and mixtures of different compounds of the invention are included within the present invention. Thus, compounds of the present invention may occur as racemates, racemic mixtures and as individual diastereomers, or enantiomers, unless a specific stereoisomer enantiomer or diastereomer is identified, with all isomeric forms being included in the present invention. A racemate or racemic mixture does not imply a 50:50 mixture of stereoisomers. Unless otherwise noted, the phrase “stereoisomerically substantially pure” generally refers to those asymmetric carbon atoms that are described or illustrated in the structural formulae for that compound.

The definition of stereoisomeric purity (or optical purity or chiral purity) and related terminology and their methods of determination (e.g., Optical rotation, circular dichroism etc.) are well known in the art (see e.g., E. L. Eliel and S. H. Wilen, in Stereochemistry of Organic Compounds; John Wiley & Sons: New York, 1994; and references cited therein). The phrase “stereoisomerically substantially pure” generally refers to the enrichment of one of the stereoisomers (e.g., enantiomers or diastereomers) over the other stereoisomers in a sample, leading to chiral enrichment and increase in optical rotation activity of the sample. Enantiomer is one of a pair of molecular species that are mirror images of each other and not superposable. They are ‘mirror-image’ stereoisomers. Diastereomers generally refer to stereoisomers not related as mirror-images. Enantiomer excess (ee) and diastereomer excess (de) are terms generally used to refer the stereoisomeric purity (or optical purity or chiral purity) of a sample of the compound of interest. Their definition and methods of determination are well known in the art and can be found e.g., in E. L. Eliel and S. H. Wilen, in Stereochemistry of Organic Compounds; John Wiley & Sons: New York, 1994; and references cited therein. “Stereoselectively making” refers to making the compound having enantiomer excess (ee) or diastereomer excel (de).

For the present invention, enantiomer excess (ee) or diastereomer excess (de) in the range of about 50% to about 100% is contemplated. A preferred range of enantiomer excess (ee) or diastereomer excess (de) is about 60% to about 100%. Another preferred range of enantiomer excess (ee) or diastereomer excess (de) is about 70% to about 100%. A more preferred range of enantiomer excess (ee) or diastereomer excess (de) is about 80% to about 100%. Another more preferred range of enantiomer excess (ee) or diastereomer excess (de) is about 85% to about 100%. An even more preferred range of enantiomer excess (ee) or diastereomer excess (de) is about 90% to about 100%. Another even more preferred range of enantiomer excess (ee) or diastereomer excess (de) is about 95% to about 100%. It is understood that the phrase “about 50% to about 100%” includes but is not limited to all the possible percentage numbers and fractions of a number from 50% to 100%. Similarly, the phrase “about 60% to about 100%” includes but is not limited to all the possible percentage numbers and fractions of a number from 60% to 100%; the phrase “about 70% to about 100%” includes but is not limited to all the possible percentage numbers and fractions of a number from 70% to 100%; the phrase “about 80% to about 100%” includes but is not limited to all the possible percentage numbers and fractions of a number from 80% to 100%; the phrase “about 85% to about 100%” includes all but is not limited to the possible percentage numbers and fractions of a number from 85% to 100%; the phrase “about 90% to about 100%” includes but is not limited to all the possible percentage numbers and fractions of a number from 90% to 100%; the phrase “about 95% to about 100%” includes all but is not limited to the possible percentage numbers and fractions of a number from 95% to 100%.

As an example, and in no way limiting the generality of the above, a compound designated with the formula 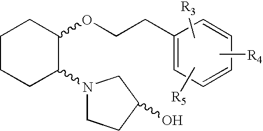
includes at least three chiral centers (the cyclohexyl carbon bonded to the oxygen, the cyclohexyl carbon bonded to the nitrogen, and the pyrrolidinyl carbon bonded to the oxygen) and therethore has at least eight separate stereoisomers, which are (1R,2R)-2-[(3R)-Hydroxypyrrolidinyl]-1-(R₃, R₄ and R₅ substituted phenethoxy)-cyclohexane; (1R,2R)-2-[(3S)-Hydroxypyrrolidinyl]-1-(R₃, R₄ and R₅ substituted phenethoxy)-cyclohexane; (1S,2S)-2-[(3R)-Hydroxypyrrolidinyl]-1-(R₃, R₄ and R₅ substituted phenethoxy)-cyclohexane; (1S,2S)-2-[(3S)-Hydroxypyrrolidinyl]-1-(R₃, R₄ and R₅ substituted phenethoxy)-cyclohexane; (1R,2S)-2-[(3R)-Hydroxypyrrolidinyl]-1-(R₃, R₄ and R₅ substituted phenethoxy)-cyclohexane; (1R,2S)-2-[(3S)-Hydroxypyrrolidinyl]-1-(R₃, R₄ and R₅ substituted phenethoxy)-cyclohexane; (1S,2R)-2-[(3R)-Hydroxypyrrolidinyl]-1-(R₃, R₄ and R₅ substituted phenethoxy)-cyclohexane; and (1S,2R)-2-[(3S)-Hydroxypyrrolidinyl]-1-(R₃, R₄ and R₅ substituted phenethoxy)-cyclohexane; and, unless the context make plain otherwise as used in this patent a compound of the formula 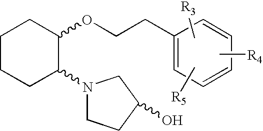
   means a composition that includes a component that is either one of the eight pure enantiomeric forms of the indicated compound or is a mixture of any two or more of the pure enantiomeric forms, where the mixture can include any number of the enantiomeric forms in any ratio.

As an example, and in no way limiting the generality of the above, unless the context make plain otherwise as used in this patent a compound designated with the chemical formula (1R,2R)/(1S,2S)-2-[(3R)-Hydroxypyrrolidinyl]-1-(3,4-dimethoxyphenethoxy)-cyclohexane means a composition that includes a component that is either one of the two pure enantiomeric forms of the indicated compound (i.e., (1R,2R)-2-[(3R)-Hydroxypyrrolidinyl]-1-(3,4-dimethoxyphenethoxy)-cyclohexane or (1S,2S)-2-[(3R)-Hydroxypyrrolidinyl]-1-(3,4-dimethoxyphenethoxy)-cyclohexane) or is a racemic mixture of the two pure enantiomeric forms, where the racemic mixture can include any relative amount of the two enantiomers.

The phrase “independently at each occurrence” is intended to mean (i) when any variable occurs more than one time in a compound of the invention, the definition of that variable at each occurrence is independent of its definition at every other occurrence; and (ii) the identity of any one of two different variables (e.g., R₁ within the set R₁ and R₂) is selected without regard the identity of the other member of the set. However, combinations of substituents and/or variables are permissible only if such combinations result in compounds that do not violate the standard rules of chemical valency.

In accordance with the present invention and as used herein, the following terms are defined to have following meanings, unless explicitly stated otherwise:

“Acid addition salts” refers to those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like, and include but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002.

“Acyl” refers to branched or unbranched hydrocarbon fragments terminated by a carbonyl —(C═O)— group containing the specified number of carbon atoms. Examples include acetyl (Ac) [CH₃C(═O)—, a C₂acyl] and propionyl [CH₃CH₂C(═O)—, a C₃acyl].

“Alkanoyloxy” refers to an ester substituent wherein the non-carbonyl oxygen is the point of attachment to the molecule. Examples include propanoyloxy [(CH₃CH₂C(═O)—O—, a C₃alkanoyloxy] and ethanoyloxy [CH₃C(═O)—O—, a C₂alkanoyloxy].

“Alkoxy” refers to an oxygen (O)-atom substituted by an alkyl group, for example, alkoxy can include but is not limited to methoxy, which may also be denoted as —OCH₃, —OMe or a C₁alkoxy.

“Alkoxyalkyl” refers to a alkylene group substituted with an alkoxy group. For example, methoxyethyl [CH₃OCH₂CH₂—] and ethoxymethyl (CH₃CH₂OCH₂—] are both C₃alkoxyalkyl groups.

“Alkoxycarbonyl” refers to an ester substituent wherein the carbonyl carbon is the point of attachment to the molecule. Examples include ethoxycarbonyl [CH₃CH₂OC(═O)—, a C₃alkoxycarbonyl] and methoxycarbonyl [CH₃OC(═O)—, a C₂alkoxycarbonyl].

“Alkyl” refers to a branched or unbranched hydrocarbon fragment containing the specified number of carbon atoms and having one point of attachment. Examples include n-propyl (a C₃alkyl), iso-propyl (also a C₃alkyl), and t-butyl (a C₄alkyl). Methyl is represented by the symbol Me or CH₃.

“Alkylene” refers to a divalent radical which is a branched or unbranched hydrocarbon fragment containing the specified number of carbon atoms, and having two points of attachment. An example is propylene [—CH₂CH₂CH₂—, a C₃alkylene].

“Alkylcarboxy” refers to a branched or unbranched hydrocarbon fragment terminated by a carboxylic acid group [—COOH]. Examples include carboxymethyl [HOOC—CH₂—, a C₂alkylcarboxy] and carboxyethyl [HOOC—CH₂CH₂—, a C₃alkylcarboxy].

“Aryl” refers to aromatic groups which have at least one ring having a conjugated pi electron system and includes carbocyclic aryl, heterocyclic aryl (also known as heteroaryl groups) and biaryl groups, all of which may be optionally substituted. Carbocyclic aryl groups are generally preferred in the compounds of the present invention, where phenyl and naphthyl groups are preferred carbocyclic aryl groups.

“Aralkyl” refers to an alkylene group wherein one of the points of attachment is to an aryl group. An example of an aralkyl group is the benzyl group (Bn) [C₆H₅CH₂—, a C₇aralkyl group].

“Cycloalkyl” refers to a ring, which may be saturated or unsaturated and monocyclic, bicyclic, or tricyclic formed entirely from carbon atoms. An example of a cycloalkyl group is the cyclopentenyl group (C₅H₇—), which is a five carbon (C₅) unsaturated cycloalkyl group.

“Carbocyclic” refers to a ring which may be either an aryl ring or a cycloalkyl ring, both as defined above.

“Carbocyclic aryl” refers to aromatic groups wherein the atoms which form the aromatic ring are carbon atoms. Carbocyclic aryl groups include monocyclic carbocyclic aryl groups such as phenyl, and bicyclic carbocyclic aryl groups such as naphthyl, all of which may be optionally substituted.

“Heteroatom” refers to a non-carbon atom, where boron, nitrogen, oxygen, sulfur and phosphorus are preferred heteroatoms, with nitrogen, oxygen and sulfur being particularly preferred heteroatoms in the compounds of the present invention.

“Heteroaryl” refers to aryl groups having from 1 to 9 carbon atoms and the remainder of the atoms are heteroatoms, and includes those heterocyclic systems described in “Handbook of Chemistry and Physics,” 49th edition, 1968, R. C. Weast, editor; The Chemical Rubber Co., Cleveland, Ohio. See particularly Section C, Rules for Naming Organic Compounds, B. Fundamental Heterocyclic Systems. Suitable heteroaryls include furanyl, thienyl, pyridyl, pyrrolyl, pyrimidyl, pyrazinyl, imidazolyl, and the like.

“Hydroxyalkyl” refers to a branched or unbranched hydrocarbon fragment bearing an hydroxy (—OH) group. Examples include hydroxymethyl (—CH₂OH, a C₁hydroxyalkyl) and 1-hydroxyethyl (—CHOHCH₃, a C₂hydroxyalkyl).

“Thioalkyl” refers to a sulfur atom substituted by an alkyl group, for example thiomethyl (CH₃S—, a C₁thioalkyl).

“Modulating” in connection with the activity of an ion channel means that the activity of the ion channel may be either increased or decreased in response to administration of a compound or composition or method of the present invention. Thus, the ion channel may be activated, so as to transport more ions, or may be blocked, so that fewer or no ions are transported by the channel.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). Examples of pharmaceutically acceptable salt include but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002. The compounds of the present invention may be used in either the free base or salt forms, with both forms being considered as being within the scope of the present invention.

The “therapeutically effective amount” of a compound of the present invention will depend on the route of administration, the type of warm-blooded animal being treated, and the physical characteristics of the specific warm-blooded animal under consideration. These factors and their relationship to determining this amount are well known to skilled practitioners in the medical arts. This amount and the method of administration can be tailored to achieve optimal efficacy but will depend on such factors as weight, diet, concurrent medication and other factors which those skilled in the medical arts will recognize.

Compositions described herein as “containing a compound of the present invention” encompass compositions that may contain more than one compound of the present invention formula.

The synthetic procedures described herein, especially when taken with the general knowledge in the art, provide sufficient guidance to perform the synthesis, isolation, and purification of the compounds of the present invention.

The following examples are offered by way of illustration and not by way of limitation. Unless otherwise specified, starting materials and reagents may be obtained from well-known commercial supply houses, e.g., Sigma-Aldrich Fine Chemicals (St. Louis, Mo.), and are of standard grade and purity; or may be obtained by procedures described in the art or adapted therefrom, where suitable procedures may be identified through the Chemical Abstracts and Indices therefor, as developed and published by the American Chemical Society.

Compounds that May be Prepared by the Method of the Present Invention

In one embodiment, the present invention provides a compound of formula (57), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof prepared by the method of the present invention: 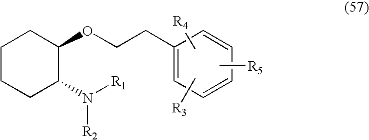
wherein, independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or
R₁ and R₂, when taken together with the nitrogen atom to which they are directly attached in formula (57), form a ring denoted by formula (I): 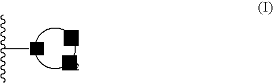
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; and any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (57), form a ring denoted by formula (II): 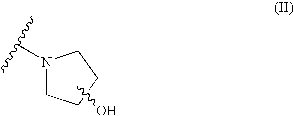
or R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl; or
R₃, R₄ and R₅ are independently hydrogen, hydroxy or C₁-C₆alkoxy; with the proviso that R₃, R₄ and R₅ cannot all be hydrogen.

In one embodiment, the present invention provides a compound of formula (75), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof prepared by the method of the present invention: 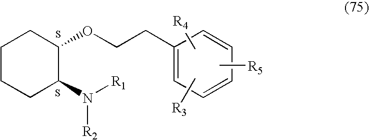
wherein, independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or
R₁ and R₂, when taken together with the nitrogen atom to which they are directly attached in formula (75), form a ring denoted by formula (I): 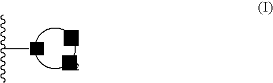
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; and any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (75), form a ring denoted by formula (II): 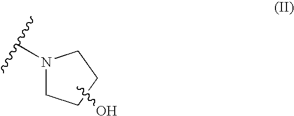
or R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl; or
R₃, R₄ and R₅ are independently hydrogen, hydroxy or C₁-C₆alkoxy; with the proviso that R₃, R₄ and R₅ cannot all be hydrogen.

In one embodiment, the present invention provides a compound of formula (14A), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof prepared by the method of the present invention: 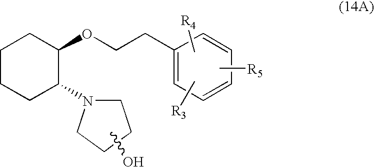
wherein R₃, R₄ and R₅ are independently hydrogen, hydroxy or C₁-C₆alkoxy, including isolated enantiomeric, diastereomeric and geometric isomers thereof, and mixtures thereof, with the proviso that R₃, R₄ and R₅ cannot all be hydrogen.

In one embodiment, the present invention provides a compound of formula (14A), or a solvate, pharmaceutically acceptable salt thereof, including isolated enantiomeric, diastereomeric and geometric isomers thereof, and mixtures, thereof prepared by the method of the present invention.

In one embodiment, the present invention provides a compound of formula (14A), or a solvate, pharmaceutically acceptable salt thereof, prepared by the method of the present invention wherein R₄ and R₅ are independently hydroxy or C₁-C₆alkoxy, including isolated enantiomeric, diastereomeric and geometric isomers thereof, and mixtures thereof.

In one embodiment, the present invention provides a compound of formula (14A), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently hydroxy or C₁-C₆alkoxy.

In one embodiment, the present invention provides a compound of formula (14A), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14A), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14A), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are C₁alkoxy.

In one embodiment, the present invention provides a compound of formula (14A), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are C₁alkoxy.

In another embodiment, the present invention provides a compound of formula (14B), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, prepared by the method of the present invention: 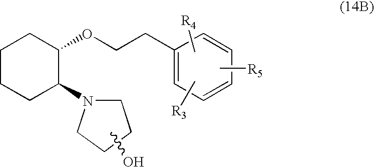
wherein R₃, R₄ and R₅ are independently hydrogen, hydroxy or C₁-C₆alkoxy, including isolated enantiomeric, diastereomeric and geometric isomers thereof, and mixtures thereof.

In one embodiment, the present invention provides a compound of formula (14B), or a solvate, pharmaceutically acceptable salt thereof, including isolated enantiomeric, diastereomeric and geometric isomers thereof, and mixtures thereof, prepared by the method of the present invention.

In one embodiment, the present invention provides a compound of formula (14B), or a solvate, pharmaceutically acceptable salt thereof, wherein R₄ and R₅ are independently hydroxy or C₁-C₆alkoxy, including isolated enantiomeric, diastereomeric and geometric isomers thereof, and mixtures thereof, prepared by the method of the present invention.

In one embodiment, the present invention provides a compound of formula (14B), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently hydroxy or C₁-C₆alkoxy.

In one embodiment, the present invention provides a compound of formula (14B), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14B), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14B), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are C₁alkoxy.

In one embodiment, the present invention provides a compound of formula (14B), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are C₁alkoxy.

In another embodiment, the present invention provides a compound of formula (IC), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, prepared by the method of the present invention: 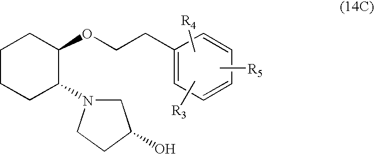
wherein R₃, R₄ and R₅ are independently hydrogen, hydroxy or C₁-C₆alkoxy, including isolated enantiomeric, diastereomeric and geometric isomers thereof, and mixtures thereof.

In one embodiment, the present invention provides a compound of formula (14C), or a solvate, pharmaceutically acceptable salt thereof, including isolated enantiomeric, diastereomeric and geometric isomers thereof, and mixtures thereof, prepared by the method of the present invention.

In one embodiment, the present invention provides a compound of formula (14C), or a solvate, pharmaceutically acceptable salt thereof, prepared by the method of the present invention wherein R₄ and R₅ are independently selected from hydroxy and C₁-C₆alkoxy, including isolated enantiomeric, diastereomeric and geometric isomers thereof, and mixtures thereof

In one embodiment, the present invention provides a compound of formula (14C), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅are independently hydroxy or C₁-C₆alkoxy.

In one embodiment, the present invention provides a compound of formula (14C), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14C), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14C), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are C₁alkoxy.

In one embodiment, the present invention provides a compound of formula (14C), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are C₁alkoxy.

In another embodiment, the present invention provides a compound of formula (14D), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, prepared by the method of the present invention: 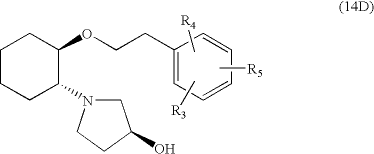
wherein R₃, R₄ and R₅ are independently hydrogen, hydroxy or C₁-C₆alkoxy, including isolated enantiomeric, diastereomeric and geometric isomers thereof, and mixtures thereof.

In one embodiment, the present invention provides a compound of formula (14D), or a solvate, pharmaceutically acceptable salt thereof, including isolated enantiomeric, diastereomeric and geometric isomers thereof, and mixtures thereof, prepared by the method of the present invention.

In one embodiment, the present invention provides a compound of formula (14D), or a solvate, pharmaceutically acceptable salt thereof, prepared by the method of the present invention wherein R₄ and R₅ are independently selected from hydroxy and C₁-C₆alkoxy, including isolated enantiomeric, diastereomeric and geometric isomers thereof, and mixtures thereof.

In one embodiment, the present invention provides a compound of formula (14D), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently hydroxy or C₁-C₆alkoxy.

In one embodiment, the present invention provides a compound of formula (14D), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14D), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are independently C₁-C₆alkoxy.

In one embodiment, the present invention provides a compound of formula (14D), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₃ is hydrogen, and R₄ and R₅ are C₁alkoxy.

In one embodiment, the present invention provides a compound of formula (14D), or a solvate, pharmaceutically acceptable salt thereof, including isolated enantiomeric, diastereomeric and geometric isomers thereof, and mixtures thereof, prepared by the method of the present invention wherein R₃ is hydrogen, and R₄ and R₅ are C₁alkoxy.

In another embodiment, the present invention provides a compound of formula (14E), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, prepared by the method of the present invention: 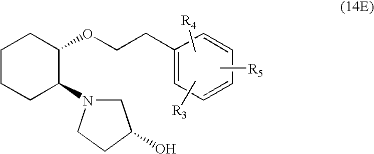
wherein R₄ and R₅ are independently hydrogen, hydroxy or C₁-C₆alkoxy, including isolated enantiomeric, diastereomeric and geometric isomers thereof, and mixtures thereof.

In one embodiment, the present invention provides a compound of formula (14E), or a solvate, pharmaceutically acceptable salt thereof, including isolated enantiomeric, diastereomeric and geometric isomers thereof, and mixtures thereof, prepared by the method of the present invention.

In one embodiment, the present invention provides a compound of formula (14E), or a solvate, pharmaceutically acceptable salt thereof, prepared by the method of the present invention wherein R₄ and R₅ are independently hydroxy or C₁-C₆alkoxy, including isolated enantiomeric, diastereomeric and geometric isomers thereof, and mixtures thereof.

In one embodiment, the present invention provides a compound of formula (14E), or a solvate, pharmaceutically acceptable salt thereof, including isolated enantiomeric, diastereomeric and geometric isomers thereof, and mixtures thereof, prepared by the method of the present invention wherein R₄ and R₅ are independently hydroxy or C₁-C₃alkoxy.

In one embodiment, the present invention provides a compound of formula (14E), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₄ and R₅ are independently C₁-C₆alkoxy.

In one embodiment, the present invention provides a compound of formula (14E), or a solvate, pharmaceutically acceptable salt thereof, including isolated enantiomeric, diastereomeric and geometric isomers thereof, and mixtures thereof, prepared by the method of the present invention wherein R₄ and R₅ are independently C₁-C₃alkoxy.

In one embodiment, the present invention provides a compound of formula (14E), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₄ and R₅ are C₁alkoxy.

In one embodiment, the present invention provides a compound of formula (14E), or a solvate, pharmaceutically acceptable salt thereof, including isolated enantiomeric, diastereomeric and geometric isomers thereof, and mixtures thereof, wherein R₄ and R₅ are C₁alkoxy.

In another embodiment, the present invention provides a compound of formula (14F), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, prepared by the method of the present invention: 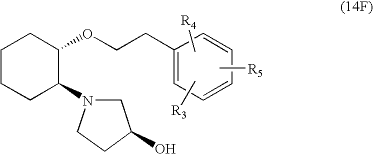
wherein R₄ and R₅ are independently selected from hydrogen, hydroxy and C₁-C₆alkoxy, including isolated enantiomeric, diastereomeric and geometric isomers thereof, and mixtures thereof.

In one embodiment, the present invention provides a compound of formula (14F), or a solvate, pharmaceutically acceptable salt thereof, including isolated enantiomeric, diastereomeric and geometric isomers thereof, and mixtures thereof, prepared by the method of the present invention.

In one embodiment, the present invention provides a compound of formula (14F), or a solvate, pharmaceutically acceptable salt thereof, prepared by the method of the present invention wherein R₄ and R₅ are independently hydroxy or C₁-C₆alkoxy, including isolated enantiomeric, diastereomeric and geometric isomers thereof, and mixtures thereof.

In one embodiment, the present invention provides a compound of formula (14F), or a solvate, pharmaceutically acceptable salt thereof, including isolated enantiomeric, diastereomeric and geometric isomers thereof, and mixtures thereof, prepared by the method of the present invention wherein R₄ and R₅ are independently hydroxy or C₁-C₃alkoxy.

In one embodiment, the present invention provides a compound of formula (14F), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₄ and R₅ are independently C₁-C₆alkoxy.

In one embodiment, the present invention provides a compound of formula (14F), or a solvate, pharmaceutically acceptable salt thereof, including isolated enantiomeric, diastereomeric and geometric isomers thereof, and mixtures thereof, prepared by the method of the present invention wherein R₄ and R₅ are independently C₁-C₃alkoxy.

In one embodiment, the present invention provides a compound of formula (14F), or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, prepared by the method of the present invention wherein R₄ and R₅ are C₁alkoxy.

In one embodiment, the present invention provides a compound of formula (14F), or a solvate, pharmaceutically acceptable salt thereof, including isolated enantiomeric, diastereomeric and geometric isomers thereof, and mixtures thereof, prepared by the method of the present invention wherein R₄ and R₅ are C₁alkoxy.

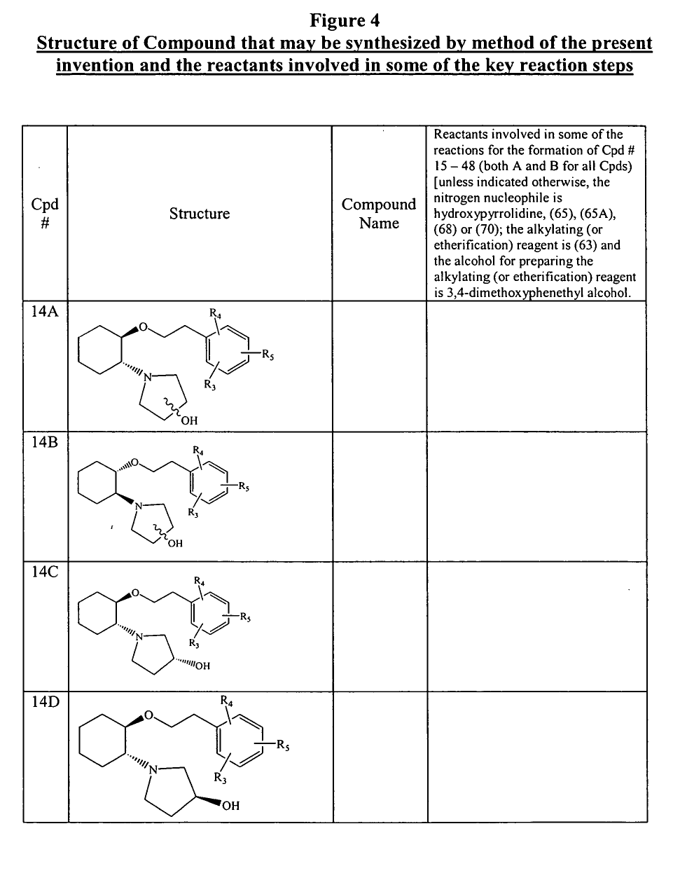

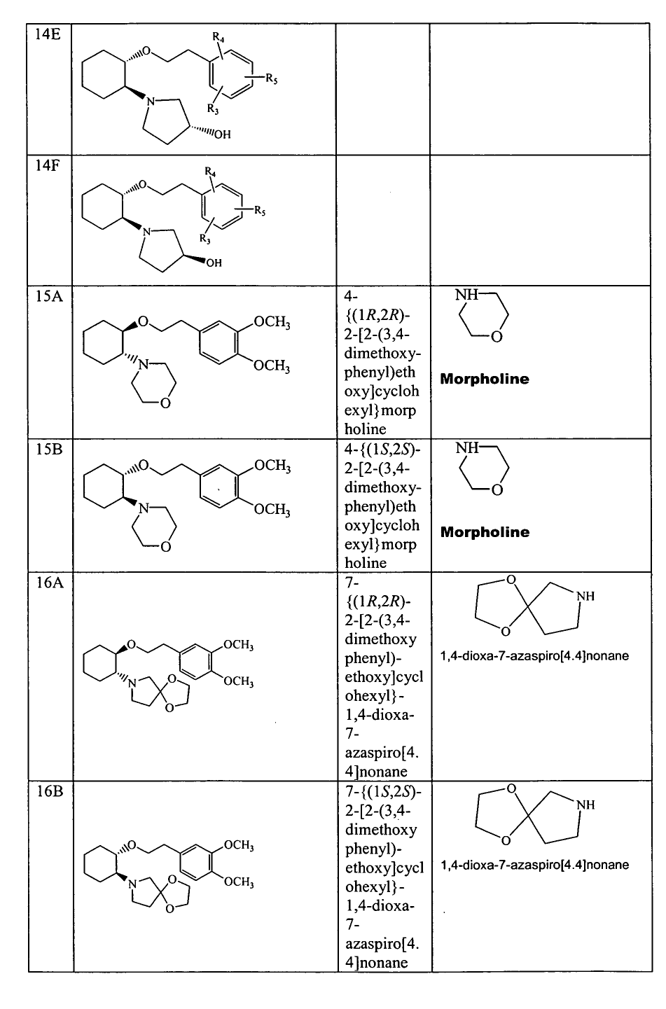

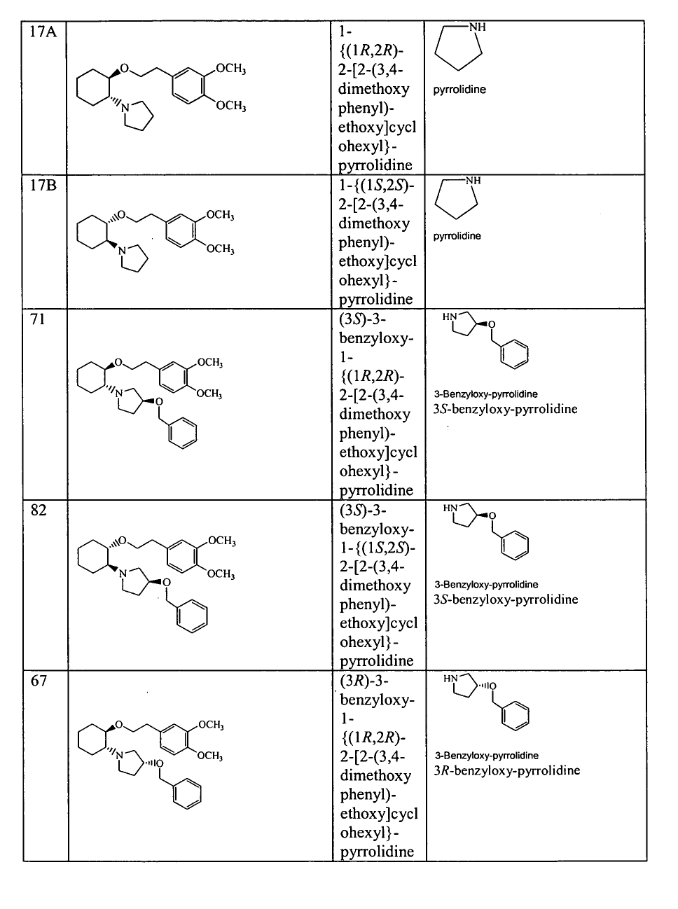

Other compounds that may be prepared by the method of the present invention may include but are not limited to those that are shown in FIGS. 4/4A [e.g., (15A), (15B), (16A), (16B), (17A), (17B), (18A), (18B), (19A), (19B), (20A), (20B), (21A), (21B), (22A), (22B), (23A), (23B), (24A), (24B), (25A), (25B), (26A), (26B), (27A), (27B), (28A), (28B), (29A), (29B), (30A), (30B), (31A), (31B), (32A), (32B), (33A), (33B), (34A), (34B), (35A), (35B), (36A), (36B), (37A), (37B), (38A), (38B), (39A), (39B), (40A), (40B), (41A), (41B), (42A), (42B), (43A), (43B), (44A), (44B), (45A), (45B), (46A), (46B), (47A), (47B), (48A), (48B)].

In another embodiment, the present invention provides a compound or any salt thereof, or any solvate thereof, or mixture comprising one or more said compounds or any salt thereof, or any solvate thereof, that may be prepared by the method of the present invention, selected from the group consisting of:

| Structure | Chemical name |
|---|---|
| 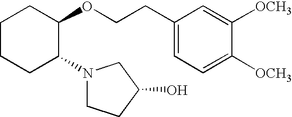 | (1R, 2R)-2-[(3R)-Hydroxypyrrolidinyl]-1-(3,4- dimethoxyphenethoxy)-cyclohexane |
| 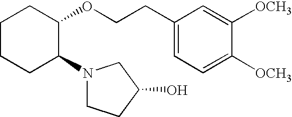 | (1S, 2S)-2-[(3R)-Hydroxypyrrolidinyl]-1-(3,4- dimethoxyphenethoxy)-cyclohexane |
| 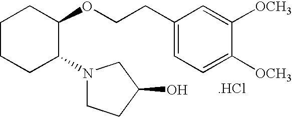 | (1R, 2R)-2-[(3S)-Hydroxypyrrolidinyl]-1-(3,4- dimethoxyphenethoxy)-cyclohexane monohydrochloride |
| 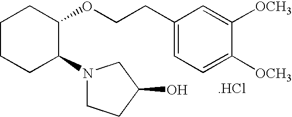 | (1S, 2S)-2-[(3S)-Hydroxypyrrolidinyl]-1-(3,4- dimethoxyphenethoxy)-cyclohexane monohydrochloride |

In another embodiment, the present invention provides a compound, or mixture comprising compounds, or any solvate thereof, selected from the group consisting of:

| Structure | Chemical name |
|---|---|
| 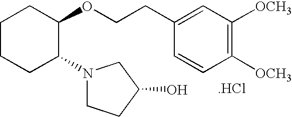 | (1R, 2R)-2-[(3R)-Hydroxypyrrolidinyl]-1-(3,4- dimethoxyphenethoxy)-cyclohexane monohydrochloride |
| 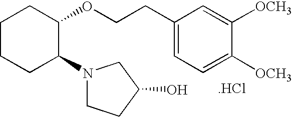 | (1S, 2S)-2-[(3R)-Hydroxypyrrolidinyl]-1-(3,4- dimethoxyphenethoxy)-cyclohexane monohydrochloride |
| 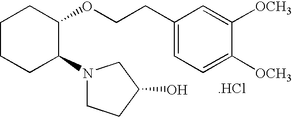 | (1S, 2S)-2-[(3R)-Hydroxypyrrolidinyl]-1-(3,4- dimethoxyphenethoxy)-cyclohexane monohydrochloride |
| 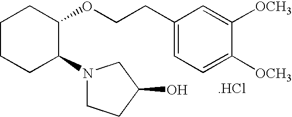 | (1S, 2S)-2-[(3S)-Hydroxypyrrolidinyl]-1-(3,4- dimethoxyphenethoxy)-cyclohexane monohydrochloride |

In another embodiment, the present invention provides a composition that includes one or more of the compounds listed above that may be prepared by the method of the present invention, or includes a solvate or a pharmaceutically acceptable salt of one or more of the compounds. The composition may or may not include additional components as is described elsewhere in detail in this patent.

In one embodiment, the present invention provides a compound which is (1R,2R)-2-[(3R)-Hydroxypyrrolidinyl]-1-(3,4-dimethoxyphenethoxy)-cyclohexane free base or any salt thereof, or any solvate thereof, that may be prepared by the method of the present invention.

In one embodiment, the present invention provides a compound which is (1R,2R)-2-[(3S)-Hydroxypyrrolidinyl]-1-(3,4-dimethoxyphenethoxy)-cyclohexane free base or any salt thereof, or any solvate thereof, that may be prepared by the method of the present invention.

In one embodiment, the present invention provides a compound which is (1S,2S)-2-[(3R)-Hydroxypyrrolidinyl]-1-(3,4-dimethoxyphenethoxy)-cyclohexane free base or any salt thereof, or any solvate thereof, that may be prepared by the method of the present invention.

In one embodiment, the present invention provides a compound which is (1S,2S)-2-[(3S)-Hydroxypyrrolidinyl]-1-(3,4-dimethoxyphenethoxy)-cyclohexane free base or any salt thereof, or any solvate thereof, that may be prepared by the method of the present invention.

In one embodiment, the present invention provides a compound which is (1R,2R)-2-[(3R)-Hydroxypyrrolidinyl]-1-(3,4-dimethoxyphenethoxy)-cyclohexane monohydrochloride, or any solvate thereof, that may be prepared by the method of the present invention.

In one embodiment, the present invention provides a compound which is (1R,2R)-2-[(3S)-Hydroxypyrrolidinyl]-1-(3,4-dimethoxyphenethoxy)-cyclohexane monohydrochloride, or any solvate thereof, that may be prepared by the method of the present invention.

In one embodiment, the present invention provides a compound which is (1S,2S)-2-[(3R)-Hydroxypyrrolidinyl]-1-(3,4-dimethoxyphenethoxy)-cyclohexane monohydrochloride, or any solvate thereof, that may be prepared by the method of the present invention.

In one embodiment, the present invention provides a compound which is (1S,2S)-2-[(3S)-Hydroxypyrrolidinyl]-1-(3,4-dimethoxyphenethoxy)-cyclohexane monohydrochloride, or any solvate thereof, that may be prepared by the method of the present invention.

The present invention also provides protenated versions of all of the compounds described in this patent that may be prepared by the method of the present invention. That is, for each compound described in this patent, the invention also includes the quaternary protenated amine form of the compound that may be prepared by the method of the present invention. These quaternary protenated amine form of the compounds may be present in the solid phase, for example in crystalline or amorphous form, and may be present in solution. These quaternary protenated amine form of the compounds may be associated with pharmaceutically acceptable anionic counter ions, including but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002.

Method for Preparing Stereoisomerically Substantially Pure Trans-Aminocyclohexyl Ether Compounds

The aminocyclohexyl ether compounds of the present invention contain amino and ether functional groups disposed in a 1,2 arrangement on a cyclohexane ring. Accordingly, the amino and ether functional groups may be disposed in either a cis or trans relationship, relative to one another and the plane of the cyclohexane ring as shown on the page in a two dimensional representation.

The present invention provides synthetic methodology for the preparation of the aminocyclohexyl ether compounds according to the present invention as described herein. The aminocyclohexyl ether compounds described herein may be prepared from aminoalcohols and alcohols by following the general methods described below, and as illustrated in the examples. Some general synthetic processes for aminocyclohexyl ethers have been described in WO 99/50225 and references cited therein. Other processes that may be used for preparing compounds of the present invention are described in the following US provisional patent applications: U.S. 60/476,083, U.S. 60/476,447, U.S. 60/475,884, U.S. 60/475,912 and U.S. 60/489,659, upon which the present application claims priority, and references cited therein.

The present invention provides synthetic processes whereby compounds of formula (57) with trans-(1R,2R) configuration for the ether and amino functional groups may be prepared in stereoisomerically substantially pure form. Compounds of formulae (66), (67), (69) and (71) are some of the examples represented by formula (57). The present invention also provides synthetic processes whereby compounds of formulae (52), (53), and (55) may be synthesized in stereoisomerically substantially pure forms. Compounds (61) and (61A) are examples of formula (52). Compounds (62) and (62A) are examples of formula (53). Compounds (64) and (64A) are examples of formula (55).

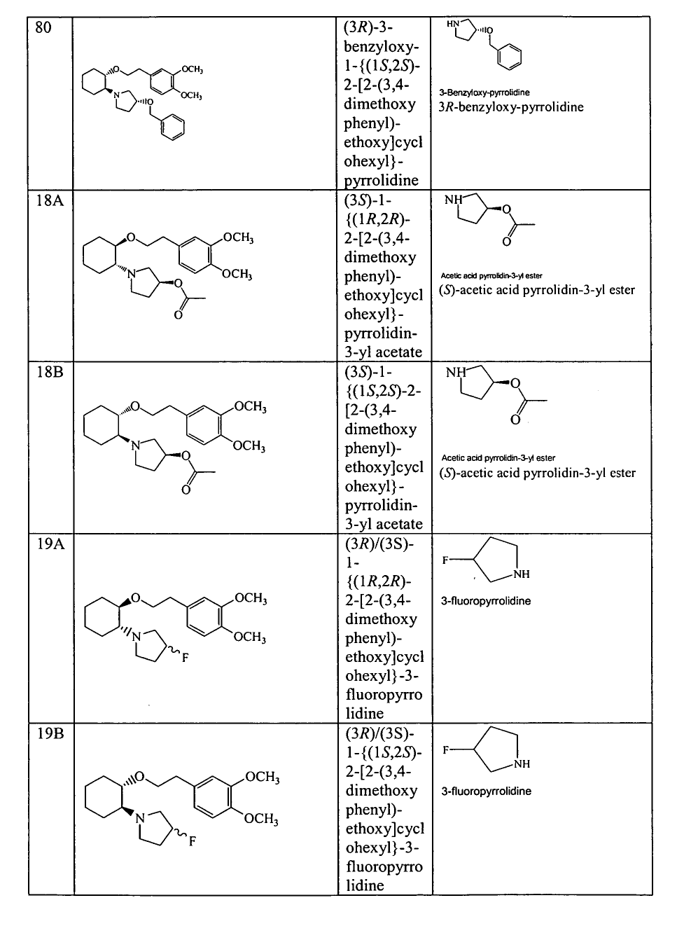

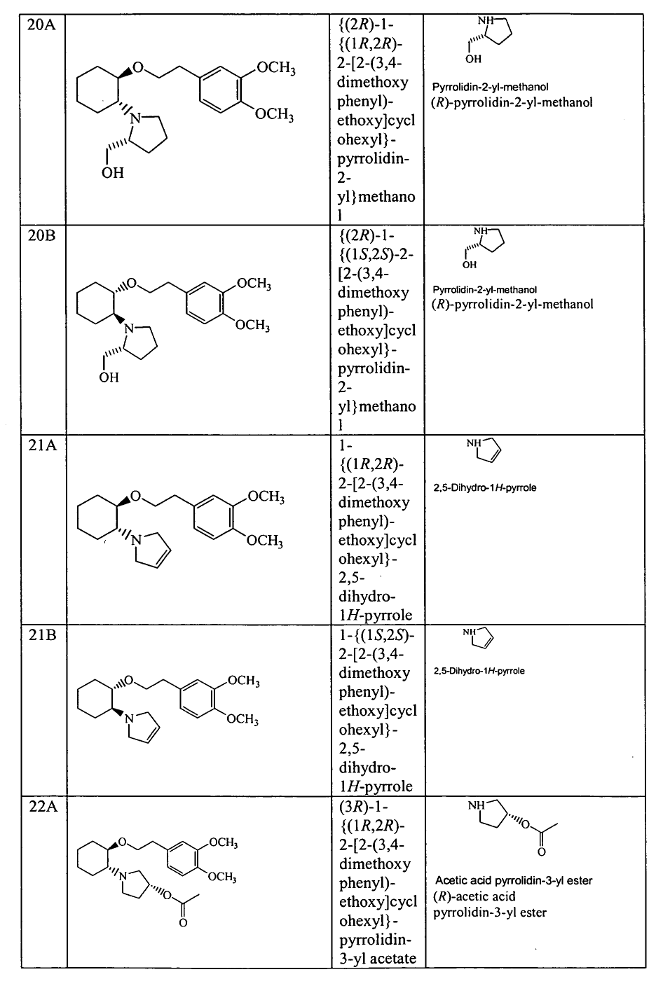

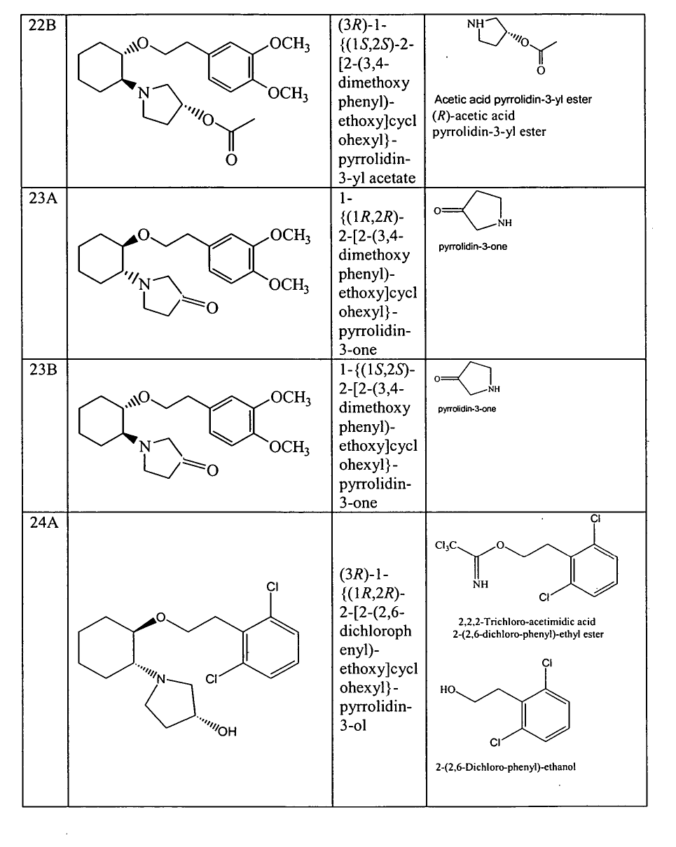

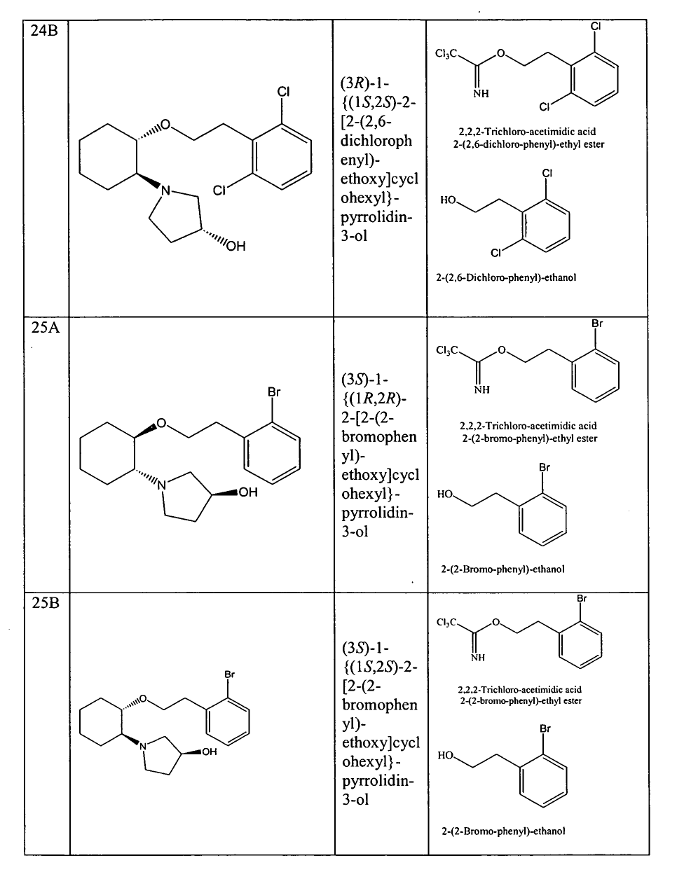

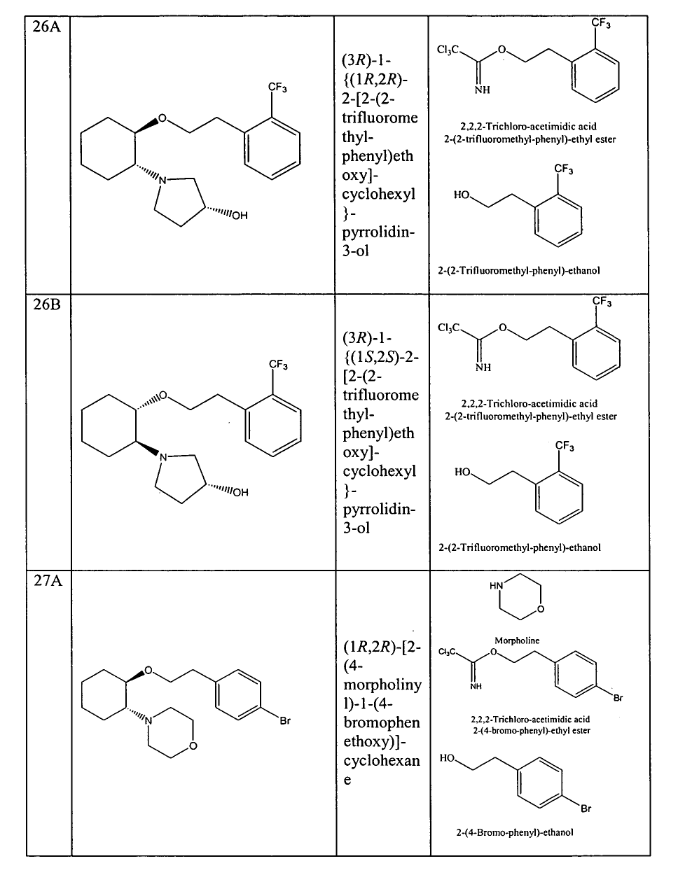

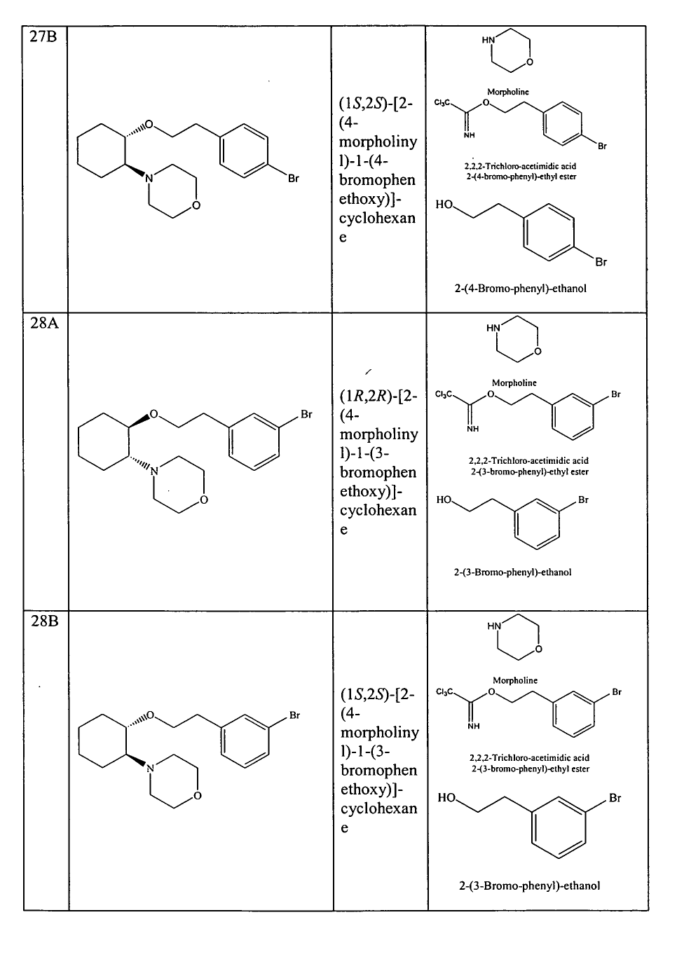

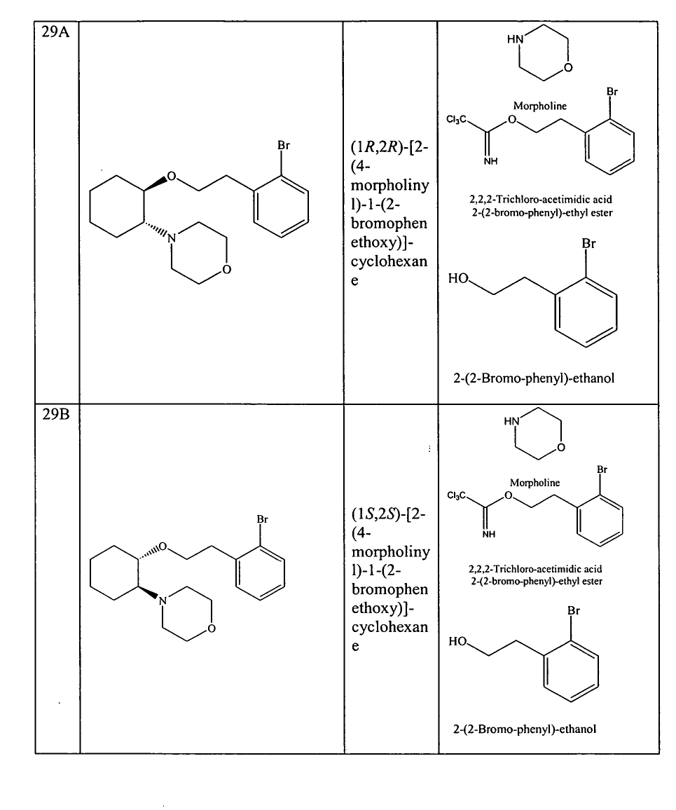

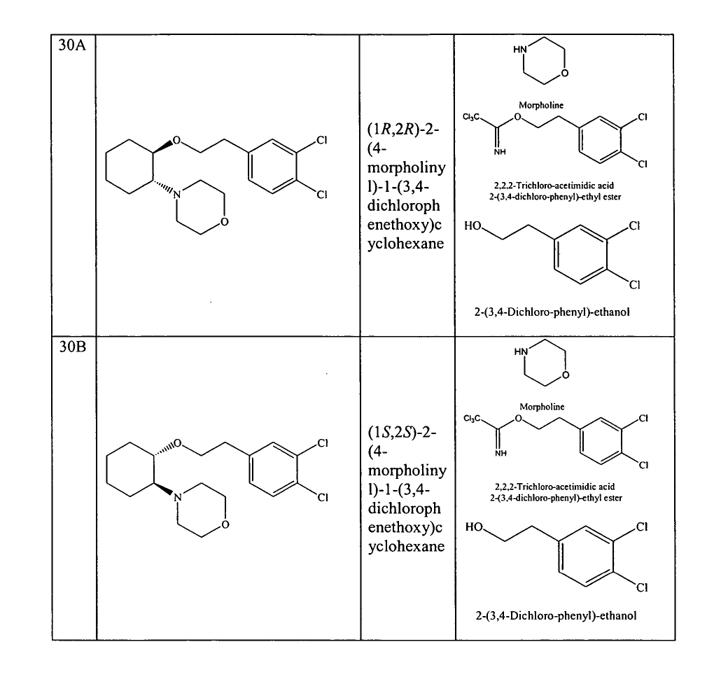

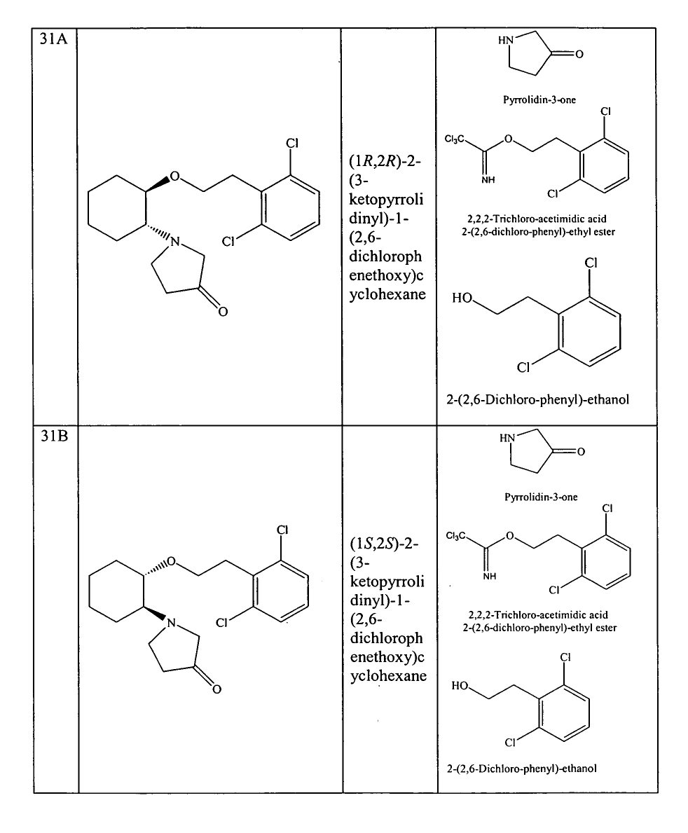

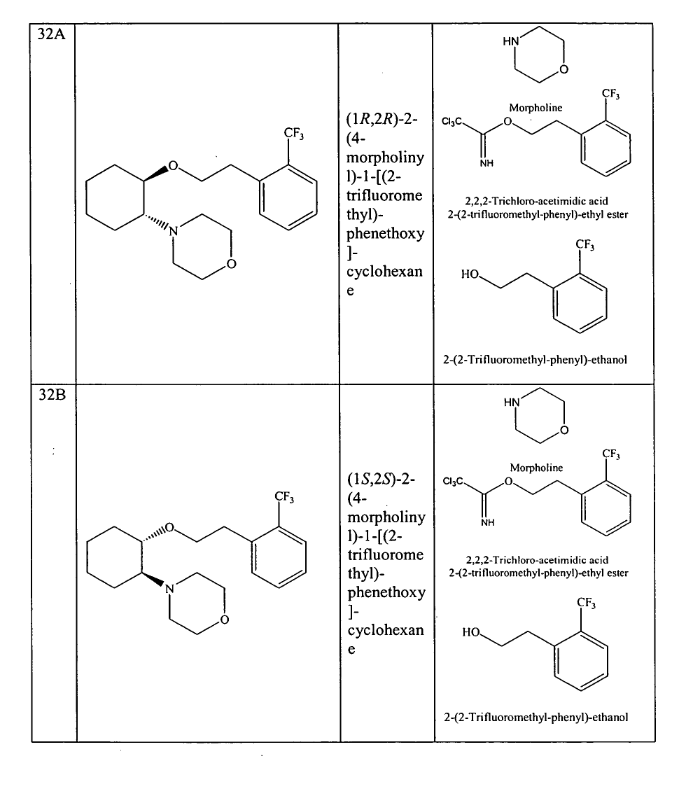

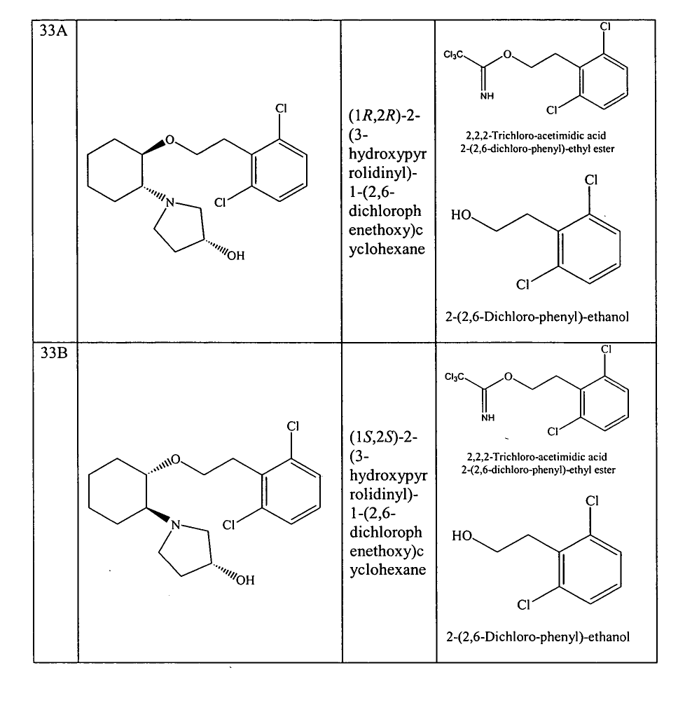

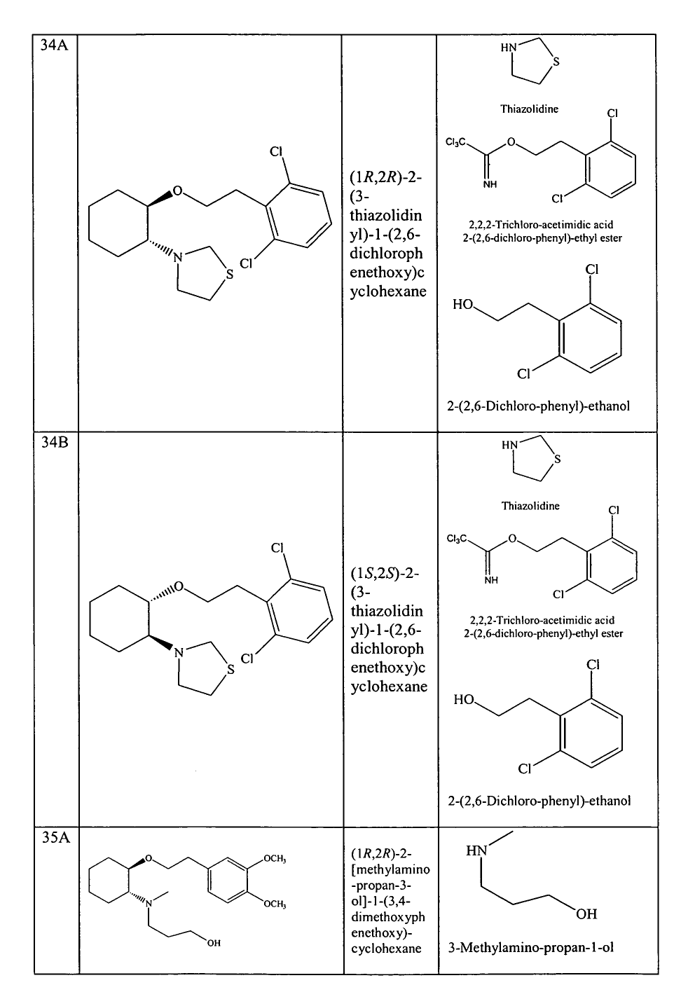

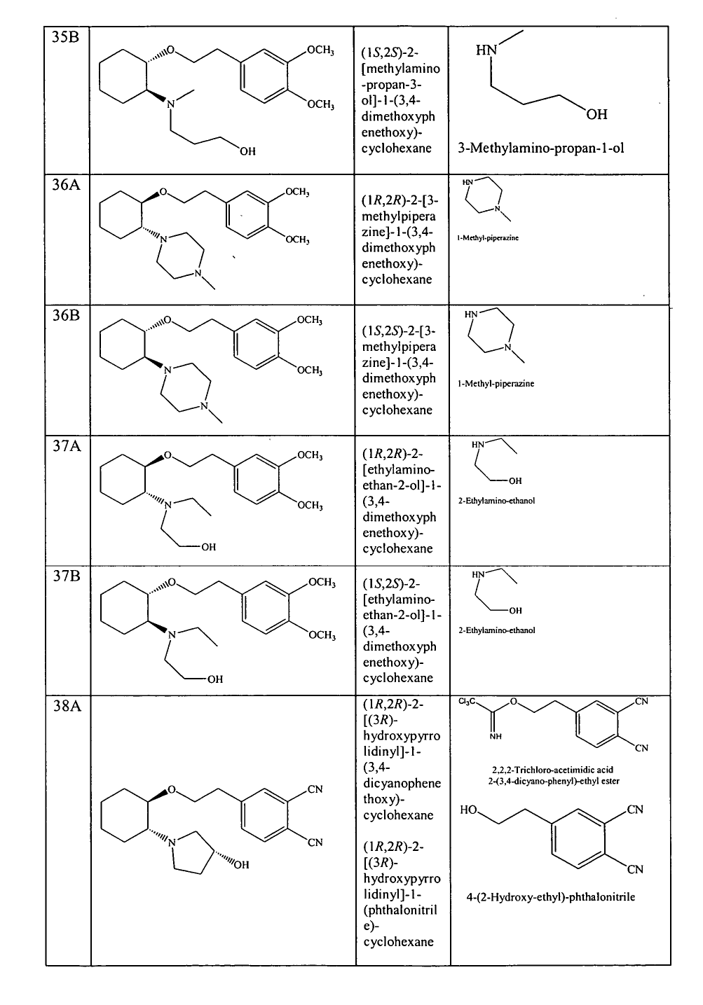

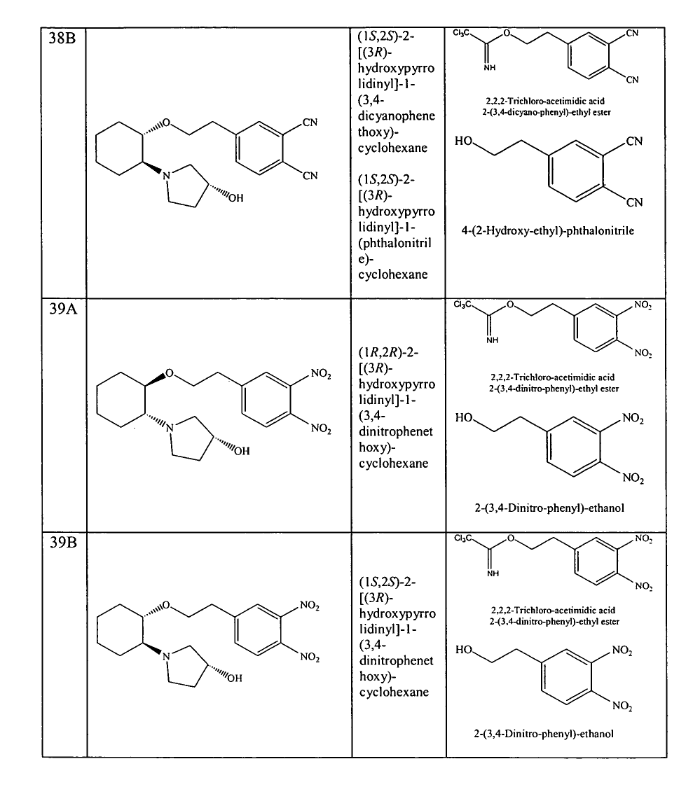

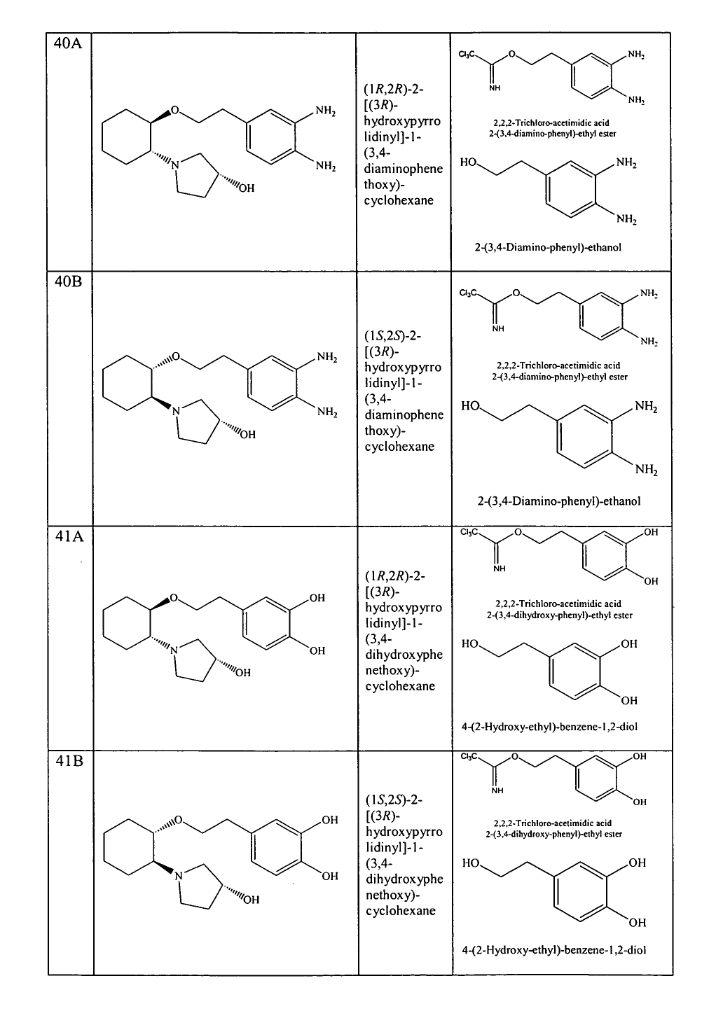

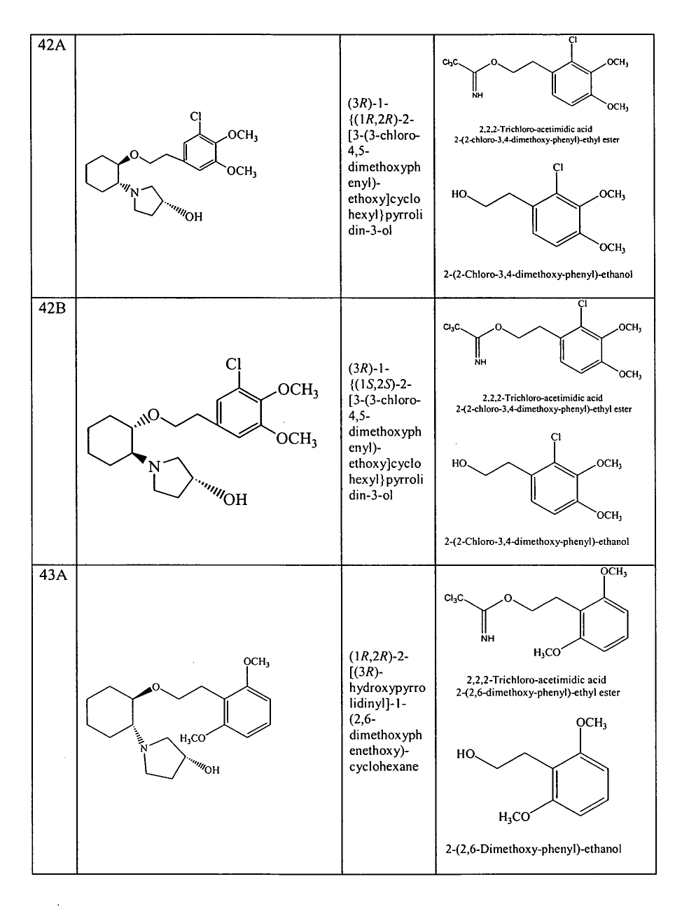

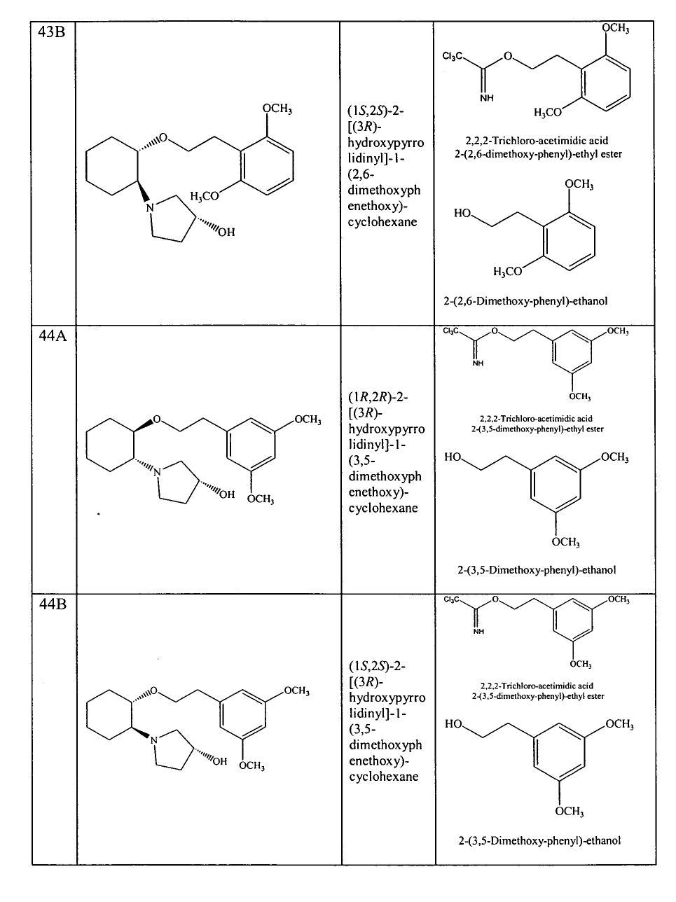

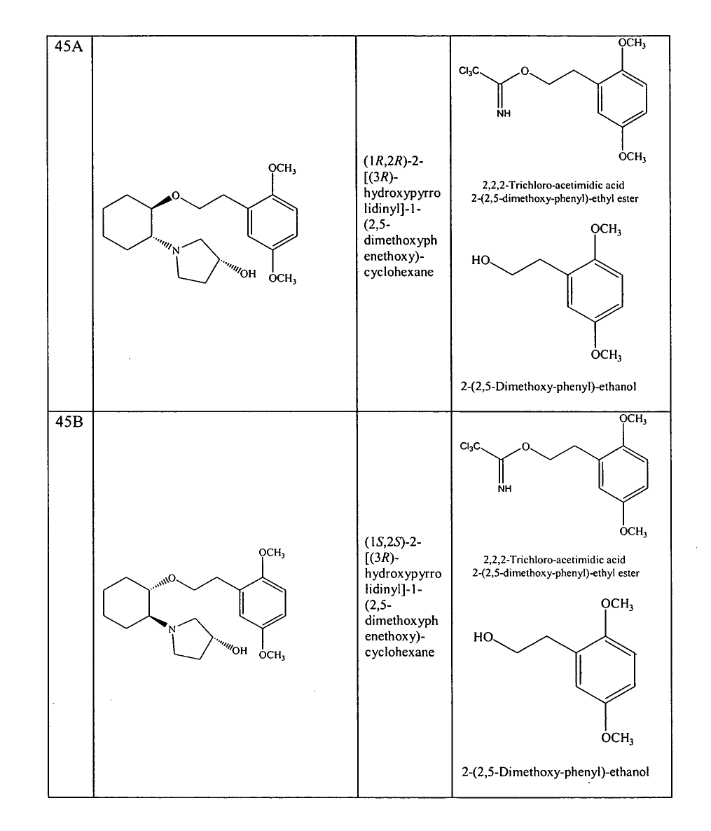

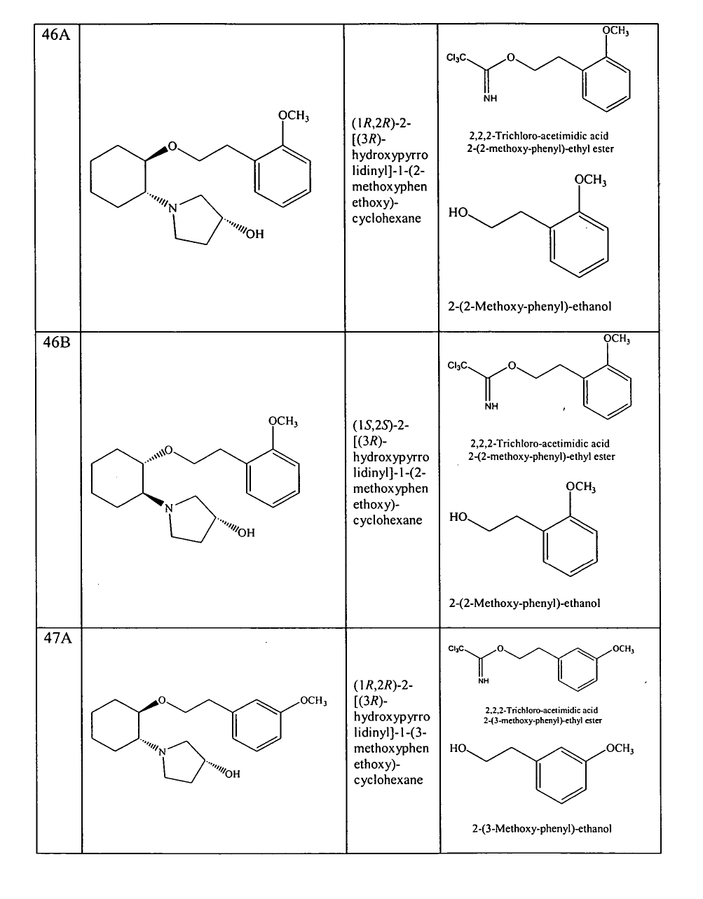

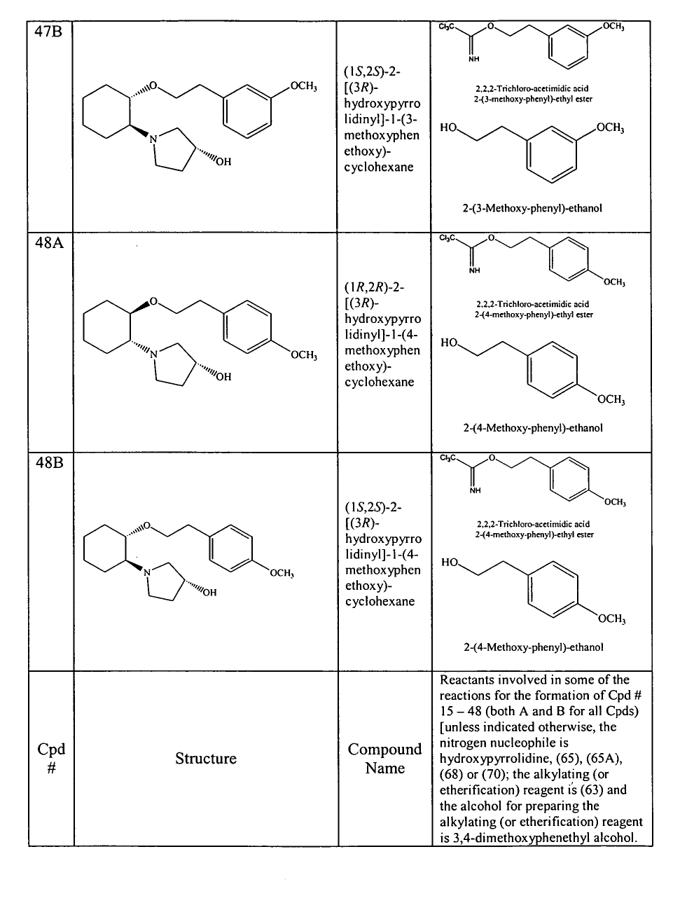

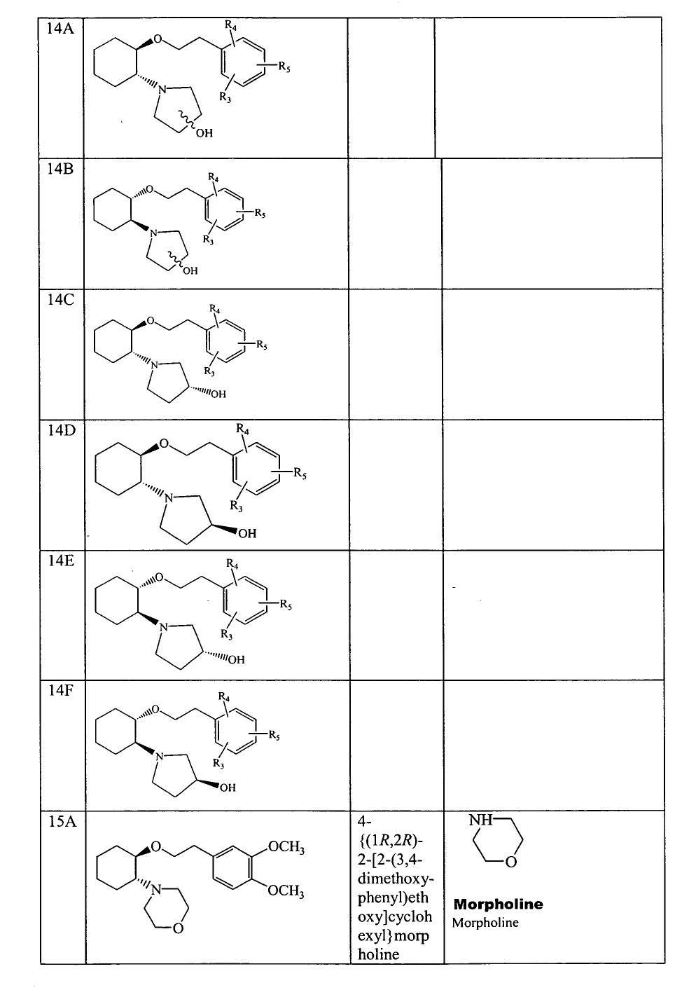

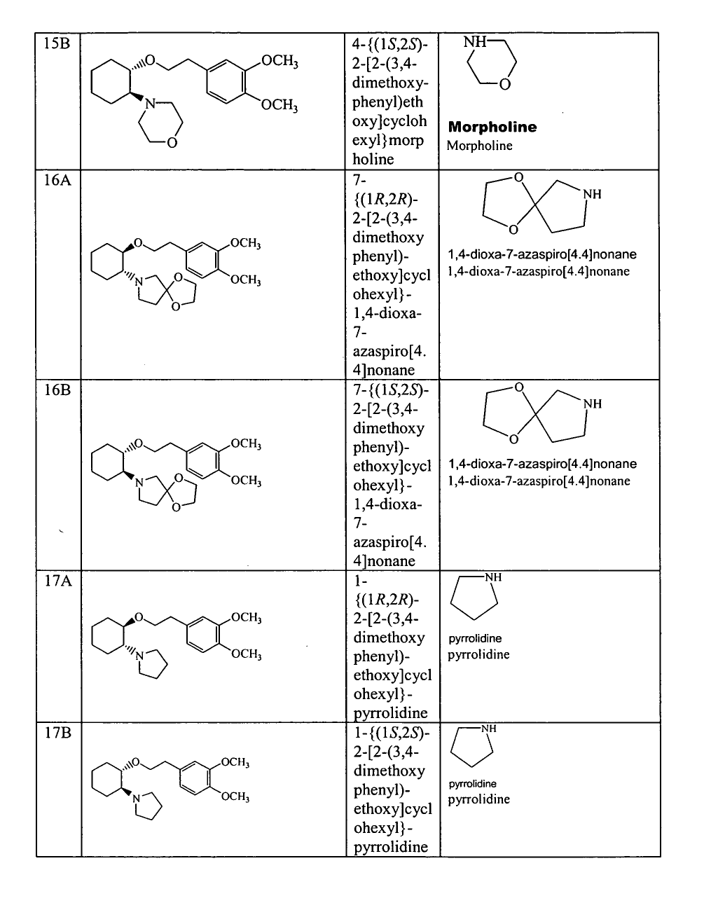

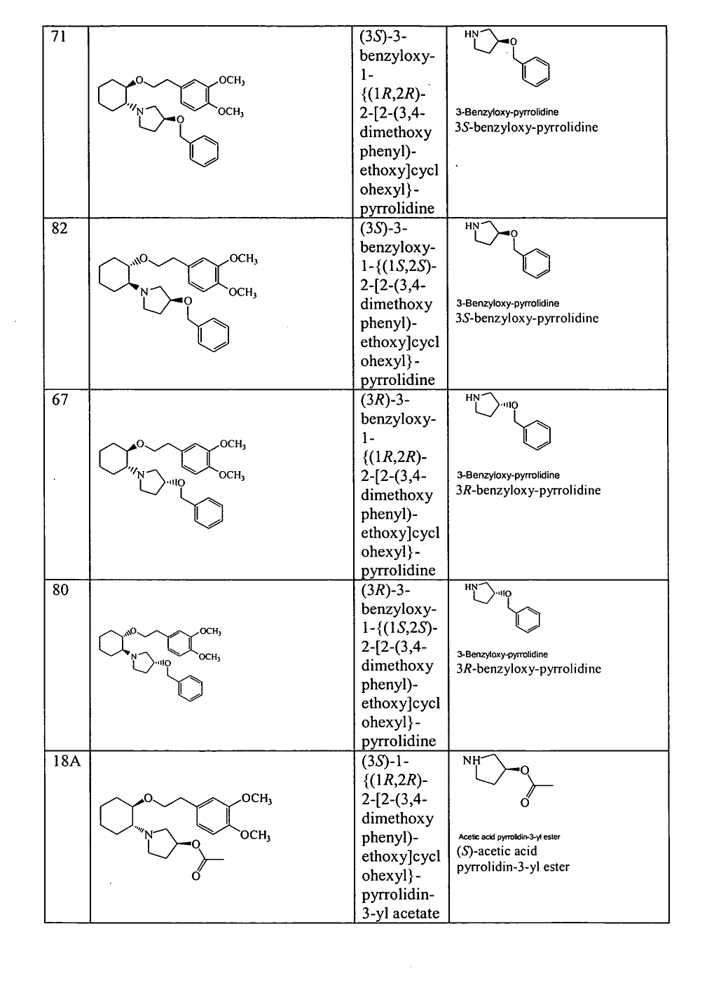

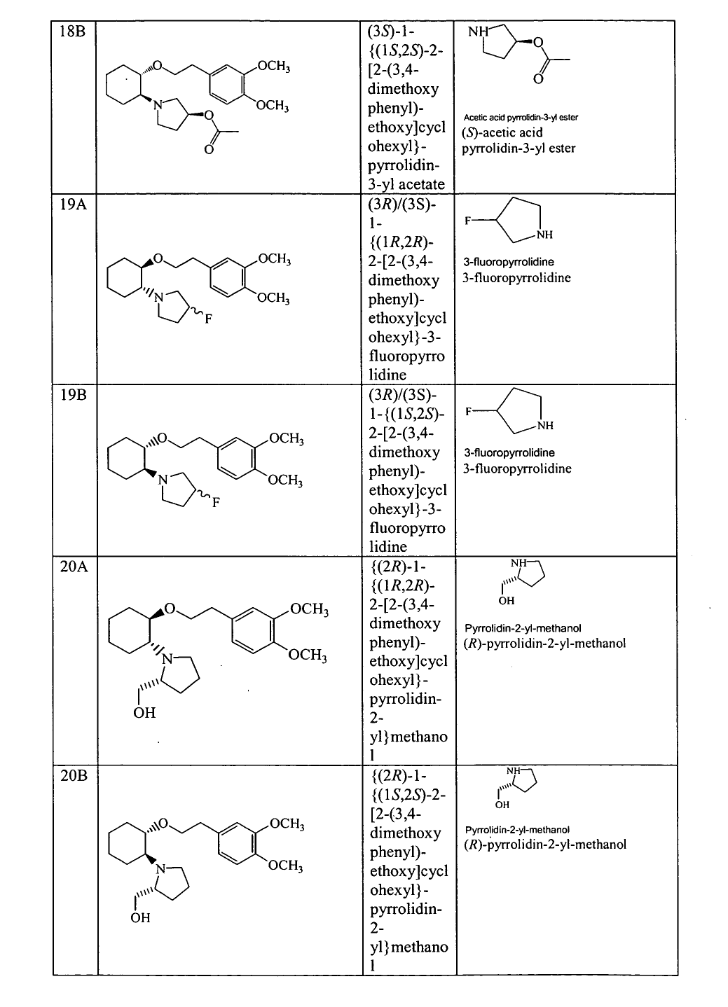

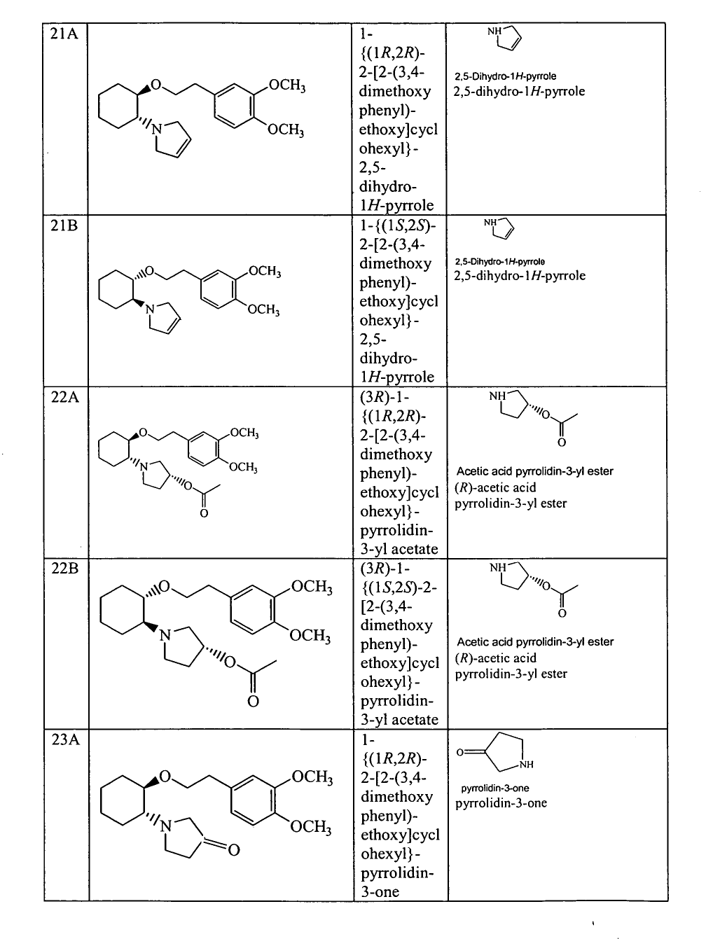

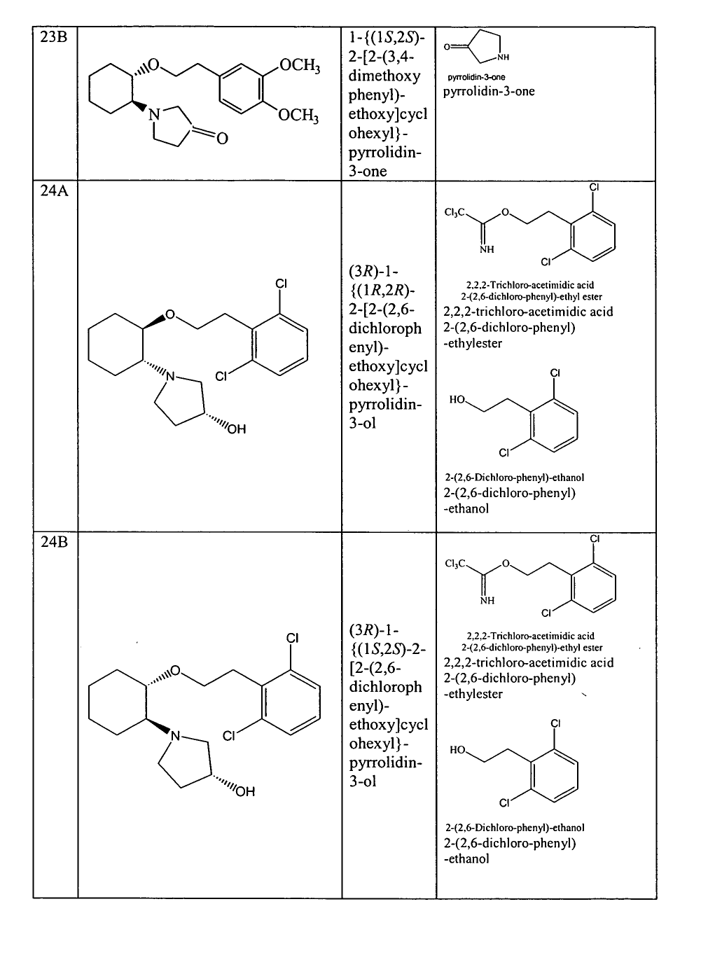

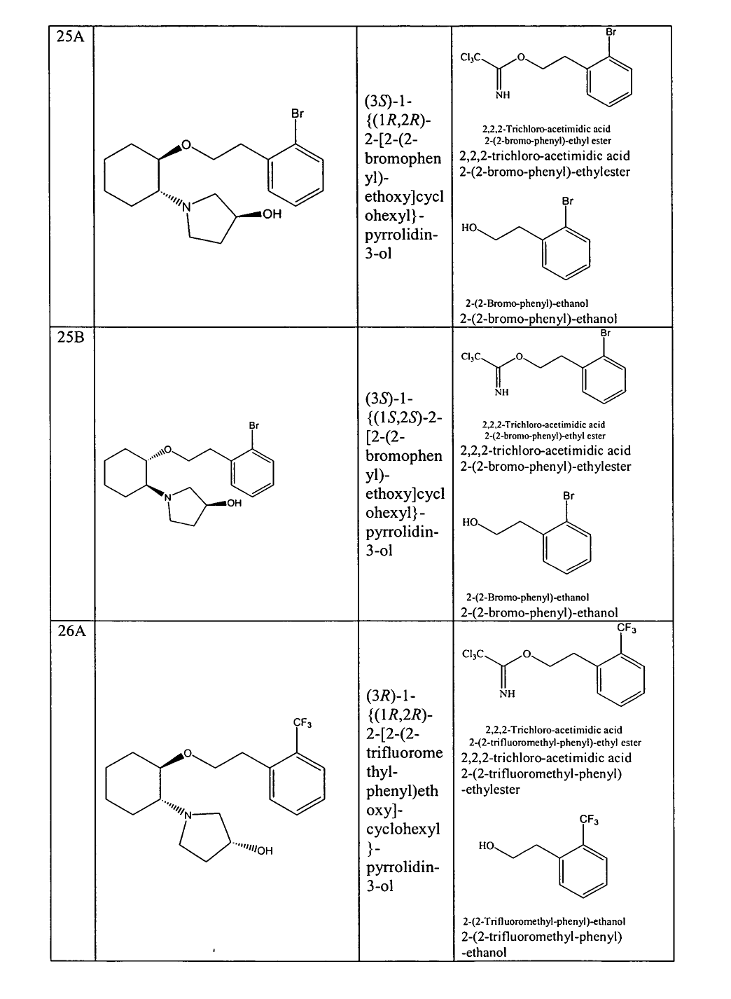

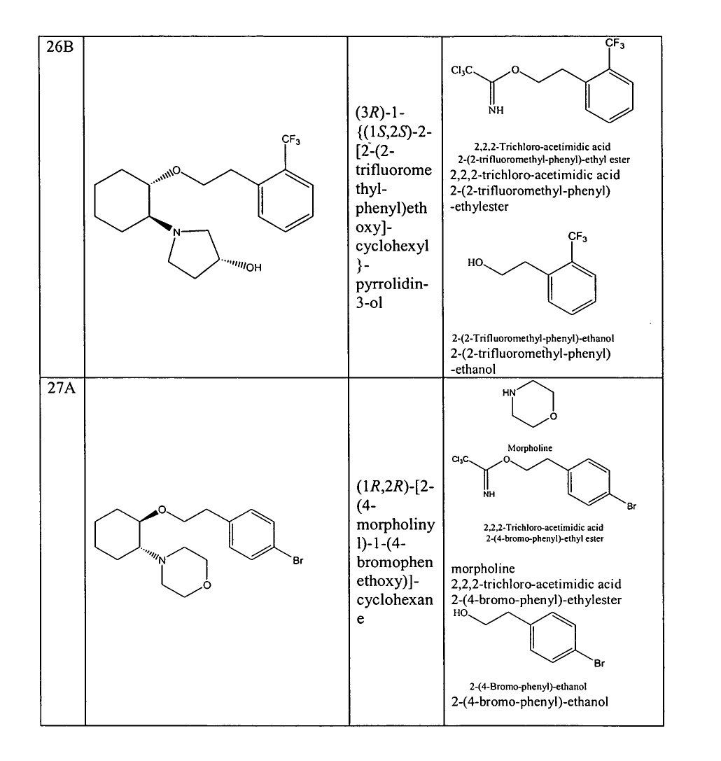

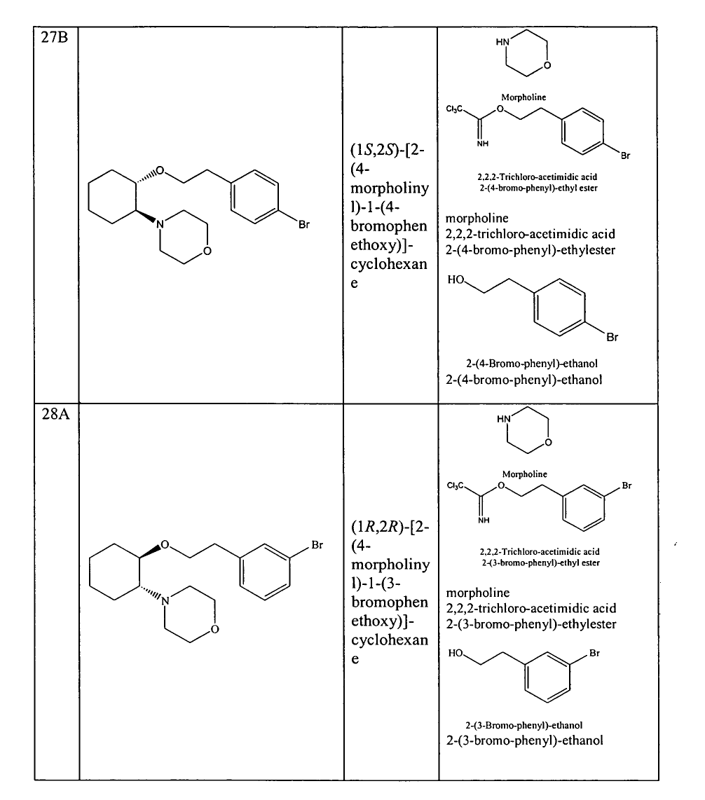

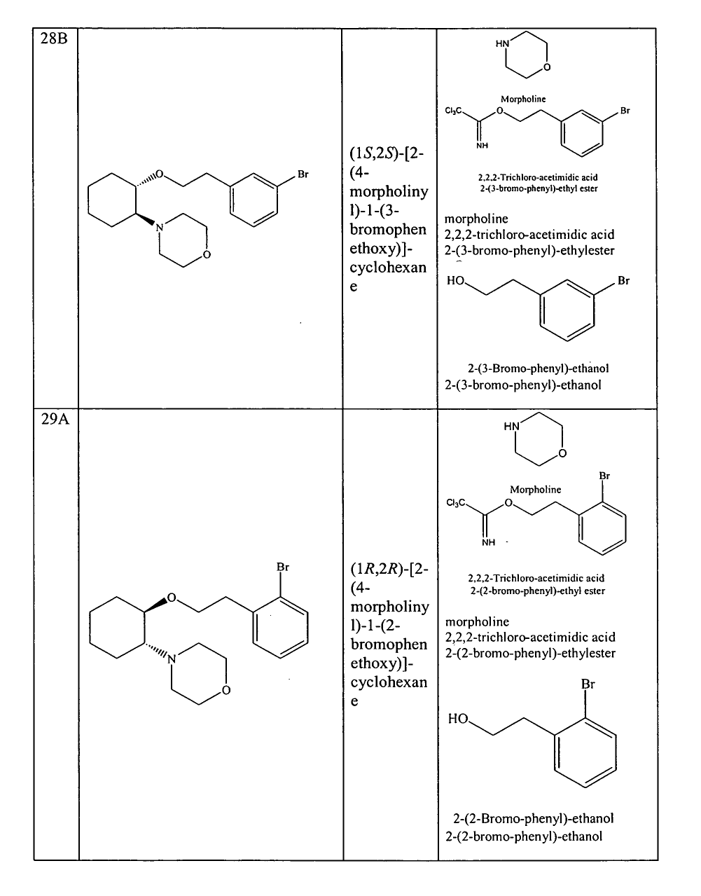

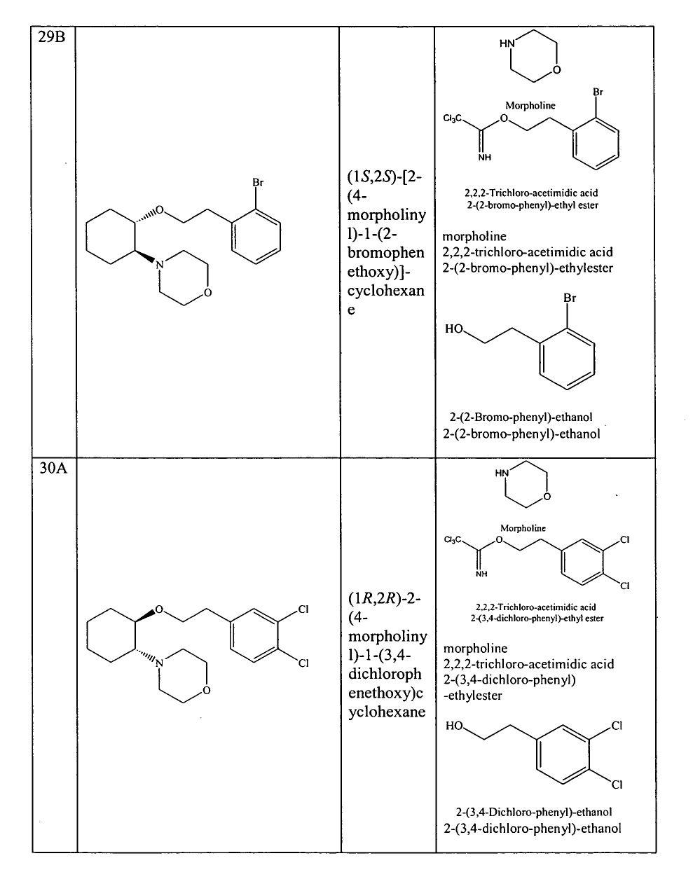

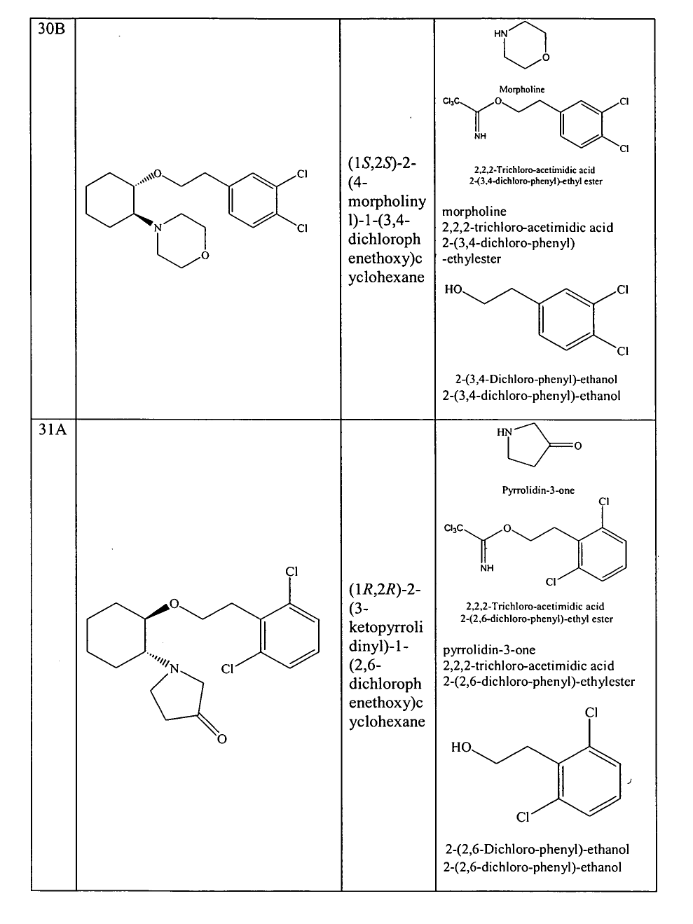

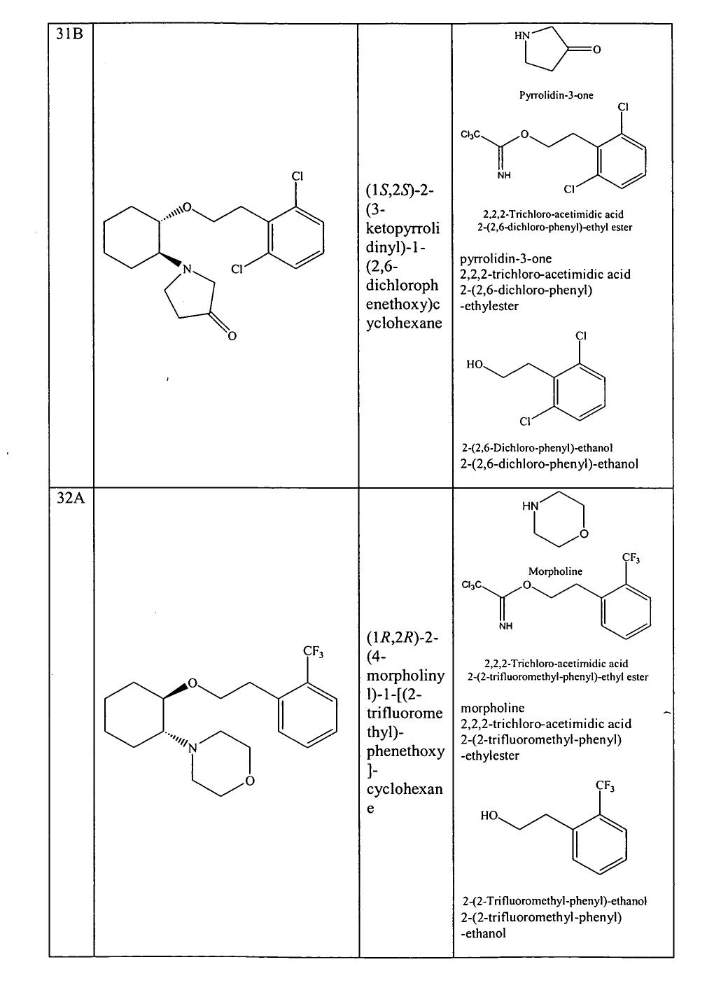

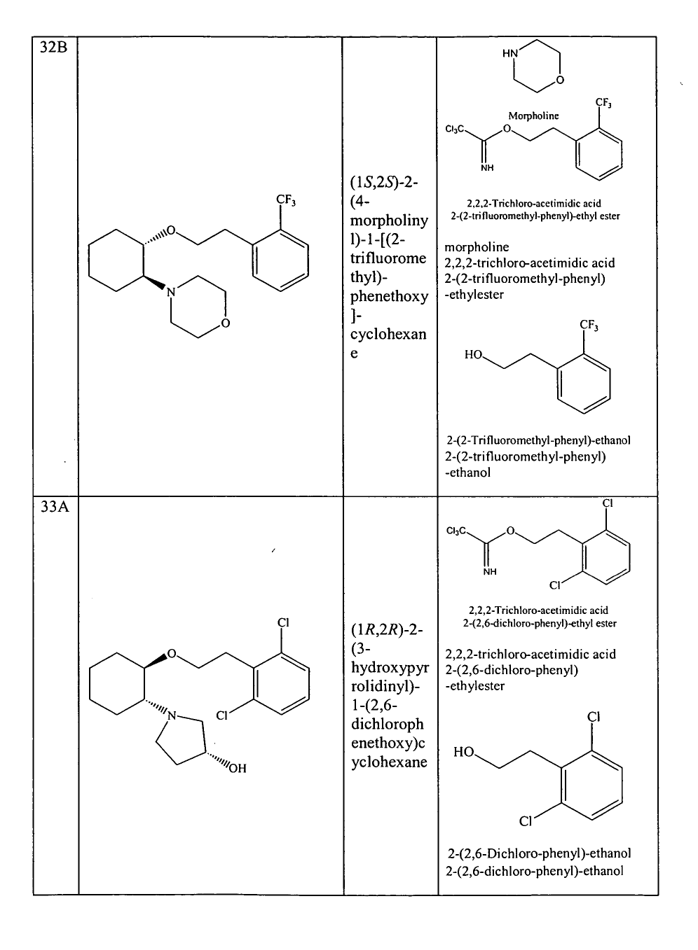

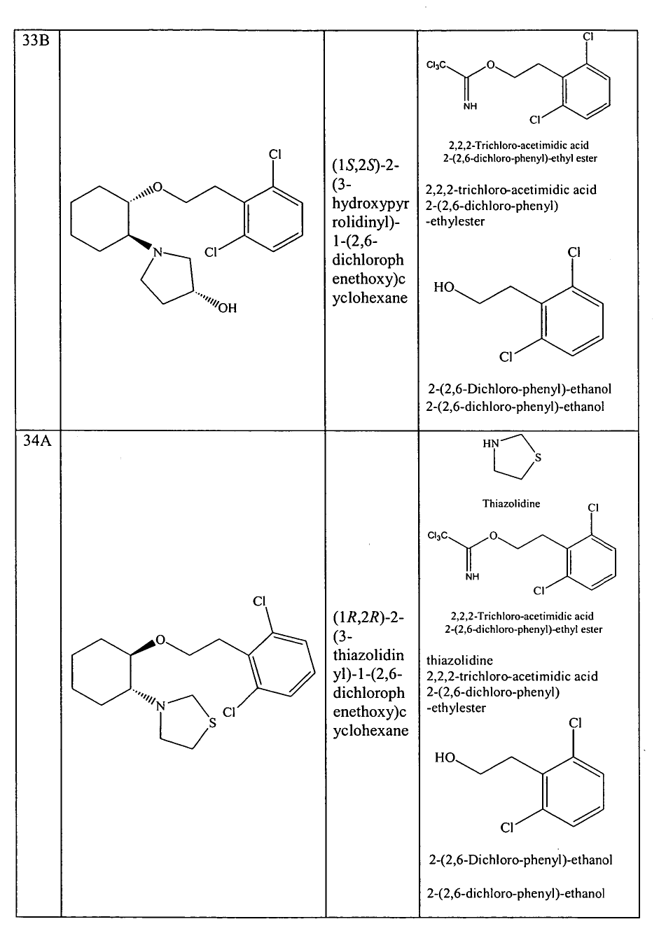

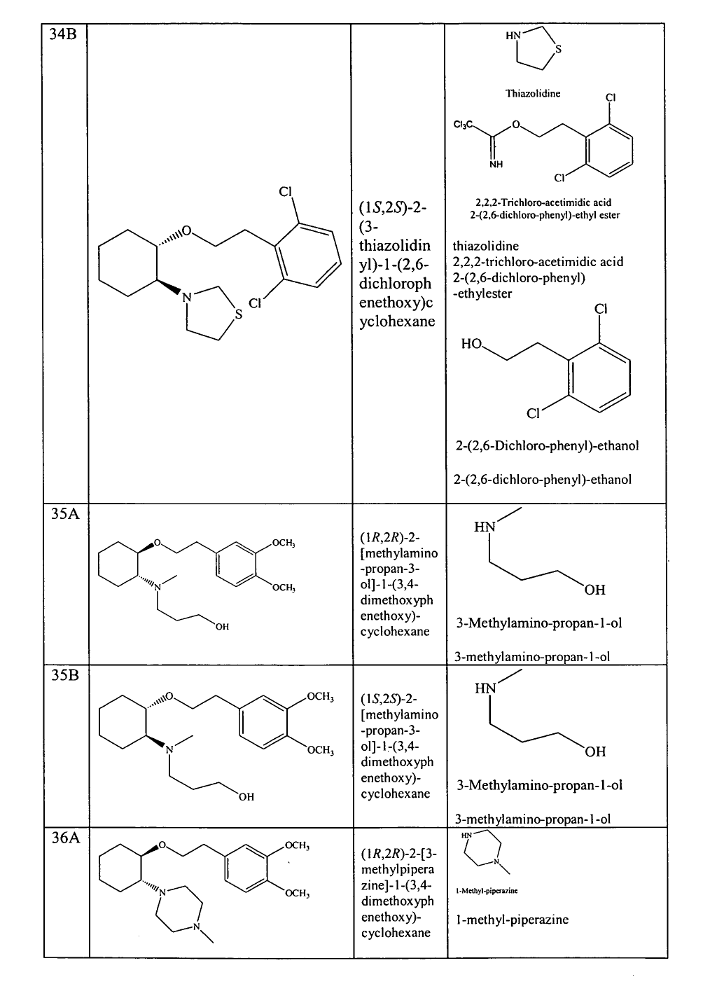

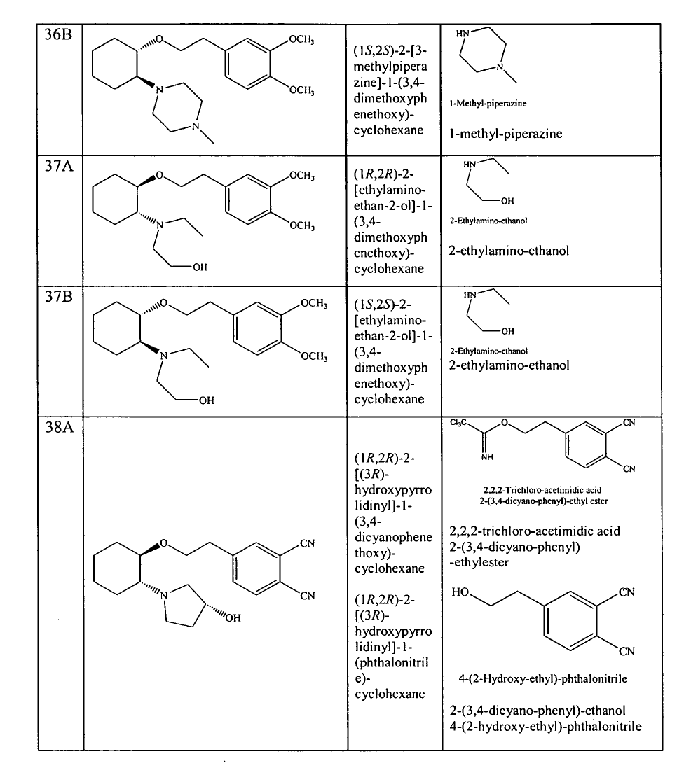

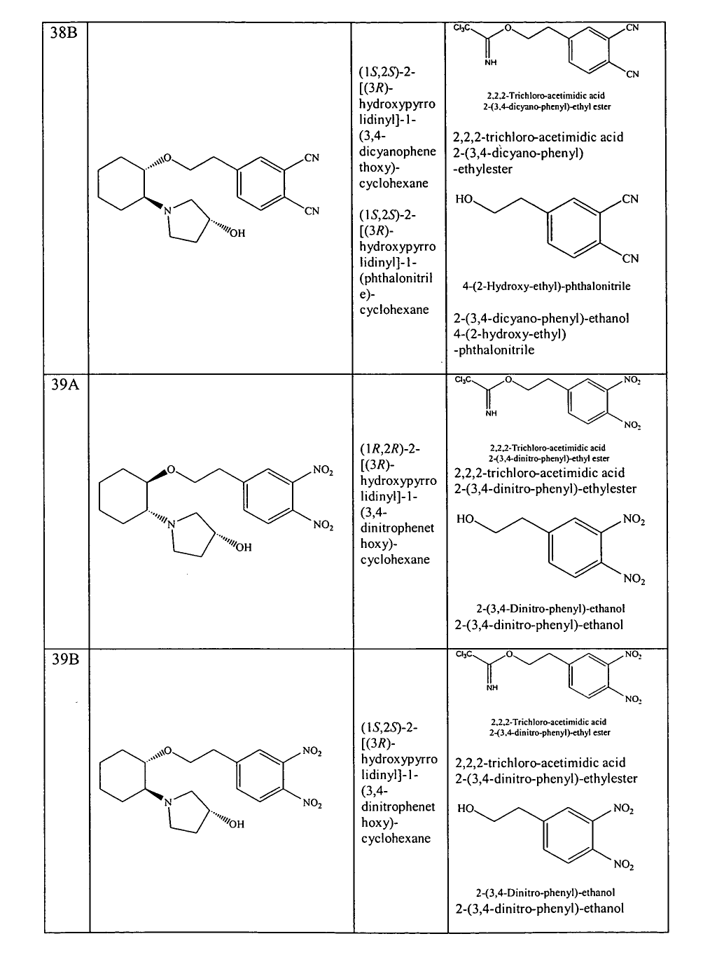

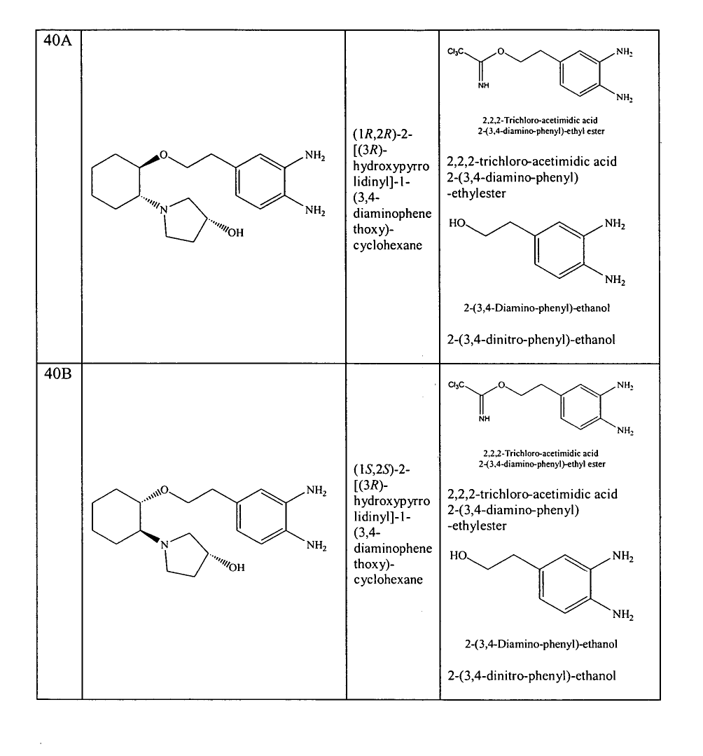

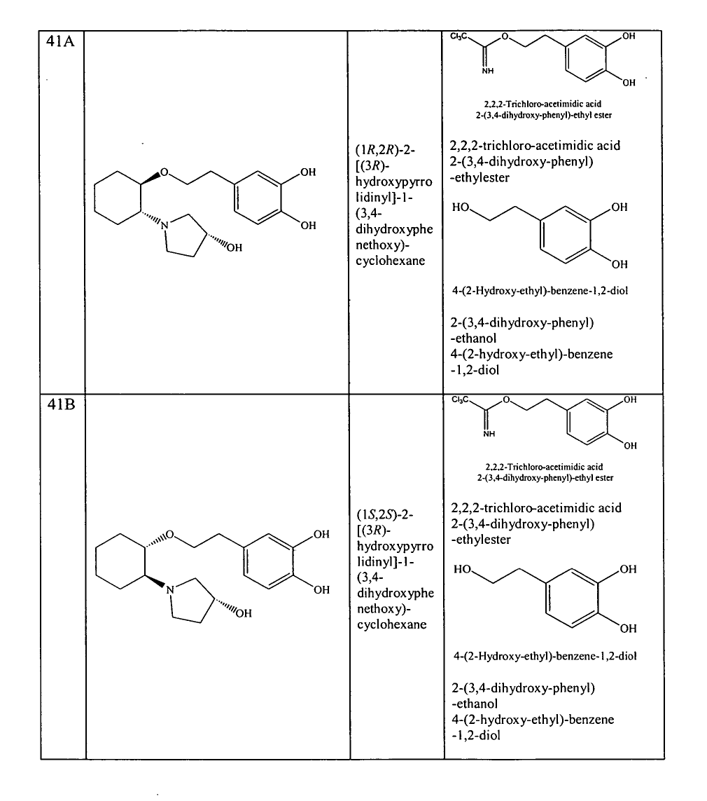

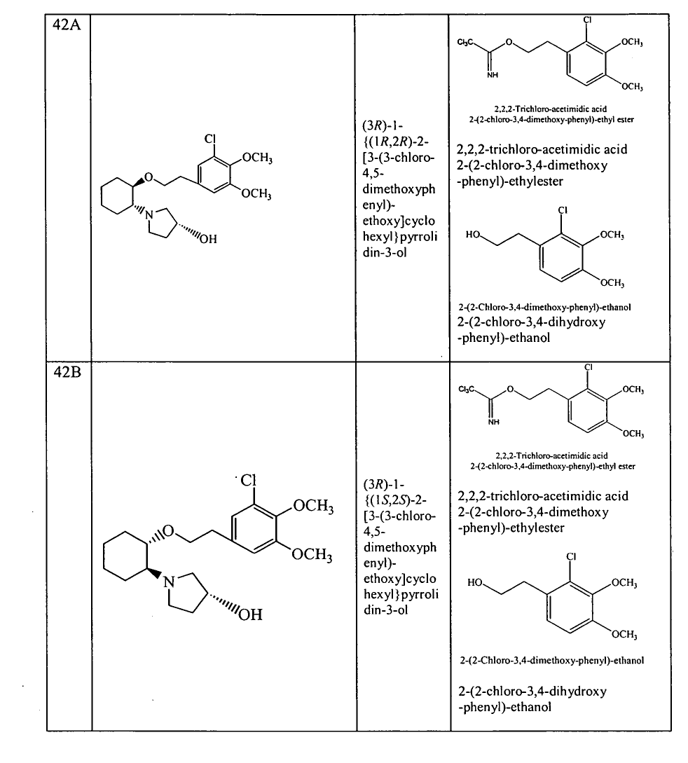

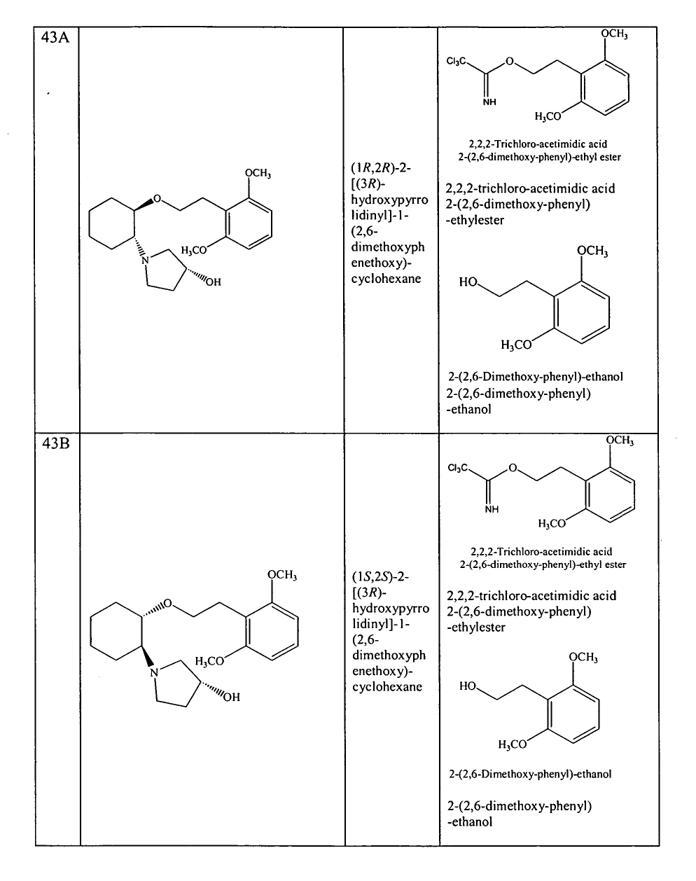

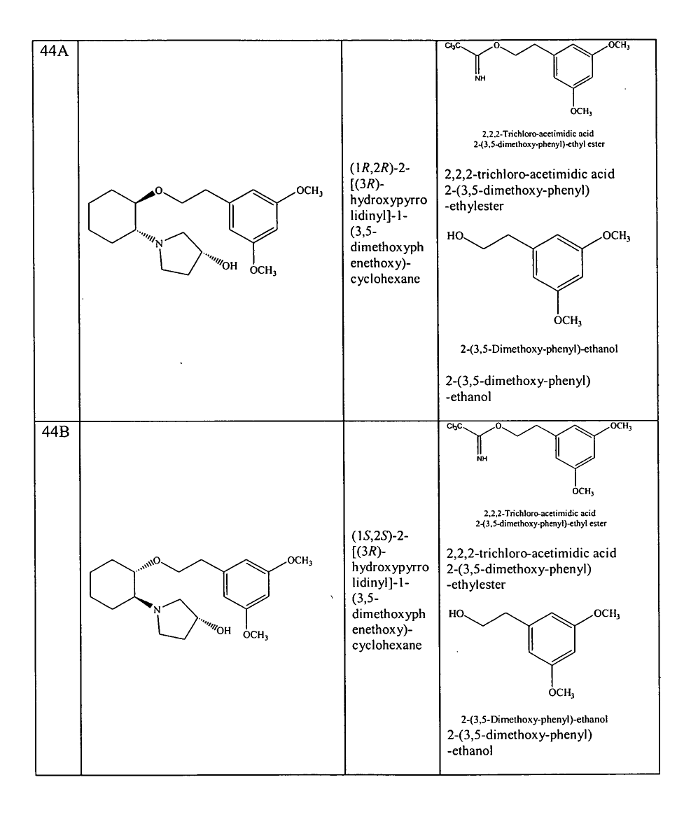

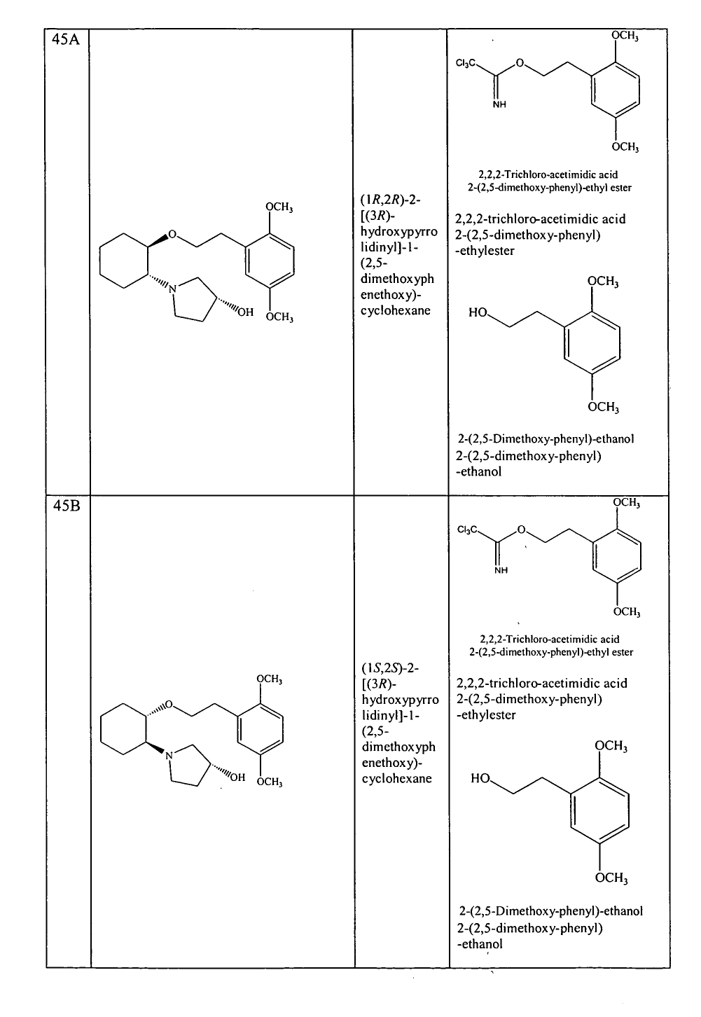

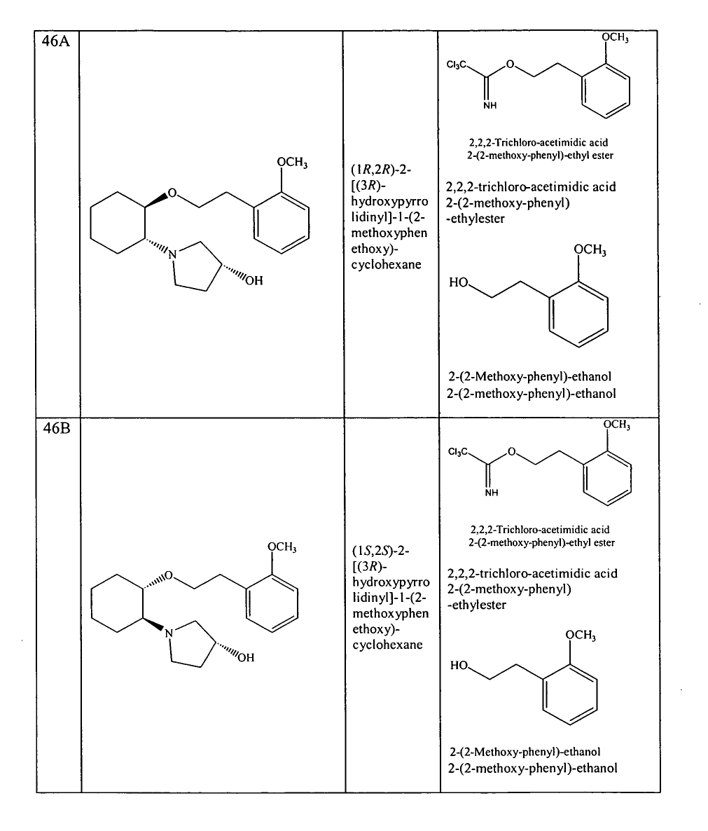

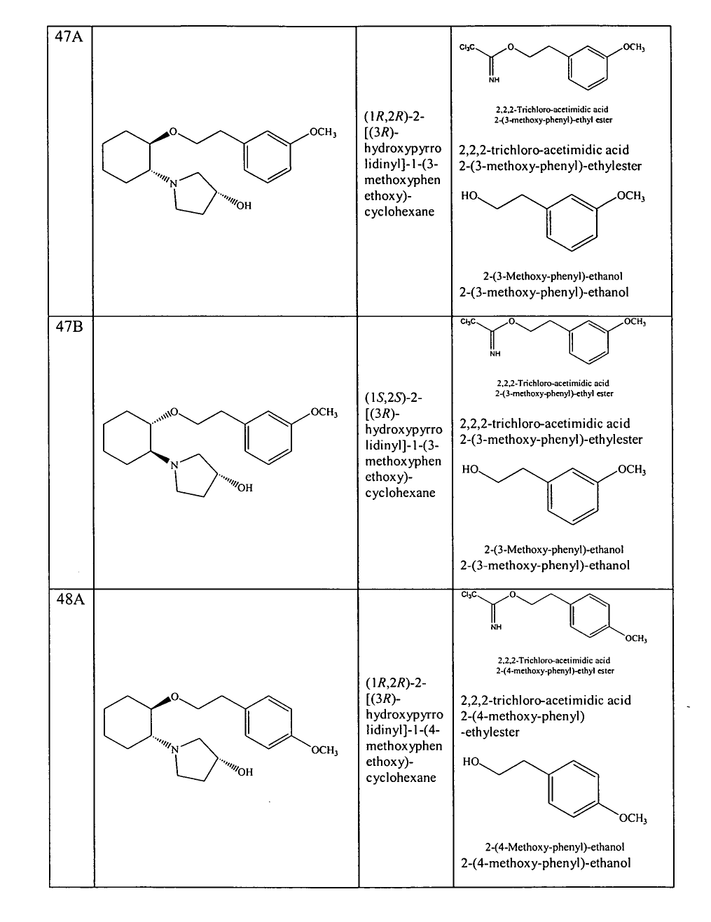

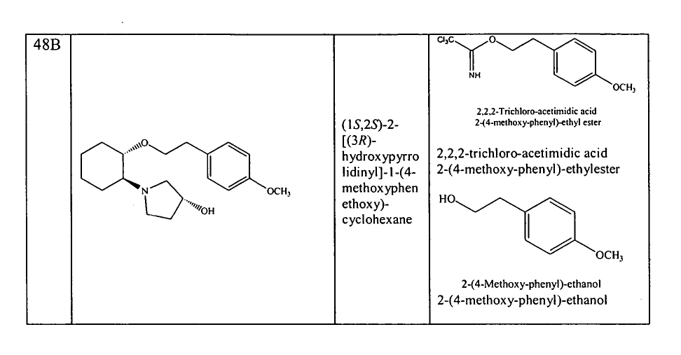

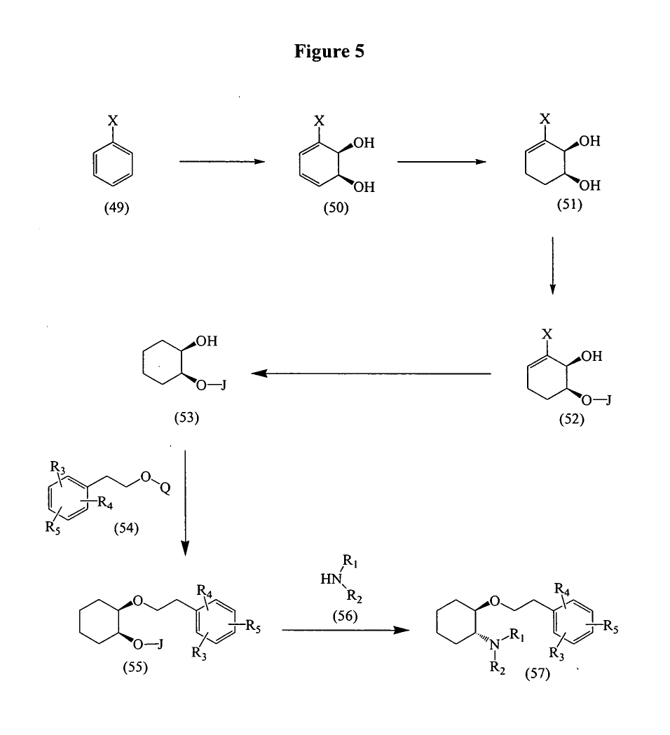

As outlined in FIG. 5, the preparation of a stereoisomerically substantially pure trans aminocyclohexyl ether compound of formula (57) may be carried out by following a process starting from a monohalobenzene (49), wherein X may be F, Cl, Br or I.

In a first step, compound (49) is transformed by well-established microbial oxidation to the cis-cyclohexandienediol (50) in stereoisomerically substantially pure form (T. Hudlicky et al., Aldrichimica Acta, 1999, 32, 35; and references cited therein). In a separate step, compound (50) may be selectively reduced under suitable conditions to compound (51) (e.g., H₂—Rh/Al₂O₃; Boyd et al. JCS Chem. Commun. 1996, 45-46; Ham and Coker, J. Org. Chem. 1964, 29, 194-198; and references cited therein). In another separate step, the less hindered hydroxy group of formula (51) is selectively converted under suitable conditions into an “activated form” as represented by formula (52). An “activated form” as used herein means that the hydroxy group is converted into a good leaving group (—O-J) which on reaction with an appropriate nucleophile (e.g., HNR₁R₂) will result in a substitution product with substantial inversion of the stereochemical configuration of the activated hydroxy group. The leaving group (—O-J) may be but is not limited to an alkyl sulfonate such as a trifluoromethanesulfonate group (CF₃SO₃—) or a mesylate group (MsO—), an aryl sulfonate such as a benzenesulfonate group (PhSO₃—), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. In a typical reaction for the formation of an alkyl sulfonate (e.g., a mesylate) or an aryl sulfonate (e.g., a tosylate or a nosylate), compound (51) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (51) may be used to maximally convert the hydroxy group into the activated form. In a separate step, transformation of compound (52) to compound (53) may be effected by hydrogenation and hydrogenolysis in the presence of a catalyst under appropriate conditions. Palladium on activated carbon is one example of the catalysts. Hydrogenolysis of alkyl or alkenyl halide such as (52) may be conducted under basic conditions. The presence of a base such as sodium ethoxide, sodium bicarbonate, sodium acetate or calcium carbonate are some possible examples. The base may be added in one portion or incrementally during the course of the reaction.

In a separate step, alkylation of the free hydroxy group in compound (53) to form compound (55) is carried out under appropriate conditions with an alkylating reagent such as compound (54), where —O-Q represents a good leaving group which on reaction with a hydroxy function will result in the formation of an ether compound with retention of the stereochemical configuration of the hydroxy function. Haloacetimidate (e.g., trifluoroacetimidate or trichloroacetimidate) is one example for the —O-Q function. For some compounds of the formula (54), it may be necessary to introduce appropriate protection groups prior to this step being performed. Suitable protecting groups are set forth in, for example, Greene, “Protective Groups in Organic Chemistry”, John Wiley & Sons, New York N.Y. (1991).

Table A below provides additional examples of formula (54) that may be applied in the method of the present invention. (For a review of the application of various examples of formula (54) in the formation of an ether compound with an alcohol see for example, Toshima K. and Tatsuta K. Chem. Rev. 1993, 93, 1503, Tsuda T., Nakamura S. and Hashimoto S. Tetrahedron Lett. 2003, 44, 6453, Martichonok V. and Whitesides G. M. J. Org. Chem., 1996, 61, 1702 and references cited therein.)

In addition to haloacetimidate (e.g. trihaloacetimidate such as trifluoroacetimidate or trichloroacetimidate) and other imidate esters (e.g. pentafluorobenzimidate), other examples of formula (54) are 0-carbonate and S-carbonate derivatives which include, an imidazole carbonate derivative, an imidazolethiocarbonate. Phosphate derivatives which include a diphenyl phosphate, a diphenylphosphineimidate, or a phosphoroamidate and other classes of compounds such as O-sulfonyl derivative are shown in Table A below. “Derivatives” includes those compounds capable of functioning as a leaving group in compound (54).

TABLE A
Examples of Formula (54)* where Ar =

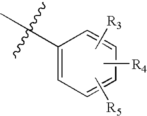

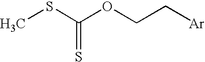

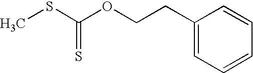

Dithiocarbonic acid S-methyl ester O-phenethyl ester

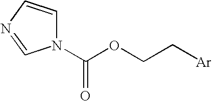

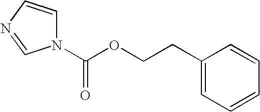

Imidazole-1-carboxylic acid phenethyl ester

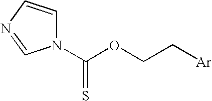

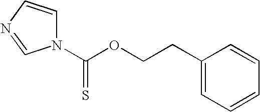

Imidazole-1-carbothioic acid O-phenethyl ester

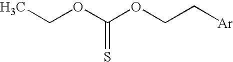

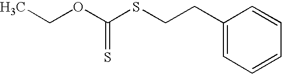

Dithiocarbonic acid O-ethyl ester S-phenethyl ester

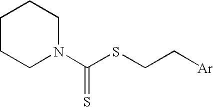

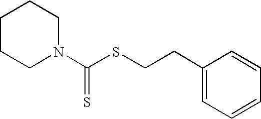

Piperidine-1-carbadithioic acid phenethyl ester
Phosphate Derivatives

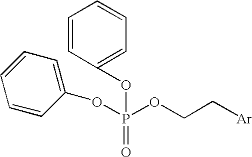

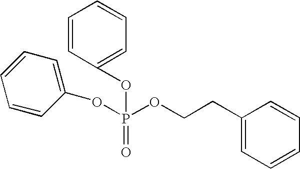

Phosphoric acid phenethyl ester diphenyl ester

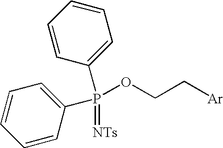

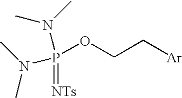

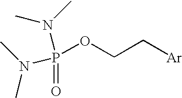

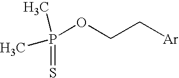

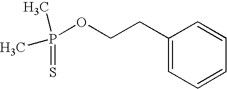

Dimethyl-phosphinothioic acid O-phenethyl ester
Other Examples of Formula (54)*

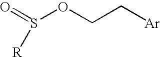

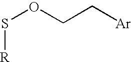

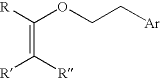

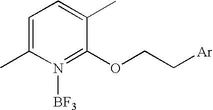

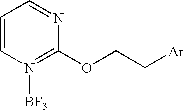

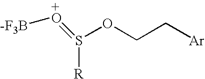

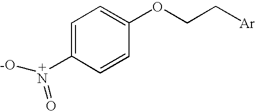

*For a review of the application of various examples of formula (54)* in the formation of an ether compound with an alcohol see for example, Toshima K. and Tatsuta K. Chem. Rev. 1993, 93, 1503, Tsuda T., Nakamura S. and Hashimoto S. Tetrahedron Lett. 2003, 44, 6453, Martichonok V. and Whitesides G. M. J. Org. Chem., 1996, 61, 1702 and references cited therein.

In a separate step, the resulted compound (55) is treated under suitable conditions with an amino compound of formula (56) to form compound (57) as the product. The reaction may be carried out with or without a solvent and at an appropriate temperature range that allows the formation of the product (57) at a suitable rate. An excess of the amino compound (56) may be used to maximally convert compound (55) to the product (57). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 5) generates the compound of formula (57) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

In one embodiment, the present invention provides a process for the preparation of a stereoisomerically substantially pure compound of formula (57): 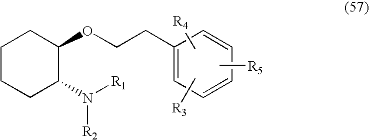
wherein, independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or
R₁ and R₂, when taken together with the nitrogen atom to which they are directly attached in formula (57), form a ring denoted by formula (I): 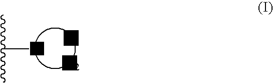
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; and any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (57), form a ring denoted by formula (II): 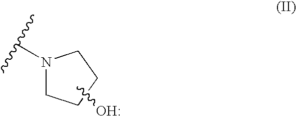
or in another embodiment R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl; or
preferably R₃, R₄ and R₅ are independently hydrogen, hydroxy or C₁-C₆alkoxy; with the proviso that R₃, R₄ and R₅ cannot all be hydrogen;
comprising the steps of starting with a monohalobenzene (49), wherein X may be F, Cl, Br or I; and following a reaction sequence as outlined in FIG. 5 under suitable conditions, wherein
—O-Q represents a good leaving group which on reaction with a hydroxy function will result in the formation of an ether compound with retention of the stereochemical configuration of the hydroxy function; and
—O-J represents a good leaving group on reaction with a nucleophilic reactant will result in a substitution product with substantial inversion of the stereochemical configuration of the activated hydroxy group as shown in FIG. 5; and all the formulae and symbols are as described above.

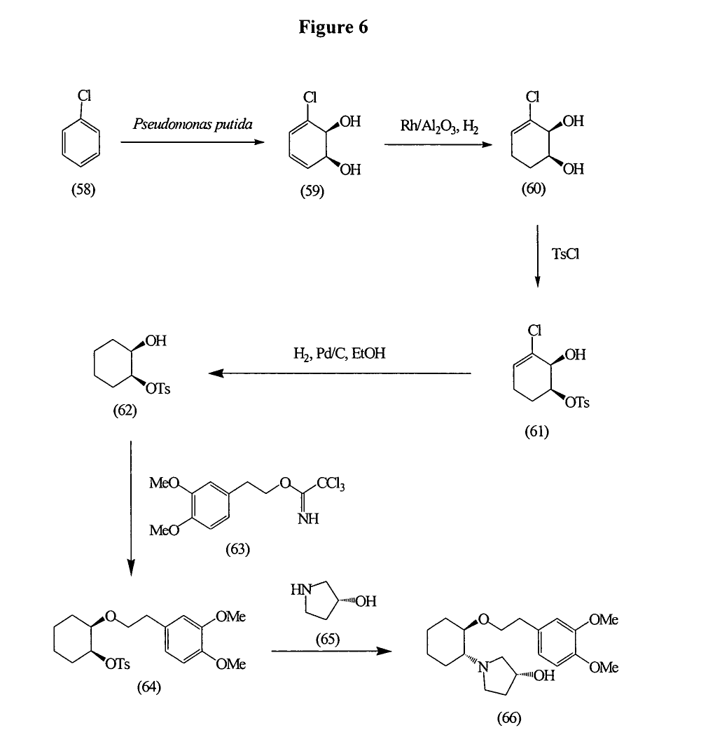

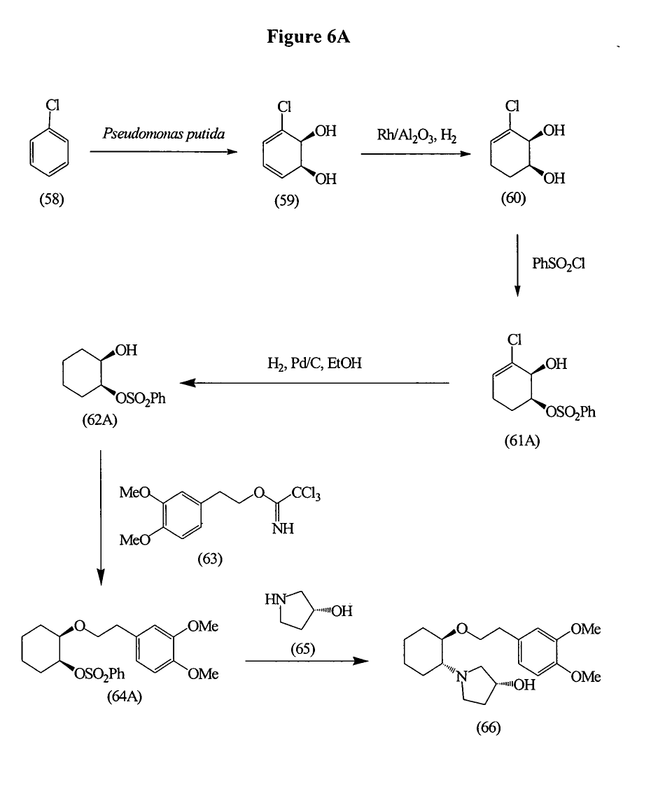

In another embodiment, the present invention provides a process for the preparation of a stereoisomerically substantially pure compound of formula (66), comprising the steps under suitable conditions as shown in FIG. 6, wherein all the formulae and symbols are as described above. As outlined in FIG. 6, the preparation of a stereoisomerically substantially pure trans aminocyclohexyl ether compound of formula (66) may be carried out by starting with a biotransformation of chlorobenzene (58) to compound (59) by microorganism such as Pseudomonas putida 39/D. Experimental conditions for the biotransformation are well established (Organic Synthesis, Vol. 76, 77 and T. Hudlicky et al., Aldrichimica Acta, 1999, 32, 35; and references cited therein). In a separate step, compound (59) is selectively reduced under suitable conditions to compound (60) (e.g., H₂—Rh/Al₂O₃; Boyd et al. JCS Chem. Commun. 1996, 45-46; Ham and Coker, J. Org. Chem. 1964, 29, 194-198; and references cited therein). In another separate step, the less hindered hydroxy group of formula (60) is selectively converted under suitable conditions into an activated form such as the tosylate (TsO—) of formula (61) (e.g., TsCl in the presence of pyridine). In a separate step, compound (61) is converted to compound (62) by reduction such as hydrogenation and hydrogenolysis in the presence of a catalyst under appropriate conditions. Palladium on activated carbon is one example of the catalysts. The reduction of compound (61) may be conducted under basic conditions e.g., in the presence of a base such as sodium ethoxide, sodium bicarbonate, sodium acetate or calcium carbonate. The base may be added in one portion or incrementally during the course of the reaction. In another separate step, the free hydroxy group in compound (62) is alkylated under appropriate conditions to form compound (64). The trichloroacetimidate (63) is readily prepared from the corresponding alcohol, 3,4-dimethoxyphenethyl alcohol which is commercially available (e.g., Aldrich), by treatment with trichloroacetonitrile. The alkylation of compound (62) by trichloroacetimidate (63) may be carried out in the presence of a Bronsted acid or Lewis acid such as HBF₄. In a separate step, the tosylate group of formula (64) is displaced by an amino compound such as 3R-pyrrolidinol (65) with inversion of configuration. 3R-pyrrolidinol (65) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

In another embodiment, the preparation of a stereoisomerically substantially pure trans-aminocyclohexyl ether compound of formula (66) may be carried out under suitable conditions by a process as outlined in FIG. 6A, comprising the steps of starting with a compound of formula (58) and following a reaction sequence analogous to the applicable portion that is described in FIG. 6. In FIG. 6A, the less hindered hydroxyl group of compound (60) is selectively converted under suitable conditions into an activated benzene sulfonic acid compound of formula (61A). In a separate step, compound (61A) is converted to compound (62A) by methods described in FIG. 6. Compound (62A) is reacted with compound (63) by methods described in FIG. 6 to provide compound (64A). In a separate step, the benzenesulfonate group of compound (64A) is displaced as described in FIG. 6 to provide compound (66).

The reaction sequences described above (FIG. 6 and FIG. 6A) in general generates the compound of formula (66) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

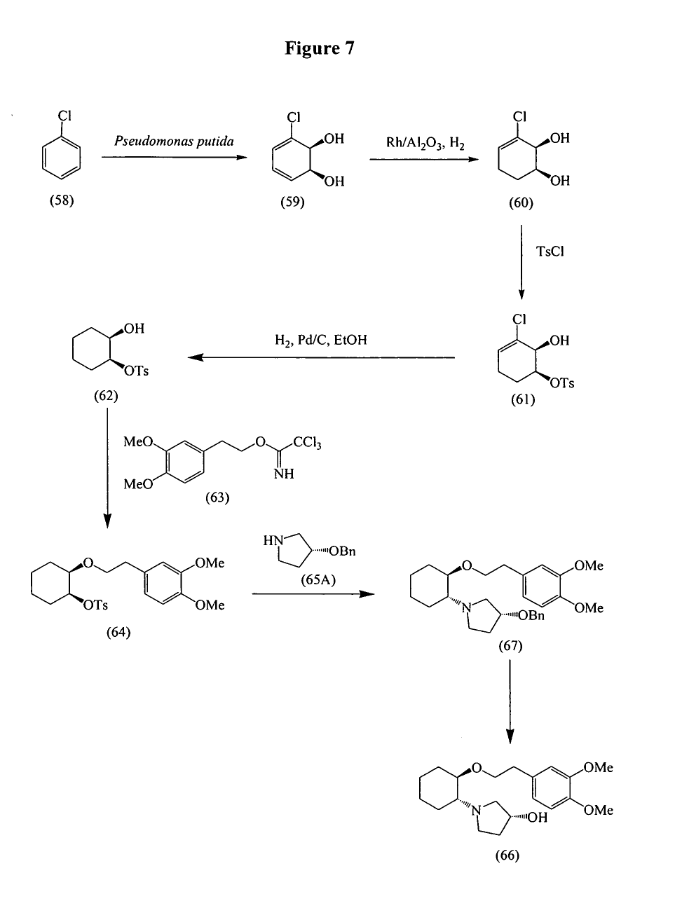

In another embodiment, the preparation of a stereoisomerically substantially pure trans aminocyclohexyl ether compound of formula (66) may be carried out under suitable conditions by a process as outlined in FIG. 7, comprising the steps of starting from chlorobenzene (58) and following a reaction sequence analogous to the applicable portion (i.e., rom compound (58) to compound (64)) that is described in FIG. 6 above leading to compound of formula (64). The latter is reacted under suitable conditions with an amino compound of formula (65A) wherein Bn represents a benzyl protection group of the hydroxy function of 3R-pyrrolidinol to form compound (67). Compound (65A) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (67) at a suitable rate. An excess of the amino compound (65A) may be used to maximally convert compound (64) to the product (67). The reaction may be performed in the presence of a base that can facilitate the formation of the product. Generally the additional base is non-nucleophilic in chemical reactivity. The benzyl (Bn) protection group of compound (67) may be removed by standard procedure (e.g., hydrogenation in the presence of a catalyst under appropriate conditions. Palladium on activated carbon is one example of the catalysts. Other suitable conditions are as described in Greene, “Protective Groups in Organic Chemistry”, John Wiley & Sons, New York N.Y. (1991)). The product is a stereoisomerically substantially pure trans aminocyclohexyl ether compound of formula (66) and is generally formed as the free base. The free base may be converted, if desired, to the monohydrochloride salt by known methodologies, or alternatively, if desired, to other acid addition salts by reaction with an inorganic or organic acids under appropriate conditions. Acid addition salts can also be prepared metathetically by reaction of one acid addition salt with an acid that is stronger than that giving rise to the initial salt.

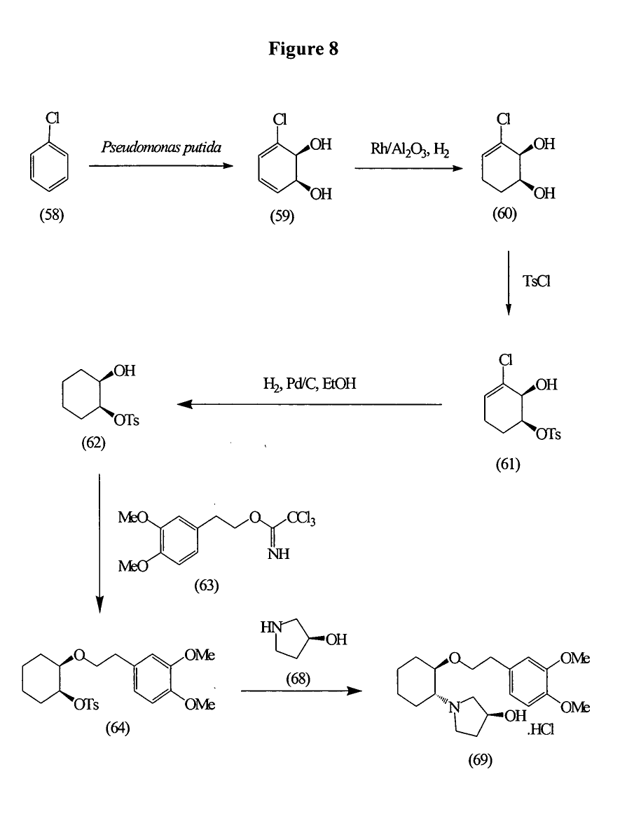

In another embodiment, the preparation of a stereoisomerically substantially pure trans aminocyclohexyl ether compound of formula (69) may be carried out under suitable conditions by a process as outlined in FIG. 8, comprising the steps of starting from chlorobenzene (58) and following a reaction sequence analogous to the applicable portion that is described in FIG. 6 above leading to compound of formula (64). The latter is reacted with an amino compound of formula (68). Compound (68), 3S-pyrrolidinol, is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (69) at a suitable rate. An excess of the amino compound (68) may be used to maximally convert compound (64) to the product (69). The reaction may be performed in the presence of a base that can facilitate the formation of the product. Generally the additional base is non-nucleophilic in chemical reactivity. The product is a stereoisomerically substantially pure trans aminocyclohexyl ether compound of formula (69) and is formed as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

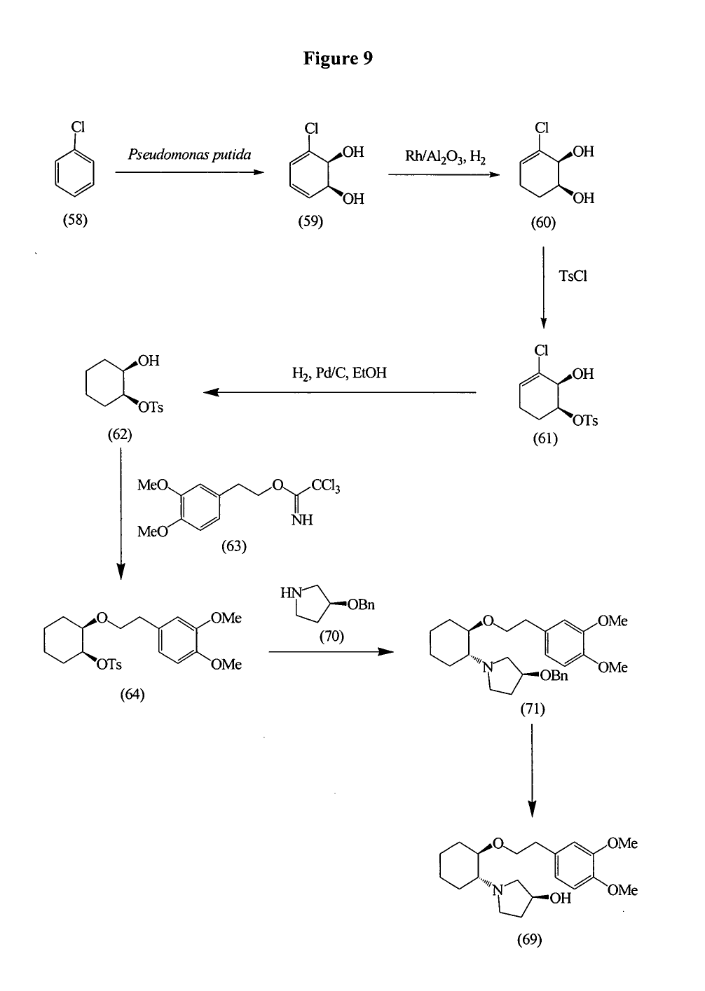

In another embodiment, the preparation of a stereoisomerically substantially pure trans aminocyclohexyl ether compound of formula (69) may be carried out under suitable conditions by a process as outlined in FIG. 9, comprising the steps of starting from chlorobenzene (58) and following a reaction sequence analogous to the applicable portion that is described in FIG. 7 above leading to compound of formula (64). The latter is reacted with an amino compound of formula (70) wherein Bn represents a benzyl protection group of the hydroxy function of 3S-pyrrolidinol to form compound (71). Compound (70) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (71) at a suitable rate. An excess of the amino compound (70) may be used to maximally convert compound (64) to the product (71). The reaction may be performed in the presence of a base that can facilitate the formation of the product. Generally the additional base is non-nucleophilic in chemical reactivity. The benzyl (Bn) protection group of compound (71) may be removed by standard procedure (e.g., hydrogenation in the presence of a catalyst under appropriate conditions. Palladium on activated carbon is one example of the catalysts. Other suitable conditions are as described in Greene, “Protective Groups in Organic Chemistry”, John Wiley & Sons, New York N.Y. (1991)). The product is a stereoisomerically substantially pure trans aminocyclohexyl ether compound of formula (69) and is generally formed as the free base. The free base may be converted, if desired, to the monohydrochloride salt by known methodologies, or alternatively, if desired, to other acid addition salts by reaction with an inorganic or organic acids under appropriate conditions. Acid addition salts can also be prepared metathetically by reaction of one acid addition salt with an acid that is stronger than that giving rise to the initial salt.

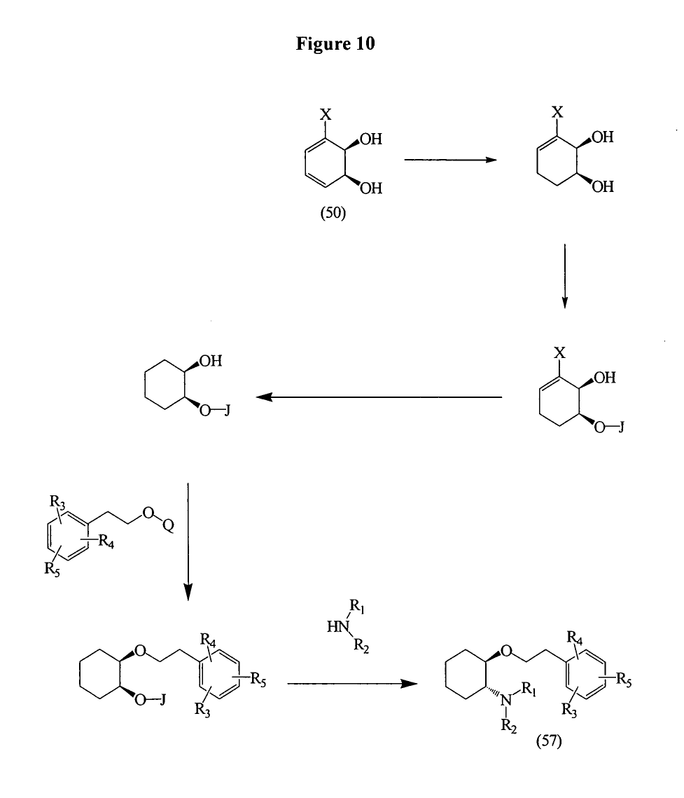

In another embodiment, the preparation of a stereoisomerically substantially pure trans aminocyclohexyl ether compound of formula (57) may be carried out under suitable conditions by a process as outlined in FIG. 10, comprising the steps of starting with compound of formula (50) and following a reaction sequence analogous to the applicable portion that is described in FIG. 5, wherein all the formulae and symbols are as described above.

In another embodiment, the preparation of a stereoisomerically substantially pure trans aminocyclohexyl ether compound of formula (66) may be carried out under suitable conditions by a process as outlined in FIG. 11, comprising the steps of starting with compound of formula (59) and following a reaction sequence analogous to the applicable portion that is described in FIG. 6, wherein all the formulae and symbols are as described above. 3-Chloro-(1S,2S)-3,5-cyclohexadiene-1,2-diol of formula (59) is a commercially available product (e.g., Aldrich) or synthesized according to published procedure (e.g., Organic Synthesis, Vol. 76, 77 and T. Hudlicky et al., Aldrichimica Acta, 1999, 32, 35; and references cited therein).

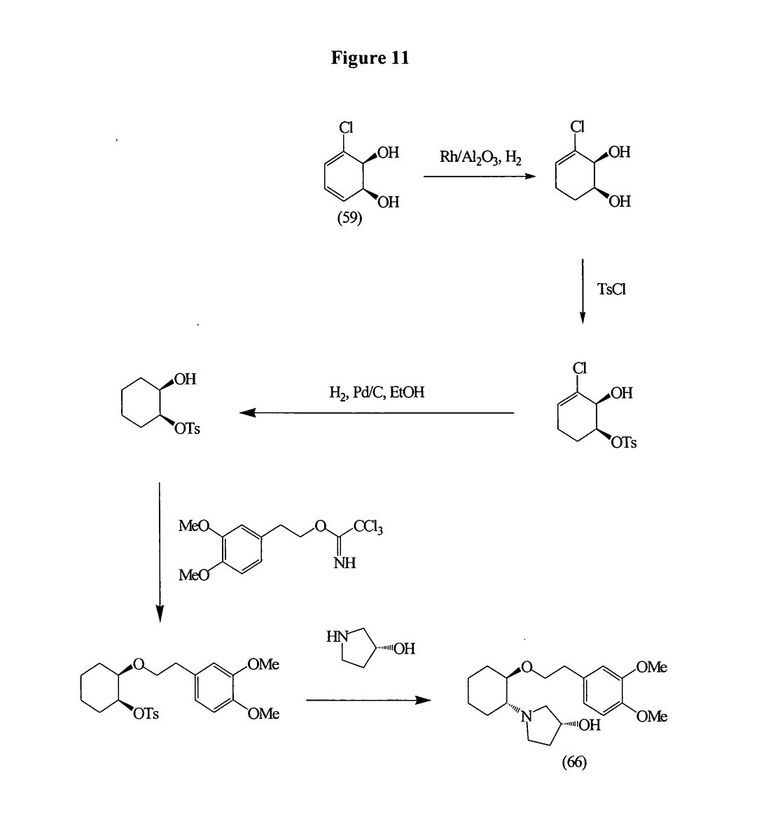

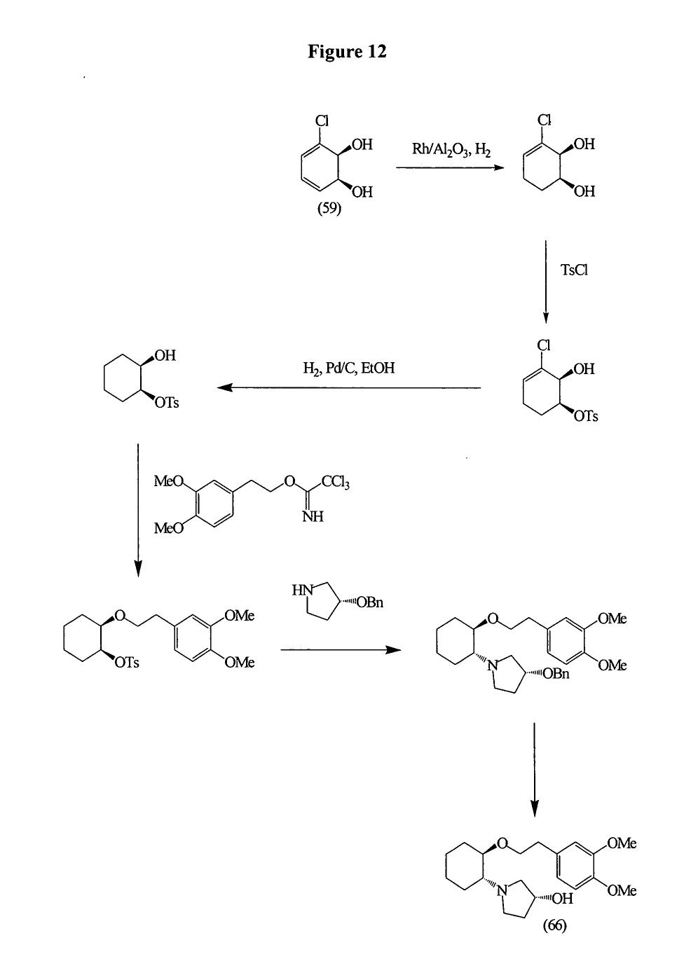

In another embodiment, the preparation of a stereoisomerically substantially pure trans aminocyclohexyl ether compound of formula (66) may be carried out under suitable conditions by a process as outlined in FIG. 12, comprising the steps of starting with compound of formula (59) and following a reaction sequence analogous to the applicable portion that is described in FIG. 7, wherein all the formulae and symbols are as described above.

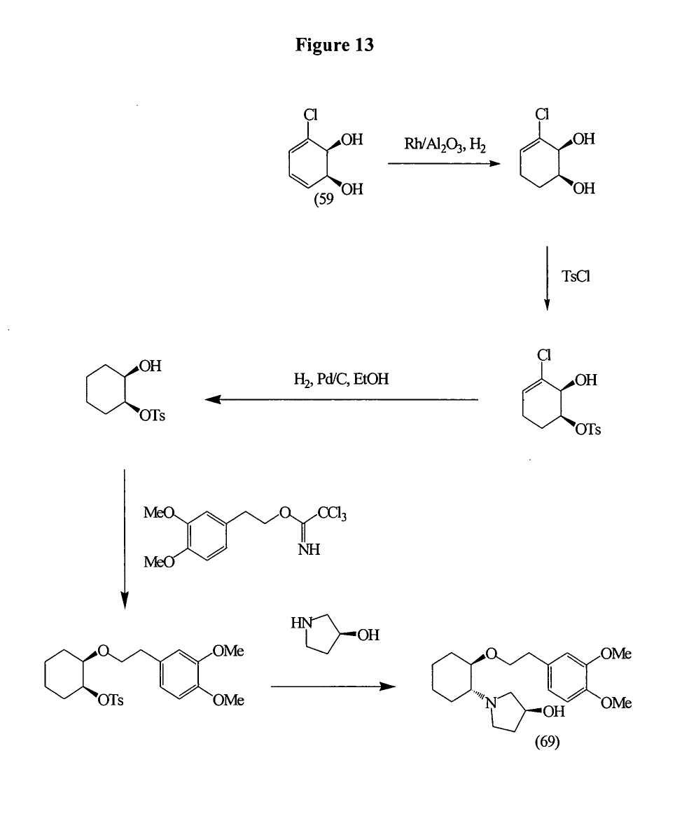

In another embodiment, the preparation of a stereoisomerically substantially pure trans aminocyclohexyl ether compound of formula (69) may be carried out under suitable conditions by a process as outlined in FIG. 13, comprising the steps of starting with compound of formula (59) and following a reaction sequence analogous to the applicable portion that is described in FIG. 8, wherein all the formulae and symbols are as described above.

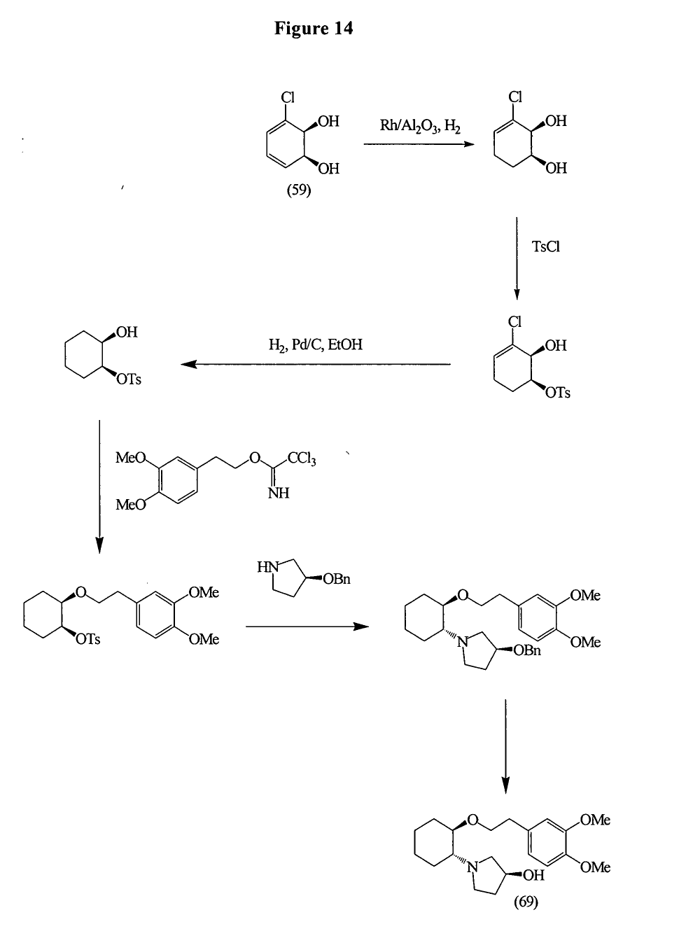

In another embodiment, the preparation of a stereoisomerically substantially pure trans aminocyclohexyl ether compound of formula (69) may be carried out under suitable conditions by a process as outlined in FIG. 14, comprising the steps of starting with compound of formula (59) and following a reaction sequence analogous to the applicable portion that is described in FIG. 9, wherein all the formulae and symbols are as described above.

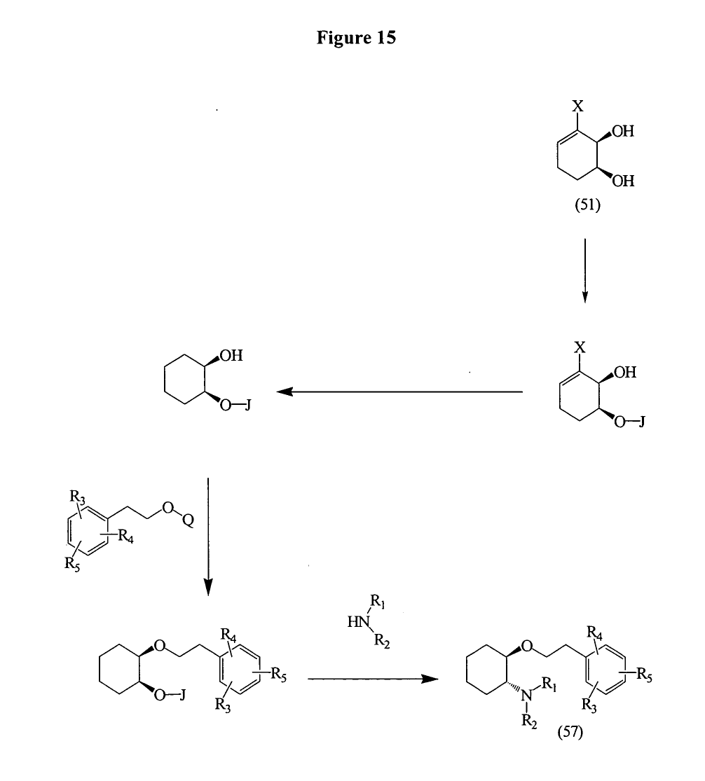

In another embodiment, the preparation of a stereoisomerically substantially pure trans aminocyclohexyl ether compound of formula (57) may be carried out under suitable conditions by a process as outlined in FIG. 15, comprising the steps of starting with compound of formula (51) and following a reaction sequence analogous to the applicable portion that is described in FIG. 5, wherein all the formulae and symbols are as described above.

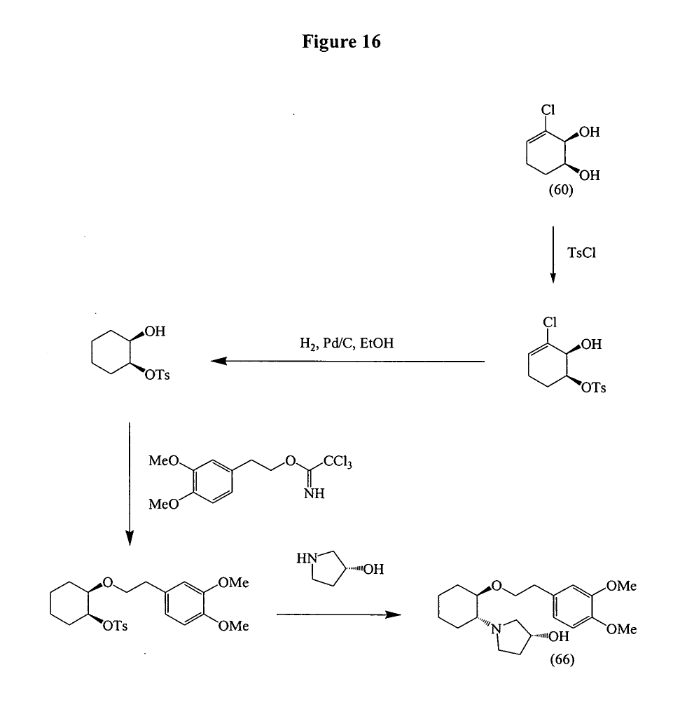

In another embodiment, the preparation of a stereoisomerically substantially pure trans aminocyclohexyl ether compound of formula (66) may be carried out under suitable conditions by a process as outlined in FIG. 16, comprising the steps of starting with compound of formula (60) and following a reaction sequence analogous to the applicable portion that is described in FIG. 6, wherein all the formulae and symbols are as described above.

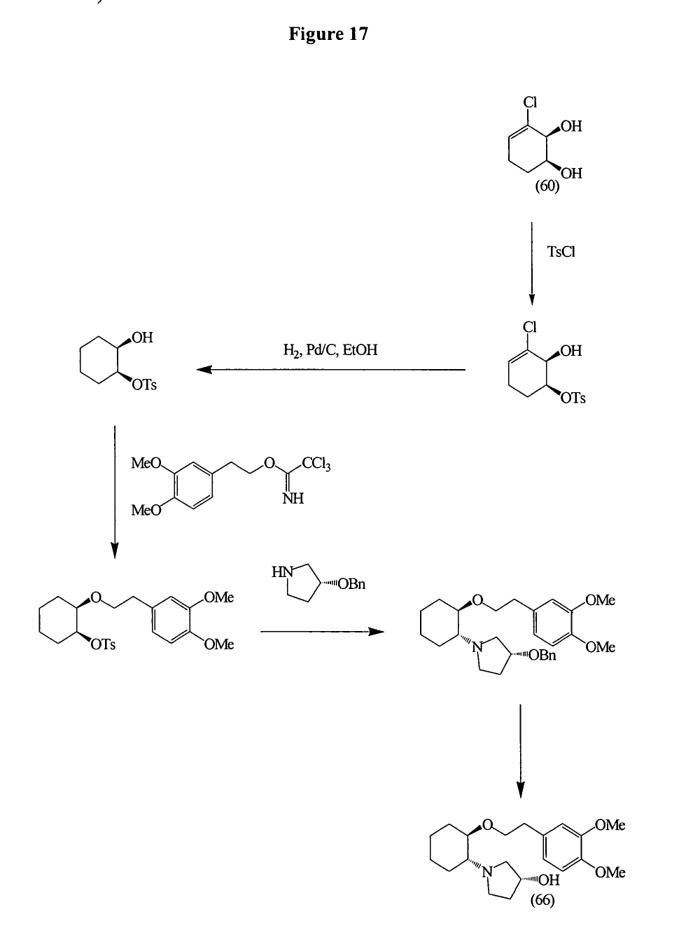

In another embodiment, the preparation of a stereoisomerically substantially pure trans aminocyclohexyl ether compound of formula (66) may be carried out under suitable conditions by a process as outlined in FIG. 17, comprising the steps of starting with compound of formula (60) and following a reaction sequence analogous to the applicable portion that is described in FIG. 7, wherein all the formulae and symbols are as described above.

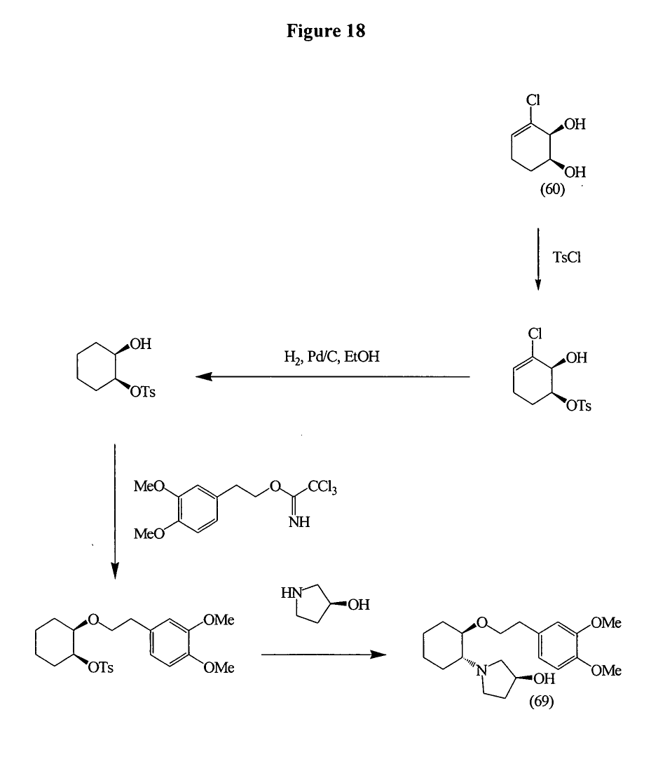

In another embodiment, the preparation of a stereoisomerically substantially pure trans aminocyclohexyl ether compound of formula (69) may be carried out under suitable conditions by a process as outlined in FIG. 18, comprising the steps of starting with compound of formula (60) and following a reaction sequence analogous to the applicable portion that is described in FIG. 8, wherein all the formulae and symbols are as described above.

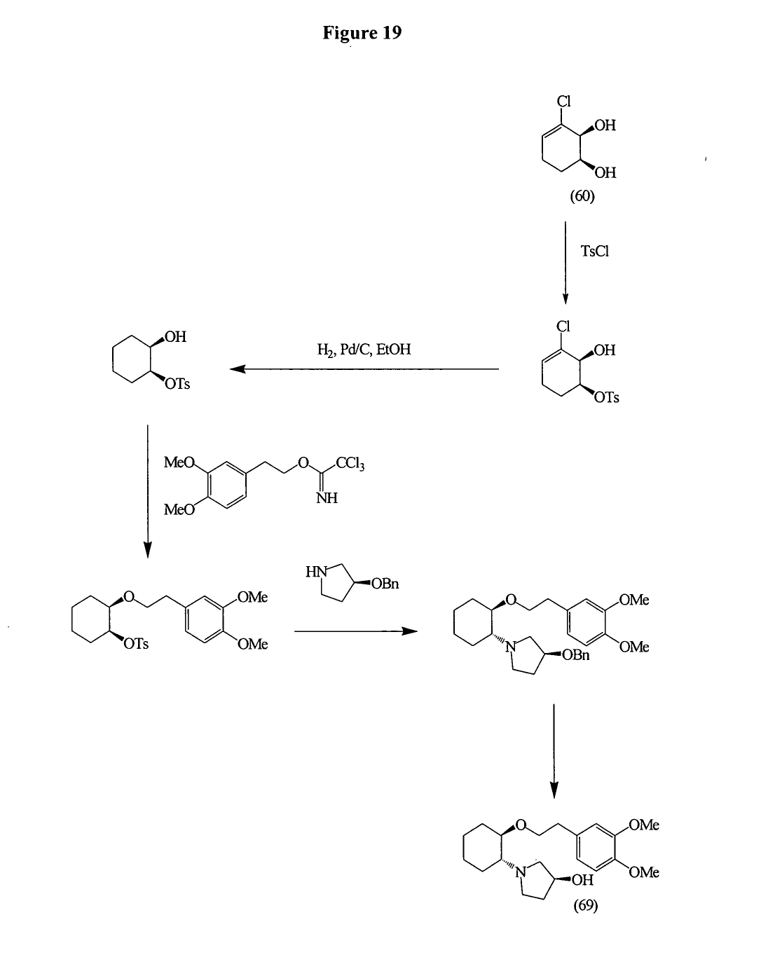

In another embodiment, the preparation of a stereoisomerically substantially pure trans aminocyclohexyl ether compound of formula (69) may be carried out under suitable conditions by a process as outlined in FIG. 19, comprising the steps of starting with compound of formula (60) and following a reaction sequence analogous to the applicable portion that is described in FIG. 9, wherein all the formulae and symbols are as described above.

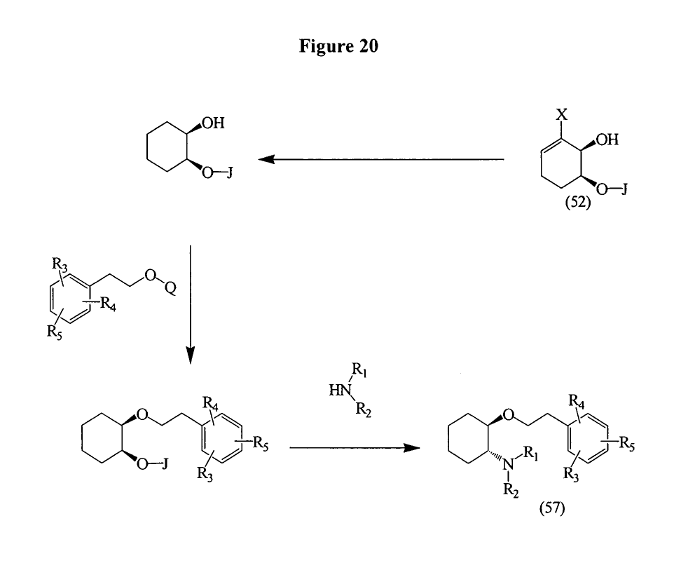

In another embodiment, the preparation of a stereoisomerically substantially pure trans aminocyclohexyl ether compound of formula (57) may be carried out under suitable conditions by a process as outlined in FIG. 20, comprising the steps of starting with compound of formula (52) and following a reaction sequence analogous to the applicable portion that is described in FIG. 5, wherein all the formulae and symbols are as described above.

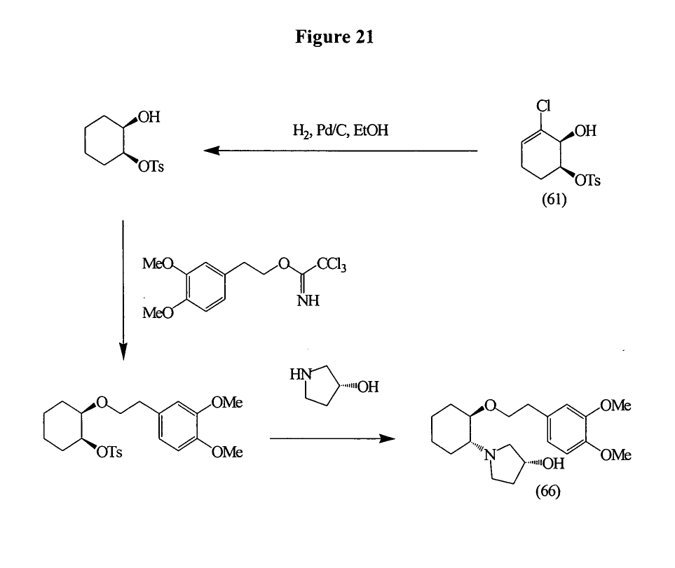

In another embodiment, the preparation of a stereoisomerically substantially pure trans aminocyclohexyl ether compound of formula (66) may be carried out under suitable conditions by a process as outlined in FIG. 21, comprising the steps of starting with compound of formula (61) and following a reaction sequence analogous to the applicable portion that is described in FIG. 6, wherein all the formulae and symbols are as described above.

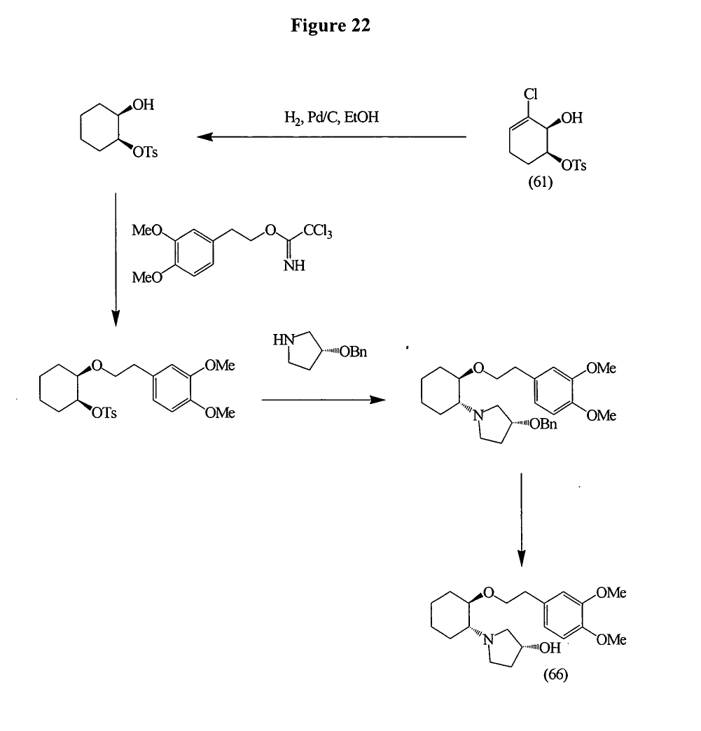

In another embodiment, the preparation of a stereoisomerically substantially pure trans aminocyclohexyl ether compound of formula (66) may be carried out under suitable conditions by a process as outlined in FIG. 22, comprising the steps of starting with compound of formula (61) and following a reaction sequence analogous to the applicable portion that is described in FIG. 7, wherein all the formulae and symbols are as described above.

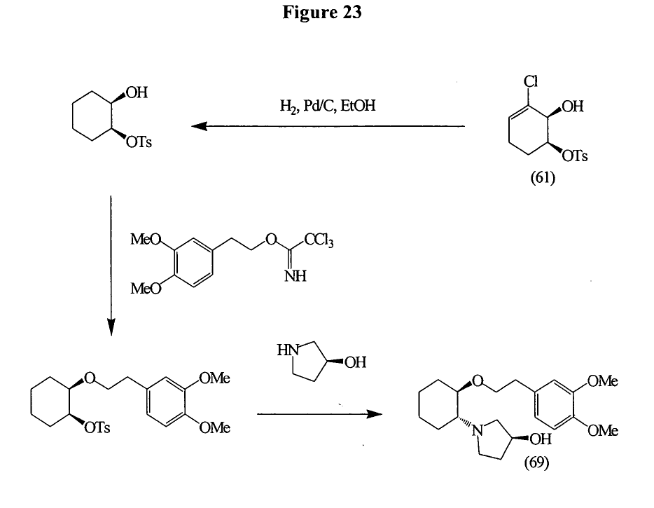

In another embodiment, the preparation of a stereoisomerically substantially pure trans aminocyclohexyl ether compound of formula (69) may be carried out under suitable conditions by a process as outlined in FIG. 23, comprising the steps of starting with compound of formula (61) and following a reaction sequence analogous to the applicable portion that is described in FIG. 8, wherein all the formulae and symbols are as described above.

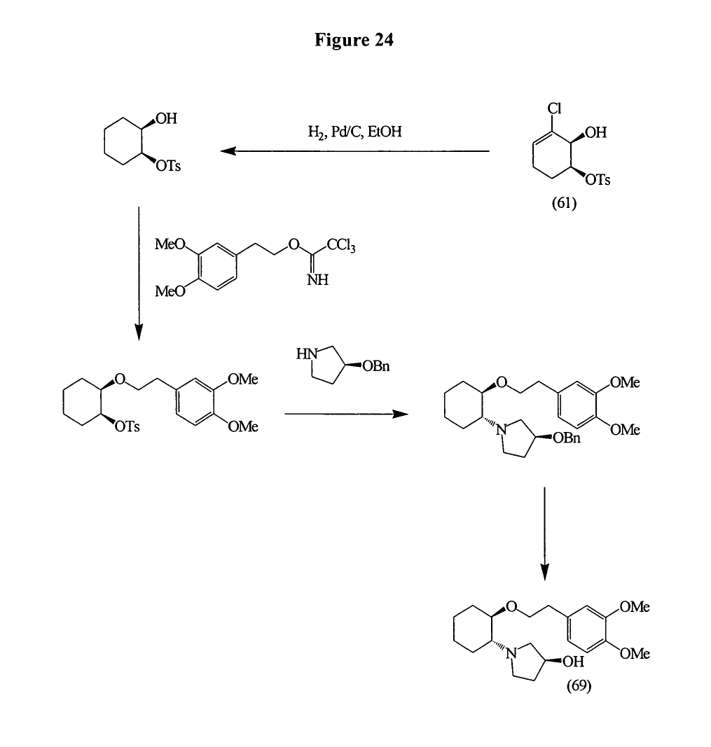

In another embodiment, the preparation of a stereoisomerically substantially pure trans aminocyclohexyl ether compound of formula (69) may be carried out under suitable conditions by a process as outlined in FIG. 24, comprising the steps of starting with compound of formula (61) and following a reaction sequence analogous to the applicable portion that is described in FIG. 9, wherein all the formulae and symbols are as described above.

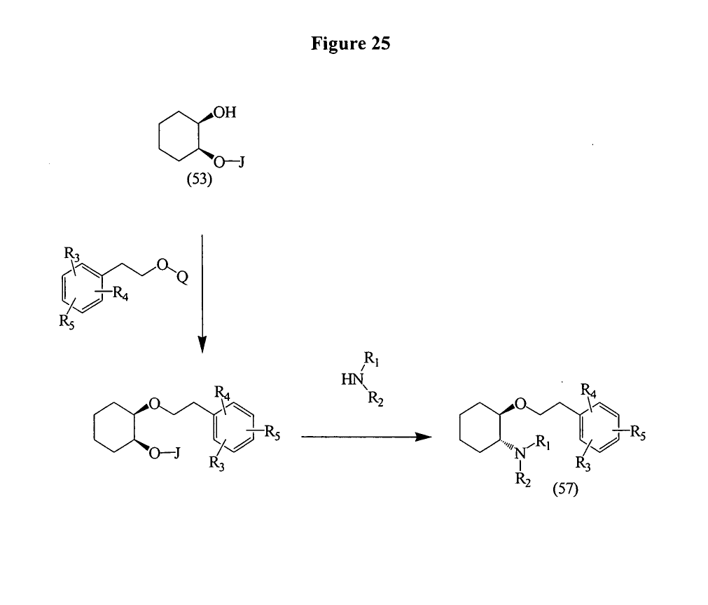

In another embodiment, the preparation of a stereoisomerically substantially pure trans aminocyclohexyl ether compound of formula (57) may be carried out under suitable conditions by a process as outlined in FIG. 25, comprising the steps of starting with compound of formula (53) and following a reaction sequence analogous to the applicable portion that is described in FIG. 5, wherein all the formulae and symbols are as described above.

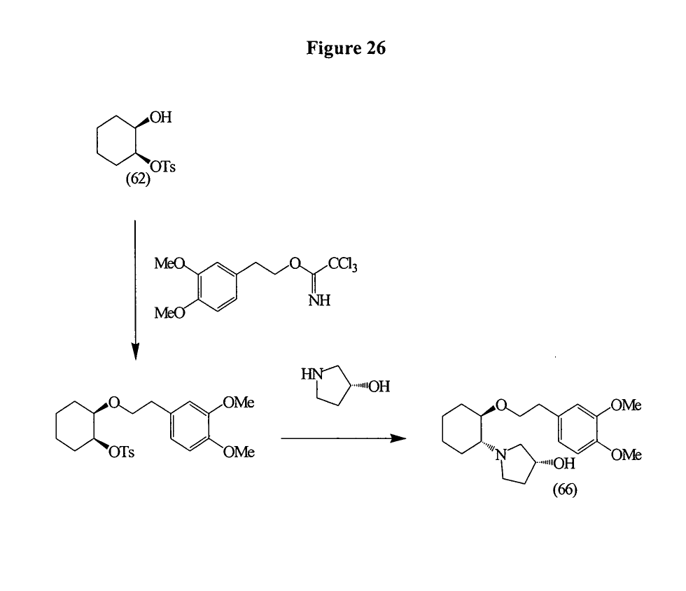

In another embodiment, the preparation of a stereoisomerically substantially pure trans aminocyclohexyl ether compound of formula (66) may be carried out under suitable conditions by a process as outlined in FIG. 26, comprising the steps of starting with compound of formula (62) and following a reaction sequence analogous to the applicable portion that is described in FIG. 6, wherein all the formulae and symbols are as described above.

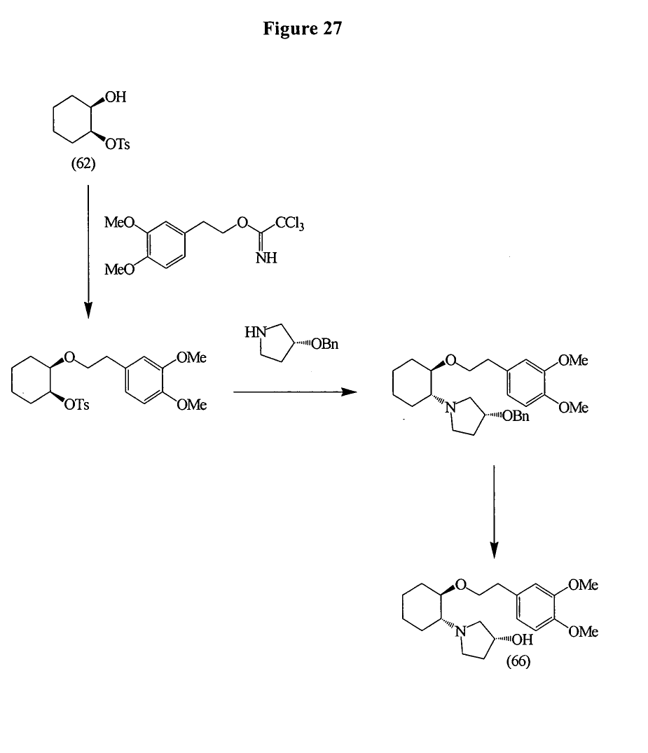

In another embodiment, the preparation of a stereoisomerically substantially pure trans aminocyclohexyl ether compound of formula (66) may be carried out under suitable conditions by a process as outlined in FIG. 27, comprising the steps of starting with compound of formula (62) and following a reaction sequence analogous to the applicable portion that is described in FIG. 7, wherein all the formulae and symbols are as described above.

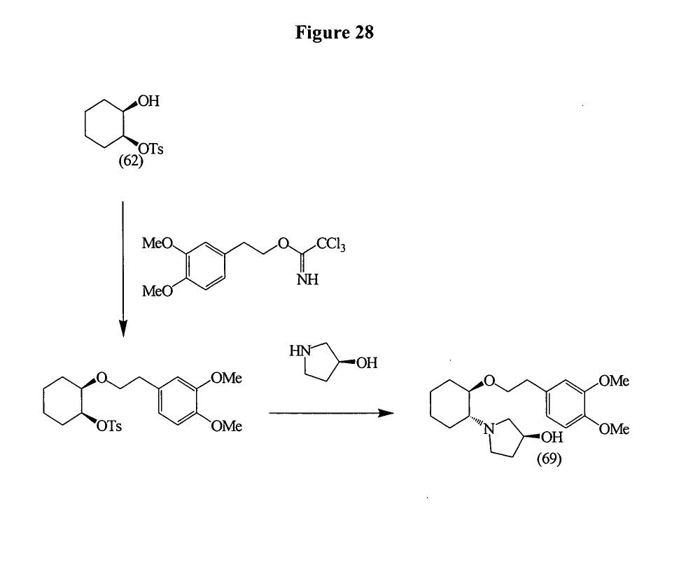

In another embodiment, the preparation of a stereoisomerically substantially pure trans aminocyclohexyl ether compound of formula (69) may be carried out under suitable conditions by a process as outlined in FIG. 28, comprising the steps of starting with compound of formula (62) and following a reaction sequence analogous to the applicable portion that is described in FIG. 8, wherein all the formulae and symbols are as described above.

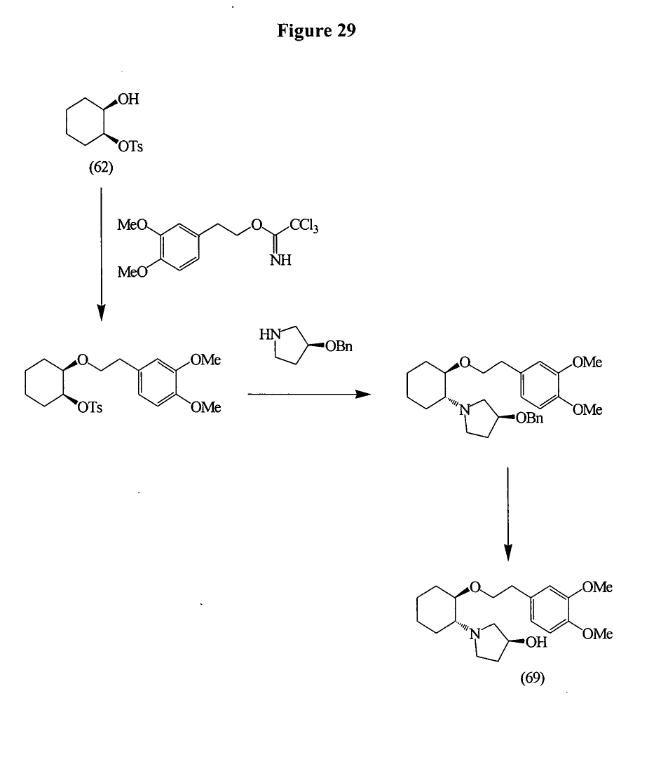

In another embodiment, the preparation of a stereoisomerically substantially pure trans aminocyclohexyl ether compound of formula (69) may be carried out under suitable conditions by a process as outlined in FIG. 29, comprising the steps of starting with compound of formula (62) and following a reaction sequence analogous to the applicable portion that is described in FIG. 9, wherein all the formulae and symbols are as described above.

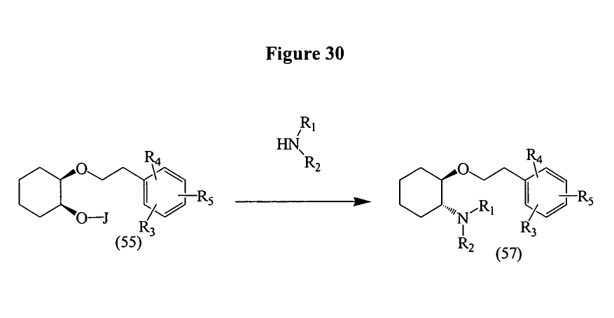

In another embodiment, the preparation of a stereoisomerically substantially pure trans aminocyclohexyl ether compound of formula (57) may be carried out under suitable conditions by a process as outlined in FIG. 30, comprising the steps of starting with compound of formula (55) and following a reaction sequence analogous to the applicable portion that is described in FIG. 5, wherein all the formulae and symbols are as described above.

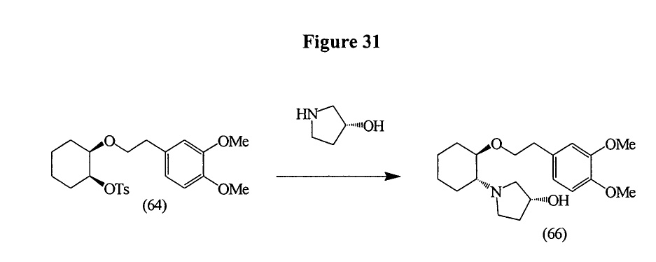

In another embodiment, the preparation of a stereoisomerically substantially pure trans aminocyclohexyl ether compound of formula (66) may be carried out under suitable conditions by a process as outlined in FIG. 31, comprising the steps of starting with compound of formula (64) and following a reaction sequence analogous to the applicable portion that is described in FIG. 6, wherein all the formulae and symbols are as described above.

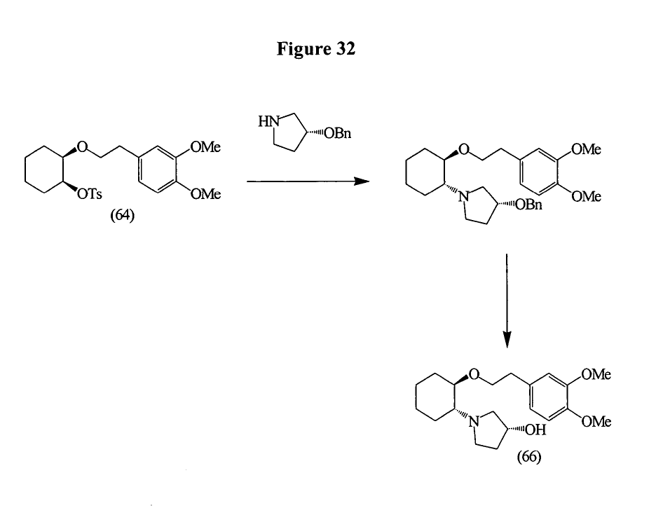

In another embodiment, the preparation of a stereoisomerically substantially pure trans aminocyclohexyl ether compound of formula (66) may be carried out under suitable conditions by a process as outlined in FIG. 32, comprising the steps of starting with compound of formula (64) and following a reaction sequence analogous to the applicable portion that is described in FIG. 7, wherein all the formulae and symbols are as described above.

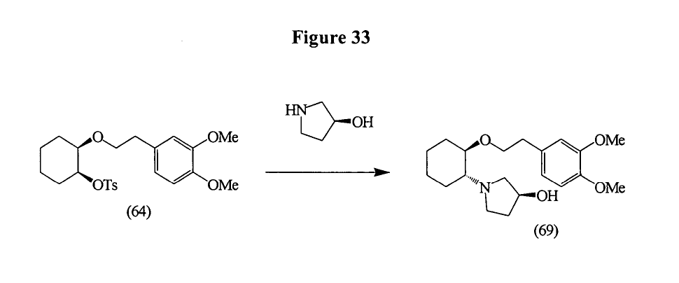

In another embodiment, the preparation of a stereoisomerically substantially pure trans aminocyclohexyl ether compound of formula (69) may be carried out under suitable conditions by a process as outlined in FIG. 33, comprising the steps of starting with compound of formula (64) and following a reaction sequence analogous to the applicable portion that is described in FIG. 8, wherein all the formulae and symbols are as described above.

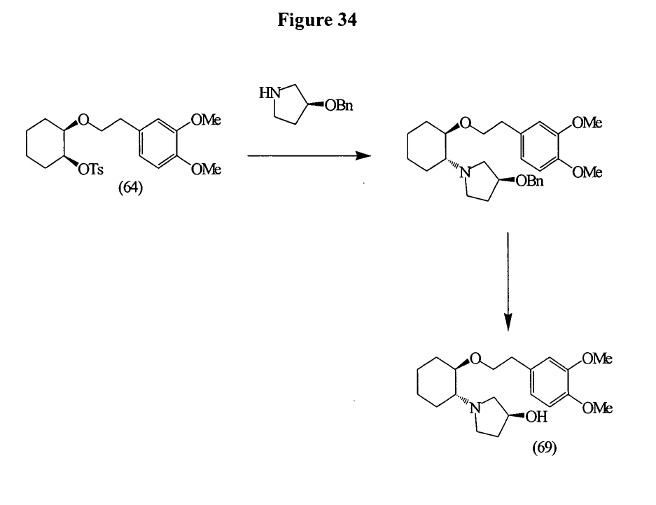

In another embodiment, the preparation of a stereoisomerically substantially pure trans aminocyclohexyl ether compound of formula (69) may be carried out under suitable conditions by a process as outlined in FIG. 34, comprising the steps of starting with compound of formula (64) and following a reaction sequence analogous to the applicable portion that is described in FIG. 9, wherein all the formulae and symbols are as described above.

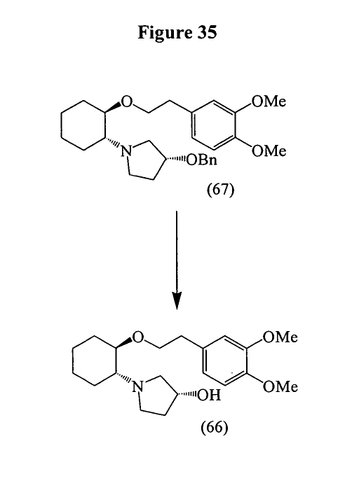

In another embodiment, the preparation of a stereoisomerically substantially pure trans aminocyclohexyl ether compound of formula (66) may be carried out under suitable conditions by a process as outlined in FIG. 35, comprising the steps of starting with compound of formula (67) and following a reaction sequence analogous to the applicable portion that is described in FIG. 7, wherein all the formulae and symbols are as described above.

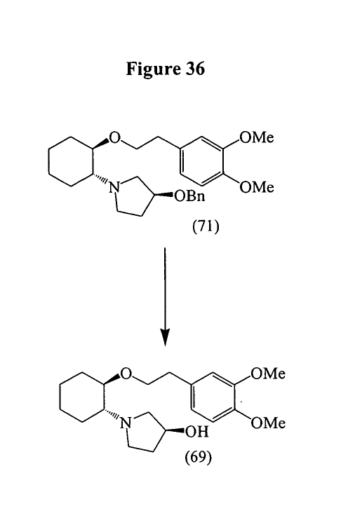

In another embodiment, the preparation of a stereoisomerically substantially pure trans aminocyclohexyl ether compound of formula (69) may be carried out under suitable conditions by a process as outlined in FIG. 36, comprising the steps of starting with compound of formula (71) and following a reaction sequence analogous to the applicable portion that is described in FIG. 9, wherein all the formulae and symbols are as described above.

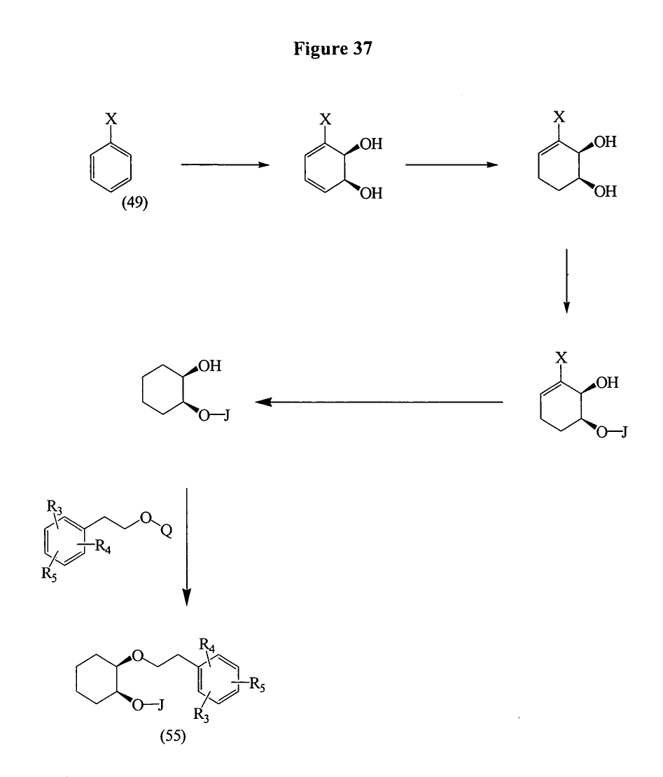

In another embodiment, the preparation of a stereoisomerically substantially pure compound of formula (55) may be carried out under suitable conditions by a process as outlined in FIG. 37, comprising the steps of starting with compound of formula (49) and following a reaction sequence analogous to the applicable portion that is described in FIG. 5, wherein all the formulae and symbols are as described above.

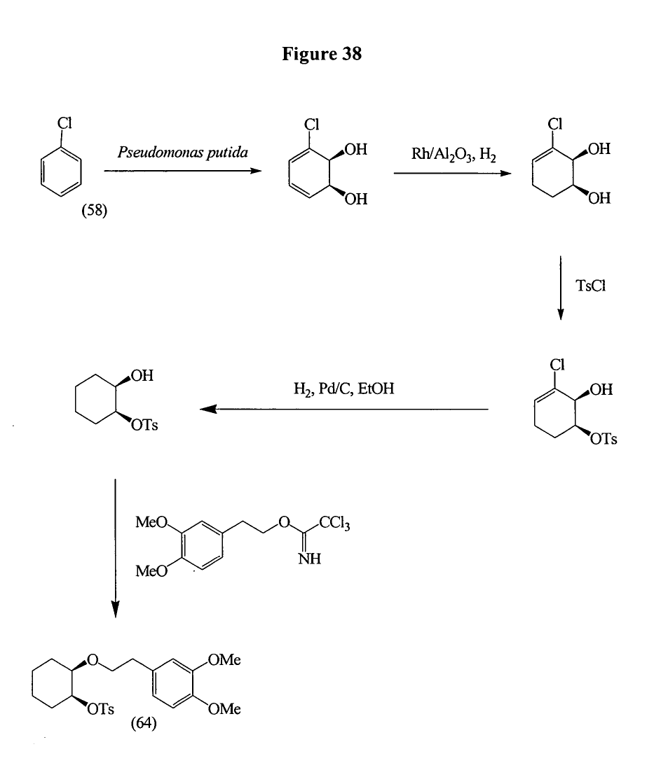

In another embodiment, the preparation of a stereoisomerically substantially pure compound of formula (64) may be carried out under suitable conditions by a process as outlined in FIG. 38, comprising the steps of starting with compound of formula (58) and following a reaction sequence analogous to the applicable portion that is described in FIG. 6, wherein all the formulae and symbols are as described above.

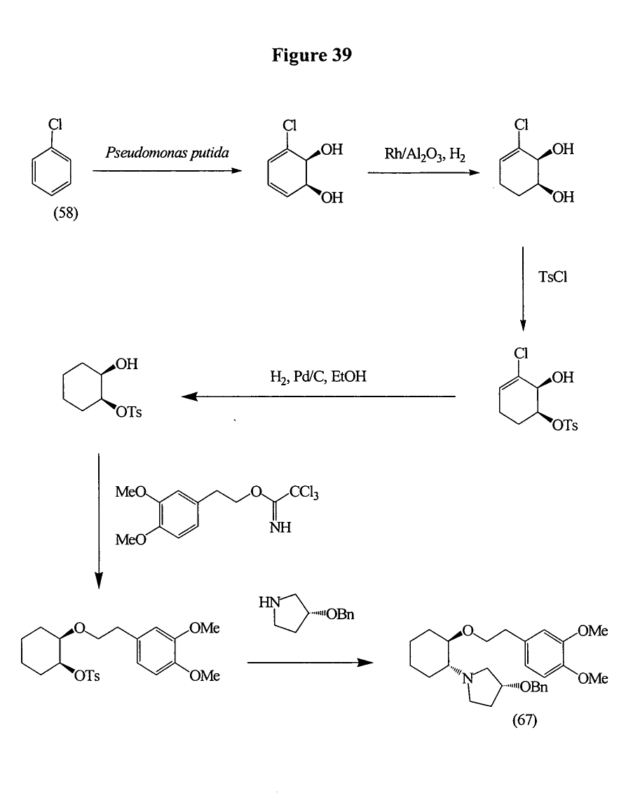

In another embodiment, the preparation of a stereoisomerically substantially pure trans aminocyclohexyl ether compound of formula (67) may be carried out under suitable conditions by a process as outlined in FIG. 39, comprising the steps of starting with compound of formula (58) and following a reaction sequence analogous to the applicable portion that is described in FIG. 7, wherein all the formulae and symbols are as described above.

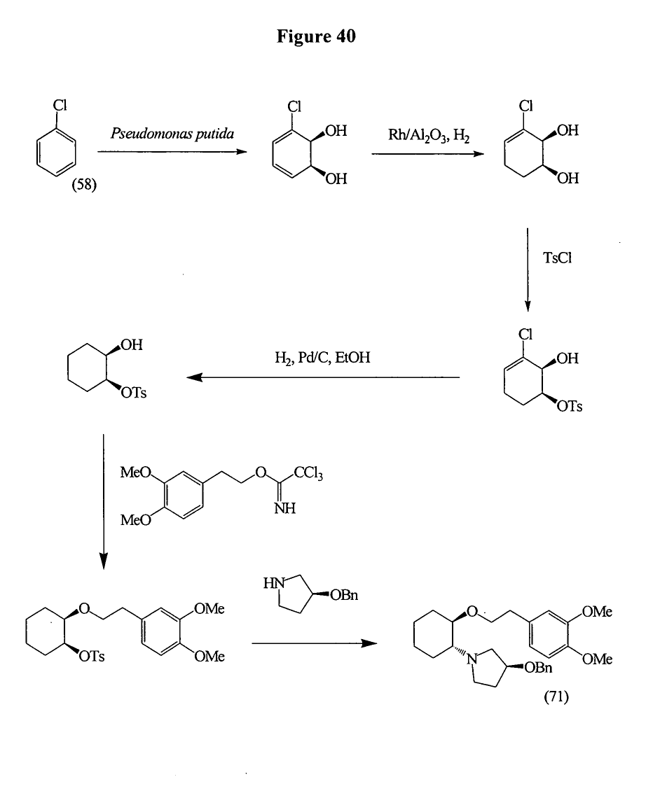

In another embodiment, the preparation of a stereoisomerically substantially pure trans aminocyclohexyl ether compound of formula (71) may be carried out under suitable conditions by a process as outlined in FIG. 40, comprising the steps of starting with compound of formula (58) and following a reaction sequence analogous to the applicable portion that is described in FIG. 9, wherein all the formulae and symbols are as described above.

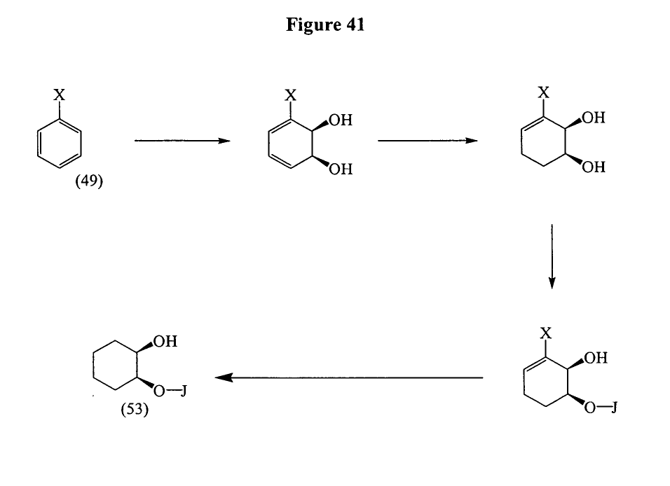

In another embodiment, the preparation of a stereoisomerically substantially pure compound of formula (53) may be carried out under suitable conditions by a process as outlined in FIG. 41, comprising the steps of starting with compound of formula (49) and following a reaction sequence analogous to the applicable portion that is described in FIG. 5, wherein all the formulae and symbols are as described above.

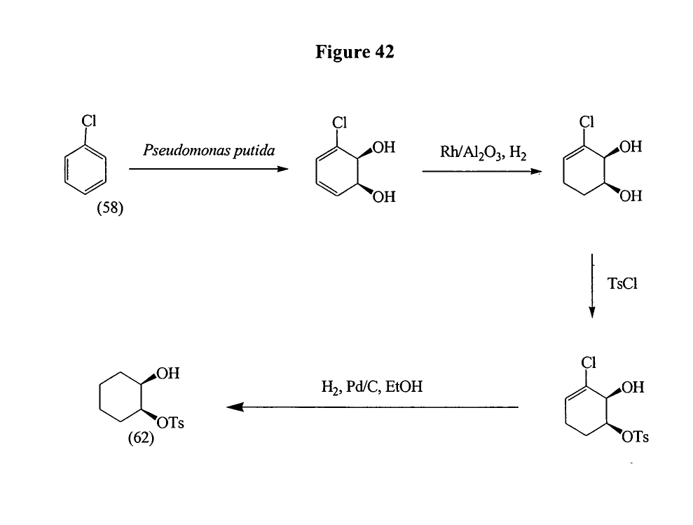

In another embodiment, the preparation of a stereoisomerically substantially pure compound of formula (62) may be carried out under suitable conditions by a process as outlined in FIG. 42, comprising the steps of starting with compound of formula (58) and following a reaction sequence analogous to the applicable portion that is described in FIG. 6, wherein all the formulae and symbols are as described above.

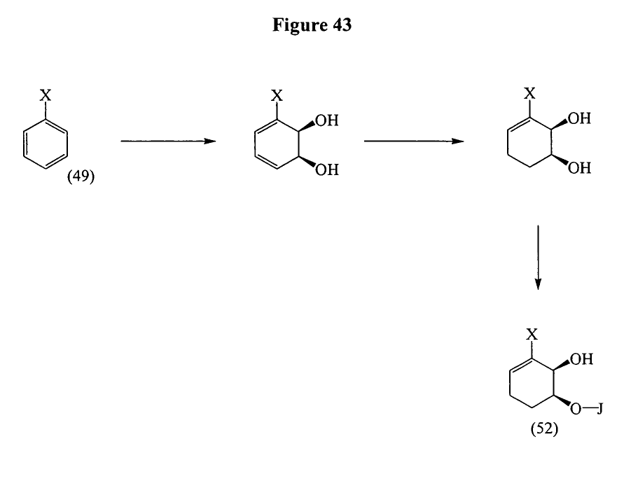

In another embodiment, the preparation of a stereoisomerically substantially pure compound of formula (52) may be carried out under suitable conditions by a process as outlined in FIG. 43, comprising the steps of starting with compound of formula (49) and following a reaction sequence analogous to the applicable portion that is described in FIG. 5, wherein all the formulae and symbols are as described above.

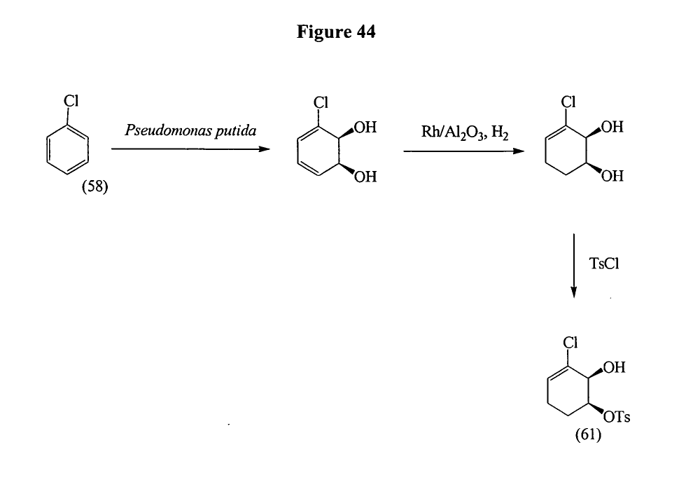

In another embodiment, the preparation of a stereoisomerically substantially pure compound of formula (61) may be carried out under suitable conditions by a process as outlined in FIG. 44, comprising the steps of starting with compound of formula (58) and following a reaction sequence analogous to the applicable portion that is described in FIG. 6, wherein all the formulae and symbols are as described above.

In another embodiment, the present invention provides a compound of formula (52), or a solvate or pharmaceutically acceptable salt thereof; wherein all the formulae and symbols are as described above.

In another embodiment, the present invention provides a compound of formula (53), or a solvate or pharmaceutically acceptable salt thereof; wherein all the formulae and symbols are as described above with the proviso that J is not a methanesulfonyl group or a tosyl group.

In another embodiment, the present invention provides a compound of formula (54), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (55), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (61) or (61A), or a solvate or pharmaceutically acceptable salt thereof; wherein all the formulae and symbols are as described above.

In another embodiment, the present invention provides a compound of formula (62A), or a solvate or pharmaceutically acceptable salt thereof; wherein all the formulae and symbols are as described above.

In another embodiment, the present invention provides a compound of formula (64) or (64A), or a solvate or pharmaceutically acceptable salt thereof; wherein all the formulae and symbols are as described above.

In another embodiment, the present invention provides a compound of formula (67) or (71), or a solvate or pharmaceutically acceptable salt thereof; wherein all the formulae and symbols are as described above.

In another embodiment, the present invention provides synthetic processes whereby compounds of formula (75) with trans-(1S,2S) configuration for the ether and amino functional groups may be prepared in stereoisomerically substantially pure form. Compounds of formulae (79), (80), (81) and (82) are some of the examples represented by formula (75). The present invention also provides synthetic processes whereby compounds of formulae (72), (73) and (74) may be synthesized in stereoisomerically substantially pure forms. Compounds (76), (77) and (78) are examples of formulae (72), (73) and (74) respectively.

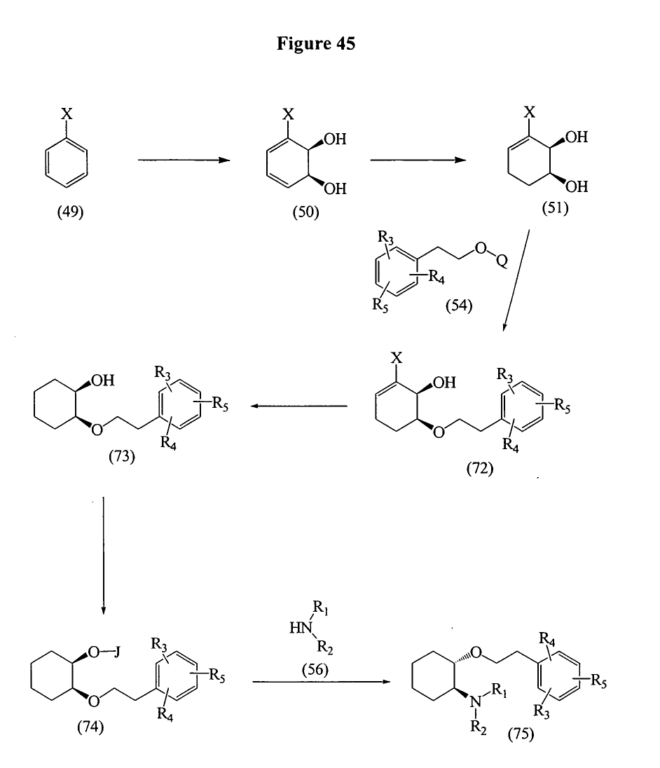

As outlined in FIG. 45, the preparation of a stereoisomerically substantially pure trans aminocyclohexyl ether compound of formula (75) may be carried out by following a process starting from a monohalobenzene (49), wherein X may be F, Cl, Br or I.

In a first step, compound (49) is transformed by well-established microbial oxidation to the cis-cyclohexandienediol (50) in stereoisomerically substantially pure form (T. Hudlicky et al., Aldrichimica Acta, 1999, 32, 35; and references cited therein). In a separate step, compound (50) may be selectively reduced under suitable conditions to compound (51) (e.g., H₂—Rh/Al₂O₃; Boyd et al. JCS Chem. Commun. 1996, 45-46; Ham and Coker, J. Org. Chem. 1964, 29, 194-198; and references cited therein). In another separate step, compound (51) is converted to compound (72) by reaction under appropriate conditions with an alkylating reagent such as compound (54), where —O-Q represents a good leaving group which on reaction with a hydroxy function will result in the formation of an ether compound with retention of the stereochemical configuration of the hydroxy function. Haloacetimidate (e.g., trifluoroacetimidate or trichloroacetimidate) is one example for the —O-Q function. For some compound (72), it may be necessary to introduce appropriate protection groups prior to this step being performed. Suitable protecting groups are set forth in, for example, Greene, “Protective Groups in Organic Chemistry”, John Wiley & Sons, New York N.Y. (1991).

In a separate step, transformation of compound (72) to compound (73) may be effected by hydrogenation and hydrogenolysis in the presence of a catalyst under appropriate conditions. Palladium on activated carbon is one example of the catalysts. Hydrogenolysis of alkyl or alkenyl halide such as (72) may be conducted under basic conditions. The presence of a base such as sodium ethoxide, sodium bicarbonate, sodium acetate or calcium carbonate is some possible examples. The base may be added in one portion or incrementally during the course of the reaction.

In another separate step, the hydroxy group of compound (73) is selectively converted under suitable conditions into an activated form as represented by compound (74). An “activated form” as used herein means that the hydroxy group is converted into a good leaving group (—O-J) which on reaction with an appropriate nucleophile (e.g., HNR₁R₂) will result in a substitution product with substantial inversion of the stereochemical configuration of the activated hydroxy group. The leaving group (—O-J) may be but is not limited to an alkyl sulfonate such as a trifluoromethanesulfonate group (CF₃SO₃—) or a mesylate group (MsO—), an aryl sulfonate such as a benzenesulfonate group (PhSO₃—), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. In a typical reaction for the formation of an alkyl sulfonate (e.g., a mesylate) or an aryl sulfonate (e.g., a tosylate or a nosylate), compound (73) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (73) may be used to maximally convert the hydroxy group into the activated form.

In a separate step, the resulted compound (74) is treated under suitable conditions with an amino compound of formula (56) to form compound (75) as the product. The reaction may be carried out with or without a solvent and at an appropriate temperature range that allows the formation of the product (75) at a suitable rate. An excess of the amino compound (56) may be used to maximally convert compound (74) to the product (75). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 45) generates the compound of formula (75) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

In one embodiment, the present invention provides a process for the preparation of a stereoisomerically substantially pure compound of formula (75): 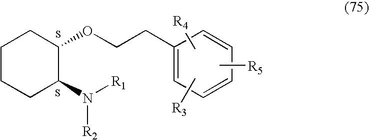
wherein, independently at each occurrence, R₁ and R₂ are independently selected from hydrogen, C₁-C₈alkyl, C₃-C₈alkoxyalkyl, C₁-C₈hydroxyalkyl, and C₇-C₁₂aralkyl; or R₁ and R₂ are independently selected from C₃-C₈alkoxyalkyl, C₁-C₈hydroxyalkyl, and C₇-C₁₂aralkyl; or
R₁ and R₂, when taken together with the nitrogen atom to which they are directly attached in formula (75), form a ring denoted by formula (I): 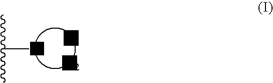
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from hydrogen, hydroxy, C₁-C₃hydroxyalkyl, oxo, C₂-C₄acyl, C₁-C₃alkyl, C₂-C₄alkylcarboxy, C₁-C₃alkoxy, C₁-C₂₀alkanoyloxy, or may be substituted to form a spiro five- or six-membered heterocyclic ring containing one or two heteroatoms selected from oxygen and sulfur; and any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (75), form a ring denoted by formula (II): 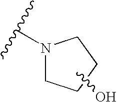
or in another embodiment R₁ and R₂, when taken together with the nitrogen atom to which they are directly attached in formula (I), may form a bicyclic ring system selected from 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently selected from bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl and N(R₆,R₇) where R₆ and R₇ are independently selected from hydrogen, acetyl, methanesulfonyl, and C₁-C₆alkyl; or
—O-Q represents a good leaving group which on reaction with a hydroxy function will result in the formation of an ether compound with retention of the stereochemical configuration of the hydroxy function; and
—O-J represents a good leaving group on reaction with a nucleophilic reactant will result in a substitution product with substantial inversion of the stereochemical configuration of the activated hydroxy group as shown in FIG. 45; and all the formulae and symbols are as described above.

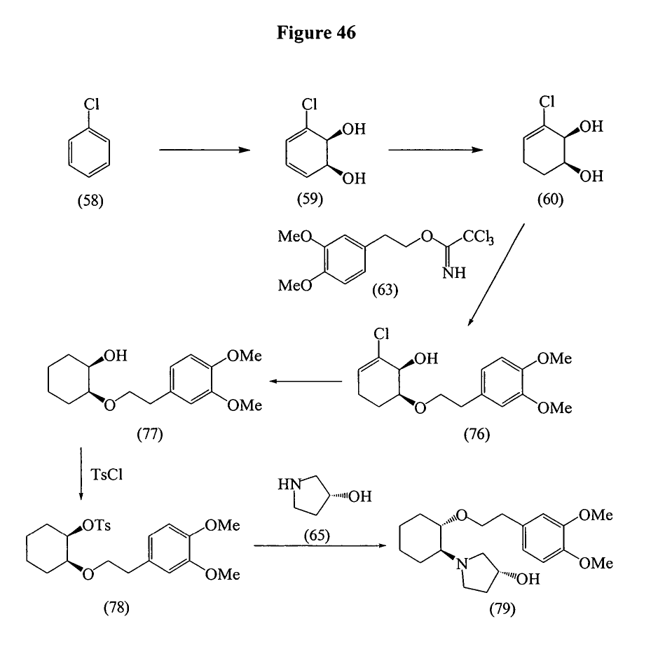

In another embodiment, the present invention provides a process for the preparation of a stereoisomerically substantially pure compound of formula (79), comprising the steps under suitable conditions as shown in FIG. 46, wherein all the formulae and symbols are as described above. As outlined in FIG. 46, the preparation of a stereoisomerically substantially pure trans aminocyclohexyl ether compound of formula (79) may be carried out by starting with a biotransformation of chlorobenzene (58) to compound (59) by microorganism such as Pseudomonas putida 39/D. Experimental conditions for the biotransformation are well established (Organic Synthesis, Vol. 76, 77 and T. Hudlicky et al., Aldrichimica Acta, 1999, 32, 35; and references cited therein). In a separate step, compound (59) is selectively reduced under suitable conditions to compound (60) (e.g., H₂—Rh/Al₂O₃; Boyd et al. JCS Chem. Commun. 1996, 45-46; Ham and Coker, J. Org. Chem. 1964, 29, 194-198; and references cited therein). In another separate step, compound (60) is converted to compound (76) by reaction with compound (63) under appropriate conditions. The trichloroacetimidate (63) is readily prepared from the corresponding alcohol, 3,4-dimethoxyphenethyl alcohol which is commercially available (e.g., Aldrich), by treatment with trichloroacetonitrile. The alkylation of compound (60) by trichloroacetimidate (63) may be carried out in the presence of a Bronsted acid or Lewis acid such as HBF₄. The reaction temperature may be adjusted as required to maximize the yields of the desired product. In a separate step, compound (76) is converted to compound (77) by reduction such as hydrogenation and hydrogenolysis in the presence of a catalyst under appropriate conditions. Palladium on activated carbon is one example of the catalysts. The reduction of compound (76) may be conducted under basic conditions e.g., in the presence of a base such as sodium ethoxide, sodium bicarbonate, sodium acetate or calcium carbonate. The base may be added in one portion or incrementally during the course of the reaction. In another separate step, the hydroxy group of compound (77) is converted under suitable conditions into an activated form such as the tosylate (TsO—) of formula (78) (e.g., TsCl in the presence of pyridine). In a separate step, the tosylate group of formula (78) is displaced by an amino compound such as 3R-pyrrolidinol (65) with inversion of configuration. 3R-pyrrolidinol (65) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (79) at a suitable rate. An excess of the amino compound (65) may be used to maximally convert compound (78) to the product (79). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

The reaction sequence described above (FIG. 46) in general generates the compound of formula (79) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

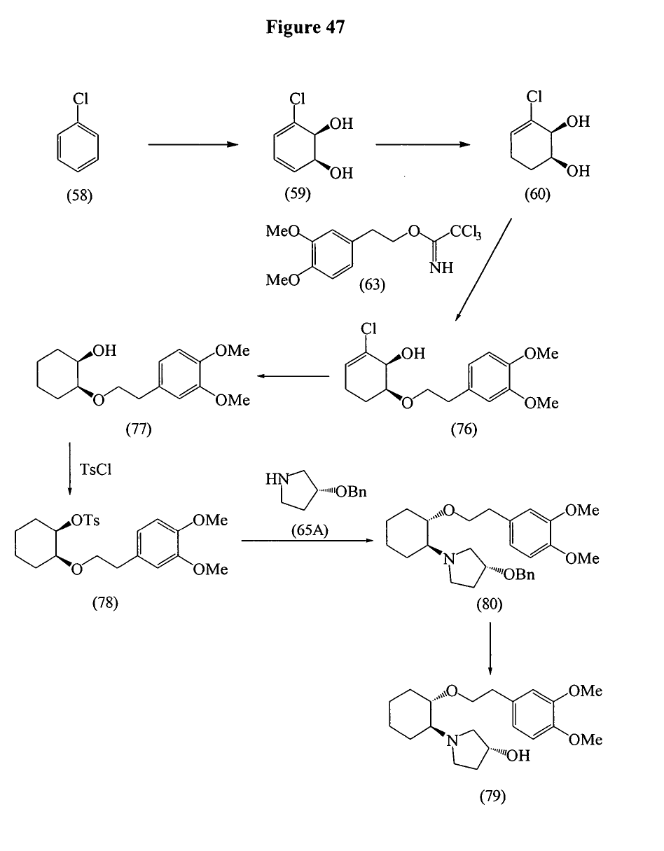

In another embodiment, the preparation of a stereoisomerically substantially pure trans aminocyclohexyl ether compound of formula (79) may be carried out under suitable conditions by a process as outlined in FIG. 47, comprising the steps of starting from chlorobenzene (58) and following a reaction sequence analogous to the applicable portion (i.e., rom compound (58) to compound (78)) that is described in FIG. 46 above leading to compound of formula (78). The latter is reacted under suitable conditions with an amino compound of formula (65A) wherein Bn represents a benzyl protection group of the hydroxy function of 3S-pyrrolidinol to form compound (80). Compound (65A) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (80) at a suitable rate. An excess of the amino compound (65A) may be used to maximally convert compound (78) to the product (80). The reaction may be performed in the presence of a base that can facilitate the formation of the product. Generally the additional base is non-nucleophilic in chemical reactivity. The benzyl (Bn) protection group of compound (80) may be removed by standard procedure (e.g., hydrogenation in the presence of a catalyst under appropriate conditions. Palladium on activated carbon is one example of the catalysts. Other suitable conditions are as described in Greene, “Protective Groups in Organic Chemistry”, John Wiley & Sons, New York N.Y. (1991)). The product is a stereoisomerically substantially pure trans aminocyclohexyl ether compound of formula (79) and is generally formed as the free base. The free base may be converted, if desired, to the monohydrochloride salt by known methodologies, or alternatively, if desired, to other acid addition salts by reaction with an inorganic or organic acids under appropriate conditions. Acid addition salts can also be prepared metathetically by reaction of one acid addition salt with an acid that is stronger than that giving rise to the initial salt.

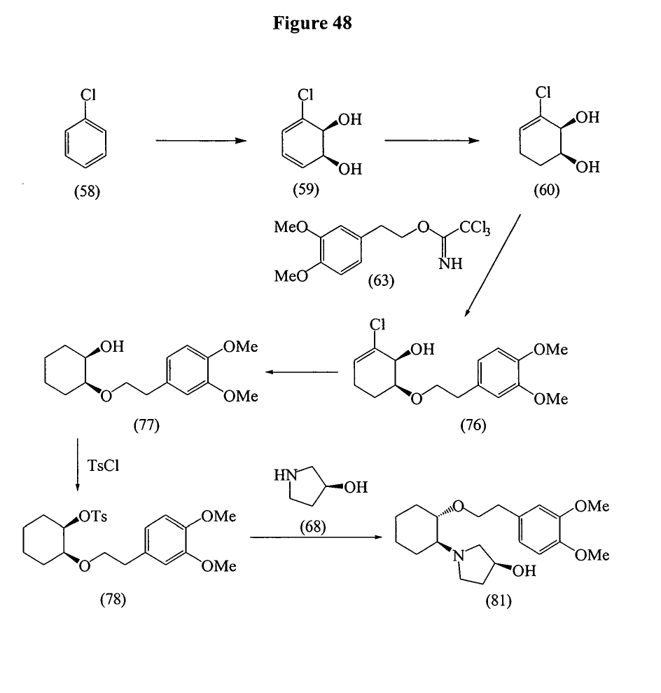

In another embodiment, the preparation of a stereoisomerically substantially pure trans aminocyclohexyl ether compound of formula (81) may be carried out under suitable conditions by a process as outlined in FIG. 48, comprising the steps of starting from chlorobenzene (58) and following a reaction sequence analogous to the applicable portion that is described in FIG. 46 above leading to compound of formula (78). The latter is reacted with an amino compound of formula (68). Compound (68), 3S-pyrrolidinol, is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (81) at a suitable rate. An excess of the amino compound (68) may be used to maximally convert compound (78) to the product (81). The reaction may be performed in the presence of a base that can facilitate the formation of the product. Generally the additional base is non-nucleophilic in chemical reactivity. The product is a stereoisomerically substantially pure trans aminocyclohexyl ether compound of formula (81) and is formed as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

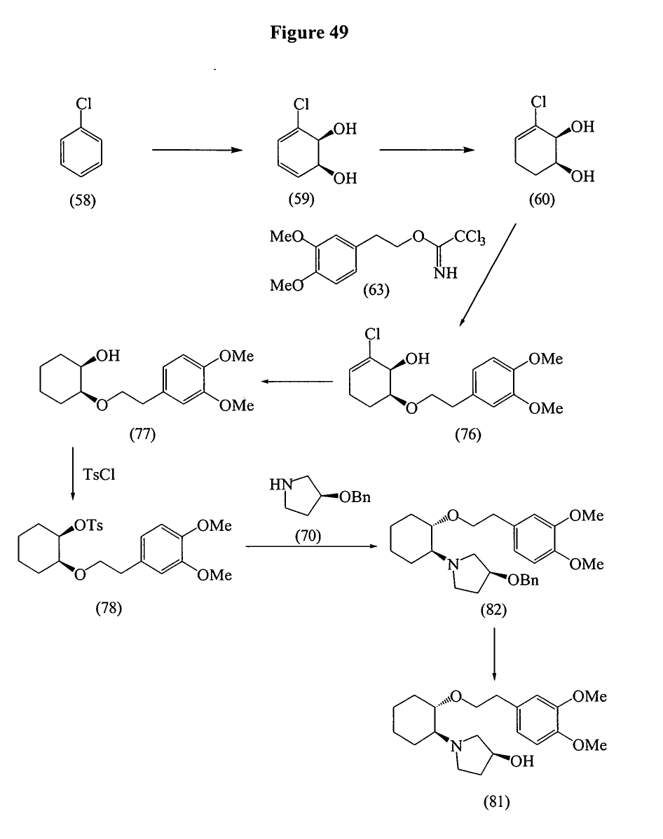

In another embodiment, the preparation of a stereoisomerically substantially pure trans aminocyclohexyl ether compound of formula (81) may be carried out under suitable conditions by a process as outlined in FIG. 49, comprising the steps of starting from chlorobenzene (58) and following a reaction sequence analogous to the applicable portion that is described in FIG. 47 above leading to compound of formula (78). The latter is reacted with an amino compound of formula (70) wherein Bn represents a benzyl protection group of the hydroxy function of 3S-pyrrolidinol to form compound (82). Compound (70) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (82) at a suitable rate. An excess of the amino compound (70) may be used to maximally convert compound (78) to the product (82). The reaction may be performed in the presence of a base that can facilitate the formation of the product. Generally the additional base is non-nucleophilic in chemical reactivity. The benzyl (Bn) protection group of compound (82) may be removed by standard procedure (e.g., hydrogenation in the presence of a catalyst under appropriate conditions. Palladium on activated carbon is one example of the catalysts. Other suitable conditions are as described in Greene, “Protective Groups in Organic Chemistry”, John Wiley & Sons, New York N.Y. (1991)). The product is a stereoisomerically substantially pure trans aminocyclohexyl ether compound of formula (81) and is generally formed as the free base. The free base may be converted, if desired, to the monohydrochloride salt by known methodologies, or alternatively, if desired, to other acid addition salts by reaction with an inorganic or organic acids under appropriate conditions. Acid addition salts can also be prepared metathetically by reaction of one acid addition salt with an acid that is stronger than that giving rise to the initial salt.

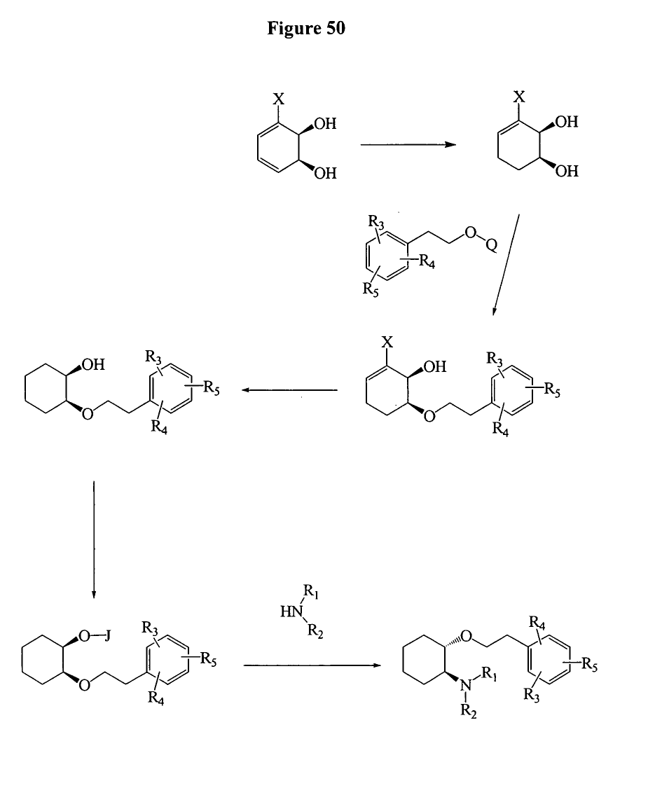

In another embodiment, the preparation of a stereoisomerically substantially pure trans aminocyclohexyl ether compound of formula (75) may be carried out under suitable conditions by a process as outlined in FIG. 50, comprising the steps of starting with compound of formula (50) and following a reaction sequence analogous to the applicable portion that is described in FIG. 45, wherein all the formulae and symbols are as described above.

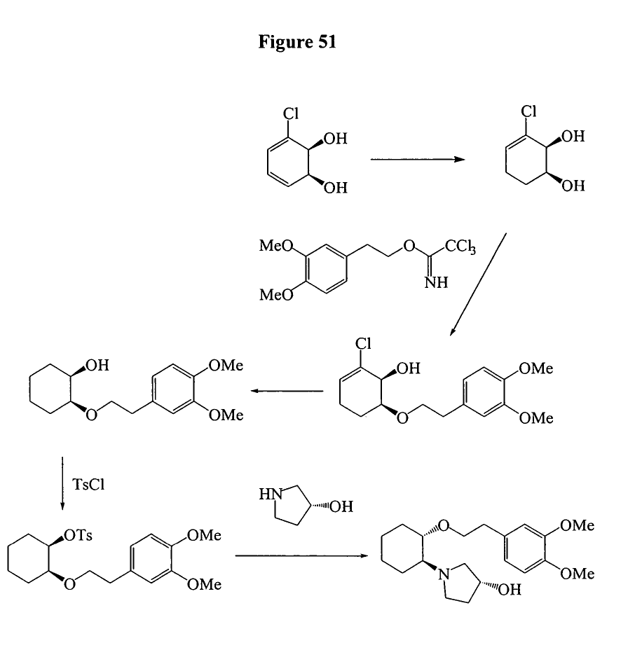

In another embodiment, the preparation of a stereoisomerically substantially pure trans aminocyclohexyl ether compound of formula (79) may be carried out under suitable conditions by a process as outlined in FIG. 51, comprising the steps of starting with compound of formula (59) and following a reaction sequence analogous to the applicable portion that is described in FIG. 46, wherein all the formulae and symbols are as described above. 3-Chloro-(1S,2S)-3,5-cyclohexadiene-1,2-diol of formula (59) is a commercially available product (e.g., Aldrich) or synthesized according to published procedure (e.g., Organic Synthesis, Vol. 76, 77 and T. Hudlicky et al., Aldrichimica Acta, 1999, 32, 35; and references cited therein).

In another embodiment, the preparation of a stereoisomerically substantially pure trans aminocyclohexyl ether compound of formula (79) may be carried out under suitable conditions by a process as outlined in FIG. 52, comprising the steps of starting with compound of formula (59) and following a reaction sequence analogous to the applicable portion that is described in FIG. 47, wherein all the formulae and symbols are as described above.

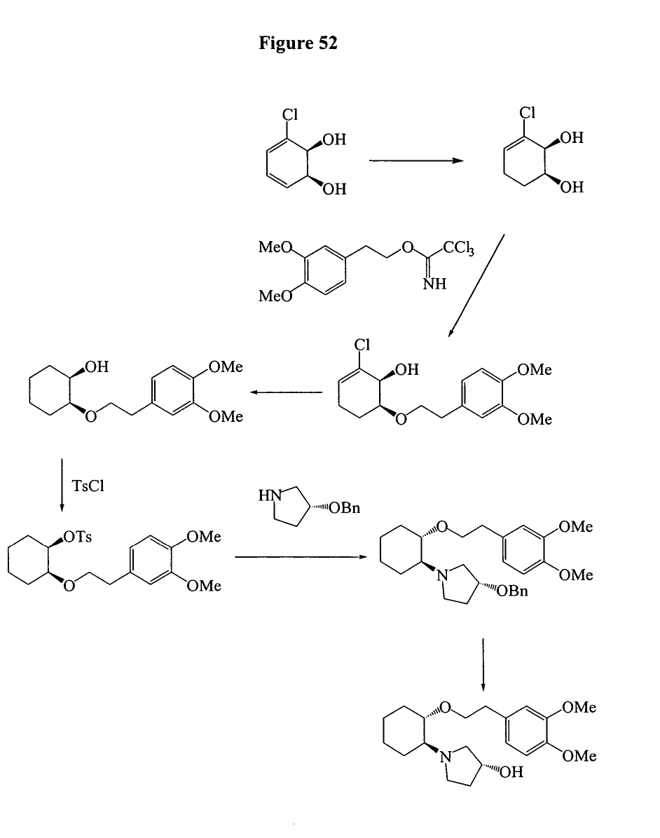

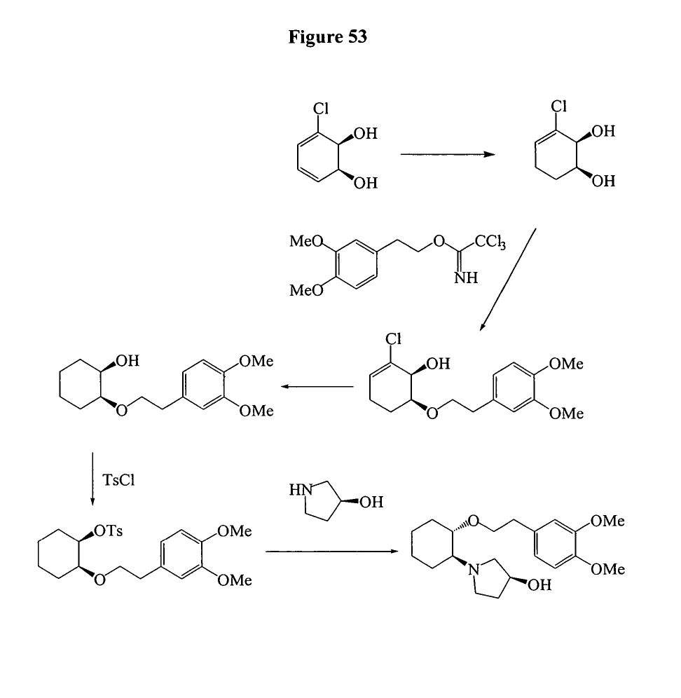

In another embodiment, the preparation of a stereoisomerically substantially pure trans aminocyclohexyl ether compound of formula (81) may be carried out under suitable conditions by a process as outlined in FIG. 53, comprising the steps of starting with compound of formula (59) and following a reaction sequence analogous to the applicable portion that is described in FIG. 48, wherein all the formulae and symbols are as described above.

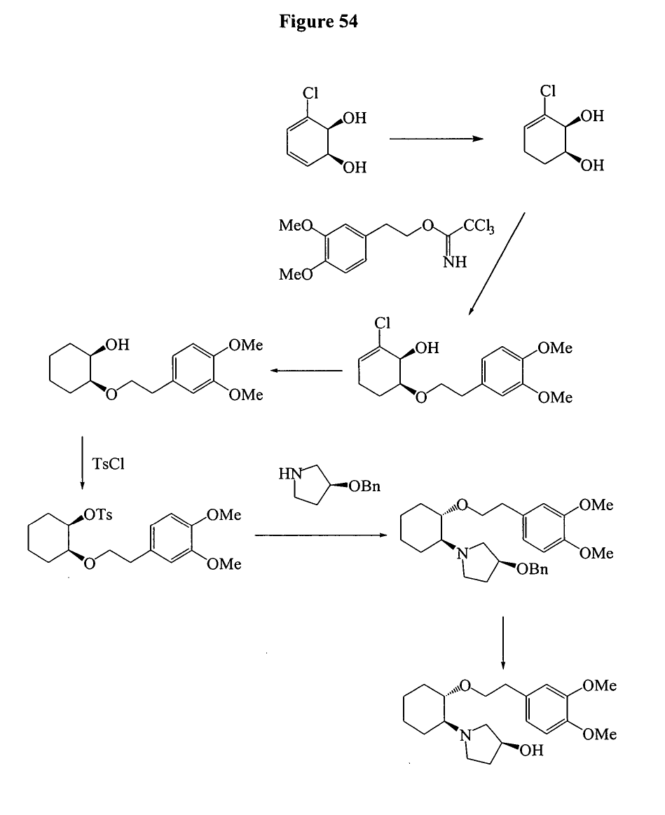

In another embodiment, the preparation of a stereoisomerically substantially pure trans aminocyclohexyl ether compound of formula (81) may be carried out under suitable conditions by a process as outlined in FIG. 54, comprising the steps of starting with compound of formula (59) and following a reaction sequence analogous to the applicable portion that is described in FIG. 49, wherein all the formulae and symbols are as described above.

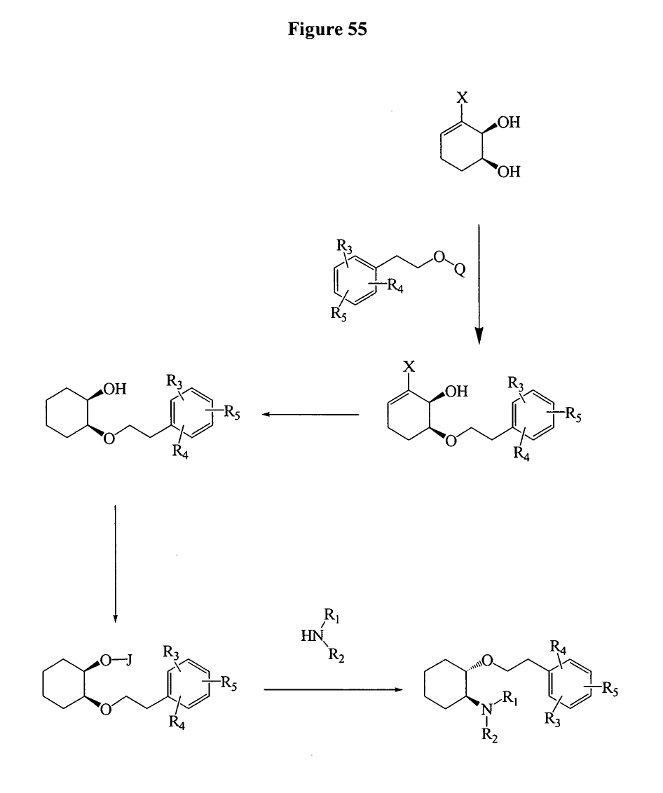

In another embodiment, the preparation of a stereoisomerically substantially pure trans aminocyclohexyl ether compound of formula (75) may be carried out under suitable conditions by a process as outlined in FIG. 55, comprising the steps of starting with compound of formula (51) and following a reaction sequence analogous to the applicable portion that is described in FIG. 45, wherein all the formulae and symbols are as described above.

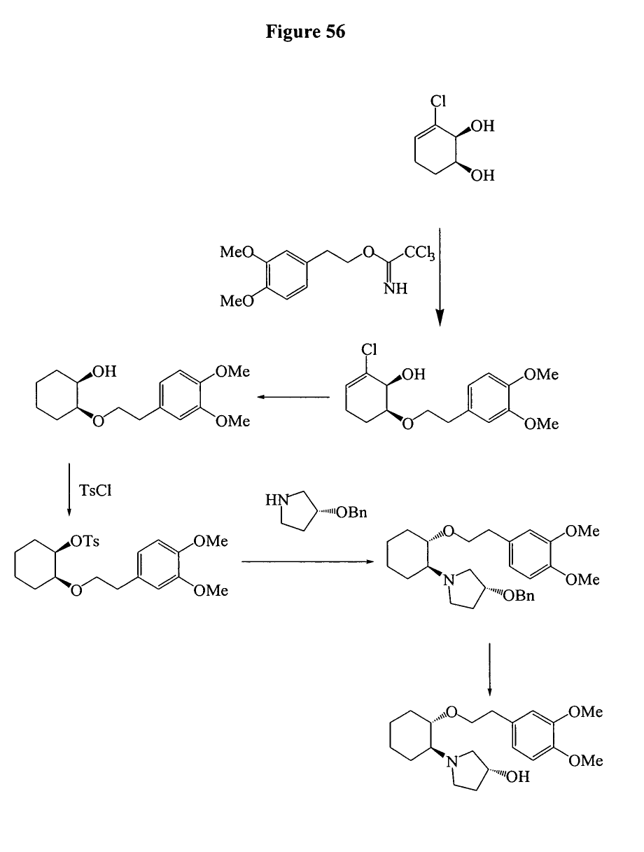

In another embodiment, the preparation of a stereoisomerically substantially pure trans aminocyclohexyl ether compound of formula (79) may be carried out under suitable conditions by a process as outlined in FIG. 56, comprising the steps of starting with compound of formula (60) and following a reaction sequence analogous to the applicable portion that is described in FIG. 46, wherein all the formulae and symbols are as described above.

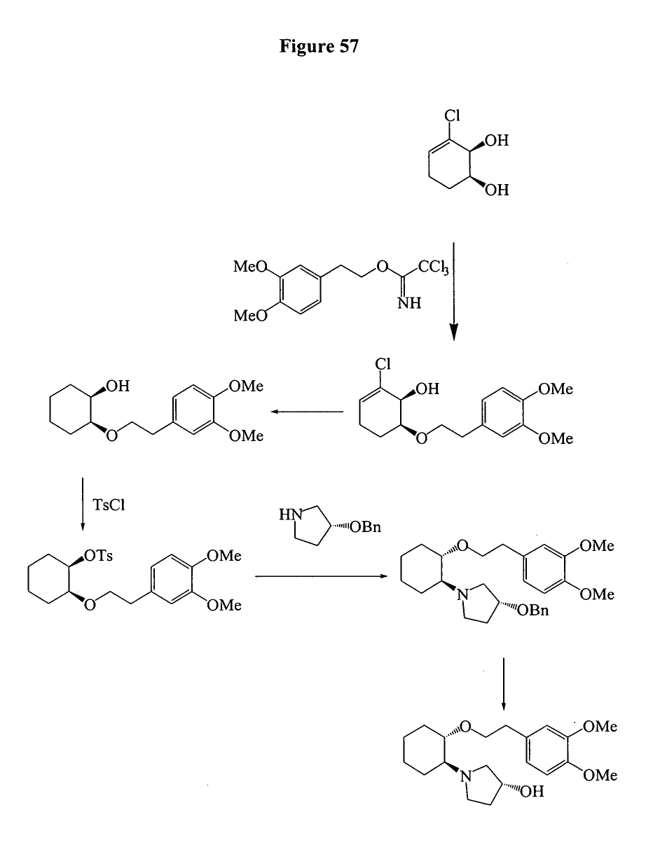

In another embodiment, the preparation of a stereoisomerically substantially pure trans aminocyclohexyl ether compound of formula (79) may be carried out under suitable conditions by a process as outlined in FIG. 57, comprising the steps of starting with compound of formula (60) and following a reaction sequence analogous to the applicable portion that is described in FIG. 47, wherein all the formulae and symbols are as described above.

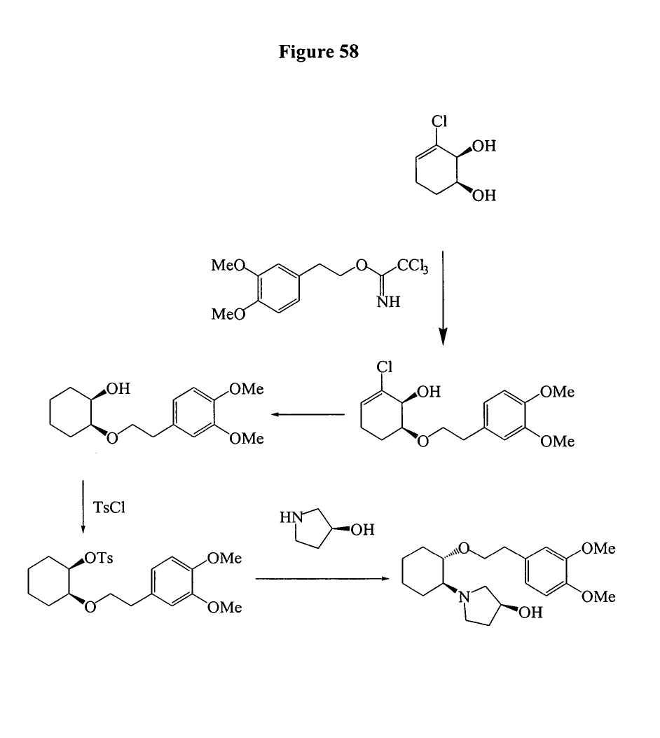

In another embodiment, the preparation of a stereoisomerically substantially pure trans aminocyclohexyl ether compound of formula (81) may be carried out under suitable conditions by a process as outlined in FIG. 58, comprising the steps of starting with compound of formula (60) and following a reaction sequence analogous to the applicable portion that is described in FIG. 48, wherein all the formulae and symbols are as described above.

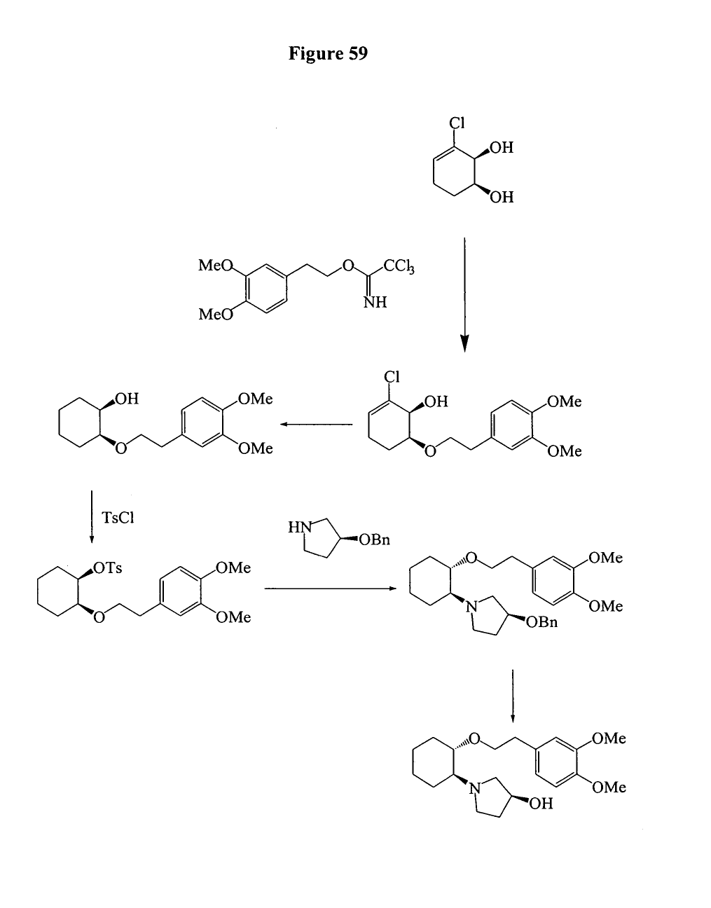

In another embodiment, the preparation of a stereoisomerically substantially pure trans aminocyclohexyl ether compound of formula (81) may be carried out under suitable conditions by a process as outlined in FIG. 59, comprising the steps of starting with compound of formula (60) and following a reaction sequence analogous to the applicable portion that is described in FIG. 49, wherein all the formulae and symbols are as described above.

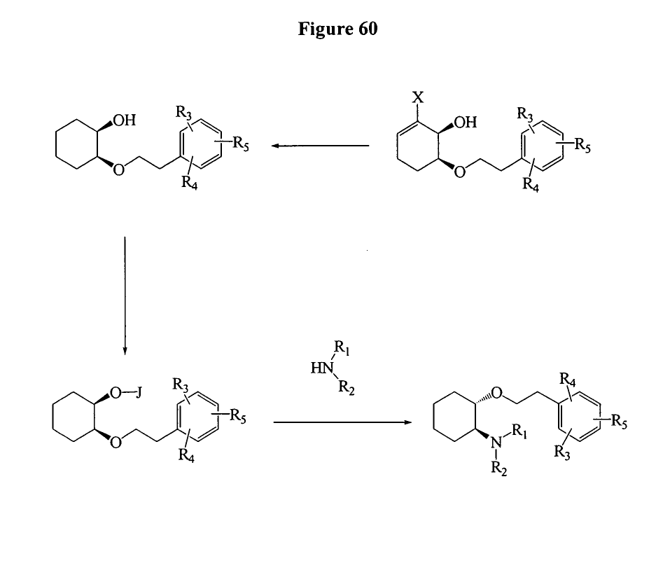

In another embodiment, the preparation of a stereoisomerically substantially pure trans aminocyclohexyl ether compound of formula (75) may be carried out under suitable conditions by a process as outlined in FIG. 60, comprising the steps of starting with compound of formula (72) and following a reaction sequence analogous to the applicable portion that is described in FIG. 45, wherein all the formulae and symbols are as described above.

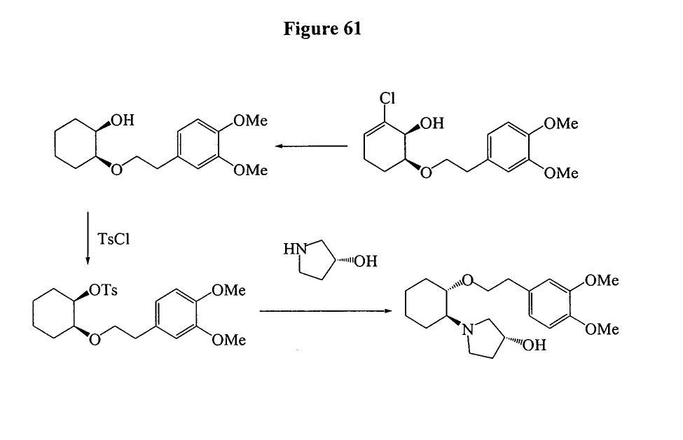

In another embodiment, the preparation of a stereoisomerically substantially pure trans aminocyclohexyl ether compound of formula (79) may be carried out under suitable conditions by a process as outlined in FIG. 61, comprising the steps of starting with compound of formula (76) and following a reaction sequence analogous to the applicable portion that is described in FIG. 46, wherein all the formulae and symbols are as described above.

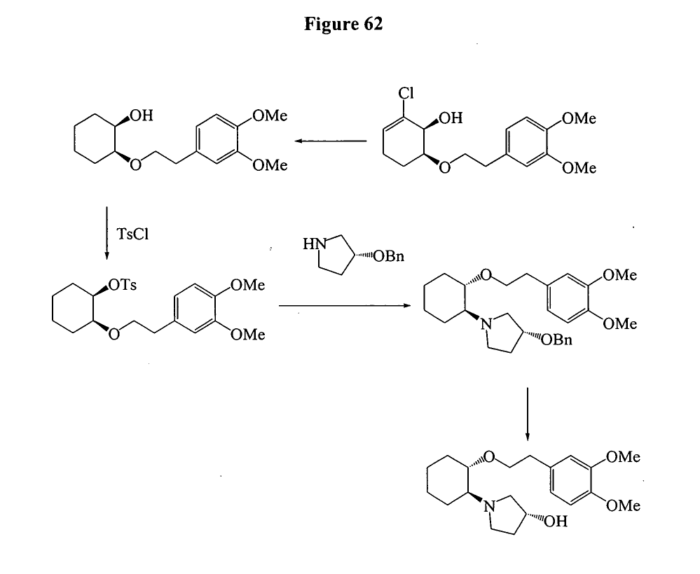

In another embodiment, the preparation of a stereoisomerically substantially pure trans aminocyclohexyl ether compound of formula (79) may be carried out under suitable conditions by a process as outlined in FIG. 62, comprising the steps of starting with compound of formula (76) and following a reaction sequence analogous to the applicable portion that is described in FIG. 47, wherein all the formulae and symbols are as described above.

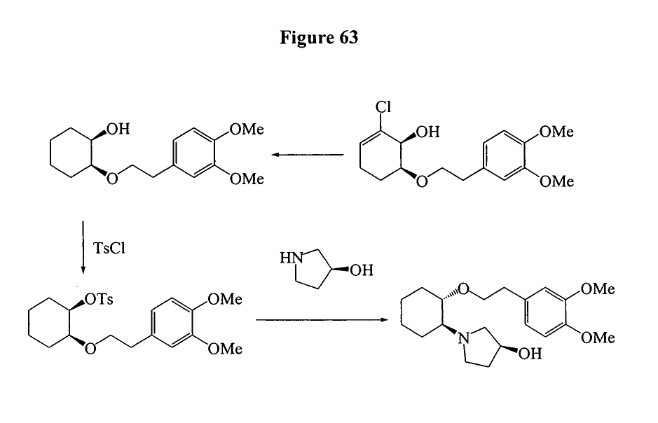

In another embodiment, the preparation of a stereoisomerically substantially pure trans aminocyclohexyl ether compound of formula (81) may be carried out under suitable conditions by a process as outlined in FIG. 63, comprising the steps of starting with compound of formula (76) and following a reaction sequence analogous to the applicable portion that is described in FIG. 48, wherein all the formulae and symbols are as described above.

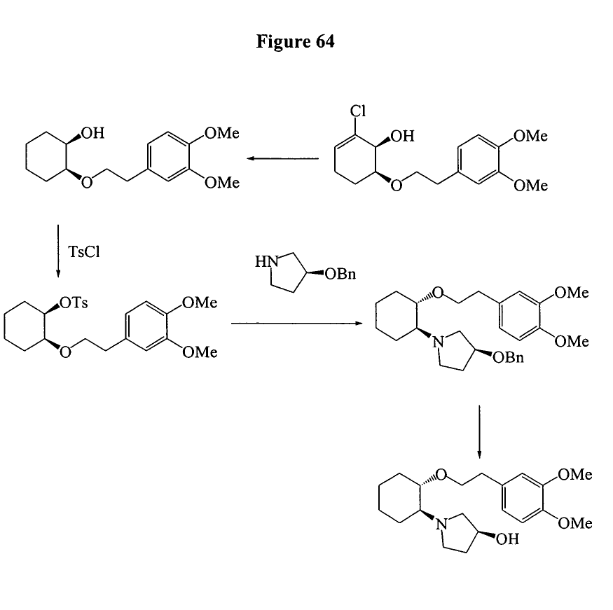

In another embodiment, the preparation of a stereoisomerically substantially pure trans aminocyclohexyl ether compound of formula (81) may be carried out under suitable conditions by a process as outlined in FIG. 64, comprising the steps of starting with compound of formula (76) and following a reaction sequence analogous to the applicable portion that is described in FIG. 49, wherein all the formulae and symbols are as described above.

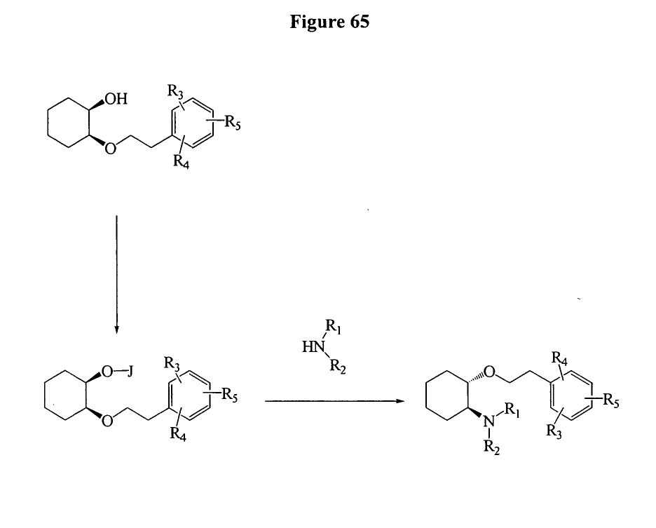

In another embodiment, the preparation of a stereoisomerically substantially pure trans aminocyclohexyl ether compound of formula (75) may be carried out under suitable conditions by a process as outlined in FIG. 65, comprising the steps of starting with compound of formula (73) and following a reaction sequence analogous to the applicable portion that is described in FIG. 45, wherein all the formulae and symbols are as described above.

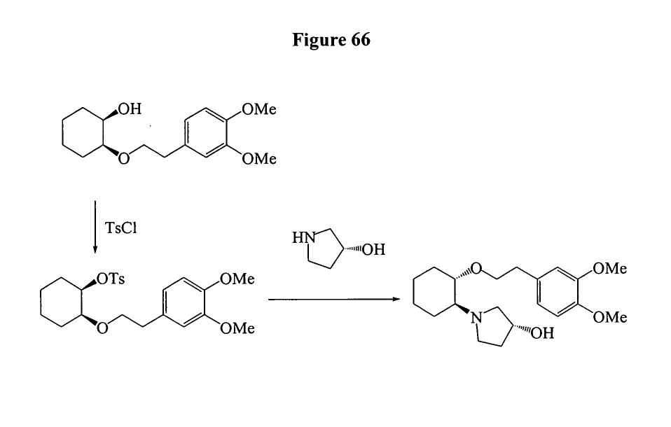

In another embodiment, the preparation of a stereoisomerically substantially pure trans aminocyclohexyl ether compound of formula (79) may be carried out under suitable conditions by a process as outlined in FIG. 66, comprising the steps of starting with compound of formula (77) and following a reaction sequence analogous to the applicable portion that is described in FIG. 46, wherein all the formulae and symbols are as described above.

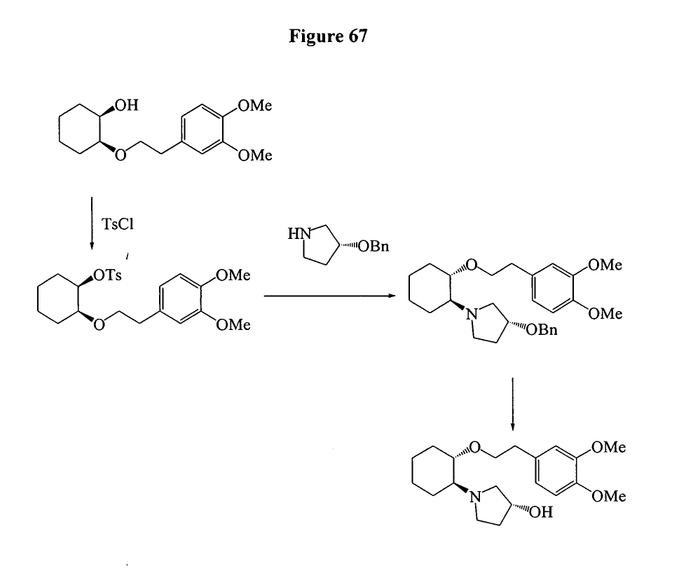

In another embodiment, the preparation of a stereoisomerically substantially pure trans aminocyclohexyl ether compound of formula (79) may be carried out under suitable conditions by a process as outlined in FIG. 67, comprising the steps of starting with compound of formula (77) and following a reaction sequence analogous to the applicable portion that is described in FIG. 47, wherein all the formulae and symbols are as described above.

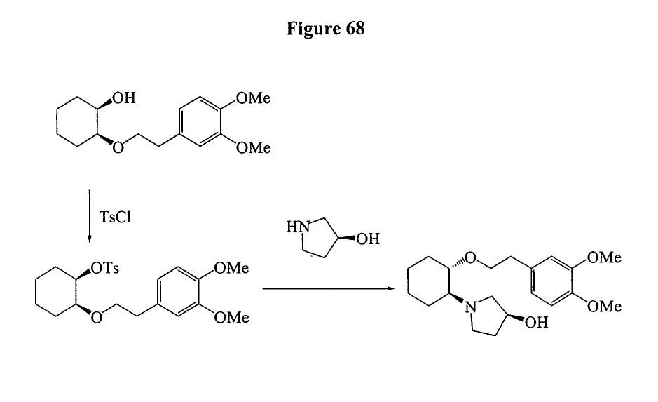

In another embodiment, the preparation of a stereoisomerically substantially pure trans aminocyclohexyl ether compound of formula (81) may be carried out under suitable conditions by a process as outlined in FIG. 68, comprising the steps of starting with compound of formula (77) and following a reaction sequence analogous to the applicable portion that is described in FIG. 48, wherein all the formulae and symbols are as described above.

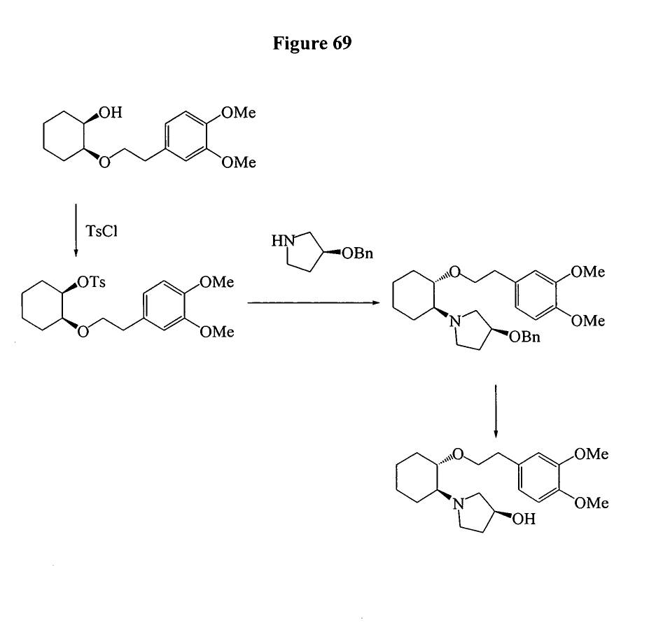

In another embodiment, the preparation of a stereoisomerically substantially pure trans aminocyclohexyl ether compound of formula (81) may be carried out under suitable conditions by a process as outlined in FIG. 69, comprising the steps of starting with compound of formula (77) and following a reaction sequence analogous to the applicable portion that is described in FIG. 49, wherein all the formulae and symbols are as described above.

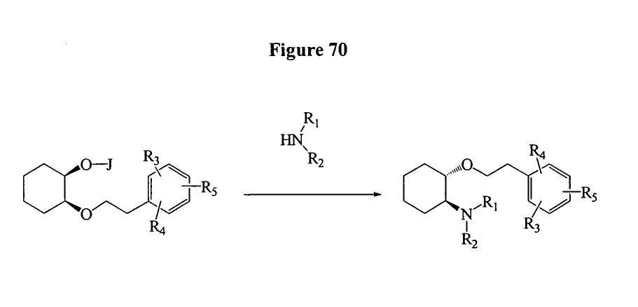

In another embodiment, the preparation of a stereoisomerically substantially pure trans aminocyclohexyl ether compound of formula (75) may be carried out under suitable conditions by a process as outlined in FIG. 70, comprising the steps of starting with compound of formula (74) and following a reaction sequence analogous to the applicable portion that is described in FIG. 45, wherein all the formulae and symbols are as described above.

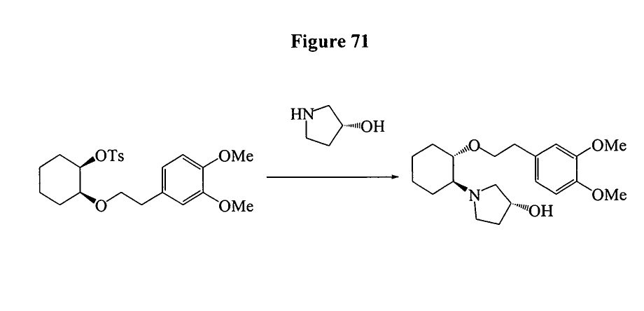

In another embodiment, the preparation of a stereoisomerically substantially pure trans aminocyclohexyl ether compound of formula (79) may be carried out under suitable conditions by a process as outlined in FIG. 71, comprising the steps of starting with compound of formula (78) and following a reaction sequence analogous to the applicable portion that is described in FIG. 46, wherein all the formulae and symbols are as described above.

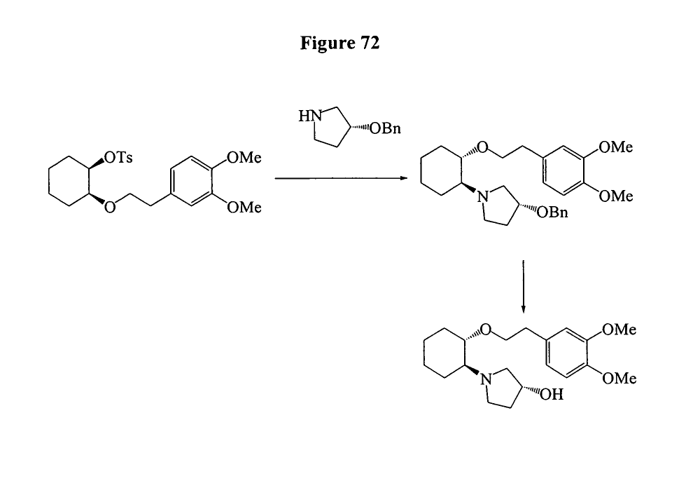

In another embodiment, the preparation of a stereoisomerically substantially pure trans aminocyclohexyl ether compound of formula (79) may be carried out under suitable conditions by a process as outlined in FIG. 72, comprising the steps of starting with compound of formula (78) and following a reaction sequence analogous to the applicable portion that is described in FIG. 47, wherein all the formulae and symbols are as described above.

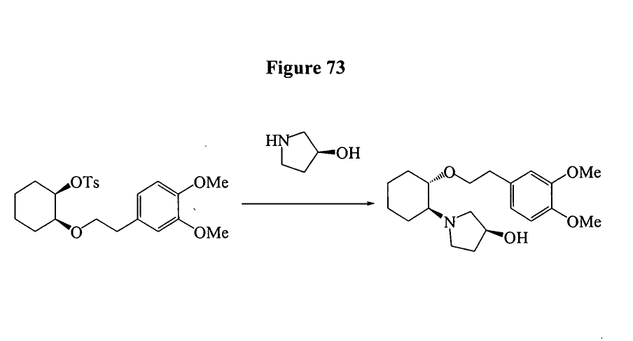

In another embodiment, the preparation of a stereoisomerically substantially pure trans aminocyclohexyl ether compound of formula (81) may be carried out under suitable conditions by a process as outlined in FIG. 73, comprising the steps of starting with compound of formula (78) and following a reaction sequence analogous to the applicable portion that is described in FIG. 48, wherein all the formulae and symbols are as described above.

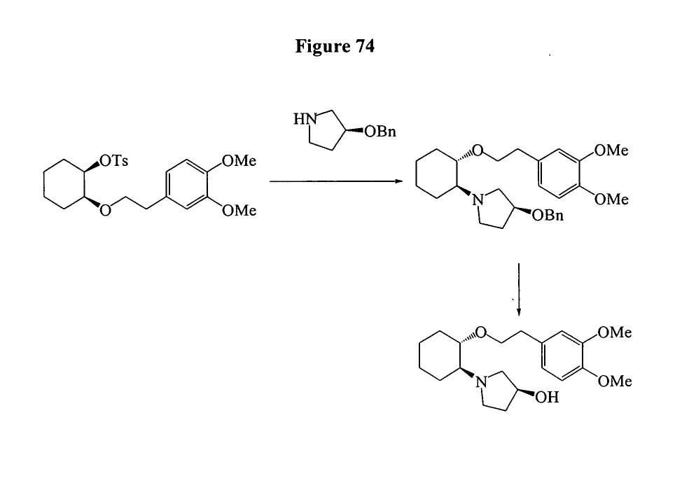

In another embodiment, the preparation of a stereoisomerically substantially pure trans aminocyclohexyl ether compound of formula (81) may be carried out under suitable conditions by a process as outlined in FIG. 74, comprising the steps of starting with compound of formula (78) and following a reaction sequence analogous to the applicable portion that is described in FIG. 49, wherein all the formulae and symbols are as described above.

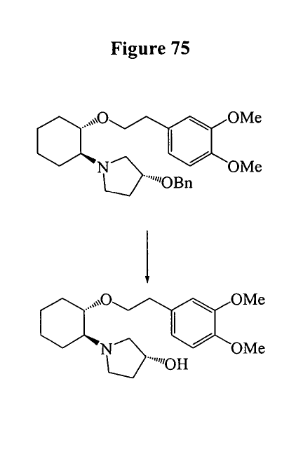

In another embodiment, the preparation of a stereoisomerically substantially pure trans aminocyclohexyl ether compound of formula (79) may be carried out under suitable conditions by a process as outlined in FIG. 75, comprising the steps of starting with compound of formula (80) and following a reaction sequence analogous to the applicable portion that is described in FIG. 47, wherein all the formulae and symbols are as described above.

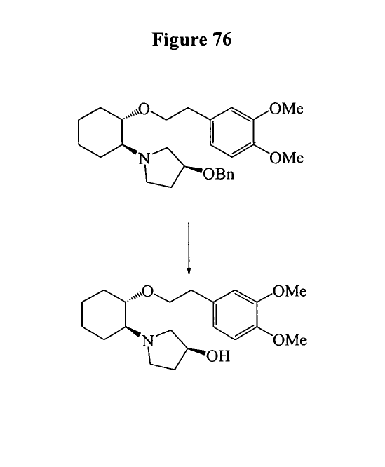

In another embodiment, the preparation of a stereoisomerically substantially pure trans aminocyclohexyl ether compound of formula (81) may be carried out under suitable conditions by a process as outlined in FIG. 76, comprising the steps of starting with compound of formula (82) and following a reaction sequence analogous to the applicable portion that is described in FIG. 49, wherein all the formulae and symbols are as described above.

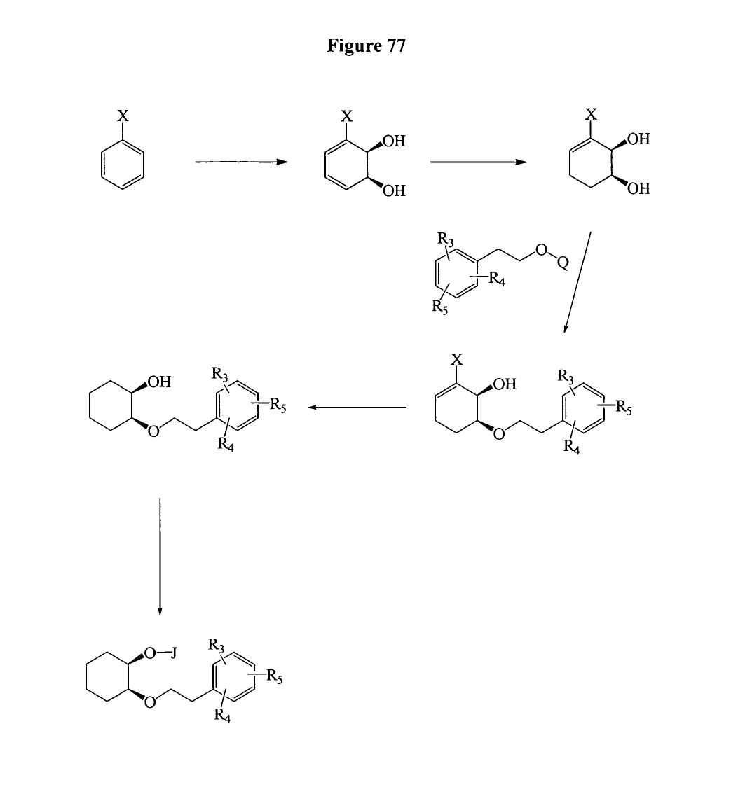

In another embodiment, the preparation of a stereoisomerically substantially pure compound of formula (74) may be carried out under suitable conditions by a process as outlined in FIG. 77, comprising the steps of starting with compound of formula (49) and following a reaction sequence analogous to the applicable portion that is described in FIG. 45, wherein all the formulae and symbols are as described above.

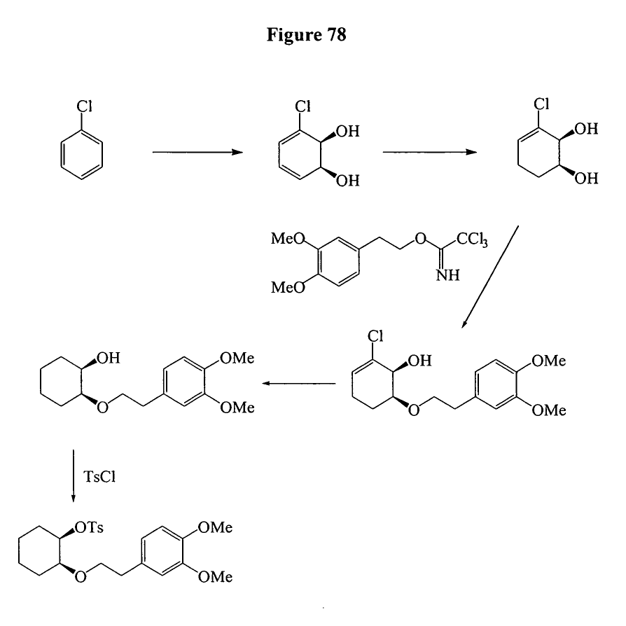

In another embodiment, the preparation of a stereoisomerically substantially pure compound of formula (78) may be carried out under suitable conditions by a process as outlined in FIG. 78, comprising the steps of starting with compound of formula (58) and following a reaction sequence analogous to the applicable portion that is described in FIG. 46, wherein all the formulae and symbols are as described above.

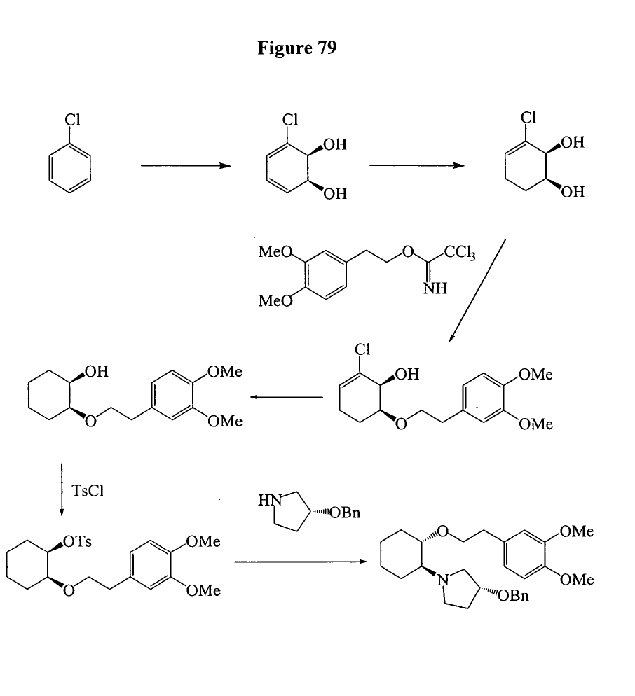

In another embodiment, the preparation of a stereoisomerically substantially pure trans aminocyclohexyl ether compound of formula (80) may be carried out under suitable conditions by a process as outlined in FIG. 79, comprising the steps of starting with compound of formula (58) and following a reaction sequence analogous to the applicable portion that is described in FIG. 47, wherein all the formulae and symbols are as described above.

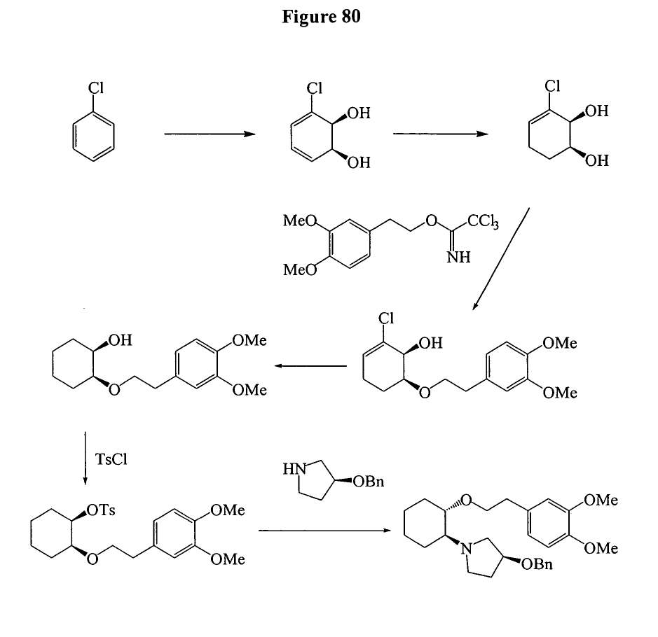

In another embodiment, the preparation of a stereoisomerically substantially pure trans aminocyclohexyl ether compound of formula (82) may be carried out under suitable conditions by a process as outlined in FIG. 80, comprising the steps of starting with compound of formula (58) and following a reaction sequence analogous to the applicable portion that is described in FIG. 49, wherein all the formulae and symbols are as described above.

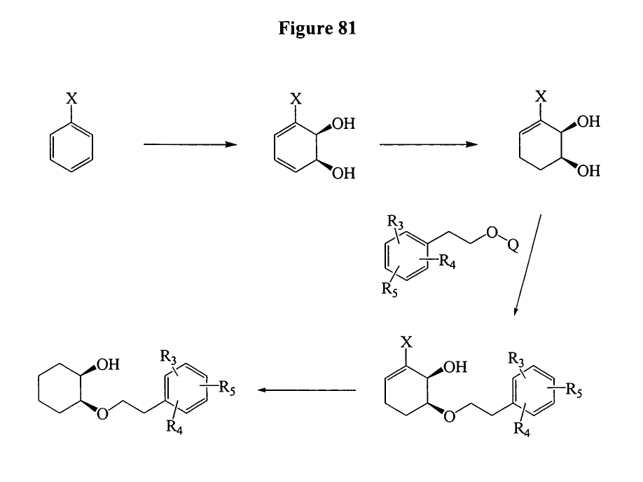

In another embodiment, the preparation of a stereoisomerically substantially pure compound of formula (73) may be carried out under suitable conditions by a process as outlined in FIG. 81, comprising the steps of starting with compound of formula (49) and following a reaction sequence analogous to the applicable portion that is described in FIG. 45, wherein all the formulae and symbols are as described above.

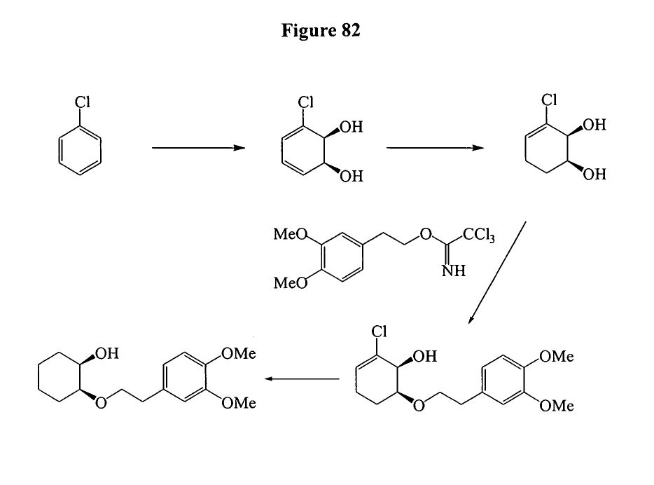

In another embodiment, the preparation of a stereoisomerically substantially pure compound of formula (77) may be carried out under suitable conditions by a process as outlined in FIG. 82, comprising the steps of starting with compound of formula (58) and following a reaction sequence analogous to the applicable portion that is described in FIG. 46, wherein all the formulae and symbols are as described above.

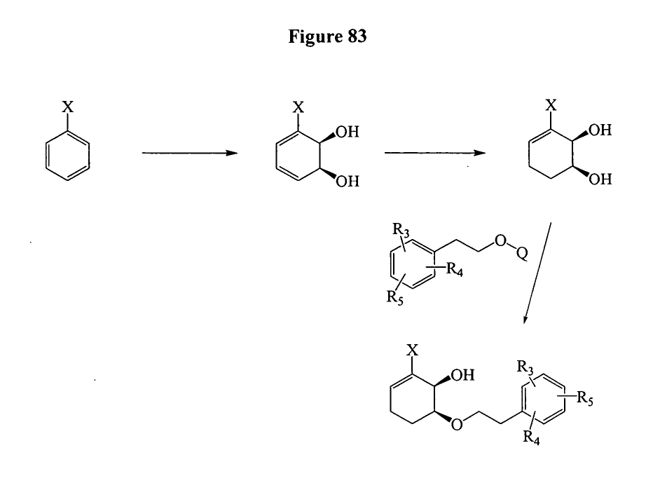

In another embodiment, the preparation of a stereoisomerically substantially pure compound of formula (72) may be carried out under suitable conditions by a process as outlined in FIG. 83, comprising the steps of starting with compound of formula (49) and following a reaction sequence analogous to the applicable portion that is described in FIG. 45, wherein all the formulae and symbols are as described above.

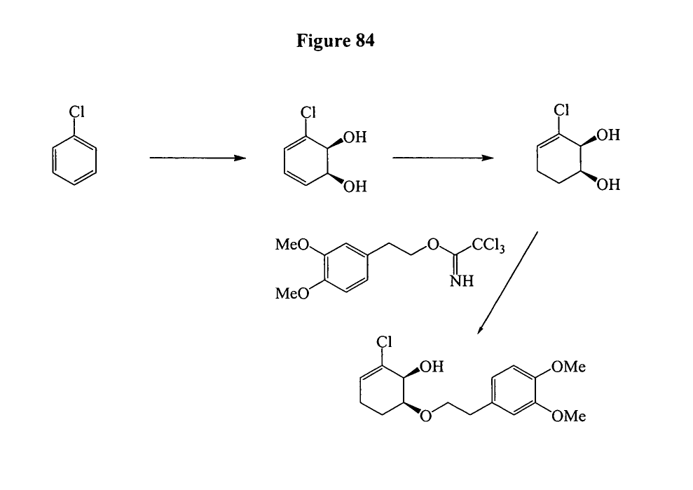

In another embodiment, the preparation of a stereoisomerically substantially pure compound of formula (76) may be carried out under suitable conditions by a process as outlined in FIG. 84, comprising the steps of starting with compound of formula (58) and following a reaction sequence analogous to the applicable portion that is described in FIG. 46, wherein all the formulae and symbols are as described above.

In another embodiment, the present invention provides a compound of formula (72), or a solvate or pharmaceutically acceptable salt thereof; wherein all the formulae and symbols are as described above.

In another embodiment, the present invention provides a compound of formula (73), or a solvate or pharmaceutically acceptable salt thereof; wherein all the formulae and symbols are as described above.

In another embodiment, the present invention provides a compound of formula (73), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (74), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (76), or a solvate or pharmaceutically acceptable salt thereof; wherein all the formulae and symbols are as described above.

In another embodiment, the present invention provides a compound of formula (77), or a solvate or pharmaceutically acceptable salt thereof; wherein all the formulae and symbols are as described above.

In another embodiment, the present invention provides a compound of formula (78), or a solvate or pharmaceutically acceptable salt thereof; wherein all the formulae and symbols are as described above.

In another embodiment, the present invention provides a compound of formula (80), or a solvate or pharmaceutically acceptable salt thereof; wherein all the formulae and symbols are as described above.

The present invention provides synthetic processes whereby compounds of formula (57) with trans-(1R,2R) configuration for the ether and amino functional groups may be prepared in stereoisomerically substantially pure form. Compound of formula (66) is an example represented by formula (57). The present invention also provides synthetic processes whereby compounds of formula (75) with trans-(1S,2S) configuration for the ether and amino functional groups may be prepared in stereoisomerically substantially pure form. Compound of formula (79) is an example represented by formula (75). The present invention further provides synthetic processes whereby compounds of formulae (85), (86), (55) and (74) may be synthesized in stereoisomerically substantially pure forms. Compounds (62) and (90) are examples of formula (85). Compounds (87) and (89) are examples of formula (86). Compound (64) is an example of formula (55). Compound (78) is an example of formula (74). The aminocyclohexyl ether compounds of the present invention may be used for medical applications, including, for example, cardiac arrhythmia, such as atrial arrhythmia and ventricular arrhythmia.

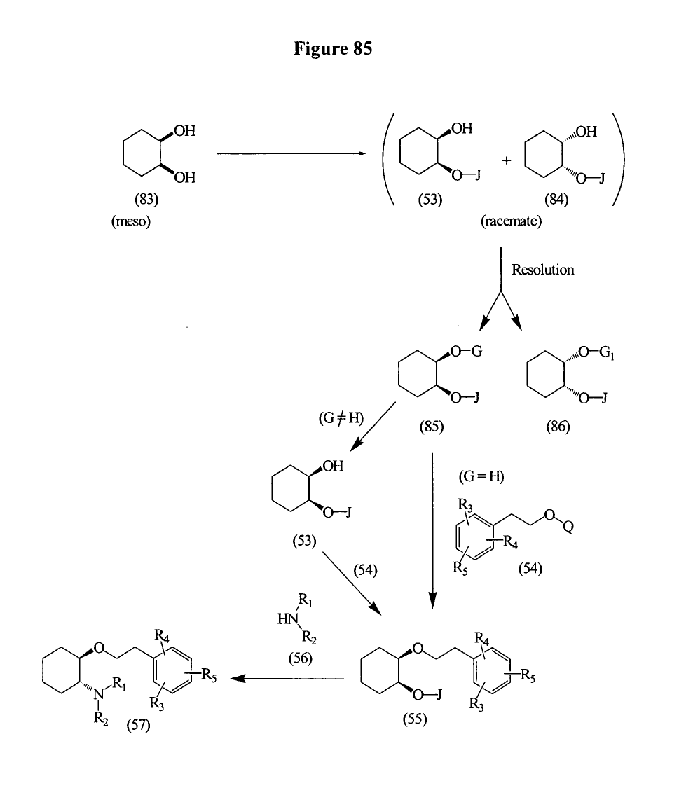

As outlined in FIG. 85, the preparation of a stereoisomerically substantially pure trans aminocyclohexyl ether compound of formula (57) may be carried out by following a process starting from a racemic mixture of meso-cis-1,2-cyclohexandiol (83). Compound (83) is commercially available (e.g., Sigma-Aldrich, St. Louis, Mo.) or can be readily synthesized by published methods (e.g., J. E. Taylor et al., Org. Process Res. & Dev., 1998, 2, 147; Organic Syntheses, CV6, 342).

In a first step, one of the hydroxy groups of compound (83) is converted under suitable conditions into an activated form as represented by the racemic mixture comprises of formulae (53) and (84). An “activated form” as used herein means that the hydroxy group is converted into a good leaving group (—O-J) which on reaction with an appropriate nucleophile (e.g., HNR₁R₂) will result in a substitution product with substantial inversion of the stereochemical configuration of the activated hydroxy group. The leaving group (—O-J) may be but is not limited to an alkyl sulfonate such as a trifluoromethanesulfonate group (CF₃SO₃—) or a mesylate group (MsO—), an aryl sulfonate such as a benzenesulfonate group (PhSO₃—), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. The leaving group may be any suitable leaving group on reaction with a nucleophilic reactant with inversion of stereochemical configuration known in the art, including but not limited to compounds disclosed in M. B. Smith and J. March in “March's Advanced Organic Chemistry”, Fifth edition, Chapter 10, John Wiley & Sons, Inc., New York, N.Y. (2001). In a typical reaction for the formation of an alkyl sulfonate (e.g., a mesylate) or an aryl sulfonate (e.g., a tosylate or a nosylate), compound (83) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (83) may be used to maximally convert the hydroxy group into the activated form. The hydroxy group may also be converted into other suitable leaving groups according to procedures well known in the art, using any suitable activating agent, including but not limited to those disclosed in M. B. Smith and J. March in “March's Advanced Organic Chemistry”, Fifth edition, Chapter 10, John Wiley & Sons, Inc., New York, N.Y. (2001). The addition of other reagents to facilitate the formation of the monotosylates may be advantageously employed (e.g., M. J. Martinelli, et al. “Selective monosulfonylation of internal 1,2-diols catalyzed by di-n-butyltin oxide” Tetrahedron Letters, 2000, 41, 3773). The racemic mixture comprises of formulae (53) and (84) is then subjected to a resolution process whereby the two optically active isomers are separated into products that are in stereoisomerically substantially pure form such as (85) and (86), wherein G and G₁ are independently selected from hydrogen, C₁-C₈acyl, or any other suitable functional groups that are introduced as part of the resolution process necessary for the separation of the two isomers. In some situations it may be adequate that the resolution process produces compounds of (85) and (86) of sufficient enrichment in their optical purity for application in the subsequent steps of the synthetic process. Methods for resolution of racemic mixtures are well know in the art (e.g., E. L. Eliel and S. H. Wilen, in Stereochemistry of Organic Compounds; John Wiley & Sons: New York, 1994; Chapter 7, and references cited therein). Suitable processes such as enzymatic resolution (e.g., lipase mediated) and chromatographic separation (e.g., HPLC with chiral stationary phase and/or with simulated moving bed technology, or supercritical fluid chromatography and related techniques) are some of the examples that may be applied (see e.g., T. J. Ward, Analytical Chemistry, 2002, 2863-2872).

For compound of formula (85) when G is hydrogen, (85) is the same as compound (53) and in a separate reaction step, alkylation of the free hydroxy group in compound (85) to form compound (55) is carried out under appropriate conditions with compound (54), where —O-Q represents a good leaving group on reaction with a hydroxy function with retention of the stereochemical configuration of the hydroxy function in the formation of an ether compound. The leaving group may be any suitable leaving group known in the art, including but not limited to compounds disclosed in Greene, “Protective Groups in Organic Chemistry”, John Wiley & Sons, New York N.Y. (1991). Specific examples of —O-Q groups include include trichloroacetimidate. For some compound (54), it may be necessary to introduce appropriate protection groups prior to this step being performed. Suitable protecting groups are set forth in, for example, Greene, “Protective Groups in Organic Chemistry”, John Wiley & Sons, New York N.Y. (1991). For compound of formula (85) when G is not hydrogen, suitable methods are used to convert (85) to compound (53). For example when G is a C₂ acyl function, a mild based-catalyzed methanolysis (G. Zemplen et al., Ber., 1936, 69, 1827) may be used to transform (85) to (53). The latter can then undergo the same reaction with (54) to produce (55) as described above.

In a separate step, the resulted compound (55) is treated under suitable conditions with an amino compound of formula (56) to form compound (57) as the product. The reaction may be carried out with or without a solvent and at an appropriate temperature range that allows the formation of the product (57) at a suitable rate. An excess of the amino compound (56) may be used to maximally convert compound (55) to the product (57). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 85) generates the compound of formula (57) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

In one embodiment, the present invention provides a process for the preparation of a stereoisomerically substantially pure compound of formula (57): 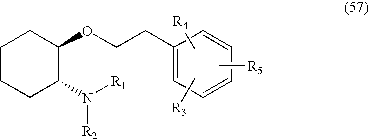
wherein, independently at each occurrence, R₁ and R₂ are independently selected from hydrogen, C₁-C₈alkyl, C₃-C₈alkoxyalkyl, C₁-C₈hydroxyalkyl, and C₇-C₁₂aralkyl; or
R₁ and R₂ are independently selected from C₃-C₈alkoxyalkyl, C₁-C₈hydroxyalkyl, and C₇-C₁₂aralkyl; or
R₁ and R₂, when taken together with the nitrogen atom to which they are directly attached in formula (57), form a ring denoted by formula (I): 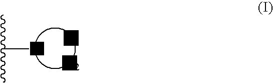
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from hydrogen, hydroxy, C₁-C₃hydroxyalkyl, oxo, C₂-C₄acyl, C₁-C₃alkyl, C₂-C₄alkylcarboxy, C₁-C₃alkoxy, C₁-C₂₀alkanoyloxy, or may be substituted to form a spiro five- or six-membered heterocyclic ring containing one or two heteroatoms selected from oxygen and sulfur; and any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (57), form a ring denoted by formula (II): 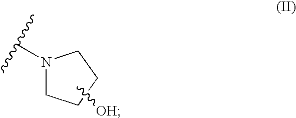
or in another embodiment R₁ and R₂, when taken together with the nitrogen atom to which they are directly attached in formula (I), may form a bicyclic ring system selected from 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently selected from bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl and N(R₆,R₇) where R₆ and R₇ are independently selected from hydrogen, acetyl, methanesulfonyl, and C₁-C₆alkyl; or
R₃, R₄ and R₅ are independently selected from hydrogen, hydroxy and C₁-C₆alkoxy; with the proviso that R₃, R₄ and R₅ cannot all be hydrogen;
comprising the steps of starting with a compound of formula (83), and following a reaction sequence as outlined in FIG. 85 under suitable conditions, wherein
G and G₁ are independently selected from hydrogen, C₁-C₈acyl, or any other suitable functional groups that are introduced as part of the resolution process necessary for the separation of the two isomers;
—O-Q represents a good leaving group on reaction with a hydroxy function with retention of the stereochemical configuration of the hydroxy function in the formation of an ether compound, including, but not limited to, those disclosed in “Protective Groups in Organic Chemistry”, John Wiley & Sons, New York N.Y. (1991); and
—O-J represents a good leaving group on reaction with a nucleophilic reactant with inversion of the stereochemical configuration, including, but not limited to, those disclosed in “Protective Groups in Organic Chemistry”, John Wiley & Sons, New York N.Y. (1991), as shown in FIG. 85 and all the formulae and symbols are as described above.

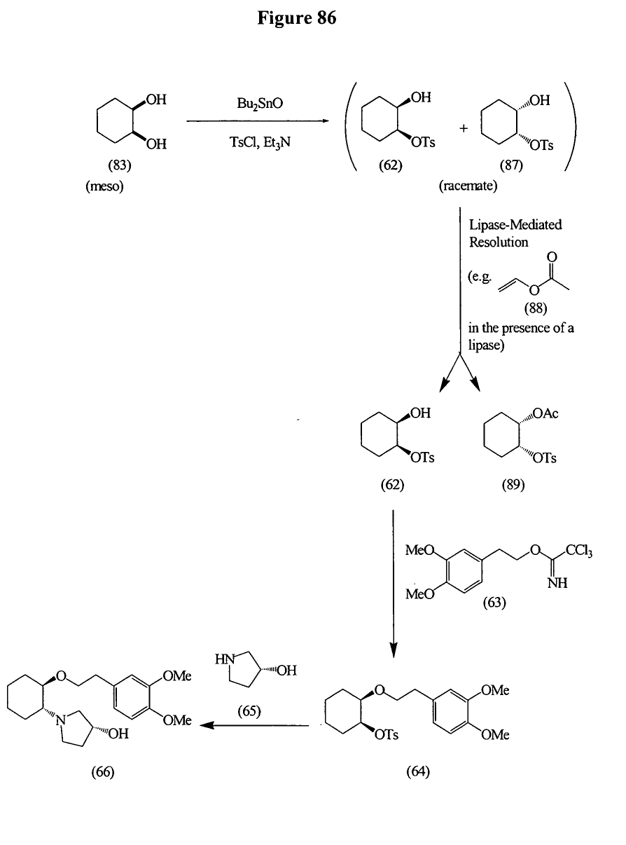

In another embodiment, the present invention provides a process for the preparation of a stereoisomerically substantially pure compound of formula (66), comprising the steps under suitable conditions as shown in FIG. 86, wherein all the formulae and symbols are as described above. As outlined in FIG. 86, the preparation of a stereoisomerically substantially pure trans aminocyclohexyl ether compound of formula (66) may be carried out by starting with the monotosylation of cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). Initial non-optimized yields of 80-90% have been achieved, and further optimization is being pursued. The resulting racemic mixture of hydroxytosylates comprises of compounds (62) and (87) is subjected to a lipase-mediated resolution process under suitable conditions such as treatment of the racemates (62) and (87) with vinyl acetate (88) in the presence of a lipase derived from Pseudomonas sp. (N. Boaz et al., Tetra. Asymmetry, 1994, 5, 153) to provide compound (62) and (89). In addition, any acylating reagent may also be used in lipase mediated reactions, such as acyl halide, and even more particularly acyl chloride. In a separate step, the stereoisomerically substantially pure compound of formula (62) obtained from the resolution process is alkylated under appropriate conditions by treatment with the trichloroacetimidate (63) to form compound (64). Initial non-optimized yields of 60-70% have been achieved, and further optimization is being pursued. The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (62) by trichloroacetimidate (63) may be carried out in the presence of a Lewis acid such as HBF₄.

In another separate step, the tosylate group of formula (64) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly. Initial non-optimized yields of approximately 40% have been achieved, and further optimization is being pursued.

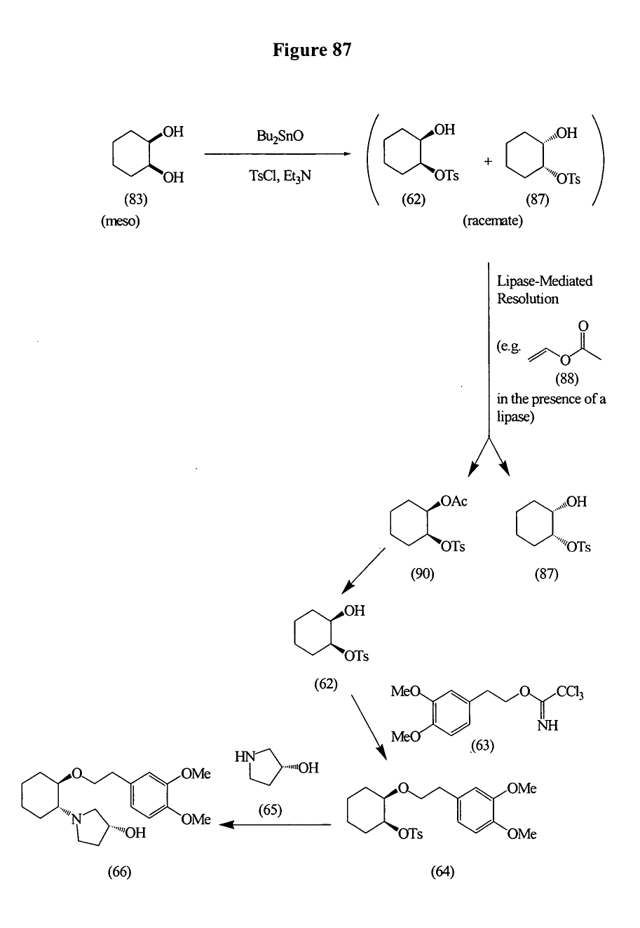

In another embodiment, the preparation of a stereoisomerically substantially pure trans aminocyclohexyl ether compound of formula (66) may be carried out under suitable conditions by a process as outlined in FIG. 87, comprising the steps under suitable conditions as shown in FIG. 87, wherein all the formulae and symbols are as described above. As outlined in FIG. 87, the preparation of a stereoisomerically substantially pure trans aminocyclohexyl ether compound of formula (66) may be carried out by starting with the monotosylation of the cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). The resulting racemic mixture of hydroxytosylates comprises of compounds (62) and (87) is subjected to a lipase-mediated resolution process under suitable conditions such as treatment of the racemates (62) and (87) with vinyl acetate (88) in the presence of a lipase derived from Pseudomonas sp. (N. Boaz et al., Tetra. Asymmetry, 1994, 5, 153) to provide compound (90) and (87).

In a separate step, the stereoisomerically substantially pure compound of formula (90) obtained from the resolution process is subjected to a mild based-catalyzed methanolysis (G. Zemplen et al., Ber., 1936, 69, 1827) to form compound (62). The latter is alkylated under appropriate conditions by treatment with the trichloroacetimidate (63) to form compound (64). The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (88) by trichloroacetimidate (63) may be carried out in the presence of a Lewis acid such as HBF.

In another separate step, the tosylate group of formula (64) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

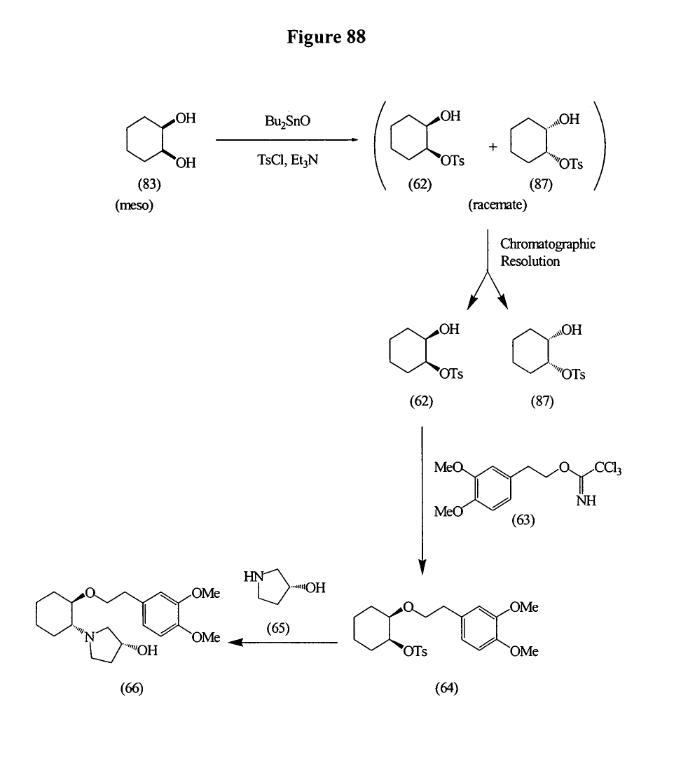

In another embodiment, the present invention provides a process for the preparation of a stereoisomerically substantially pure compound of formula (66), comprising the steps under suitable conditions as shown in FIG. 88, wherein all the formulae and symbols are as described above. As outlined in FIG. 88, the preparation of a stereoisomerically substantially pure trans aminocyclohexyl ether compound of formula (66) may be carried out by starting with the monotosylation of the cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). The resulting racemic mixture of hydroxytosylates comprises of compounds (62) and (87) is subjected to a chromatographic resolution process under suitable conditions such as HPLC with an appropriate chiral stationary phase and simulated moving bed technology to provide compounds (62) and (87) in stereoisomerically substantially pure form.

In a separate step, the stereoisomerically substantially pure compound of formula (62) obtained from the resolution process is alkylated under appropriate conditions by treatment with the trichloroacetimidate (63) to form compound (64). The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (62) by trichloroacetimidate (63) may be carried out in the presence of a Lewis acid such as HBF₄.

In another separate step, the tosylate group of formula (64) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

The reaction sequences described above (FIG. 86, FIG. 87 and FIG. 88) in general generate the compound of formula (66) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

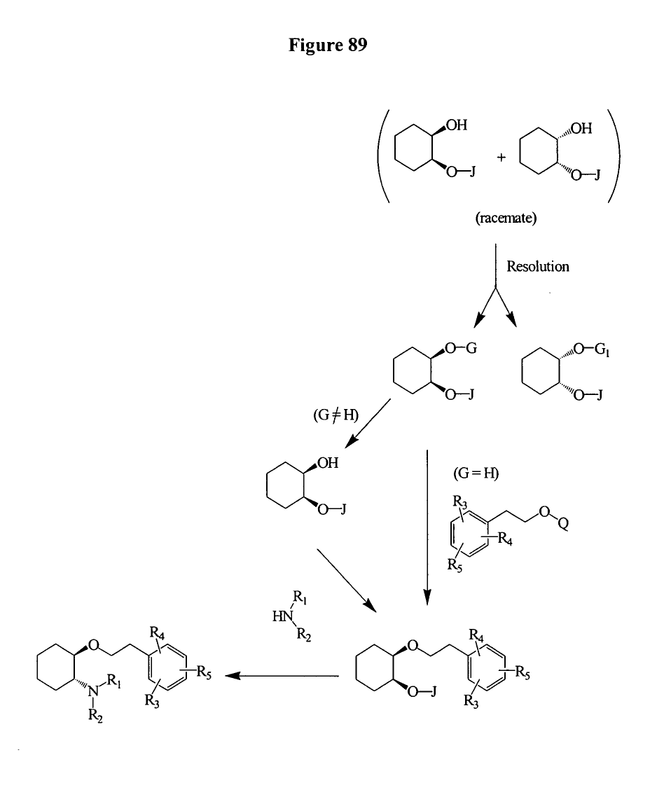

In another embodiment, the preparation of a stereoisomerically substantially pure trans aminocyclohexyl ether compound of formula (57) may be carried out under suitable conditions by a process as outlined in FIG. 89, comprising the steps of starting with a racemic mixture comprises of formulae (53) and (84) and following a reaction sequence analogous to the applicable portion that is described in FIG. 85, wherein all the formulae and symbols are as described above.

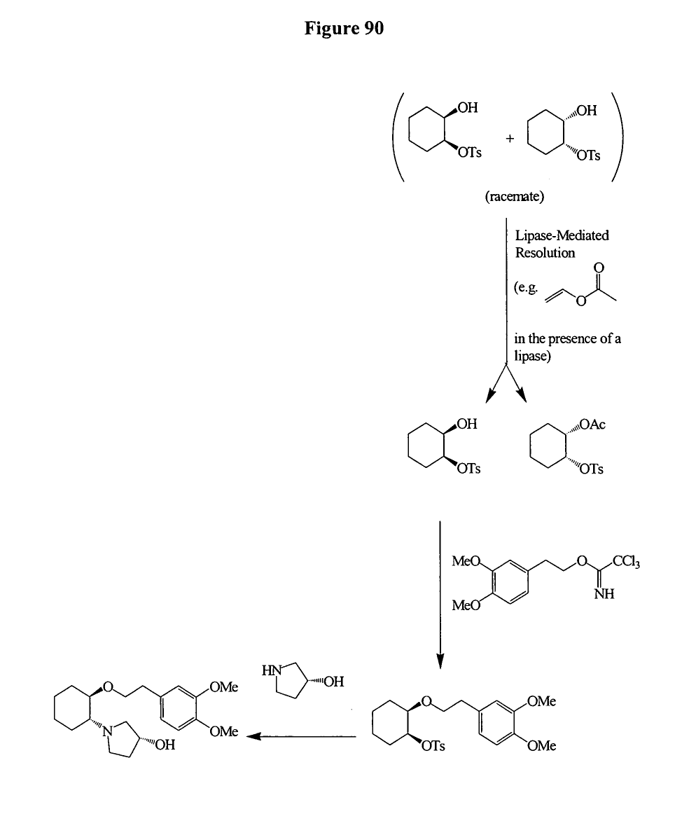

In another embodiment, the preparation of a stereoisomerically substantially pure trans aminocyclohexyl ether compound of formula (66) may be carried out under suitable conditions by a process as outlined in FIG. 90, comprising the steps of starting with a racemic mixture comprises of formulae (62) and (87) and following a reaction sequence analogous to the applicable portion that is described in FIG. 86, wherein all the formulae and symbols are as described above.

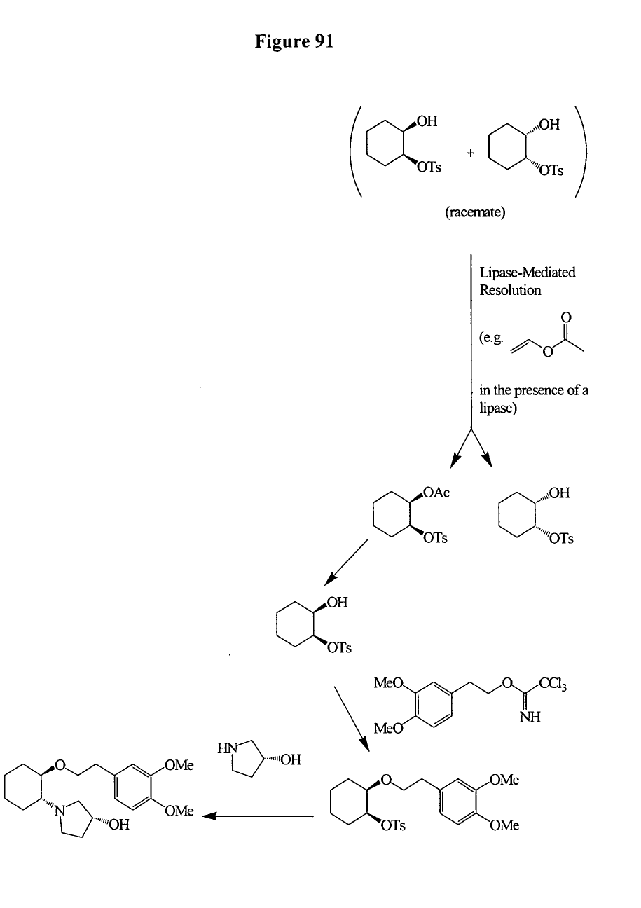

In another embodiment, the preparation of a stereoisomerically substantially pure trans aminocyclohexyl ether compound of formula (66) may be carried out under suitable conditions by a process as outlined in FIG. 91, comprising the steps of starting with a racemic mixture comprises of formulae (62) and (87) and following a reaction sequence analogous to the applicable portion that is described in FIG. 87, wherein all the formulae and symbols are as described above.

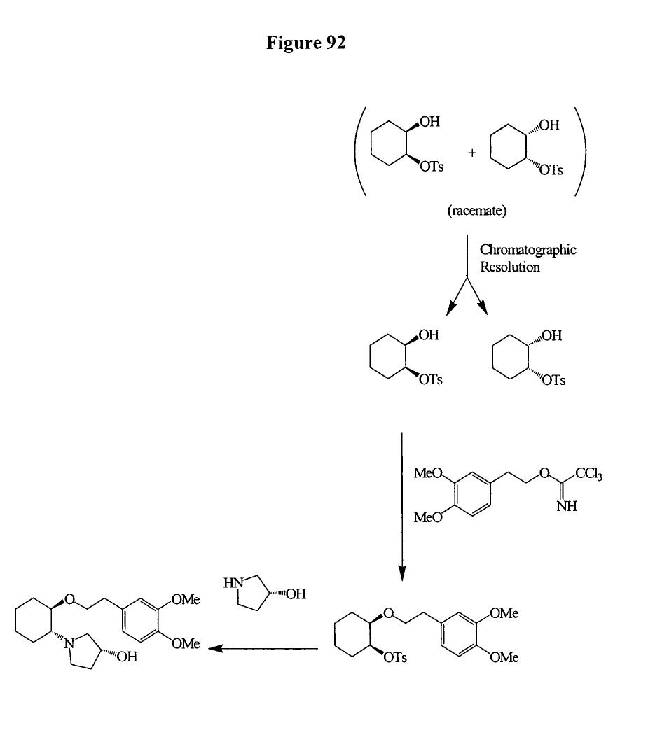

In another embodiment, the preparation of a stereoisomerically substantially pure trans aminocyclohexyl ether compound of formula (66) may be carried out under suitable conditions by a process as outlined in FIG. 92, comprising the steps of starting with a racemic mixture comprises of formulae (62) and (87) and following a reaction sequence analogous to the applicable portion that is described in FIG. 88, wherein all the formulae and symbols are as described above.

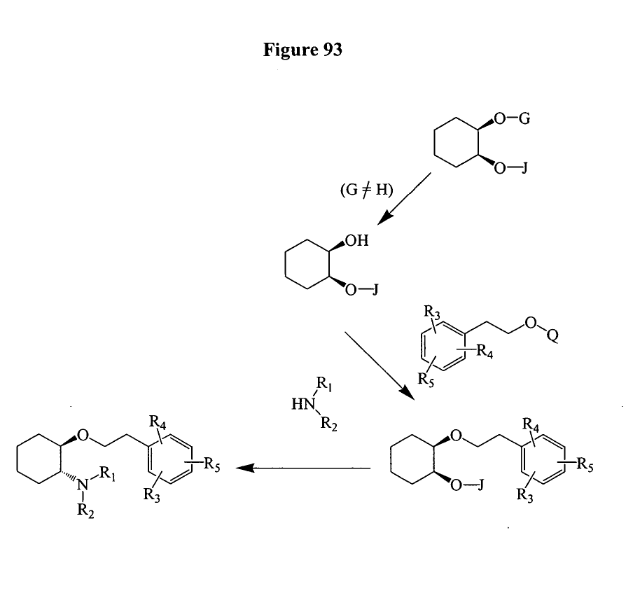

In another embodiment, the preparation of a stereoisomerically substantially pure trans aminocyclohexyl ether compound of formula (57) may be carried out under suitable conditions by a process as outlined in FIG. 93, comprising the steps of starting with a compound of formula (85) where G is not hydrogen and following a reaction sequence analogous to the applicable portion that is described in FIG. 85, wherein all the formulae and symbols are as described above.

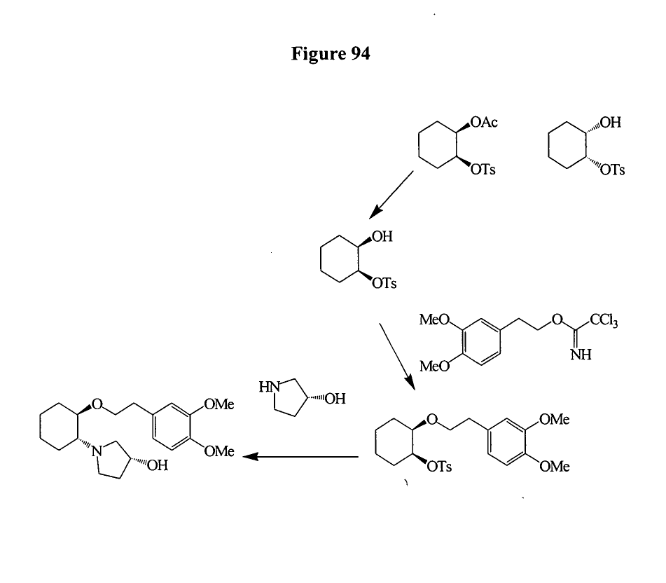

In another embodiment, the preparation of a stereoisomerically substantially pure trans aminocyclohexyl ether compound of formula (66) may be carried out under suitable conditions by a process as outlined in FIG. 94, comprising the steps of starting with a compound of formula (90) and following a reaction sequence analogous to the applicable portion that is described in FIG. 87, wherein all the formulae and symbols are as described above.

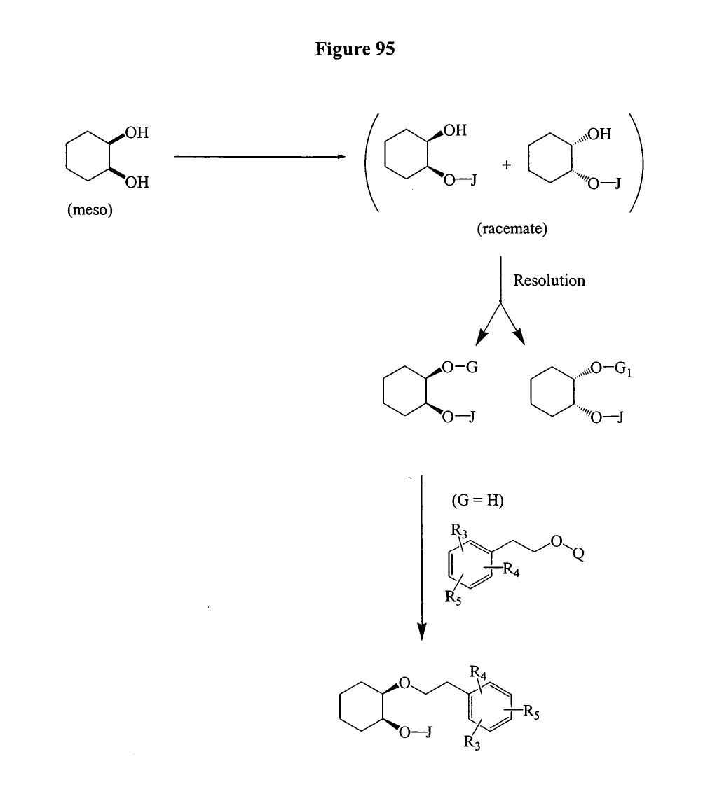

In another embodiment, the preparation of a stereoisomerically substantially pure compound of formula (55) may be carried out under suitable conditions by a process as outlined in FIG. 95, comprising the steps of starting with compound of formula (83) and following a reaction sequence analogous to the applicable portion that is described in FIG. 85, wherein all the formulae and symbols are as described above.

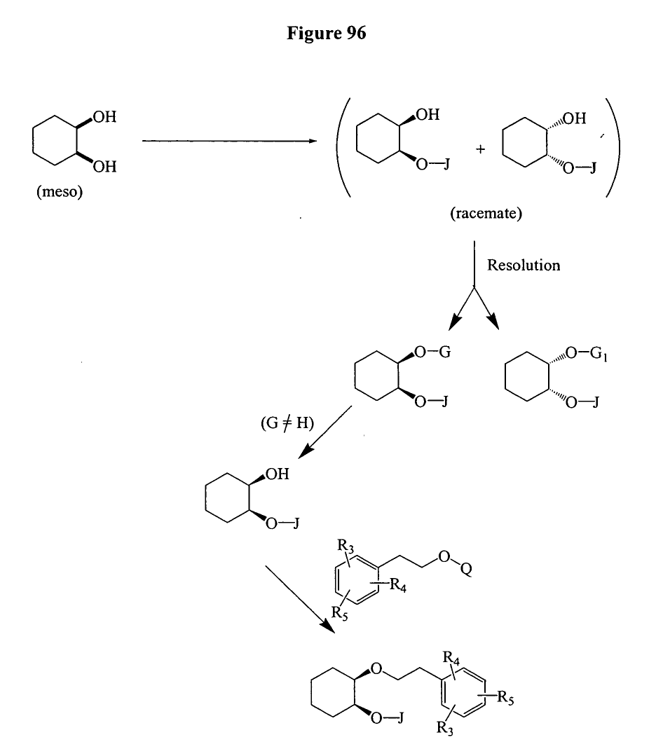

In another embodiment, the preparation of a stereoisomerically substantially pure compound of formula (55) may be carried out under suitable conditions by a process as outlined in FIG. 96, comprising the steps of starting with compound of formula (83) and following a reaction sequence analogous to the applicable portion that is described in FIG. 85, wherein all the formulae and symbols are as described above.

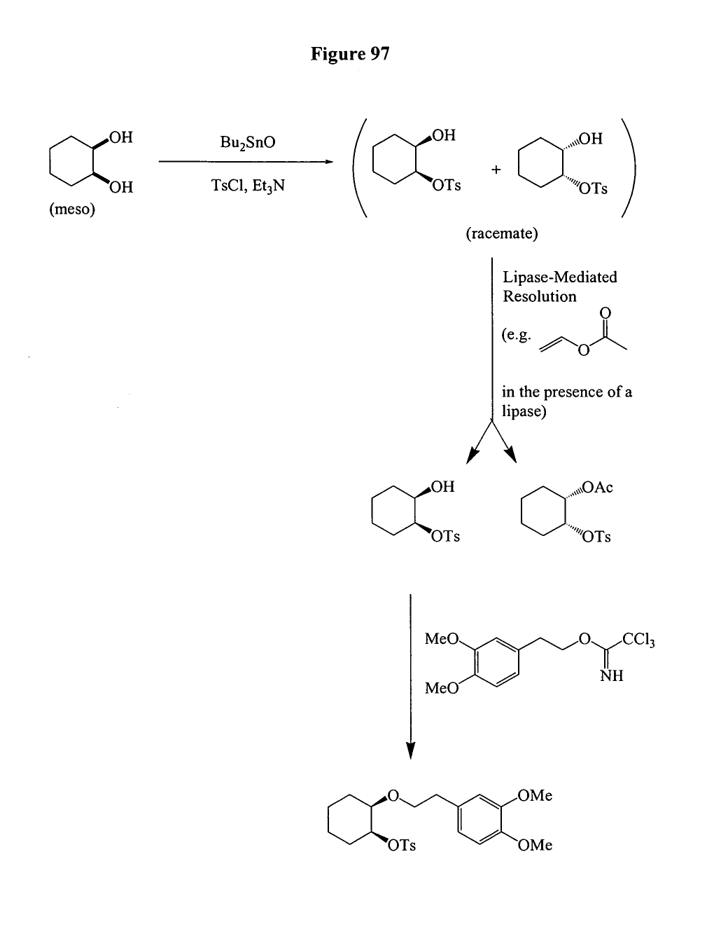

In another embodiment, the preparation of a stereoisomerically substantially pure compound of formula (64) may be carried out under suitable conditions by a process as outlined in FIG. 97, comprising the steps of starting with compound of formula (83) and following a reaction sequence analogous to the applicable portion that is described in FIG. 86, wherein all the formulae and symbols are as described above.

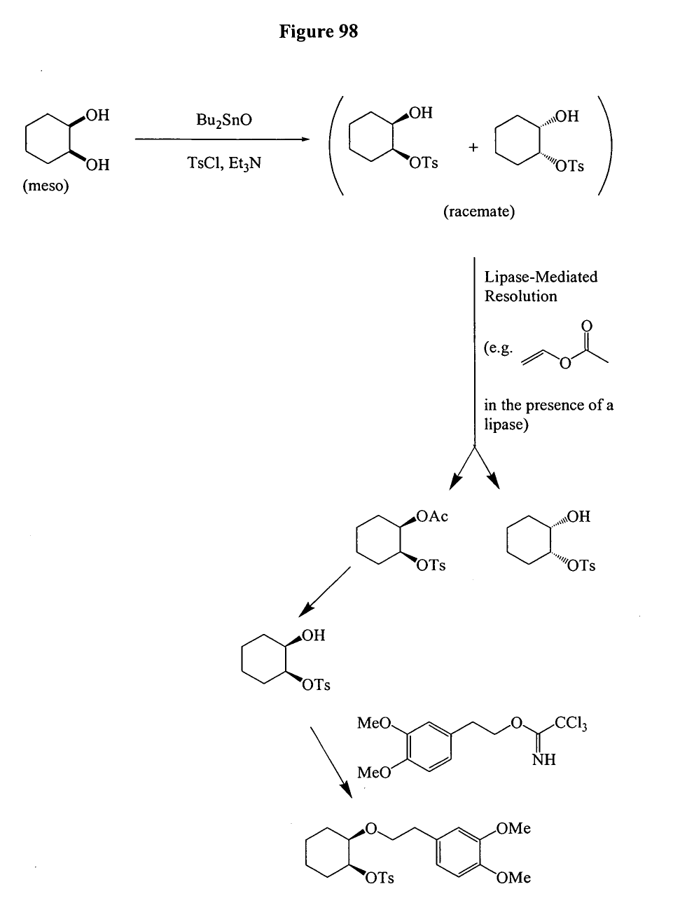

In another embodiment, the preparation of a stereoisomerically substantially pure compound of formula (64) may be carried out under suitable conditions by a process as outlined in FIG. 98, comprising the steps of starting with compound of formula (83) and following a reaction sequence analogous to the applicable portion that is described in FIG. 87, wherein all the formulae and symbols are as described above.

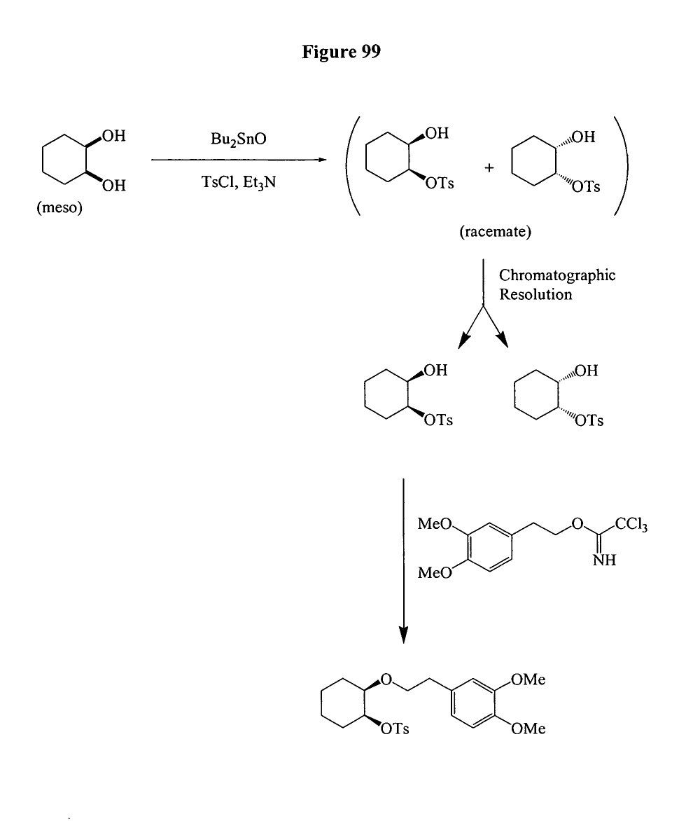

In another embodiment, the preparation of a stereoisomerically substantially pure compound of formula (64) may be carried out under suitable conditions by a process as outlined in FIG. 99, comprising the steps of starting with compound of formula (83) and following a reaction sequence analogous to the applicable portion that is described in FIG. 88, wherein all the formulae and symbols are as described above.

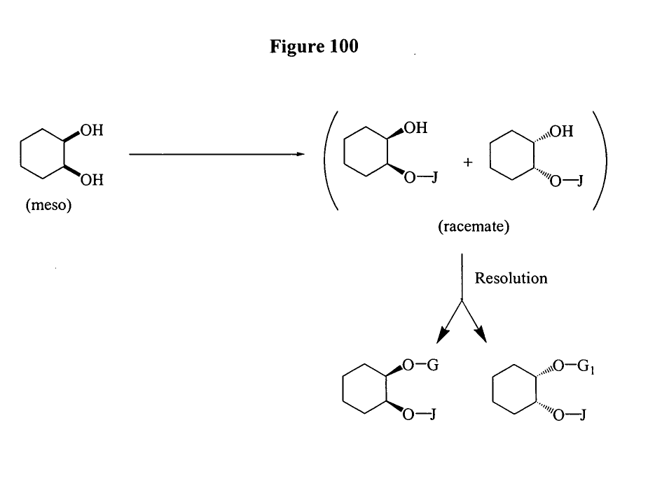

In another embodiment, the preparation of stereoisomerically substantially pure compounds of formulae (85) and (86) may be carried out under suitable conditions by a process as outlined in FIG. 100, comprising the steps of starting with compound of formula (83) and following a reaction sequence analogous to the applicable portion that is described in FIG. 85, wherein all the formulae and symbols are as described above.

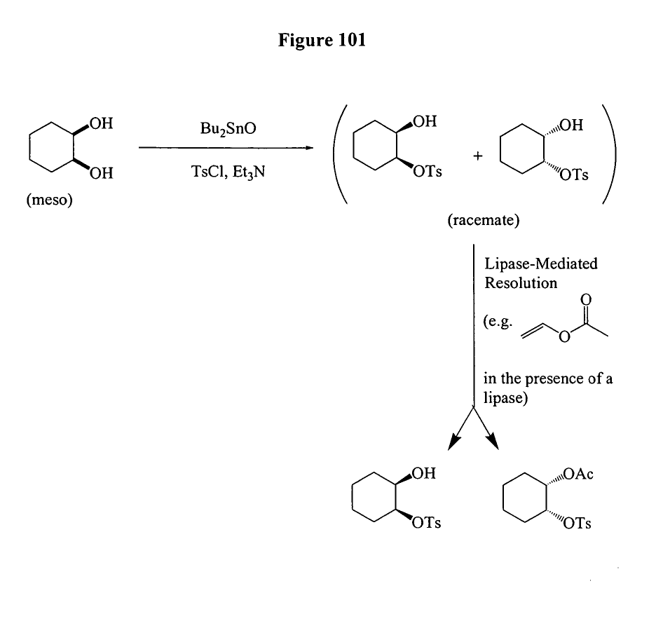

In another embodiment, the preparation of stereoisomerically substantially pure compounds of formulae (62) and (89) may be carried out under suitable conditions by a process as outlined in FIG. 101, comprising the steps of starting with compound of formula (83) and following a reaction sequence analogous to the applicable portion that is described in FIG. 86, wherein all the formulae and symbols are as described above.

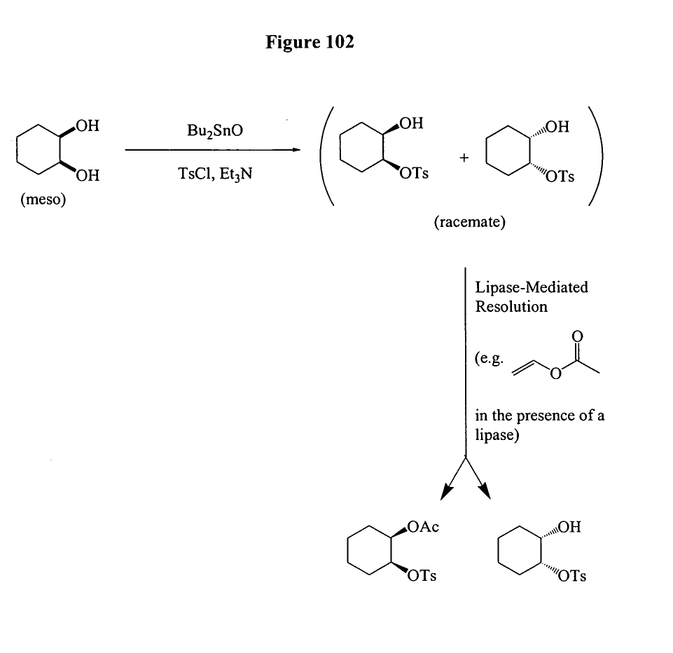

In another embodiment, the preparation of stereoisomerically substantially pure compounds of formulae (90) and (87) may be carried out under suitable conditions by a process as outlined in FIG. 102, comprising the steps of starting with compound of formula (83) and following a reaction sequence analogous to the applicable portion that is described in FIG. 87, wherein all the formulae and symbols are as described above.

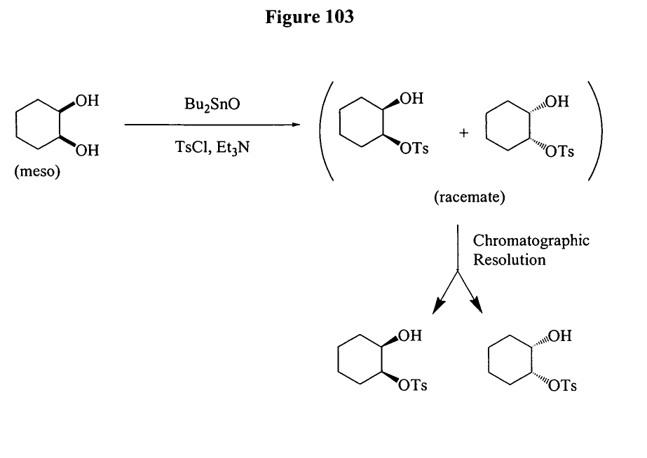

In another embodiment, the preparation of stereoisomerically substantially pure compounds of formulae (62) and (87) may be carried out under suitable conditions by a process as outlined in FIG. 103, comprising the steps of starting with compound of formula (83) and following a reaction sequence analogous to the applicable portion that is described in FIG. 88, wherein all the formulae and symbols are as described above.

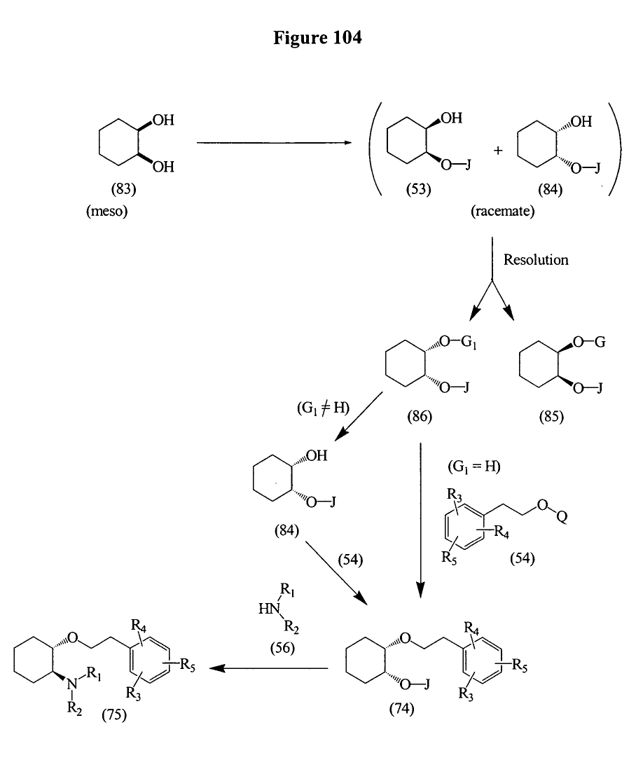

In another embodiment, the present invention further provides synthetic processes whereby compounds of formula (75) with trans-(1S,2S) configuration for the ether and amino functional groups may be prepared in stereoisomerically substantially pure form. As outlined in FIG. 104, the preparation of a stereoisomerically substantially pure trans aminocyclohexyl ether compound of formula (75) may be carried out by following a process starting from a racemic mixture of meso-cis-1,2-cyclohexandiol (83). Compound (83) is commercially available (e.g., Sigma-Aldrich, St. Louis, Mo.) or can be readily synthesized by published methods (e.g., J. E. Taylor et al., Org. Process Res. & Dev., 1998, 2, 147; Organic Syntheses, CV6, 342).

In a first step, one of the hydroxy groups of compound (83) is converted under suitable conditions into an activated form as represented by the racemic mixture comprises of formulae (53) and (84). An “activated form” as used herein means that the hydroxy group is converted into a good leaving group (—O-J) which on reaction with an appropriate nucleophile (e.g., HNR₁R₂) will result in a substitution product with substantial inversion of the stereochemical configuration of the activated hydroxy group. The leaving group (—O-J) may be but is not limited to an alkyl sulfonate such as a trifluoromethanesulfonate group (CF₃SO₃—) or a mesylate group (MsO—), an aryl sulfonate such as a benzenesulfonate group (PhSO₃—), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. The leaving group may be any suitable leaving group on reaction with a nucleophilic reactant with inversion of stereochemical configuration known in the art, including but not limited to compounds disclosed in M. B. Smith and J. March in “March's Advanced Organic Chemistry”, Fifth edition, Chapter 10, John Wiley & Sons, Inc., New York, N Y. (2001). In a typical reaction for the formation of an alkyl sulfonate (e.g., a mesylate) or an aryl sulfonate (e.g., a tosylate or a nosylate), compound (83) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (83) may be used to maximally convert the hydroxy group into the activated form. The hydroxy group may also be converted into other suitable leaving groups according to procedures well known in the art, using any suitable activating agent, including but not limited to those disclosed in M. B. Smith and J. March in “March's Advanced Organic Chemistry”, Fifth edition, Chapter 10, John Wiley & Sons, Inc., New York, N.Y. (2001). The addition of other reagents to facilitate the formation of the monotosylates may be advantageously employed (e.g., M. J. Martinelli, et al. “Selective monosulfonylation of internal 1,2-diols catalyzed by di-n-butyltin oxide” Tetrahedron Letters, 2000, 41, 3773). The racemic mixture comprises of formulae (53) and (84) is then subjected to a resolution process whereby the two optically active isomers are separated into products that are in stereoisomerically substantially pure form such as (85) and (86), wherein G and G₁ are independently selected from hydrogen, C₁-C₈acyl, or any other suitable functional groups that are introduced as part of the resolution process necessary for the separation of the two isomers. In some situations it may be adequate that the resolution process produces compounds of (85) and (86) of sufficient enrichment in their optical purity for application in the subsequent steps of the synthetic process. Methods for resolution of racemic mixtures are well know in the art (e.g., E. L. Eliel and S. H. Wilen, in Stereochemistry of Organic Compounds; John Wiley & Sons: New York, 1994; Chapter 7,. and references cited therein). Suitable processes such as enzymatic resolution (e.g., lipase mediated) and chromatographic separation (e.g., HPLC with chiral stationary phase and/or with simulated moving bed technology, or supercritical fluid chromatography and related techniques) are some of the examples that may be applied (see e.g., T. J. Ward, Analytical Chemistry, 2002, 2863-2872).

For compound of formula (86) when G₁ is hydrogen, (86) is the same as compound (84) and in a separate reaction step, alkylation of the free hydroxy group in compound (86) to form compound (74) is carried out under appropriate conditions with compound (54), where —O-Q represents a good leaving group on reaction with a hydroxy function with retention of the stereochemical configuration of the hydroxy function in the formation of an ether compound. The leaving group may be any suitable leaving group known in the art, including but not limited to compounds disclosed in Greene, “Protective Groups in Organic Chemistry”, John Wiley & Sons, New York N.Y. (1991). Trichloroacetimidate is one example for the —O-Q function. For some compound (54), it may be necessary to introduce appropriate protection groups prior to this step being performed. Suitable protecting groups are set forth in, for example, Greene, “Protective Groups in Organic Chemistry”, John Wiley & Sons, New York N.Y. (1991). For compound of formula (86) when G₁ is not hydrogen, suitable methods are used to convert (86) to compound (84). For example when G₁ is a C₂ acyl function, a mild based-catalyzed methanolysis (G. Zemplen et al., Ber., 1936, 69, 1827) may be used to transform (86) to (84). The latter can then undergo the same reaction with (54) to produce (74) as described above.

In a separate step, the resulted compound (74) is treated under suitable conditions with an amino compound of formula (56) to form compound (75) as the product. The reaction may be carried out with or without a solvent and at an appropriate temperature range that allows the formation of the product (75) at a suitable rate. An excess of the amino compound (56) may be used to maximally convert compound (74) to the product (75). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 104) generates the compound of formula (75) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

In one embodiment, the present invention provides a process for the preparation of a stereoisomerically substantially pure compound of formula (75): 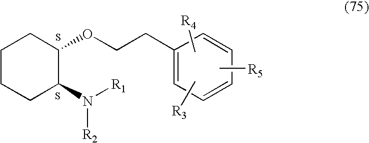
wherein, independently at each occurrence, R₁ and R₂ are independently selected from hydrogen, C₁-C₈alkyl, C₃-C₈alkoxyalkyl, C₁-C₈hydroxyalkyl, and C₇-C₁₂aralkyl; or
R₁ and R₂ are independently selected from C₃-C₈alkoxyalkyl, C₁-C₈hydroxyalkyl, and C₇-C₁₂aralkyl; or
R₁ and R₂, when taken together with the nitrogen atom to which they are directly attached in formula (75), form a ring denoted by formula (I): 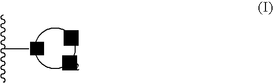
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from hydrogen, hydroxy, C₁-C₃hydroxyalkyl, oxo, C₂-C₄acyl, C₁-C₃alkyl, C₂-C₄alkylcarboxy, C₁-C₃alkoxy, C₁-C₂₀alkanoyloxy, or may be substituted to form a spiro five- or six-membered heterocyclic ring containing one or two heteroatoms selected from oxygen and sulfur; and any two adjacent additional carbon ring atoms may be fused to a C₃-C₈carbocyclic ring, and any one or more of the additional nitrogen ring atoms may be substituted with substituents selected from hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (75), form a ring denoted by formula (II): 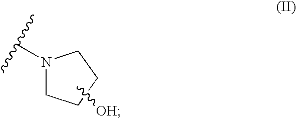
or in another embodiment R₁ and R₂, when taken together with the nitrogen atom to which they are directly attached in formula (I), may form a bicyclic ring system selected from 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently selected from bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl and N(R₆,R₇) where R₆ and R₇ are independently selected from hydrogen, acetyl, methanesulfonyl, and C₁-C₆alkyl; or
R₃, R₄ and R₅ are independently selected from hydrogen, hydroxy and C₁-C₆alkoxy; with the proviso that R₃, R₄ and R₅ cannot all be hydrogen;
comprising the steps of starting with a compound of formula (83), and following a reaction sequence as outlined in FIG. 104 under suitable conditions, wherein
G and G₁ are independently selected from hydrogen, C₁-C₈acyl, or any other suitable functional groups that are introduced as part of the resolution process necessary for the separation of the two isomers;
—O-Q represents a good leaving group which on reaction with a hydroxy function will result in the formation of an ether compound with retention of the stereochemical configuration of the hydroxy function, including, but not limited to, those disclosed in “Protective Groups in Organic Chemistry”, John Wiley & Sons, New York N.Y. (1991); and
—O-J represents a good leaving group on reaction with a nucleophilic reactant will result in a substitution product with substantial inversion of the stereochemical configuration of the activated hydroxy group as shown in FIG. 104; including, but not limited to, those disclosed in “Protective Groups in Organic Chemistry”, John Wiley & Sons, New York N.Y. (1991), and all the formulae and symbols are as described above.

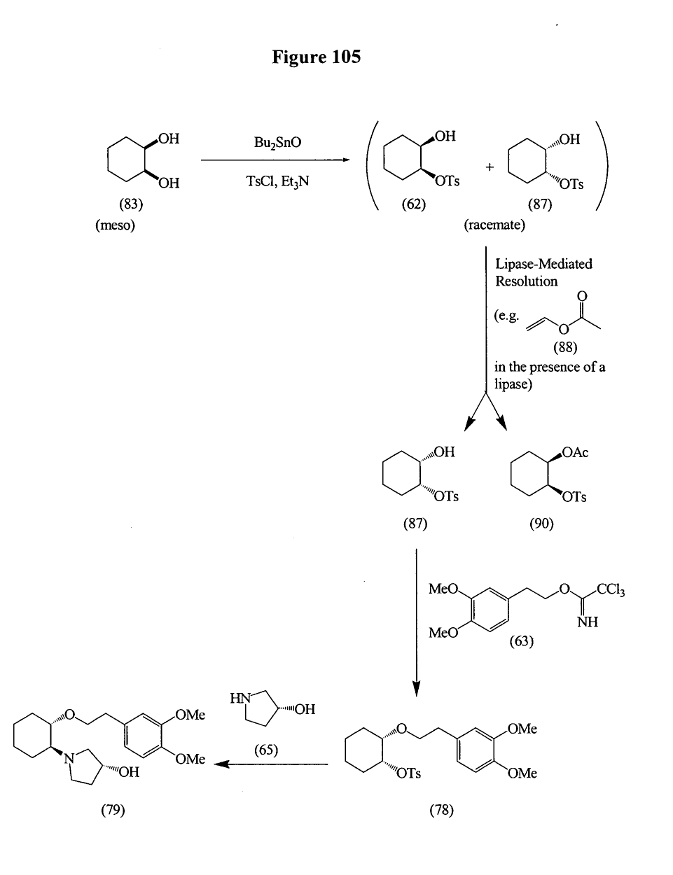

In another embodiment, the present invention provides a process for the preparation of a stereoisomerically substantially pure compound of formula (79), comprising the steps under suitable conditions as shown in FIG. 105, wherein all the formulae and symbols are as described above. As outlined in FIG. 105, the preparation of a stereoisomerically substantially pure trans aminocyclohexyl ether compound of formula (79) may be carried out by starting with the monotosylation of cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). The resulting racemic mixture of hydroxytosylates comprises of compounds (62) and (87) is subjected to a lipase-mediated resolution process under suitable conditions such as treatment of the racemates (62) and (87) with vinyl acetate (88) in the presence of a lipase derived from Pseudomnonas sp. (N. Boaz et al., Tetra. Asymmetry, 1994, 5, 153) to provide compound (87) and (90). In a separate step, the stereoisomerically substantially pure compound of formula (87) obtained from the resolution process is alkylated under appropriate conditions by treatment with the trichloroacetimidate (63) to form compound (78). The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (87) by trichloroacetimidate (63) may be carried out in the presence of a Lewis acid such as HBF₄.

In another separate step, the tosylate group of formula (78) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (79) at a suitable rate. An excess of the amino compound (65) may be used to maximally convert compound (78) to the product (79). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

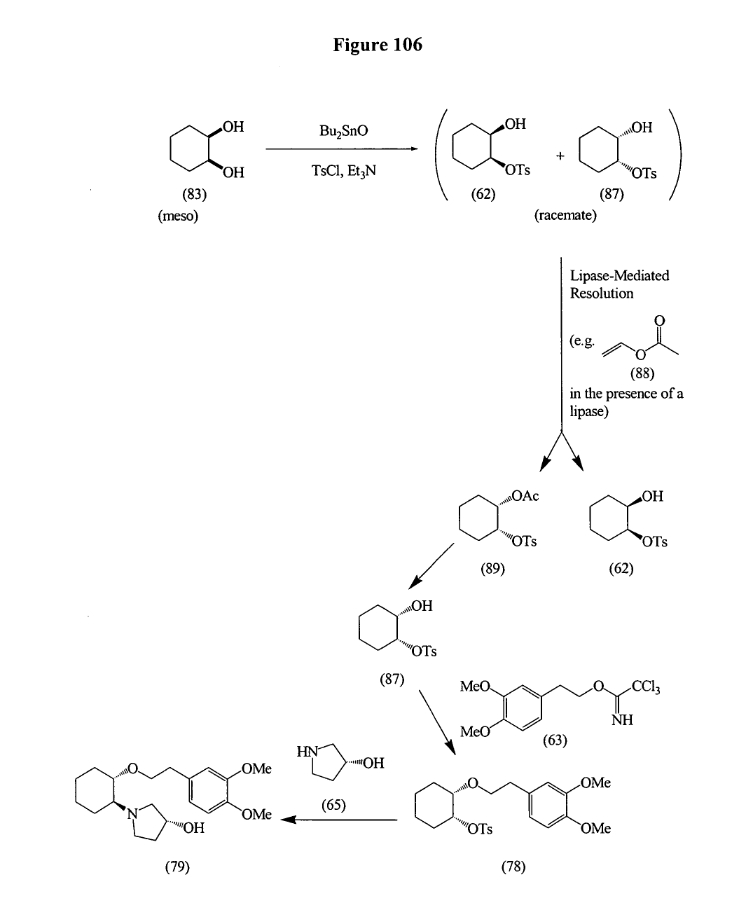

In another embodiment, the preparation of a stereoisomerically substantially pure trans aminocyclohexyl ether compound of formula (79) may be carried out under suitable conditions by a process as outlined in FIG. 106, comprising the steps under suitable conditions as shown in FIG. 106, wherein all the formulae and symbols are as described above. As outlined in FIG. 106, the preparation of a stereoisomerically substantially pure trans aminocyclohexyl ether compound of formula (79) may be carried out by starting with the monotosylation of the cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). The resulting racemic mixture of hydroxytosylates comprises of compounds (62) and (87) is subjected to a lipase-mediated resolution process under suitable conditions such as treatment of the racemates (62) and (87) with vinyl acetate (88) in the presence of a lipase derived from Pseudomonas sp. (N. Boaz et al., Tetra. Asymmetry, 1994, 5, 153) to provide compound (89) and (62).

In a separate step, the stereoisomerically substantially pure compound of formula (89) obtained from the resolution process is subjected to a mild based-catalyzed methanolysis (G. Zemplen et al., Ber., 1936, 69, 1827) to form compound (87). The latter is alkylated under appropriate conditions by treatment with the trichloroacetimidate (63) to form compound (78). The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (87) by trichloroacetimidate (63) may be carried out in the presence of a Lewis acid such as HBF₄.

In another separate step, the tosylate group of formula (78) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (79) at a suitable rate. An excess of the amino compound (65) may be used to maximally convert compound (78) to the product (79). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

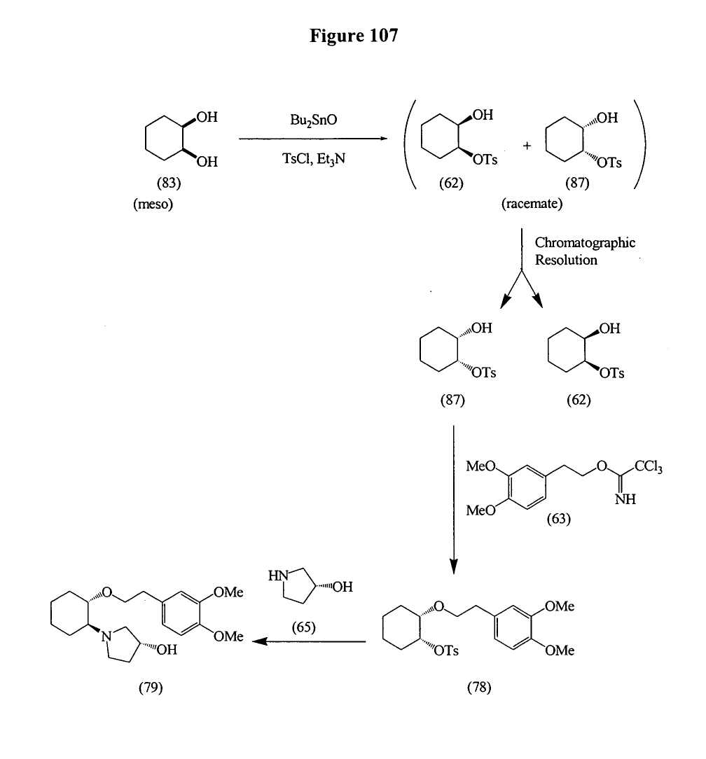

In another embodiment, the present invention provides a process for the preparation of a stereoisomerically substantially pure compound of formula (79), comprising the steps under suitable conditions as shown in FIG. 107, wherein all the formulae and symbols are as described above. As outlined in FIG. 107, the preparation of a stereoisomerically substantially pure trans aminocyclohexyl ether compound of formula (79) may be carried out by starting with the monotosylation of the cis-1,2-cyclohexandiol (83) with TsCl in the presence of Bu₂SnO and triethylamine under suitable conditions (M. J. Martinelli, et al. “Selective monosulfonylation of internal 1,2-diols catalyzed by di-n-butyltin oxide” Tetrahedron Letters, 2000, 41, 3773). The resulting racemic mixture of hydroxytosylates comprises of compounds (62) and (87) is subjected to a chromatographic resolution process under suitable conditions such as HPLC with an appropriate chiral stationary phase and simulated moving bed technology to provide compounds (62) and (87) in stereoisomerically substantially pure form.

In a separate step, the stereoisomerically substantially pure compound of formula (87) obtained from the resolution process is alkylated under appropriate conditions by treatment with the trichloroacetimidate (63) to form compound (64). The trichloroacetimidate (63) is readily prepared from the corresponding alcohol, 3,4-dimethoxyphenethyl alcohol which is commercially available (e.g., Sigma-Aldrich, St. Louis, Mo.), by treatment with trichloroacetonitrile. The alkylation of compound (87) by trichloroacetimidate (63) may be carried out in the presence of a Lewis acid such as HBF₄.

In another separate step, the tosylate group of formula (78) is displaced by an amino compound such as 3R-pyrrolidinol (65) with inversion of configuration. 3R-pyrrolidinol (65) is commercially available (e.g., Sigma-Aldrich, St. Louis, Mo.) or may be prepared according to published procedure (e.g., Chem.Ber./Recueil 1997, 130, 385-397). The reaction may be carried out with or without a solvent and at an appropriate temperature range that allows the formation of the product (79) at a suitable rate. An excess of the amino compound (65) may be used to maximally convert compound (78) to the product (79). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

The reaction sequences described above (FIG. 105, FIG. 106 and FIG. 107) in general generate the compound of formula (79) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

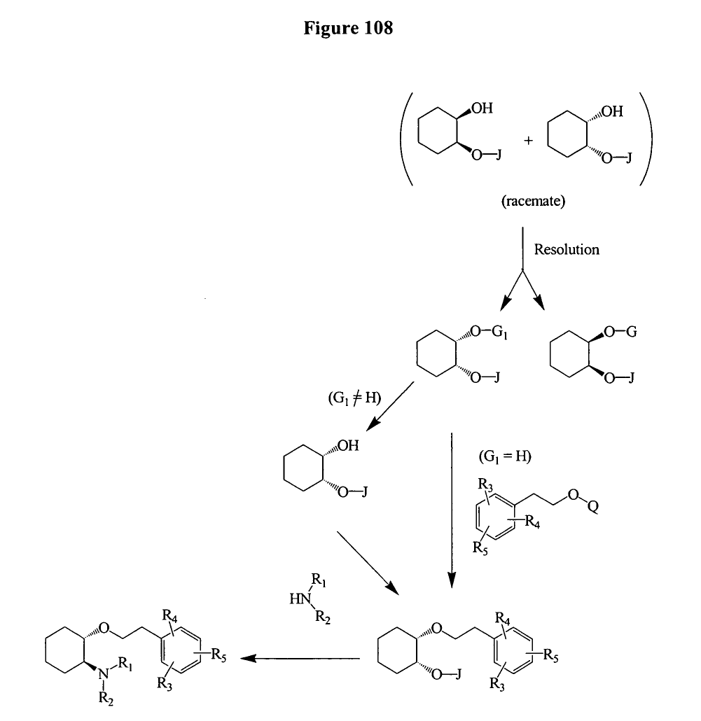

In another embodiment, the preparation of a stereoisomerically substantially pure trans aminocyclohexyl ether compound of formula (75) may be carried out under suitable conditions by a process as outlined in FIG. 108, comprising the steps of starting with a racemic mixture comprises of formulae (53) and (84) and following a reaction sequence analogous to the applicable portion that is described in FIG. 104, wherein all the formulae and symbols are as described above.

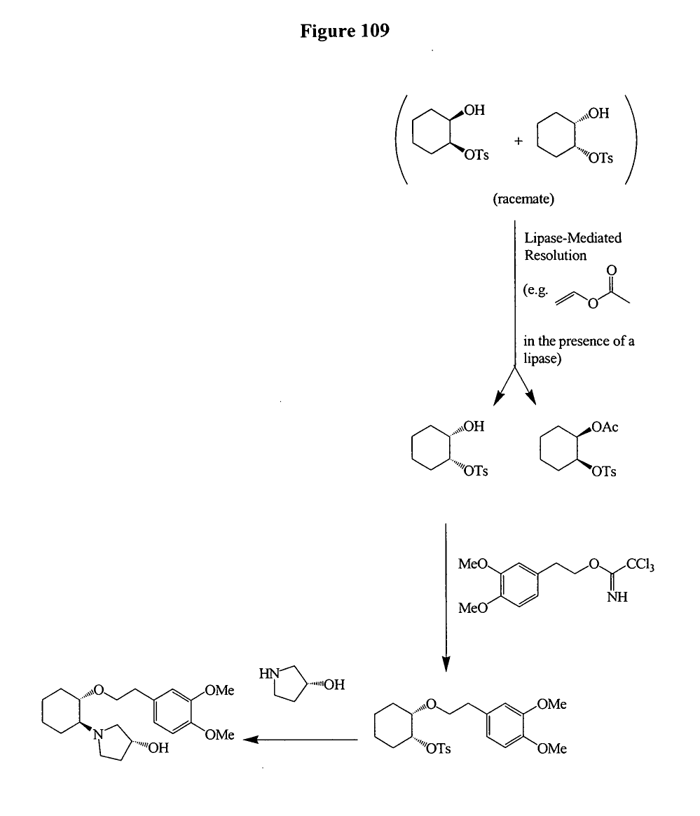

In another embodiment, the preparation of a stereoisomerically substantially pure trans aminocyclohexyl ether compound of formula (79) may be carried out under suitable conditions by a process as outlined in FIG. 109, comprising the steps of starting with a racemic mixture comprises of formulae (62) and (87) and following a reaction sequence analogous to the applicable portion that is described in FIG. 105, wherein all the formulae and symbols are as described above.

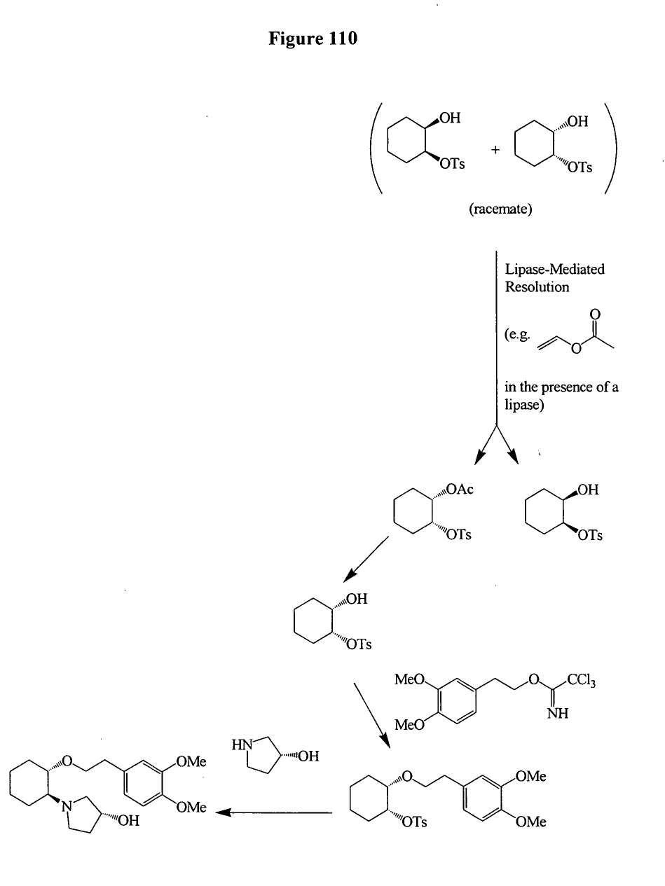

In another embodiment, the preparation of a stereoisomerically substantially pure trans aminocyclohexyl ether compound of formula (79) may be carried out under suitable conditions by a process as outlined in FIG. 110, comprising the steps of starting with a racemic mixture comprises of formulae (62) and (87) and following a reaction sequence analogous to the applicable portion that is described in FIG. 106, wherein all the formulae and symbols are as described above.

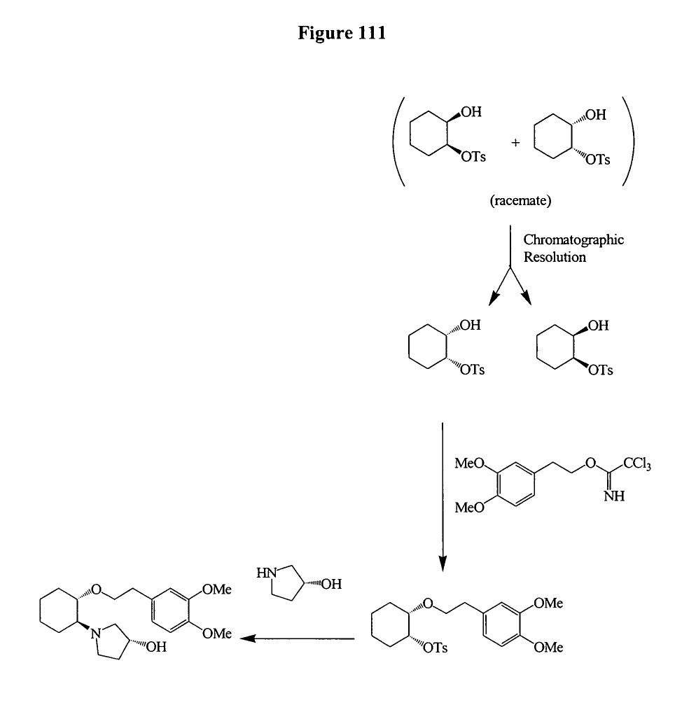

In another embodiment, the preparation of a stereoisomerically substantially pure trans aminocyclohexyl ether compound of formula (79) may be carried out under suitable conditions by a process as outlined in FIG. 111, comprising the steps of starting with a racemic mixture comprises of formulae (62) and (87) and following a reaction sequence analogous to the applicable portion that is described in FIG. 107, wherein all the formulae and symbols are as described above.

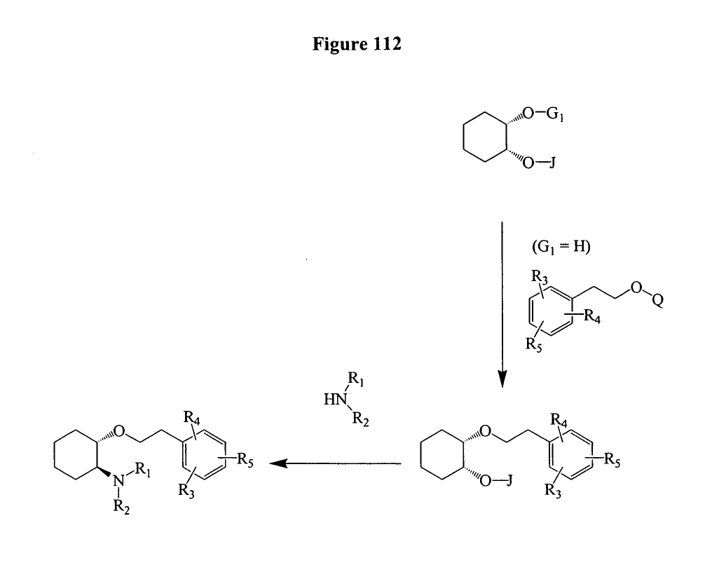

In another embodiment, the preparation of a stereoisomerically substantially pure trans aminocyclohexyl ether compound of formula (75) may be carried out under suitable conditions by a process as outlined in FIG. 112, comprising the steps of starting with a compound of formula (86) where G₁ is hydrogen and following a reaction sequence analogous to the applicable portion that is described in FIG. 104, wherein all the formulae and symbols are as described above.

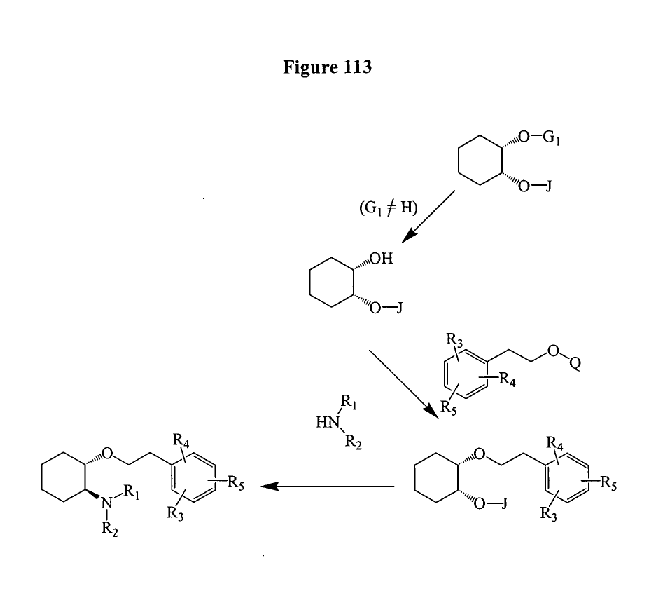

In another embodiment, the preparation of a stereoisomerically substantially pure trans aminocyclohexyl ether compound of formula (75) may be carried out under suitable conditions by a process as outlined in FIG. 113, comprising the steps of starting with a compound of formula (86) where G₁ is not hydrogen and following a reaction sequence analogous to the applicable portion that is described in FIG. 104, wherein all the formulae and symbols are as described above.

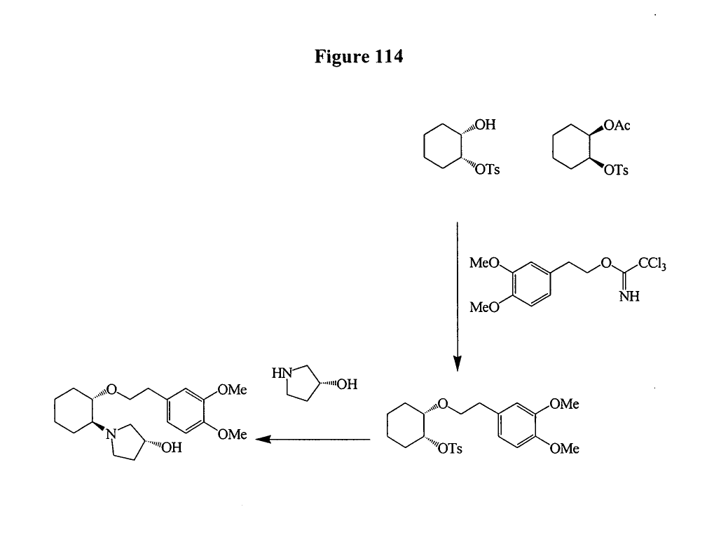

In another embodiment, the preparation of a stereoisomerically substantially pure trans aminocyclohexyl ether compound of formula (79) may be carried out under suitable conditions by a process as outlined in FIG. 114, comprising the steps of starting with a compound of formula (87) and following a reaction sequence analogous to the applicable portion that is described in FIG. 105, wherein all the formulae and symbols are as described above.

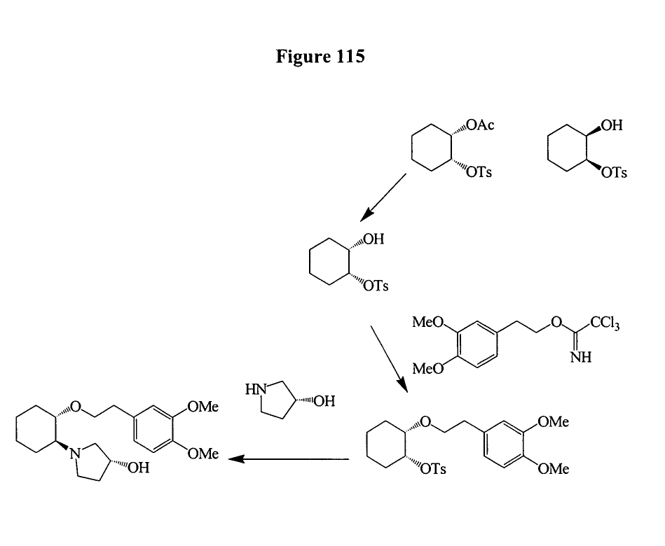

In another embodiment, the preparation of a stereoisomerically substantially pure trans aminocyclohexyl ether compound of formula (79) may be carried out under suitable conditions by a process as outlined in FIG. 115, comprising the steps of starting with a compound of formula (89) and following a reaction sequence analogous to the applicable portion that is described in FIG. 106, wherein all the formulae and symbols are as described above.

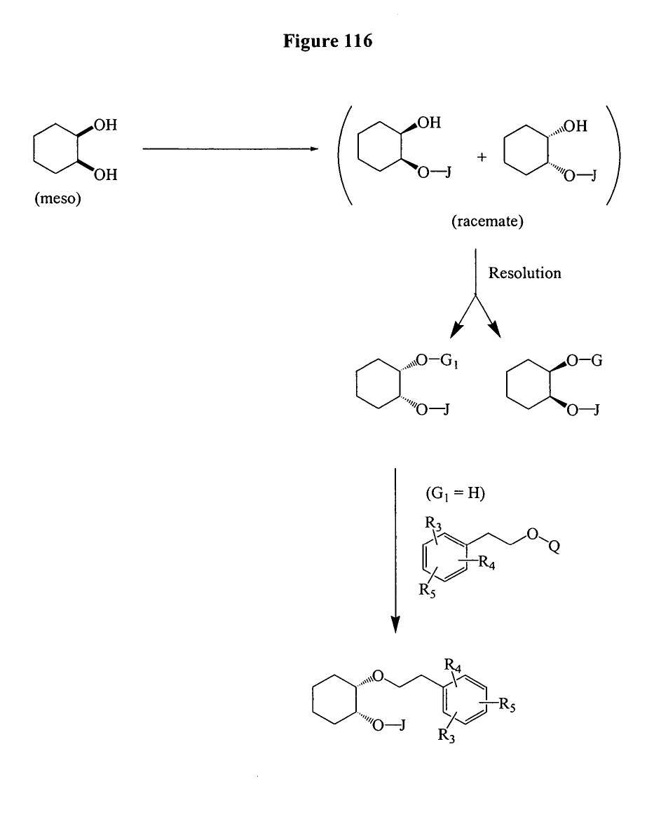

In another embodiment, the preparation of a stereoisomerically substantially pure compound of formula (74) may be carried out under suitable conditions by a process as outlined in FIG. 116, comprising the steps of starting with compound of formula (83) and following a reaction sequence analogous to the applicable portion that is described in FIG. 104, wherein all the formulae and symbols are as described above.

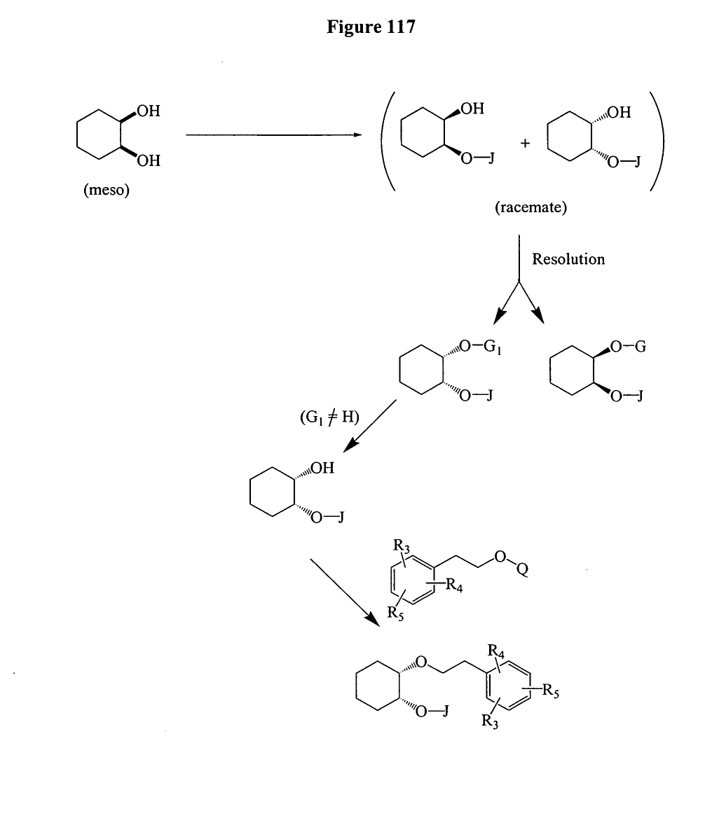

In another embodiment, the preparation of a stereoisomerically substantially pure compound of formula (74) may be carried out under suitable conditions by a process as outlined in FIG. 117, comprising the steps of starting with compound of formula (83) and following a reaction sequence analogous to the applicable portion that is described in FIG. 104, wherein all the formulae and symbols are as described above.

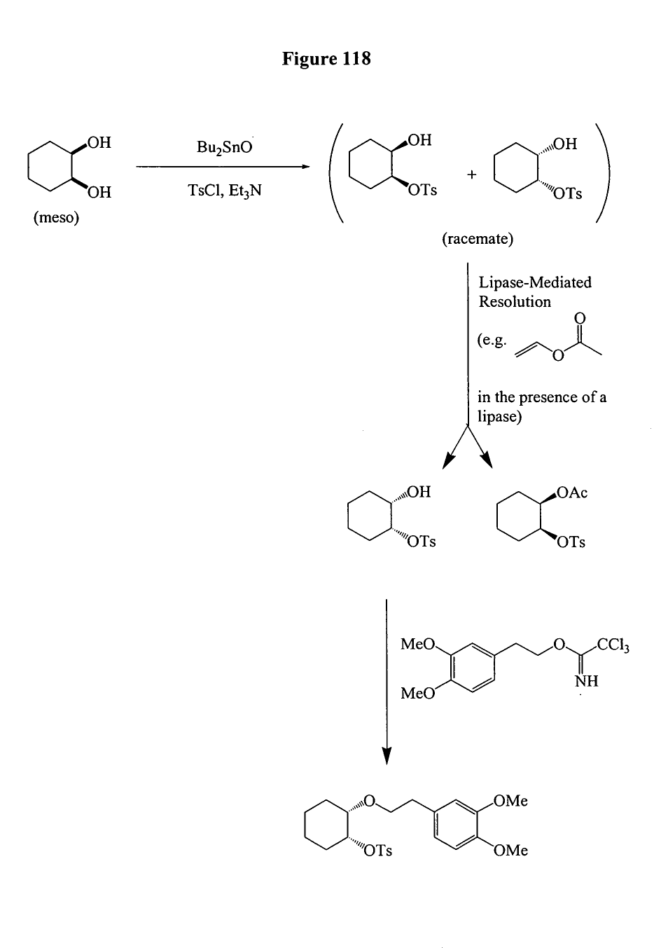

In another embodiment, the preparation of a stereoisomerically substantially pure compound of formula (78) may be carried out under suitable conditions by a process as outlined in FIG. 118, comprising the steps of starting with compound of formula (83) and following a reaction sequence analogous to the applicable portion that is described in FIG. 105, wherein all the formulae and symbols are as described above.

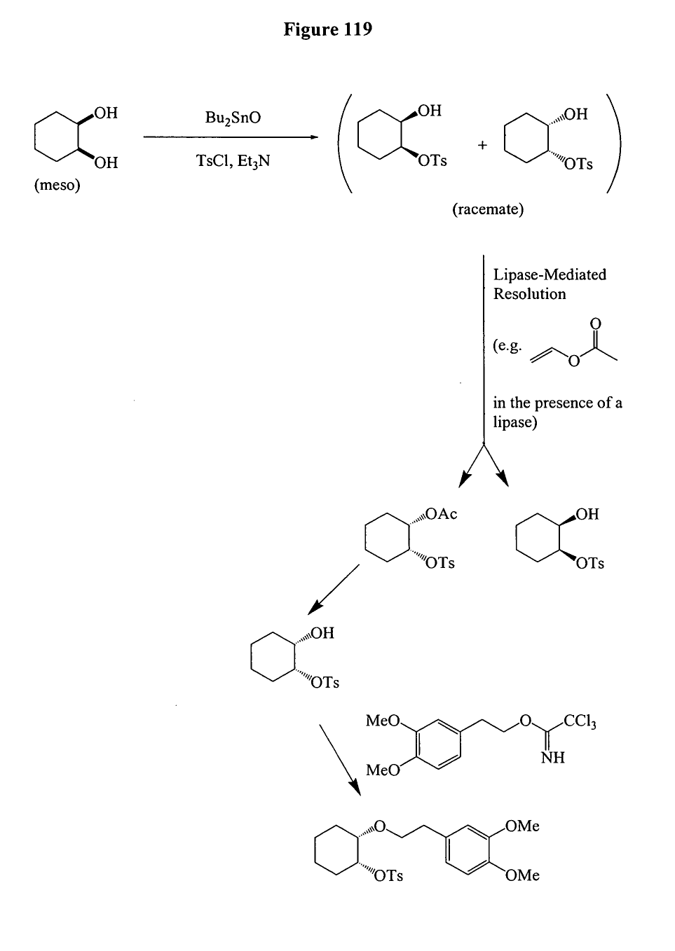

In another embodiment, the preparation of a stereoisomerically substantially pure compound of formula (78) may be carried out under suitable conditions by a process as outlined in FIG. 119, comprising the steps of starting with compound of formula (83) and following a reaction sequence analogous to the applicable portion that is described in FIG. 106, wherein all the formulae and symbols are as described above.

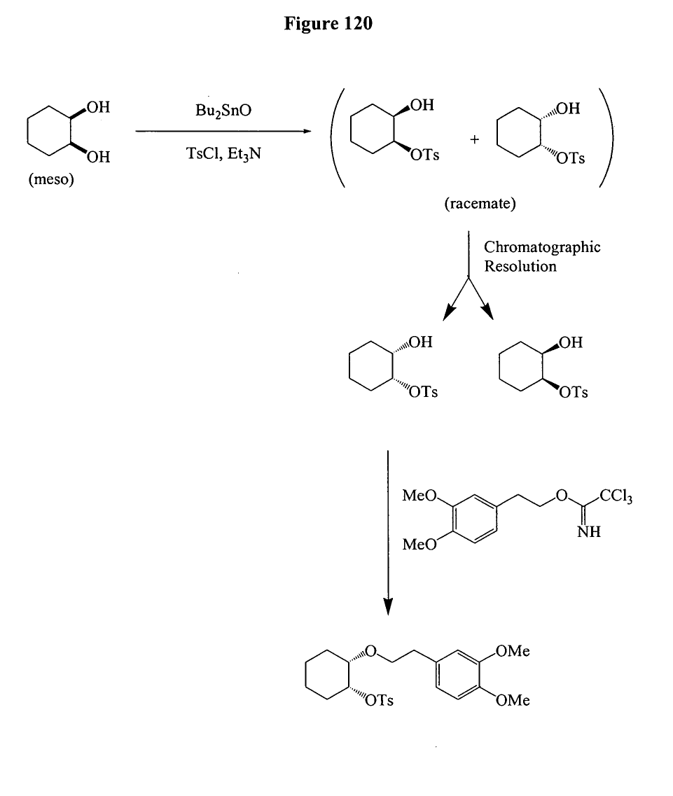

In another embodiment, the preparation of a stereoisomerically substantially pure compound of formula (78) may be carried out under suitable conditions by a process as outlined in FIG. 120, comprising the steps of starting with compound of formula (83) and following a reaction sequence analogous to the applicable portion that is described in FIG. 107, wherein all the formulae and symbols are as described above.

In another embodiment, the present invention provides a compound of formula (85), or a solvate or pharmaceutically acceptable salt thereof; wherein all the formulae and symbols are as described above.

In another embodiment, the present invention provides a compound of formula (86), or a solvate or pharmaceutically acceptable salt thereof; wherein all the formulae and symbols are as described above.

In another embodiment, the present invention provides a compound of formula (54), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (55), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (87), or a solvate or pharmaceutically acceptable salt thereof; wherein all the formulae and symbols are as described above.

In another embodiment, the present invention provides a compound of formula (62), or a solvate or pharmaceutically acceptable salt thereof; wherein all the formulae and symbols are as described above.

In another embodiment, the present invention provides a compound of formula (89), or a solvate or pharmaceutically acceptable salt thereof; wherein all the formulae and symbols are as described above.

In another embodiment, the present invention provides a compound of formula (90), or a solvate or pharmaceutically acceptable salt thereof; wherein all the formulae and symbols are as described above.

In another embodiment, the present invention provides a compound of formula (64), or a solvate or pharmaceutically acceptable salt thereof; wherein all the formulae and symbols are as described above.

In another embodiment, the present invention provides a compound of formula (74), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (78), or a solvate or pharmaceutically acceptable salt thereof; wherein all the formulae and symbols are as described above.

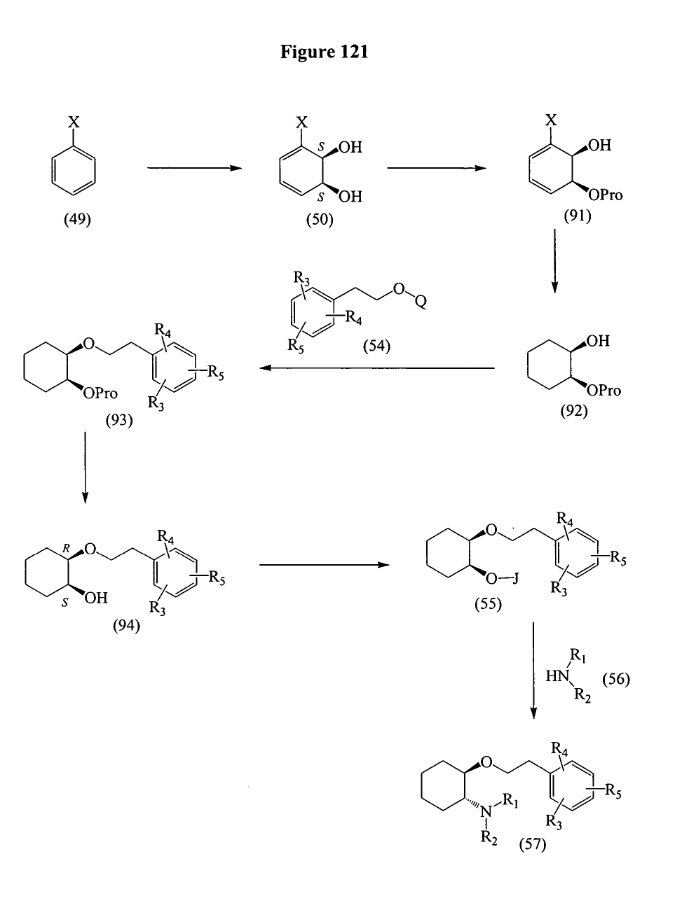

In one embodiment, the present invention provides a process for the preparation of a stereoisomerically substantially pure compound of formula (57): 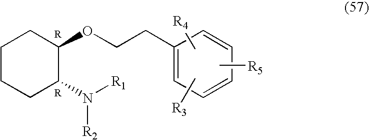
wherein, independently at each occurrence, R₁ and R₂ are independently selected from hydrogen, C₁-C₈alkyl, C₃-C₈alkoxyalkyl, C₁-C₈hydroxyalkyl, and C₇-C₁₂aralkyl; or
R₁ and R₂ are independently selected from C₃-C₈alkoxyalkyl, C₁-C₈hydroxyalkyl, and C₇-C₁₂aralkyl; or
R₁ and R₂, when taken together with the nitrogen atom to which they are directly attached in formula (57), form a ring denoted by formula (I): 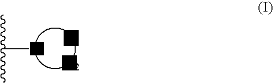
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from hydrogen, hydroxy, C₁-C₃hydroxyalkyl, oxo, C₂-C₄acyl, C₁-C₃alkyl, C₂-C₄alkylcarboxy, C₁-C₃alkoxy, C₁-C₂₀alkanoyloxy, or may be substituted to form a spiro five- or six-membered heterocyclic ring containing one or two heteroatoms selected from oxygen and sulfur; and any two adjacent additional carbon ring atoms may be fused to a C₃-C₈carbocyclic ring, and any one or more of the additional nitrogen ring atoms may be substituted with substituents selected from hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (57), form a ring denoted by formula (II): 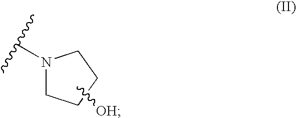
or in another embodiment R₁ and R₂, when taken together with the nitrogen atom to which they are directly attached in formula (I), may form a bicyclic ring system selected from 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently selected from bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl and N(R₆, R₇) where R₆ and R₇ are independently selected from hydrogen, acetyl, methanesulfonyl, and C₁-C₆alkyl; or
R₃, R₄ and R₅ are independently selected from hydrogen, hydroxy and C₁-C₆alkoxy; with the proviso that R₃, R₄ and R₅ cannot all be hydrogen;
comprising the steps of starting with a monohalobenzene (49), wherein X may be F, Cl, Br or I; and following a reaction sequence as outlined in FIG. 121 under suitable conditions, wherein
Pro represents the appropriate protecting group of the hydroxy function with retention of stereochemistry;
—O-Q represents a good leaving group which on reaction with a hydroxy function will result in the formation of an ether compound with retention of the stereochemical configuration of the hydroxy function; and
—O-J represents a good leaving group on reaction with a nucleophilic reactant will result in a substitution product with substantial inversion of the stereochemical configuration of the activated hydroxy group as shown in FIG. 121; and all the formulae and symbols are as described above.

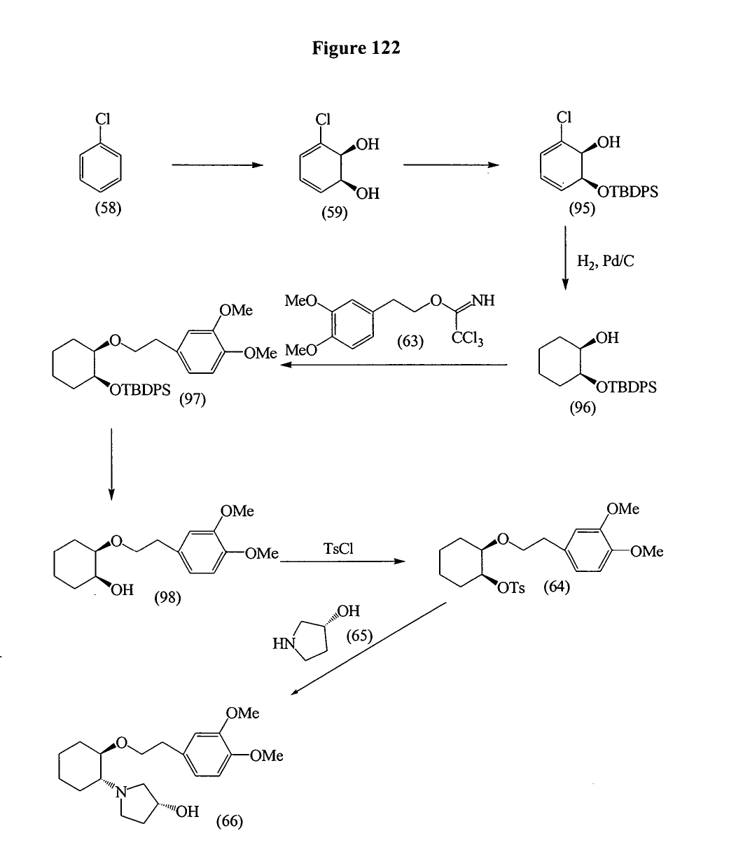

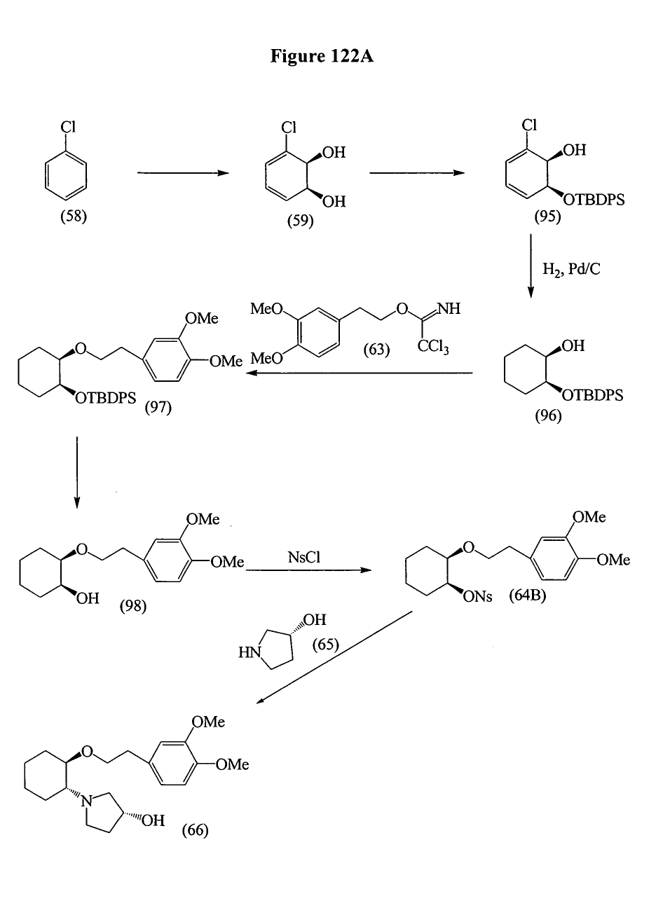

In another embodiment, the present invention provides a process for the preparation of a stereoisomerically substantially pure compound of formula (66), comprising the steps under suitable conditions as shown in FIG. 122, wherein all the formulae and symbols are as described above. As outlined in FIG. 122, the preparation of a stereoisomerically substantially pure trans aminocyclohexyl ether compound of formula (66) may be carried out by starting with a biotransformation of chlorobenzene (58) to compound (59) by microorganism such as Pseudomonas putida 39/D. Experimental conditions for the biotransformation are well established (Organic Synthesis, Vol. 76, 77 and T. Hudlicky et al., Aldrichimica Acta, 1999, 32, 35; and references cited therein). In a separate step, the less hindered hydroxy function in compound (59) is selectively monosilylated as compound (95) by reaction with silylating reagent such as t-butyldiphenylsilyl chloride (TBDPSCl) under suitable conditions (e.g., imaidazole in CH₂Cl₂) (T. Hudlicky et al., Aldrichimica Acta, 1999, 32, 35; S. M. Brown and T. Hudlicky, In Organic Synthesis: Theory and Applications; T. Hudlicky, Ed.; JAI Press: Greenwich, Conn., 1993; Vol. 2, p 113; and references cited therein). In another separate step, compound (95) is converted to compound (96) by reduction such as hydrogenation and hydrogenolysis in the presence of a catalyst under appropriate conditions. Palladium on activated carbon is one example of the catalysts. The reduction of compound (95) may be conducted under basic conditions e.g., in the presence of a base such as sodium ethoxide, sodium bicarbonate, sodium acetate or calcium carbonate. The base may be added in one portion or incrementally during the course of the reaction. In a separate step, the free hydroxy group in compound (96) is alkylated under appropriate conditions to form compound (97). The trichloroacetimidate (63) is readily prepared from the corresponding alcohol, 3,4-dimethoxyphenethyl alcohol which is commercially available (e.g., Aldrich), by treatment with trichloroacetonitrile. The alkylation of compound (96) by trichloroacetimidate (63) may be carried out in the presence of a Lewis acid such as HBF₄. In another separate step, the t-butyldiphenylsilyl (TBDPS) protection group in compound (97) may be removed by standard procedures (e.g., tetrabutylammonium fluoride in tetrahydrofuran (THF) or as described in Greene, “Protective Groups in Organic Chemistry”, John Wiley & Sons, New York N.Y. (1991)) to afford the hydroxyether compound (98). In a separate step, the hydroxy group of compound (98) is converted under suitable conditions into an activated form such as the tosylate of formula (64). In another separate step, the tosylate group of formula (64) is displaced by an amino compound such as 3R-pyrrolidinol (65) with inversion of configuration. 3R-pyrrolidinol (65) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally, the additional base is non-nucleophilic in chemical reactivity. When the reaction has proceeded to substantial completion, the desired product is recovered from the reaction mixture by conventional organic chemistry techniques, and is purified accordingly.

In another embodiment, the present invention provides a process for the preparation of a stereoisomerically substantially pure compound of formula (66), comprising the steps under suitable conditions as shown in FIG. 122A, wherein all the formulae and symbols are as described above. As outlined in FIG. 122A, the preparation of a stereoisomerically substantially pure trans aminocyclohexyl ether compound of formula (66) may be carried out by starting with a biotransformation of chlorobenzene (58) to compound (59) by microorganism such as Pseudomonas putida 39/D. Experimental conditions for the biotransformation are well established (Organic Synthesis, Vol. 76, 77 and T. Hudlicky et al., Aldrichimica Acta, 1999, 32, 35; and references cited therein). In a separate step, the less hindered hydroxy function in compound (59) is selectively monosilylated as compound (95) by reaction with silylating reagent such as t-butyldiphenylsilyl chloride (TBDPSCl) under suitable conditions (e.g., imaidazole in CH₂Cl₂) (T. Hudlicky et al., Aldrichimica Acta, 1999, 32, 35; S. M. Brown and T. Hudlicky, In Organic Synthesis: Theory and Applications; T. Hudlicky, Ed.; JAI Press: Greenwich, Conn., 1993; Vol. 2, p 113; and references cited therein). In another separate step, compound (95) is converted to compound (96) by reduction such as hydrogenation and hydrogenolysis in the presence of a catalyst under appropriate conditions. Palladium on activated carbon is one example of the catalysts. The reduction of compound (95) may be conducted under basic conditions e.g., in the presence of a base such as sodium ethoxide, sodium bicarbonate, sodium acetate or calcium carbonate. The base may be added in one portion or incrementally during the course of the reaction. In a separate step, the free hydroxy group in compound (96) is alkylated under appropriate conditions to form compound (97). The trichloroacetimidate (63) is readily prepared from the corresponding alcohol, 3,4-dimethoxyphenethyl alcohol which is commercially available (e.g., Aldrich), by treatment with trichloroacetonitrile. The alkylation of compound (96) by trichloroacetimidate (63) may be carried out in the presence of a Lewis acid such as HBF₄. In another separate step, the t-butyldiphenylsilyl (TBDPS) protection group in compound (97) may be removed by standard procedures (e.g., tetrabutylammonium fluoride in tetrahydrofuran (THF) or as described in Greene, “Protective Groups in Organic Chemistry”, John Wiley & Sons, New York N.Y. (1991)) to afford the hydroxyether compound (98). In a separate step, the hydroxy group of compound (98) is converted under suitable conditions into an activated form such as the nosylate of formula (64B). In another separate step, the nosylate group of formula (64B) is displaced by an amino compound such as 3R-pyrrolidinol (65) with inversion of configuration. 3R-pyrrolidinol (65) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (66) at a suitable rate. An excess of the amino compound (65) may be used to maximally convert compound (64) to the product (66). The reaction may be performed in the presence of a base that can facilitate the formation of the product. Generally the additional base is non-nucleophilic in chemical reactivity. When the reaction has proceeded to substantial completion, the desired product is recovered from the reaction mixture by conventional organic chemistry techniques, and is purified accordingly.

The reaction sequences described above (FIG. 122 and FIG. 122A) in general generates the compound of formula (66) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

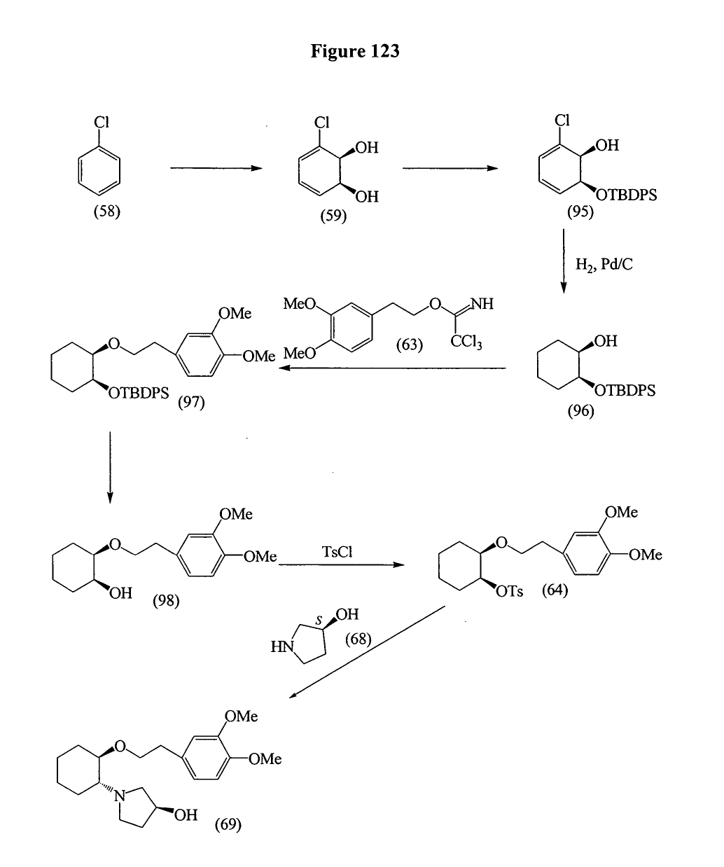

In another embodiment, the preparation of a stereoisomerically substantially pure trans aminocyclohexyl ether compound of formula (69) may be carried out by a process as outlined in FIG. 123, comprising the steps of starting with chlorobenzene (58) and following a reaction sequence under suitable conditions analogous to the applicable portion that is described in FIG. 122 above leading to compound of formula (64). The latter is reacted with an amino compound of formula (68). Compound (68), 3S-pyrrolidinol, is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (69) at a suitable rate. An excess of the amino compound (68) may be used to maximally convert compound (64) to the product (69). The reaction may be performed in the presence of a base that can facilitate the formation of the product. Generally the additional base is non-nucleophilic in chemical reactivity. The product is a stereoisomerically substantially pure trans aminocyclohexyl ether compound of formula (69) and is formed as the free base. The free base may be converted, if desired, to the monohydrochloride salt by known methodologies, or alternatively, if desired, to other acid addition salts by reaction with an inorganic or organic acids under appropriate conditions. Acid addition salts can also be prepared metathetically by reaction of one acid addition salt with an acid that is stronger than that giving rise to the initial salt.

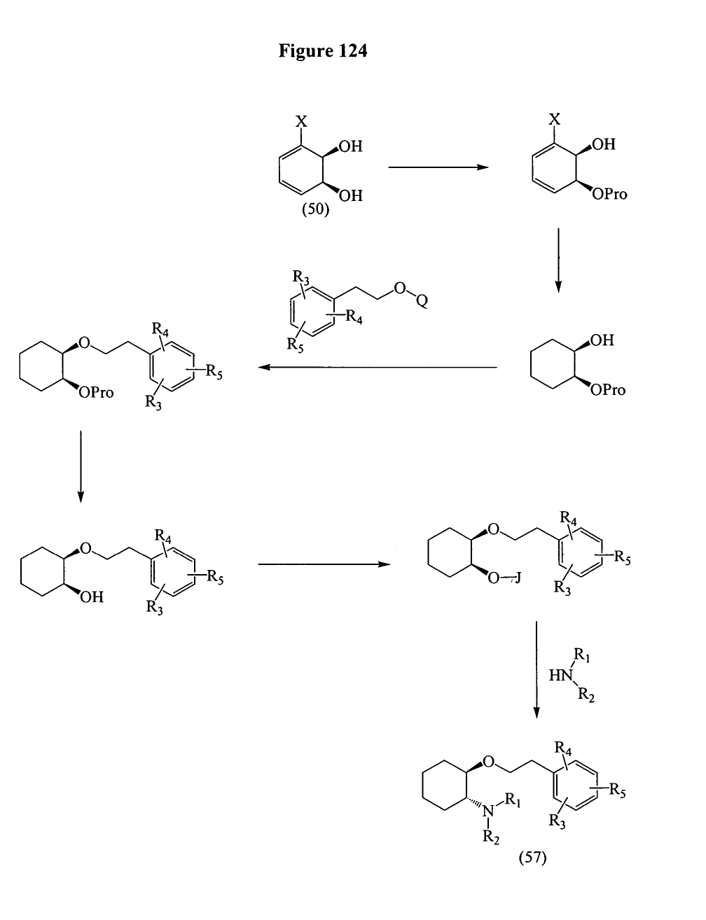

In another embodiment, the preparation of a stereoisomerically substantially pure trans aminocyclohexyl ether compound of formula (57) may be carried out by a process as outlined in FIG. 124, comprising the steps of starting with compound of formula (50) and following a reaction sequence under suitable conditions analogous to the applicable portion that is described in FIG. 121, wherein all the formulae and symbols are as described above.

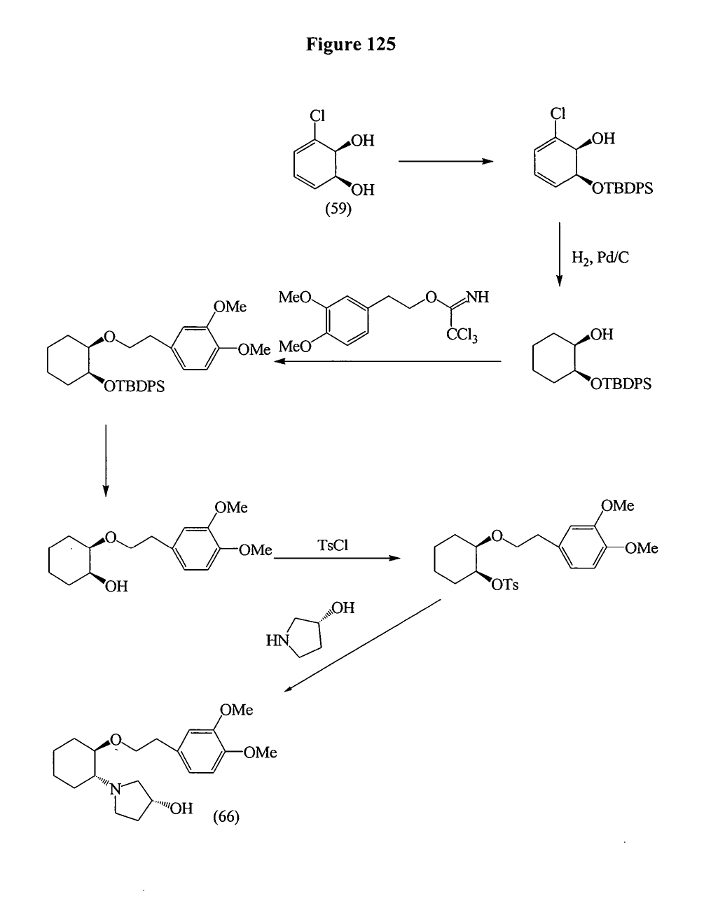

In another embodiment, the preparation of a stereoisomerically substantially pure trans aminocyclohexyl ether compound of formula (66) may be carried out by a process as outlined in FIG. 125, comprising the steps of starting with compound of formula (59) and following a reaction sequence under suitable conditions analogous to the applicable portion that is described in FIG. 122, wherein all the formulae and symbols are as described above. 3-Chloro-(1S,2S)-3,5-cyclohexadiene-1,2-diol of formula (59) is a commercially available product (e.g., Aldrich) or synthesized according to published procedure (e.g., Organic Synthesis, Vol. 76, 77 and T. Hudlicky et al., Aldrichimica Acta, 1999, 32, 35; and references cited therein).

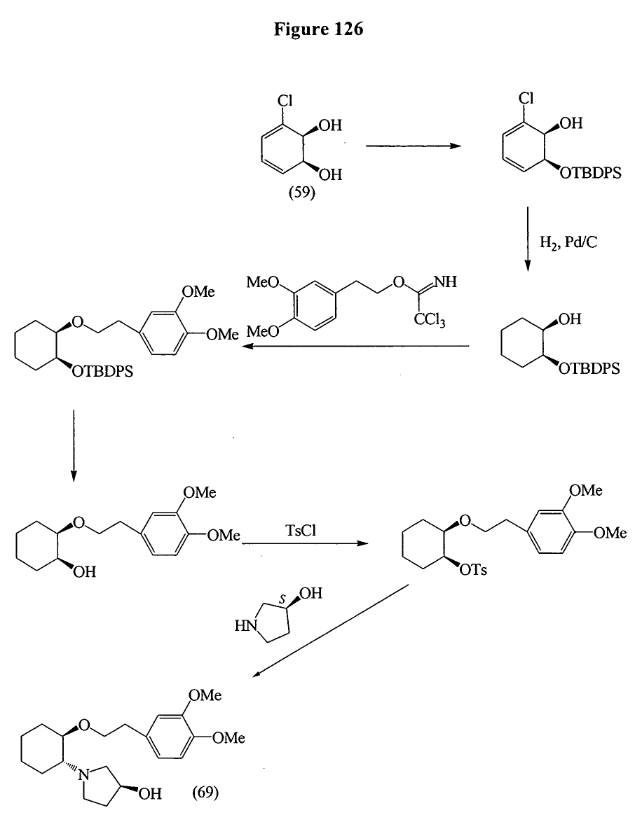

In another embodiment, the preparation of a stereoisomerically substantially pure trans aminocyclohexyl ether compound of formula (69) may be carried out by a process as outlined in FIG. 126, comprising the steps of starting with compound of formula (59) and following a reaction sequence under suitable conditions analogous to the applicable portion that is described in FIG. 123, wherein all the formulae and symbols are as described above.

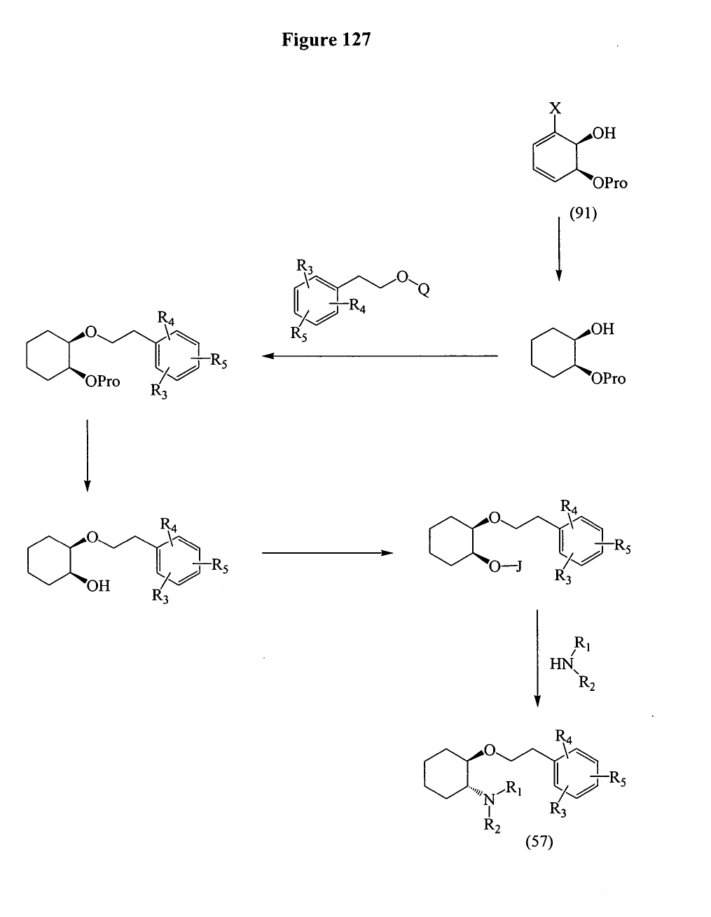

In another embodiment, the preparation of a stereoisomerically substantially pure trans aminocyclohexyl ether compound of formula (57) may be carried out by a process as outlined in FIG. 127, comprising the steps of starting with compound of formula (91) and following a reaction sequence under suitable conditions analogous to the applicable portion that is described in FIG. 121, wherein all the formulae and symbols are as described above.

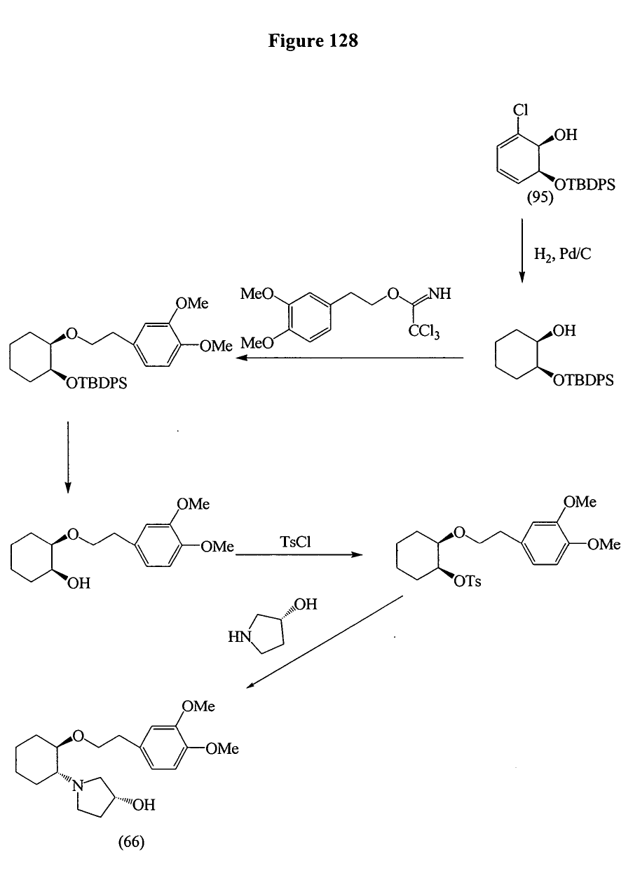

In another embodiment, the preparation of a stereoisomerically substantially pure trans aminocyclohexyl ether compound of formula (66) may be carried out by a process as outlined in FIG. 128, comprising the steps of starting with compound of formula (95) and following a reaction sequence under suitable conditions analogous to the applicable portion that is described in FIG. 122, wherein all the formulae and symbols are as described above.

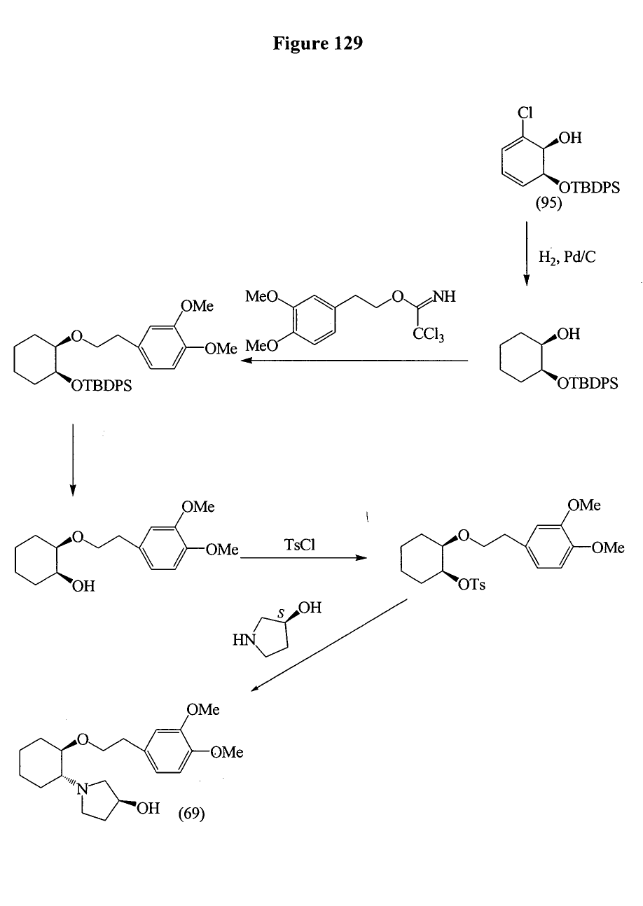

In another embodiment, the preparation of a stereoisomerically substantially pure trans aminocyclohexyl ether compound of formula (69) may be carried out by a process as outlined in FIG. 129, comprising the steps of starting with compound of formula (95) and following a reaction sequence under suitable conditions analogous to the applicable portion that is described in FIG. 123, wherein all the formulae and symbols are as described above.

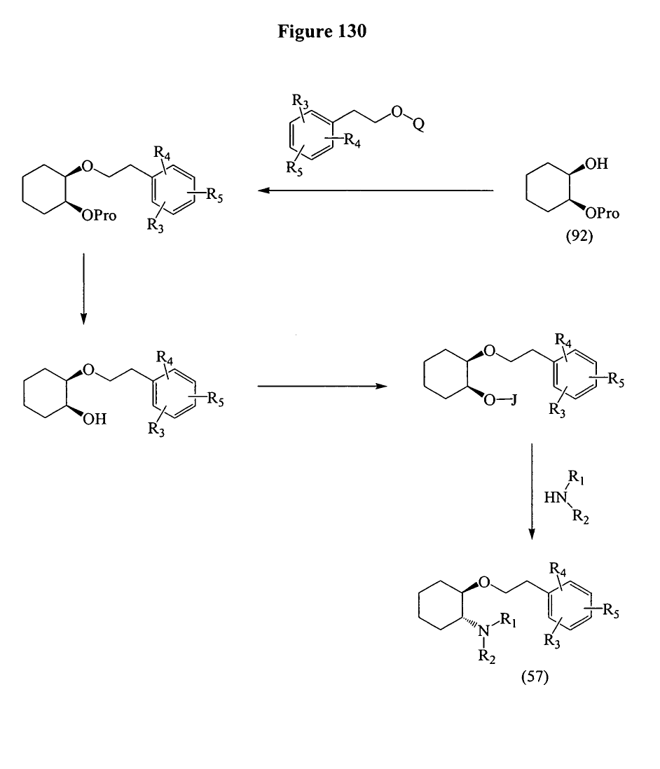

In another embodiment, the preparation of a stereoisomerically substantially pure trans aminocyclohexyl ether compound of formula (57) may be carried out by a process as outlined in FIG. 130, comprising the steps of starting with compound of formula (92) and following a reaction sequence under suitable conditions analogous to the applicable portion that is described in FIG. 121, wherein all the formulae and symbols are as described above.

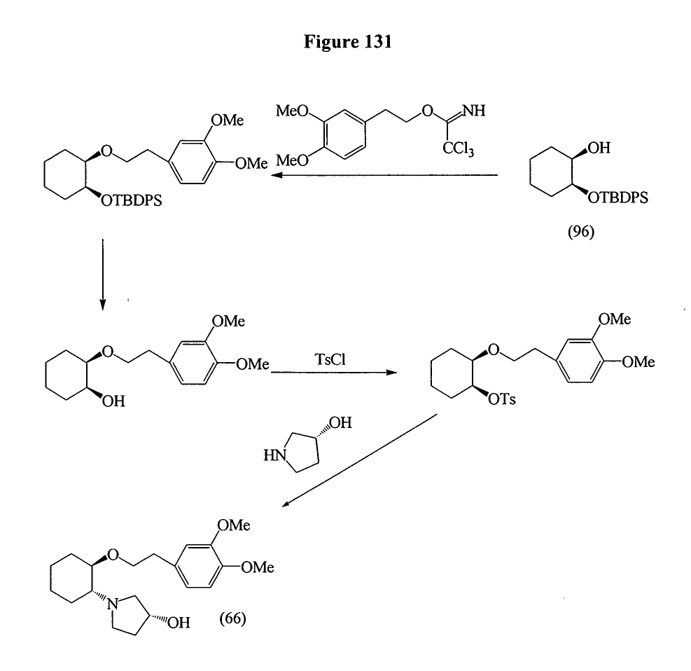

In another embodiment, the preparation of a stereoisomerically substantially pure trans aminocyclohexyl ether compound of formula (66) may be carried out by a process as outlined in FIG. 131, comprising the steps of starting with compound of formula (96) and following a reaction sequence under suitable conditions analogous to the applicable portion that is described in FIG. 122, wherein all the formulae and symbols are as described above.

In another embodiment, the preparation of a stereoisomerically substantially pure trans aminocyclohexyl ether compound of formula (69) may be carried out by a process as outlined in FIG. 132, comprising the steps of starting with compound of formula (96) and following a reaction sequence under suitable conditions analogous to the applicable portion that is described in FIG. 123, wherein all the formulae and symbols are as described above.

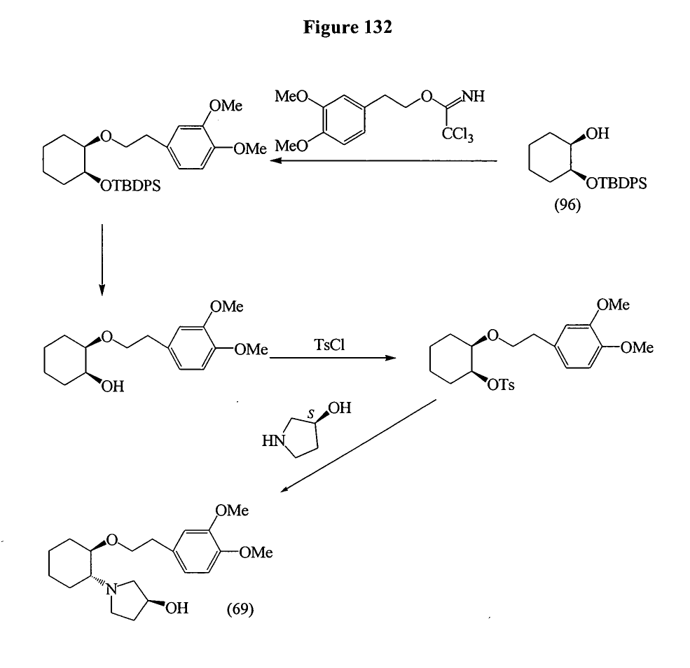

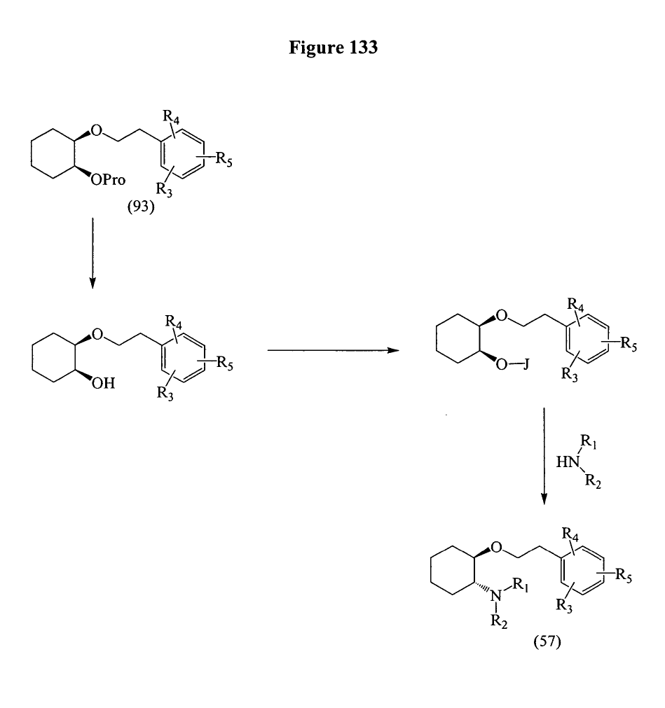

In another embodiment, the preparation of a stereoisomerically substantially pure trans aminocyclohexyl ether compound of formula (57) may be carried out by a process as outlined in FIG. 133, comprising the steps of starting with compound of formula (93) and following a reaction sequence under suitable conditions analogous to the applicable portion that is described in FIG. 121, wherein all the formulae and symbols are as described above.

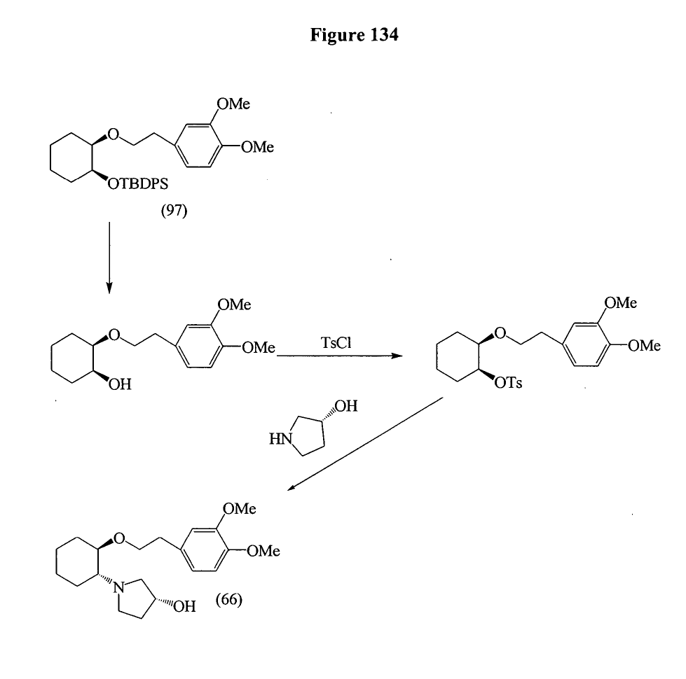

In another embodiment, the preparation of a stereoisomerically substantially pure trans aminocyclohexyl ether compound of formula (66) may be carried out by a process as outlined in FIG. 134, comprising the steps of starting with compound of formula (97) and following a reaction sequence under suitable conditions analogous to the applicable portion that is described in FIG. 122, wherein all the formulae and symbols are as described above.

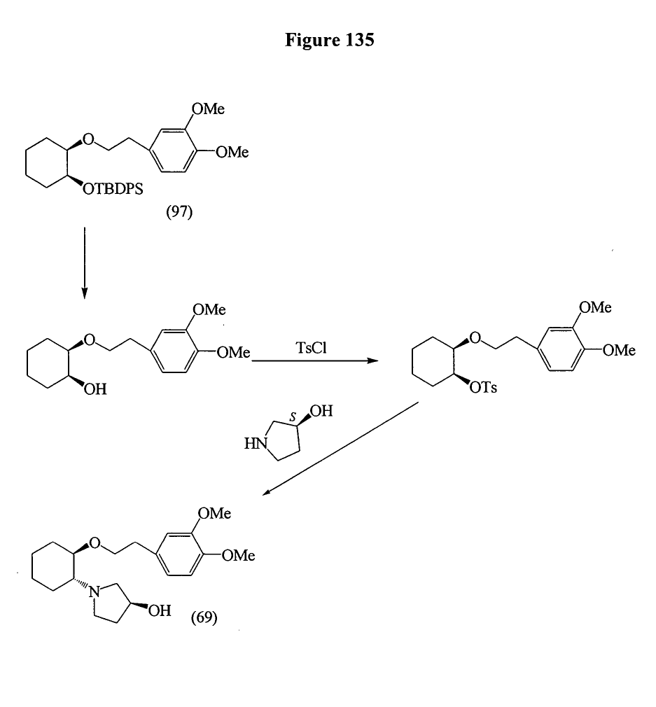

In another embodiment, the preparation of a stereoisomerically substantially pure trans aminocyclohexyl ether compound of formula (69) may be carried out by a process as outlined in FIG. 135, comprising the steps of starting with compound of formula (97) and following a reaction sequence under suitable conditions analogous to the applicable portion that is described in FIG. 123, wherein all the formulae and symbols are as described above.

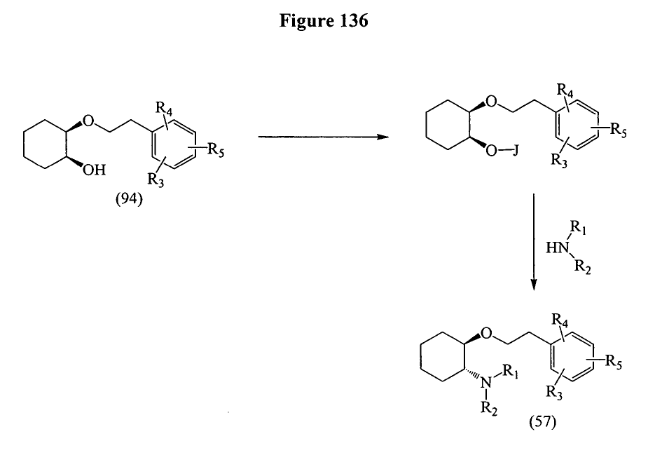

In another embodiment, the preparation of a stereoisomerically substantially pure trans aminocyclohexyl ether compound of formula (57) may be carried out by a process as outlined in FIG. 136, comprising the steps of starting with compound of formula (94) and following a reaction sequence under suitable conditions analogous to the applicable portion that is described in FIG. 121, wherein all the formulae and symbols are as described above.

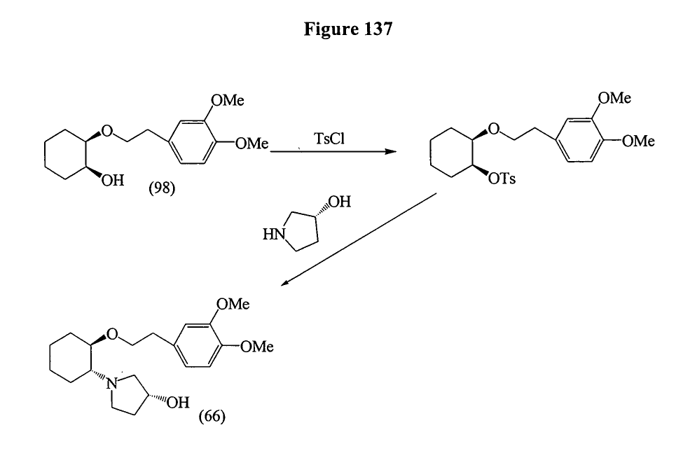

In another embodiment, the preparation of a stereoisomerically substantially pure trans aminocyclohexyl ether compound of formula (66) may be carried out by a process as outlined in FIG. 137, comprising the steps of starting with compound of formula (98) and following a reaction sequence under suitable conditions analogous to the applicable portion that is described in FIG. 122, wherein all the formulae and symbols are as described above.

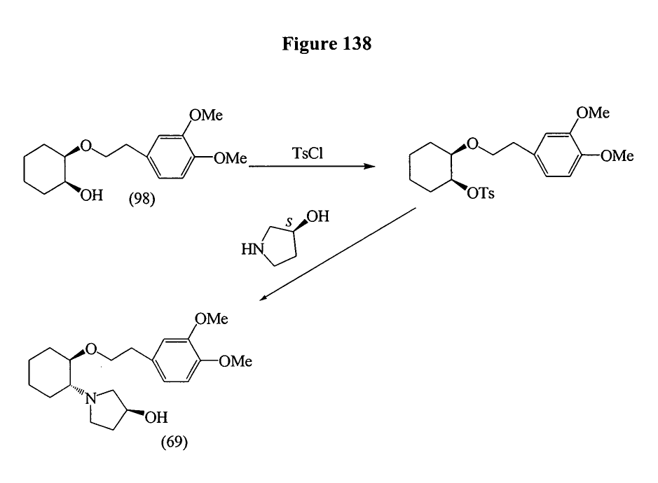

In another embodiment, the preparation of a stereoisomerically substantially pure trans aminocyclohexyl ether compound of formula (69) may be carried out by a process as outlined in FIG. 138, comprising the steps of starting with compound of formula (98) and following a reaction sequence under suitable conditions analogous to the applicable portion that is described in FIG. 123, wherein all the formulae and symbols are as described above.

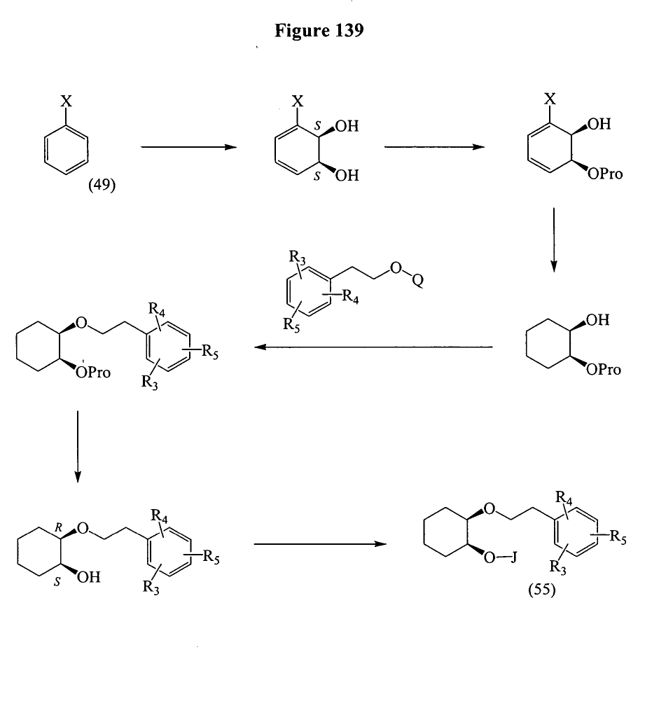

In another embodiment, the preparation of a stereoisomerically substantially pure compound of formula (55) may be carried out by a process as outlined in FIG. 139, comprising the steps of starting with compound of formula (49) and following a reaction sequence under suitable conditions analogous to the applicable portion that is described in FIG. 121, wherein all the formulae and symbols are as described above.

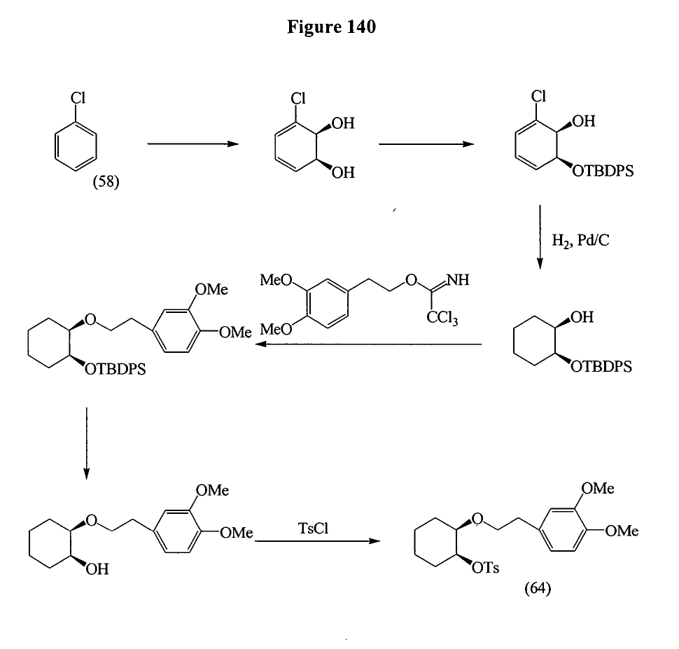

In another embodiment, the preparation of a stereoisomerically substantially pure compound of formula (64) may be carried out by a process as outlined in FIG. 140, comprising the steps of starting with compound of formula (58) and following a reaction sequence under suitable conditions analogous to the applicable portion that is described in FIG. 122, wherein all the formulae and symbols are as described above.

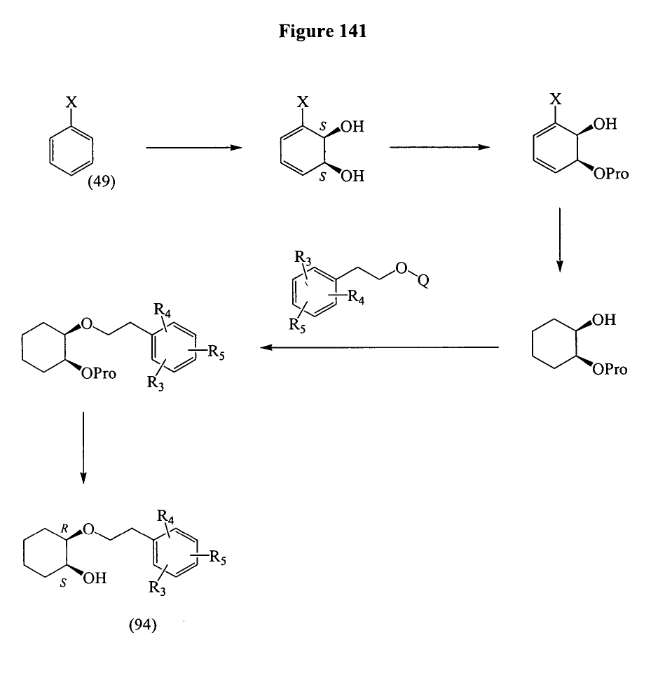

In another embodiment, the preparation of a stereoisomerically substantially pure compound of formula (94) may be carried out by a process as outlined in FIG. 141, comprising the steps of starting with compound of formula (49) and following a reaction sequence under suitable conditions analogous to the applicable portion that is described in FIG. 121, wherein all the formulae and symbols are as described above.

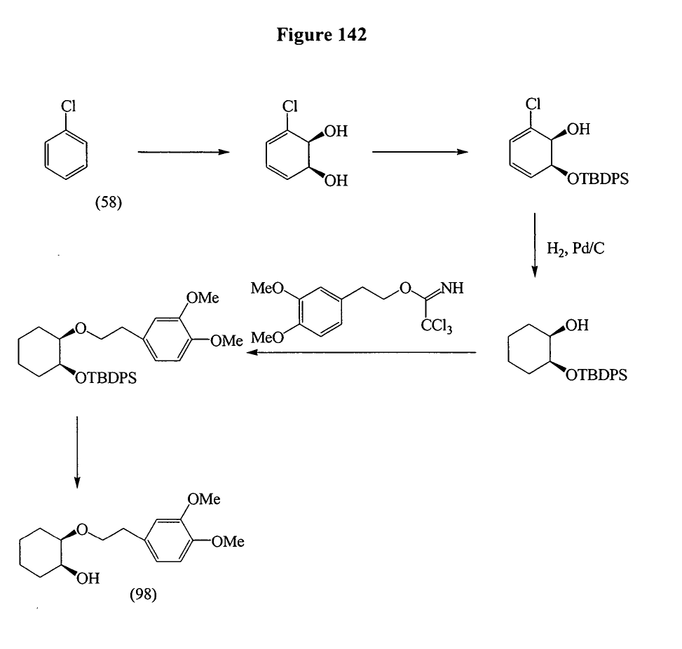

In another embodiment, the preparation of a stereoisomerically substantially pure compound of formula (98) may be carried out by a process as outlined in FIG. 142, comprising the steps of starting with compound of formula (58) and following a reaction sequence under suitable conditions analogous to the applicable portion that is described in FIG. 122, wherein all the formulae and symbols are as described above.

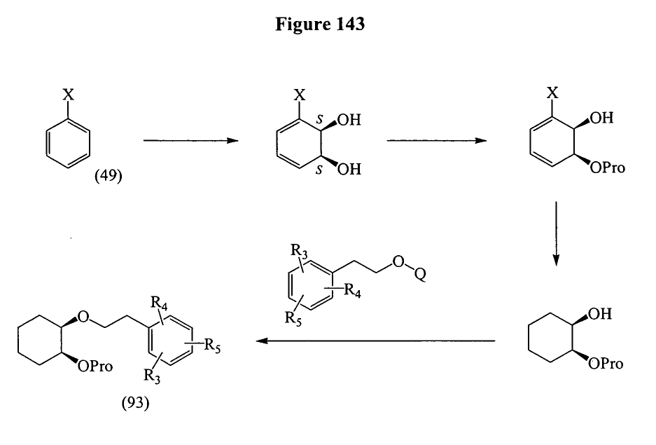

In another embodiment, the preparation of a stereoisomerically substantially pure compound of formula (93) may be carried out by a process as outlined in FIG. 143, comprising the steps of starting with compound of formula (49) and following a reaction sequence under suitable conditions analogous to the applicable portion that is described in FIG. 121, wherein all the formulae and symbols are as described above.

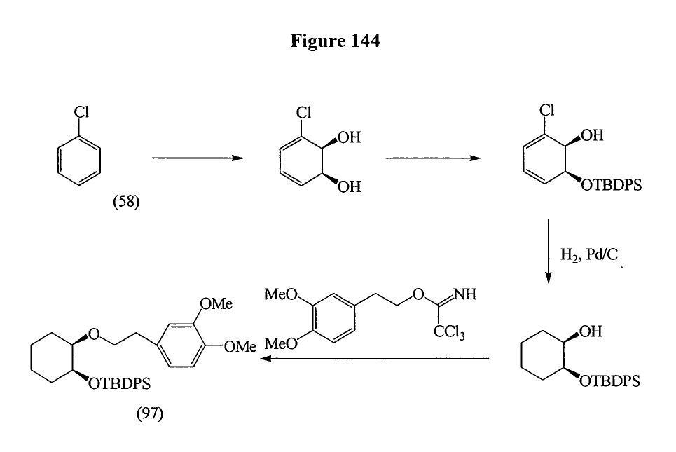

In another embodiment, the preparation of a stereoisomerically substantially pure compound of formula (97) may be carried out by a process as outlined in FIG. 144, comprising the steps of starting with compound of formula (58) and following a reaction sequence under suitable conditions analogous to the applicable portion that is described in FIG. 122, wherein all the formulae and symbols are as described above.

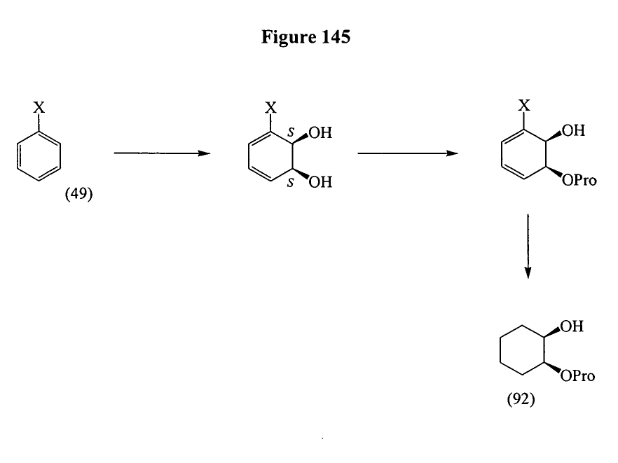

In another embodiment, the preparation of a stereoisomerically substantially pure compound of formula (92) may be carried out by a process as outlined in FIG. 145, comprising the steps of starting with compound of formula (49) and following a reaction sequence under suitable conditions analogous to the applicable portion that is described in FIG. 121, wherein all the formulae and symbols are as described above.

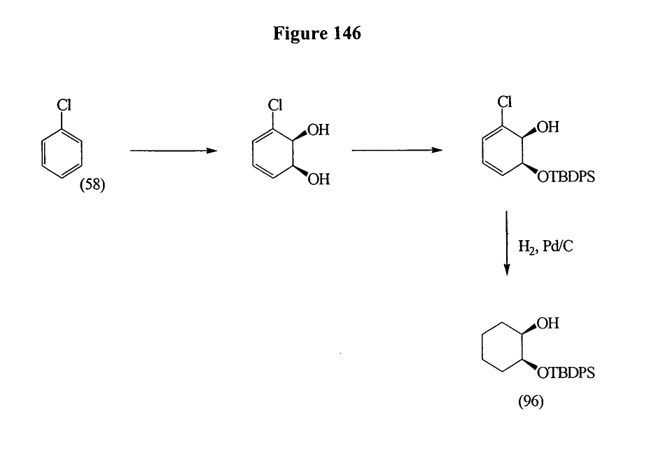

In another embodiment, the preparation of a stereoisomerically substantially pure compound of formula (96) may be carried out by a process as outlined in FIG. 146, comprising the steps of starting with compound of formula (58) and following a reaction sequence under suitable conditions analogous to the applicable portion that is described in FIG. 122, wherein all the formulae and symbols are as described above.

In another embodiment, the present invention provides a compound of formula (92), or a solvate or pharmaceutically acceptable salt thereof; wherein all the formulae and symbols are as described above.

In another embodiment, the present invention provides a compound of formula (54), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (93), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (94), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (55), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (96), or a solvate or pharmaceutically acceptable salt thereof; wherein all the formulae and symbols are as described above.

In another embodiment, the present invention provides a compound of formula (63), or a solvate or pharmaceutically acceptable salt thereof; wherein all the formulae and symbols are as described above.

In another embodiment, the present invention provides a compound of formula (97), or a solvate or pharmaceutically acceptable salt thereof; wherein all the formulae and symbols are as described above.

In another embodiment, the present invention provides a compound of formula (98), or a solvate or pharmaceutically acceptable salt thereof; wherein all the formulae and symbols are as described above.

In another embodiment, the present invention provides a compound of formula (64), or a solvate or pharmaceutically acceptable salt thereof; wherein all the formulae and symbols are as described above.

The present invention provides synthetic processes whereby compounds of formula (75) with trans-(1S,2S) configuration for the ether and amino functional groups may be prepared in stereoisomerically substantially pure form. Compounds of formulae (79) and (81) are some of the examples represented by formula (75). The present invention also provides synthetic processes whereby compounds of formulae (92), (99), (84) and (74) may be synthesized in stereoisomerically substantially pure forms. Compounds (96), (100), (62) and (78) are examples of formulae (92), (99), (84) and (74), respectively.

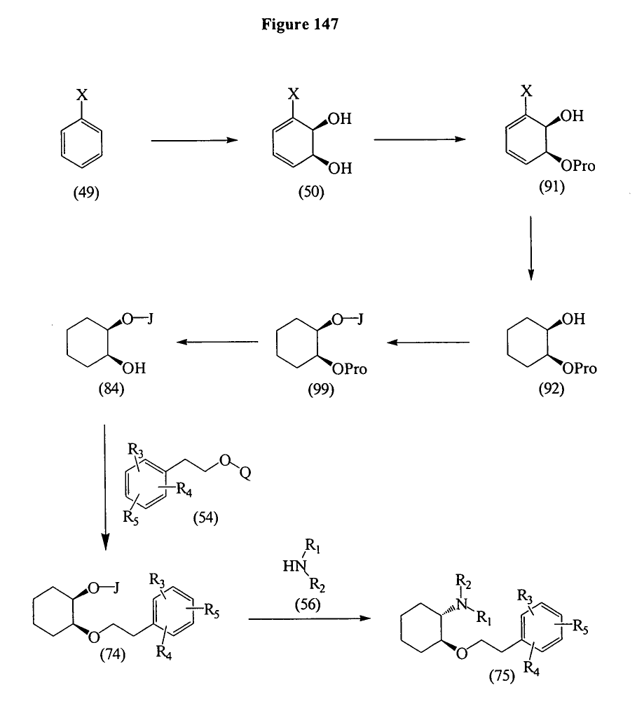

As outlined in FIG. 147, the preparation of a stereoisomerically substantially pure trans aminocyclohexyl ether compound of formula (75) may be carried out by following a process starting with a monohalobenzene (49), wherein X may be F, Cl, Br or I.

In a first step, compound (49) is transformed by well-established microbial oxidation to the cis-cyclohexandienediol (50) in stereoisomerically substantially pure form (T. Hudlicky et al., Aldrichimica Acta, 1999, 32, 35; and references cited therein). In a separate step, the less hindered hydroxy function in compound (50) may be selectively monoprotected as compound (91) where Pro represents the appropriate protecting group of the hydroxy function with retention of stereochemistry (T. Hudlicky et al., Aldrichimica Acta, 1999, 32, 35; S. M. Brown and T. Hudlicky, In Organic Synthesis: Theory and Applications; T. Hudlicky, Ed.; JAI Press: Greenwich, Conn., 1993; Vol. 2, p 113; and references cited therein). Tri-alkyl-silyl groups such as tri-isopropyl-silyl (TIPS) and t-butyldimethylsilyl (TBDMS) and alkyl-diaryl-silyl groups such as t-butyldiphenylsilyl (TBDPS) are some of the possible examples for Pro. Suitable reaction conditions are set forth in, for example, Greene, “Protective Groups in Organic Chemistry”, John Wiley & Sons, New York N.Y. (1991). In a separate step, conversion of compound (91) to compound (92) may be effected by hydrogenation and hydrogenolysis in the presence of a catalyst under appropriate conditions. Palladium on activated carbon is one example of the catalysts. Hydrogenolysis of alkyl or alkenyl halide such as (91) may be conducted under basic conditions. The presence of a base such as sodium ethoxide, sodium bicarbonate, sodium acetate or calcium carbonate is some possible examples. The base may be added in one portion or incrementally during the course of the reaction. In a separate step, the free hydroxy group of compound (92) is converted into an activated form as represented by formula (99) under suitable conditions. An “activated form” as used herein means that the hydroxy group is converted into a good leaving group (—O-J). The leaving group may be a mesylate (MsO—) group, a tosylate group (TsO—) or a nosylate (NsO—). The hydroxy group may also be converted into other suitable leaving groups according to procedures well known in the art. In a typical reaction for the formation of a tosylate, compound (92) is treated with a hydroxy activating reagent such as tosyl chloride (TsCl) in the presence of a base, such as pyridine or triethylamine. The reaction is generally satisfactorily conducted at about 0° C., but may be adjusted as required to maximize the yields of the desired product. An excess of the hydroxy activating reagent (e.g., tosyl chloride), relative to compound (92) may be used to maximally convert the hydroxy group into the activated form. In a separate step, removal of the protecting group (Pro) in compound (99) by standard procedures (e.g., tetrabutylammonium fluoride in tetrahydrofuran or as described in Greene, “Protective Groups in Organic Chemistry”, John Wiley & Sons, New York N.Y. (1991)) affords compound (84). In a separate step, alkylation of the free hydroxy group in compound (84) to form compound (74) is carried out under appropriate conditions with compound (54), where —O-Q represents a good leaving group on reaction with a hydroxy function with retention of the stereochemical configuration of the hydroxy function in the formation of an ether compound. Trichloroacetimidate is one example for the —O-Q function. For some compound (54), it may be necessary to introduce appropriate protection groups prior to this step being performed. Suitable protecting groups are set forth in, for example, Greene, “Protective Groups in Organic Chemistry”, John Wiley & Sons, New York N.Y. (1991).

In a separate step, the resulted compound (74) is treated under suitable conditions with an amino compound of formula (56) to form compound (75) as the product. The reaction may be carried out with or without a solvent and at an appropriate temperature range that allows the formation of the product (75) at a suitable rate. An excess of the amino compound (56) may be used to maximally convert compound (74) to the product (75). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 147) generates the compound of formula (75) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

In one embodiment, the present invention provides a process for the preparation of a stereoisomerically substantially pure compound of formula (75): 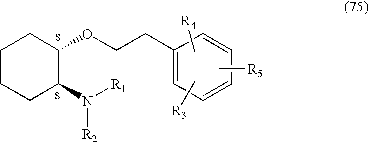
wherein, independently at each occurrence, R₁ and R₂ are independently selected from hydrogen, C₁-C₈alkyl, C₃-C₈alkoxyalkyl, C₁-C₈hydroxyalkyl, and C₇-C₁₂aralkyl; or
R₁ and R₂ are independently selected from C₃-C₈alkoxyalkyl, C₁-C₈hydroxyalkyl, and C₇-C₁₂aralkyl; or
R₁ and R₂, when taken together with the nitrogen atom to which they are directly attached in formula (57), form a ring denoted by formula (I): 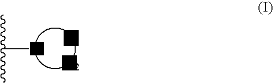
wherein the ring of formula (I) is formed from the nitrogen as shown as well as three to nine additional ring atoms independently selected from carbon, nitrogen, oxygen, and sulfur; where any two adjacent ring atoms may be joined together by single or double bonds, and where any one or more of the additional carbon ring atoms may be substituted with one or two substituents selected from hydrogen, hydroxy, C₁-C₃hydroxyalkyl, oxo, C₂-C₄acyl, C₁-C₃alkyl, C₂-C₄alkylcarboxy, C₁-C₃alkoxy, C₁-C₂₀alkanoyloxy, or may be substituted to form a spiro five- or six-membered heterocyclic ring containing one or two heteroatoms selected from oxygen and sulfur; and any two adjacent additional carbon ring atoms may be fused to a C₃-C₈carbocyclic ring, and any one or more of the additional nitrogen ring atoms may be substituted with substituents selected from hydrogen, C₁-C₆alkyl, C₂-C₄acyl, C₂-C₄hydroxyalkyl and C₃-C₈alkoxyalkyl; or
preferably R₁ and R₂, when taken together with the nitrogen atom to which they are directly attached in formula (57), form a ring denoted by formula (II): 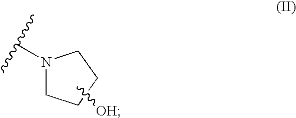
or in another embodiment R₁ and R₂, when taken together with the nitrogen atom to which they are directly attached in formula (I), may form a bicyclic ring system selected from 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
R₃, R₄ and R₅ are independently selected from bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl and N(R₆,R₇) where R₆ and R₇ are independently selected from hydrogen, acetyl, methanesulfonyl, and C₁-C₆alkyl; or
R₃, R₄ and R₅ are independently selected from hydrogen, hydroxy and C₁-C₆alkoxy; with the proviso that R₃, R₄ and R₅ cannot all be hydrogen;
comprising the steps of starting with a monohalobenzene (49), wherein X may be F, Cl, Br or I; and following a reaction sequence as outlined in FIG. 147 under suitable conditions, wherein
Pro represents the appropriate protecting group of the hydroxy function with retention of stereochemistry;
—O-Q represents a good leaving group which on reaction with a hydroxy function will result in the formation of an ether compound with retention of the stereochemical configuration of the hydroxy function; and
—O-J represents a good leaving group on reaction with a nucleophilic reactant will result in a substitution product with substantial inversion of the stereochemical configuration of the activated hydroxy group as shown in FIG. 147; and all the formulae and symbols are as described above.

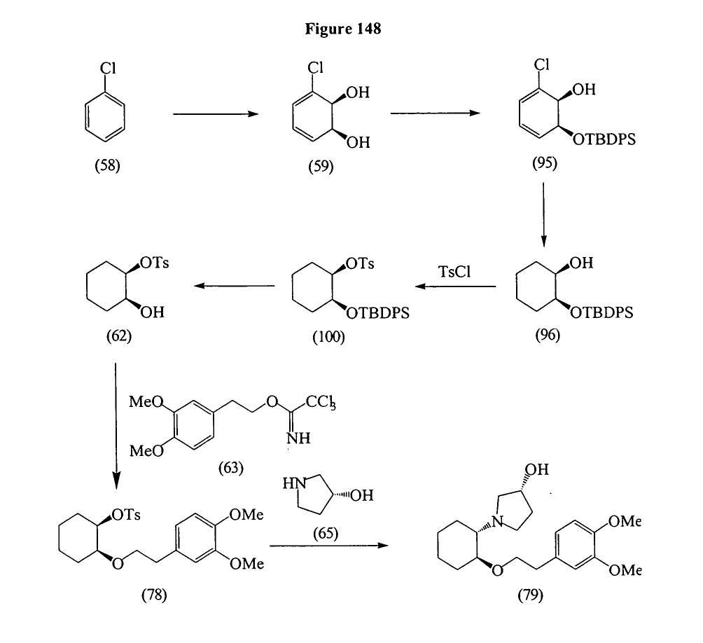

In another embodiment, the present invention provides a process for the preparation of a stereoisomerically substantially pure compound of formula (79), comprising the steps under suitable conditions as shown in FIG. 148, wherein all the formulae and symbols are as described above. As outlined in FIG. 148, the preparation of a stereoisomerically substantially pure trans aminocyclohexyl ether compound of formula (79) may be carried out by starting with a biotransformation of chlorobenzene (49) to compound (59) by microorganism such as Pseudomonas putida 39/D. Experimental conditions for the biotransformation are well established (Organic Synthesis, Vol. 76, 77 and T. Hudlicky et al., Aldrichimica Acta, 1999, 32, 35; and references cited therein). In a separate step, the less hindered hydroxy function in compound (59) is selectively monosilylated as compound (95) by reaction with silylating reagent such as t-butyldiphenylsilyl chloride (TBDPSCl) under suitable conditions (e.g., imaidazole in CH₂Cl₂) (T. Hudlicky et al., Aldrichimica Acta, 1999, 32, 35; S. M. Brown and T. Hudlicky, In Organic Synthesis: Theory and Applications; T. Hudlicky, Ed.; JAI Press: Greenwich, Conn., 1993; Vol. 2, p 113; and references cited therein). In another separate step, compound (95) is converted to compound (96) by reduction such as hydrogenation and hydrogenolysis in the presence of a catalyst under appropriate conditions. Palladium on activated carbon is one example of the catalysts. The reduction of compound (95) may be conducted under basic conditions e.g., in the presence of a base such as sodium ethoxide, sodium bicarbonate, sodium acetate or calcium carbonate. The base may be added in one portion or incrementally during the course of the reaction. In a separate step, the hydroxy group of compound (96) is converted under suitable conditions into an activated form such as the tosylate of formula (100) by treatment with tosyl chloride (TsCl) in the presence of pyridine. In another separate step, the t-butyldiphenylsilyl (TBDPS) protection group in compound (100) may be removed by standard procedures (e.g., tetrabutylammonium fluoride in tetrahydrofuran or as described in Greene, “Protective Groups in Organic Chemistry”, John Wiley & Sons, New York N.Y. (1991)) to afford the hydroxytosylate compound (62). In a separate step, the free hydroxy group in compound (62) is alkylated under appropriate conditions to form compound (78). The trichloroacetimidate (63) is readily prepared from the corresponding alcohol, 3,4-dimethoxyphenethyl alcohol which is commercially available (e.g., Aldrich), by treatment with trichloroacetonitrile. The alkylation of compound (62) by trichloroacetimidate (63) may be carried out in the presence of a Lewis acid such as HBF₄. In another separate step, the tosylate group of formula (78) is displaced by an amino compound such as 3R-pyrrolidinol (65) with inversion of configuration. 3R-pyrrolidinol (65) is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (79) at a suitable rate. An excess of the amino compound (65) may be used to maximally convert compound (78) to the product (79). The reaction may be performed in the presence of a base that can facilitate the formation and isolation of the product. Generally the additional base is non-nucleophilic in chemical reactivity. When the reaction has proceeded to substantial completion, the desired product is recovered from the reaction mixture by conventional organic chemistry techniques, and is purified accordingly.

The reaction sequence described above (FIG. 148) in general generates the compound of formula (79) as the free base. The free base may be converted, if desired, to the monohydrochloride salt by known methodologies, or alternatively, to other acid addition salts by reaction with an inorganic or organic acid under appropriate conditions. Acid addition salts can also be prepared metathetically by reaction of one acid addition salt with an acid that is stronger than that giving rise to the initial salt.

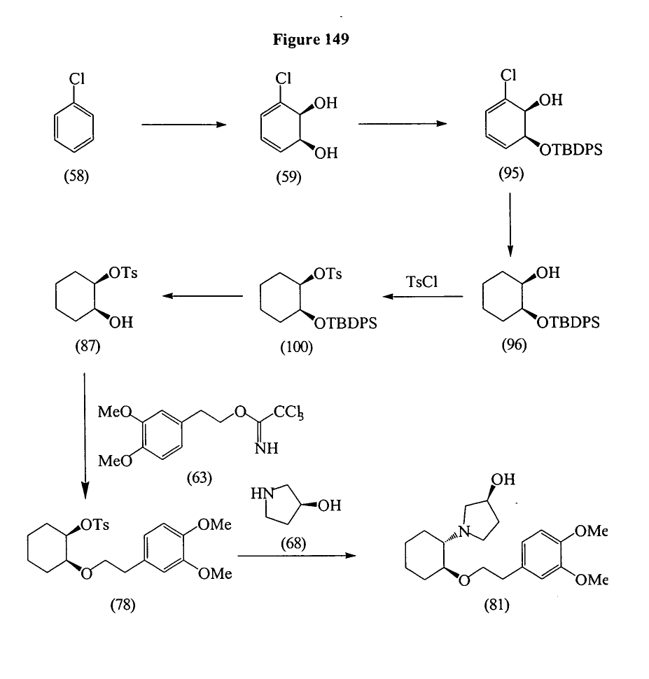

In another embodiment, the preparation of a stereoisomerically substantially pure trans aminocyclohexyl ether compound of formula (81) may be carried out by a process as outlined in FIG. 149, comprising the steps of starting with chlorobenzene (58) and following a reaction sequence under suitable conditions analogous to the applicable portion that is described in FIG. 148 above leading to compound of formula (78). The latter is reacted with an amino compound of formula (68). Compound (68), 3S-pyrrolidinol, is commercially available (e.g., Aldrich) or may be prepared according to published procedure (e.g., Chem.Ber./Recueil 1997, 130, 385-397). The reaction may be carried out with or without a solvent and at an appropriate temperature range that allows the formation of the product (81) at a suitable rate. An excess of the amino compound (68) may be used to maximally convert compound (78) to the product (81). The reaction may be performed in the presence of a base that can facilitate the formation of the product. Generally the additional base is non-nucleophilic in chemical reactivity. The product is a stereoisomerically substantially pure trans aminocyclohexyl ether compound of formula (81) and is formed as the free base. The free base may be converted, if desired, to the monohydrochloride salt by known methodologies, or alternatively, to other acid addition salts by reaction with an inorganic or organic acids under appropriate conditions. Acid addition salts can also be prepared metathetically by reaction of one acid addition salt with an acid that is stronger than that giving rise to the initial salt.

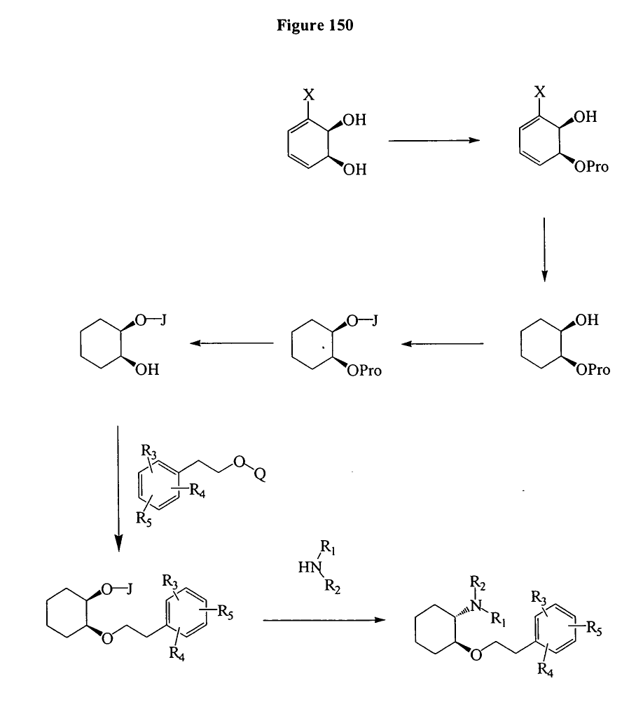

In another embodiment, the preparation of a stereoisomerically substantially pure trans aminocyclohexyl ether compound of formula (75) may be carried out by a process as outlined in FIG. 150, comprising the steps of starting with compound of formula (50) and following a reaction sequence under suitable conditions analogous to the applicable portion that is described in FIG. 147, wherein all the formulae and symbols are as described above.

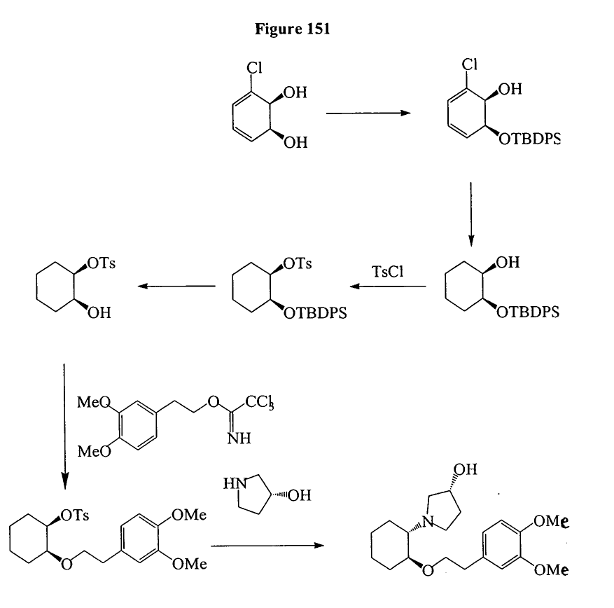

In another embodiment, the preparation of a stereoisomerically substantially pure trans aminocyclohexyl ether compound of formula (79) may be carried out by a process as outlined in FIG. 151, comprising the steps of starting with compound of formula (59) and following a reaction sequence under suitable conditions analogous to the applicable portion that is described in FIG. 148, wherein all the formulae and symbols are as described above. 3-Chloro-(1S,2S)-3,5-cyclohexadiene-1,2-diol of formula (59) is a commercially available product (e.g., Aldrich) or synthesized according to published procedure (e.g., Organic Synthesis, Vol. 76, 77 and T. Hudlicky et al., Aldrichimica Acta, 1999, 32, 35; and references cited therein).

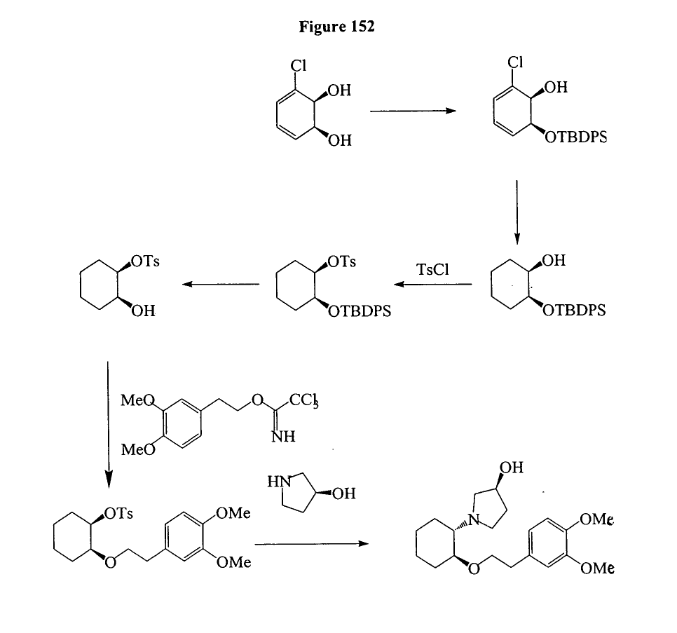

In another embodiment, the preparation of a stereoisomerically substantially pure trans aminocyclohexyl ether compound of formula (81) may be carried out by a process as outlined in FIG. 152, comprising the steps of starting with compound of formula (59) and following a reaction sequence under suitable conditions analogous to the applicable portion that is described in FIG. 149, wherein all the formulae and symbols are as described above.

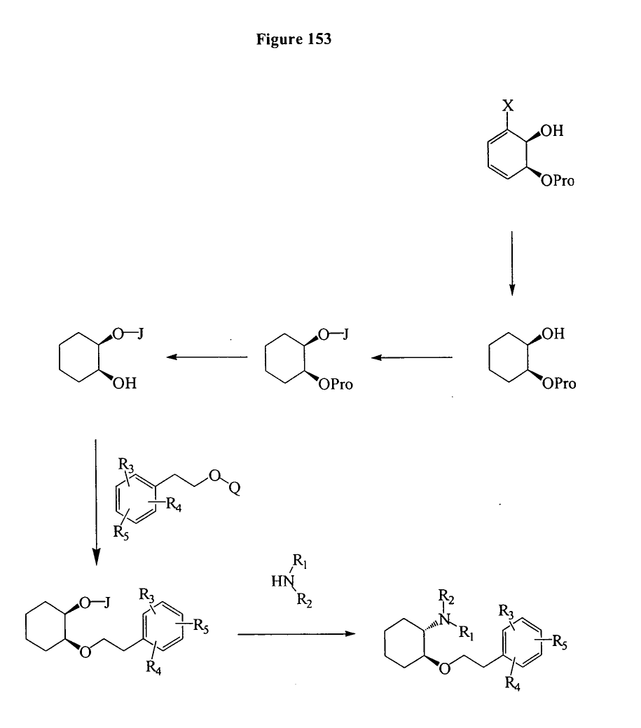

In another embodiment, the preparation of a stereoisomerically substantially pure trans aminocyclohexyl ether compound of formula (75) may be carried out by a process as outlined in FIG. 153, comprising the steps of starting with compound of formula (91) and following a reaction sequence under suitable conditions analogous to the applicable portion that is described in FIG. 147, wherein all the formulae and symbols are as described above.

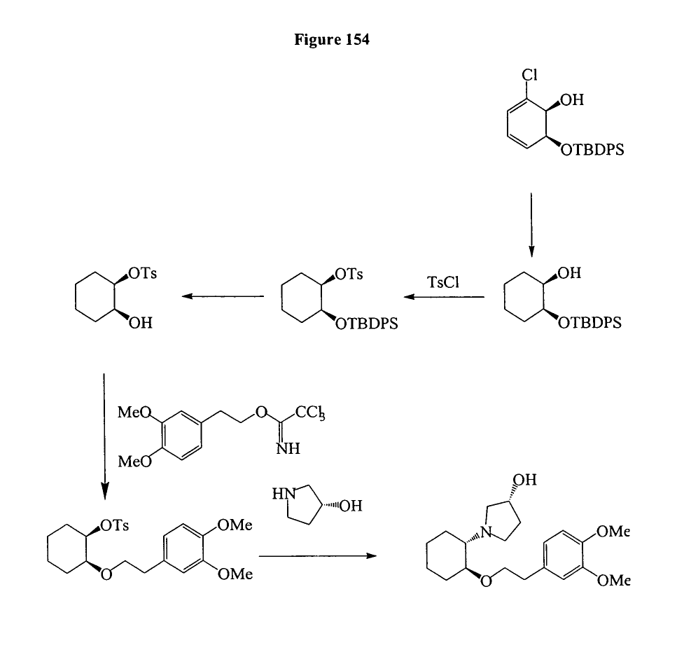

In another embodiment, the preparation of a stereoisomerically substantially pure trans aminocyclohexyl ether compound of formula (79) may be carried out by a process as outlined in FIG. 154, comprising the steps of starting with compound of formula (95) and following a reaction sequence under suitable conditions analogous to the applicable portion that is described in FIG. 148, wherein all the formulae and symbols are as described above.

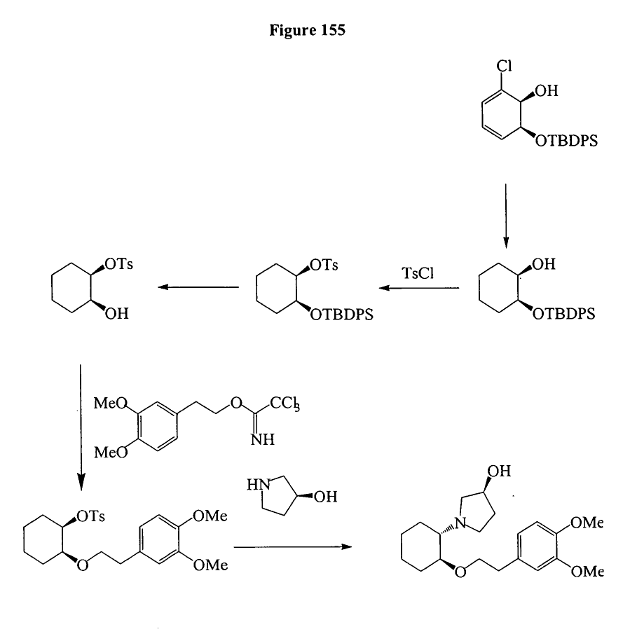

In another embodiment, the preparation of a stereoisomerically substantially pure trans aminocyclohexyl ether compound of formula (81) may be carried out by a process as outlined in FIG. 155, comprising the steps of starting with compound of formula (95) and following a reaction sequence under suitable conditions analogous to the applicable portion that is described in FIG. 149, wherein all the formulae and symbols are as described above.

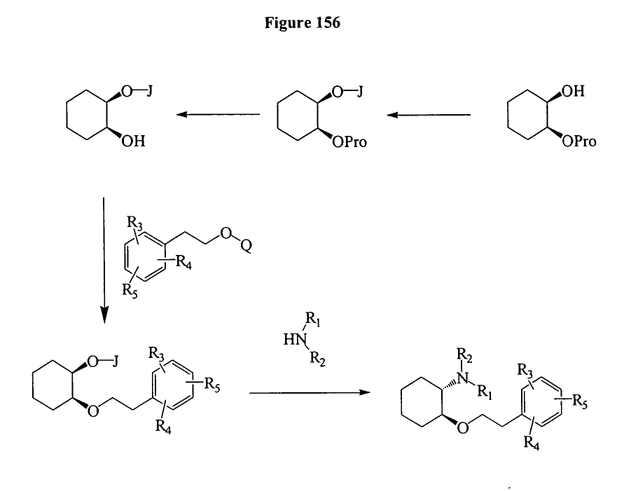

In another embodiment, the preparation of a stereoisomerically substantially pure trans aminocyclohexyl ether compound of formula (75) may be carried out by a process as outlined in FIG. 156, comprising the steps of starting with compound of formula (92) and following a reaction sequence under suitable conditions analogous to the applicable portion that is described in FIG. 147, wherein all the formulae and symbols are as described above.

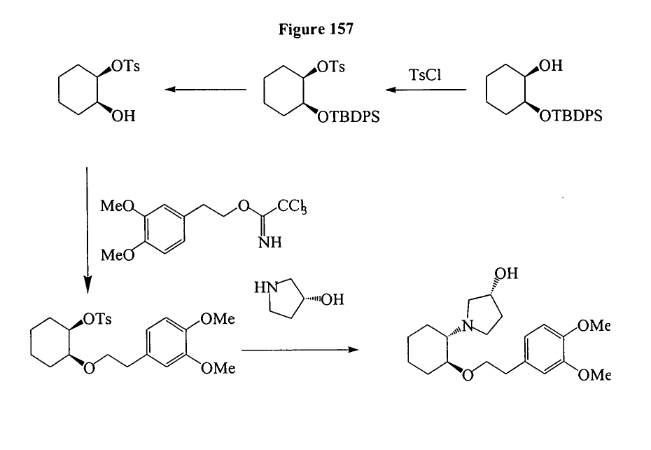

In another embodiment, the preparation of a stereoisomerically substantially pure trans aminocyclohexyl ether compound of formula (79) may be carried out by a process as outlined in FIG. 157, comprising the steps of starting with compound of formula (96) and following a reaction sequence under suitable conditions analogous to the applicable portion that is described in FIG. 148, wherein all the formulae and symbols are as described above.

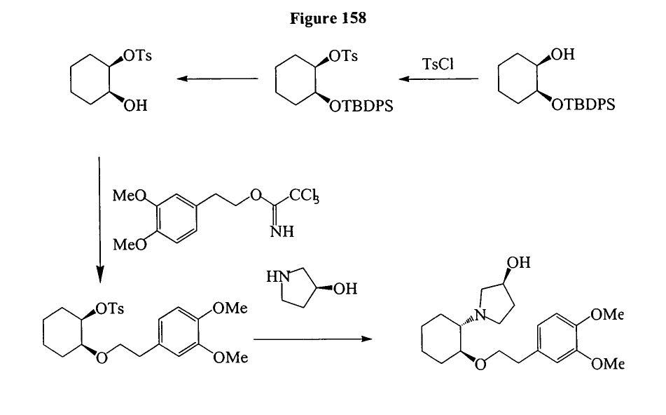

In another embodiment, the preparation of a stereoisomerically substantially pure trans aminocyclohexyl ether compound of formula (81) may be carried out by a process as outlined in FIG. 158, comprising the steps of starting with compound of formula (96) and following a reaction sequence under suitable conditions analogous to the applicable portion that is described in FIG. 149, wherein all the formulae and symbols are as described above.

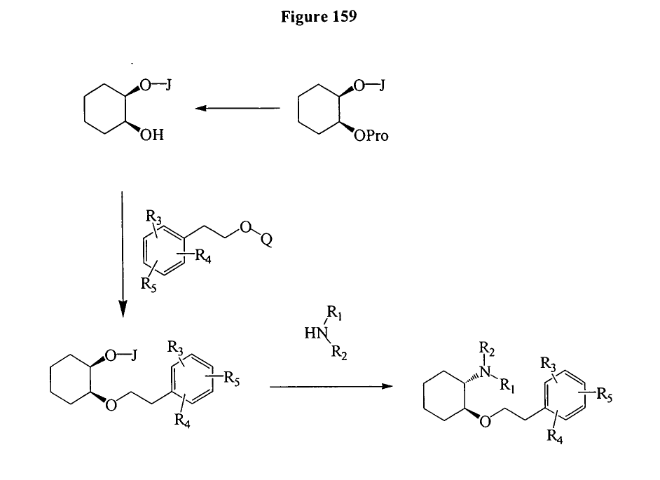

In another embodiment, the preparation of a stereoisomerically substantially pure trans aminocyclohexyl ether compound of formula (75) may be carried out by a process as outlined in FIG. 159, comprising the steps of starting with compound of formula (99) and following a reaction sequence under suitable conditions analogous to the applicable portion that is described in FIG. 147, wherein all the formulae and symbols are as described above.

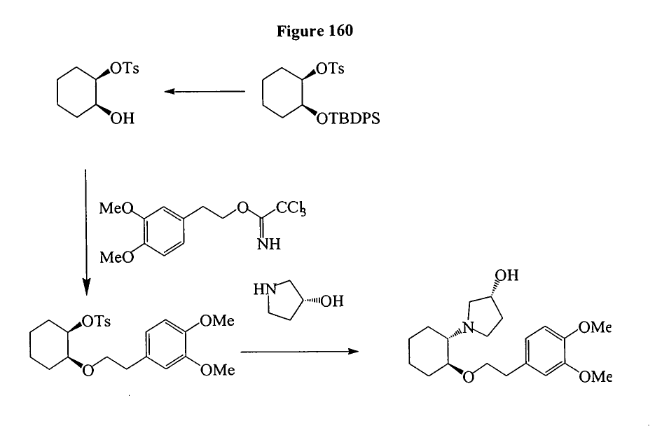

In another embodiment, the preparation of a stereoisomerically substantially pure trans aminocyclohexyl ether compound of formula (79) may be carried out by a process as outlined in FIG. 160, comprising the steps of starting with compound of formula (100) and following a reaction sequence under suitable conditions analogous to the applicable portion that is described in FIG. 148, wherein all the formulae and symbols are as described above.

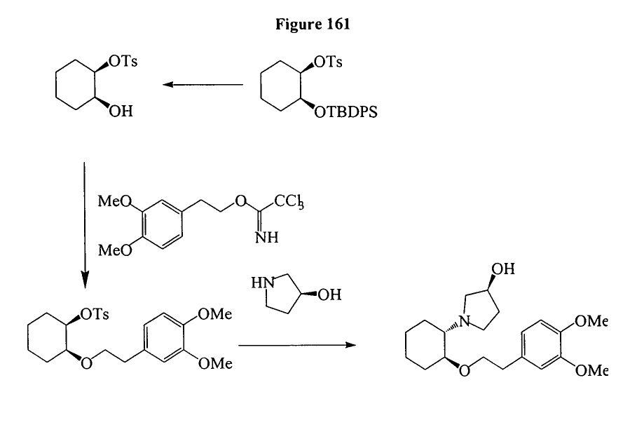

In another embodiment, the preparation of a stereoisomerically substantially pure trans aminocyclohexyl ether compound of formula (81) may be carried out by a process as outlined in FIG. 161, comprising the steps of starting with compound of formula (100) and following a reaction sequence under suitable conditions analogous to the applicable portion that is described in FIG. 149, wherein all the formulae and symbols are as described above.

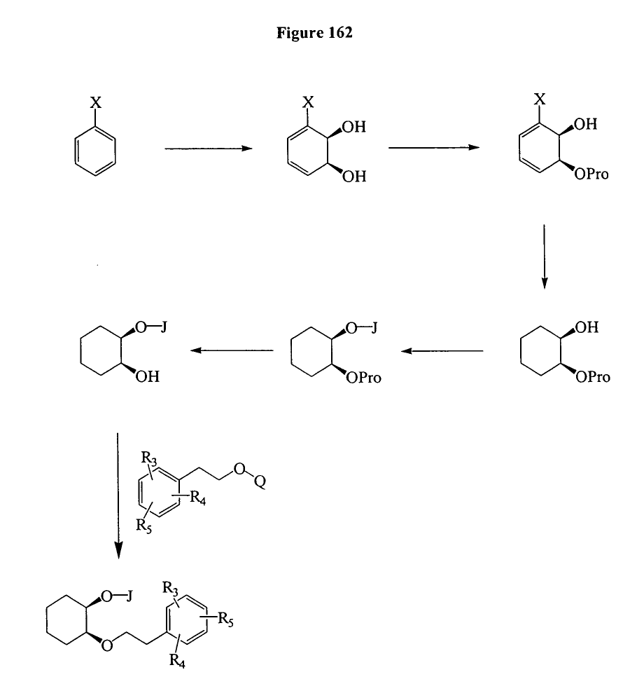

In another embodiment, the preparation of a stereoisomerically substantially pure compound of formula (74) may be carried out by a process as outlined in FIG. 162, comprising the steps of starting with compound of formula (49) and following a reaction sequence under suitable conditions analogous to the applicable portion that is described in FIG. 147, wherein all the formulae and symbols are as described above.

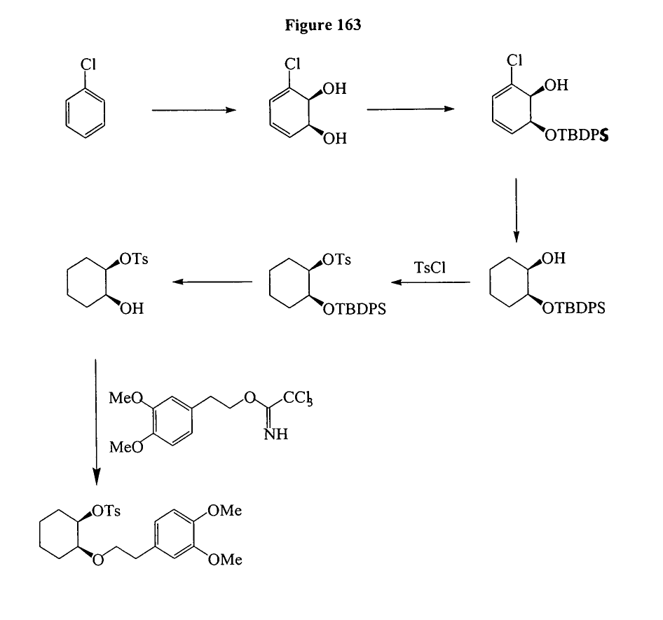

In another embodiment, the preparation of a stereoisomerically substantially pure compound of formula (78) may be carried out by a process as outlined in FIG. 163, comprising the steps of starting with compound of formula (58) and following a reaction sequence under suitable conditions analogous to the applicable portion that is described in FIG. 148, wherein all the formulae and symbols are as described above.

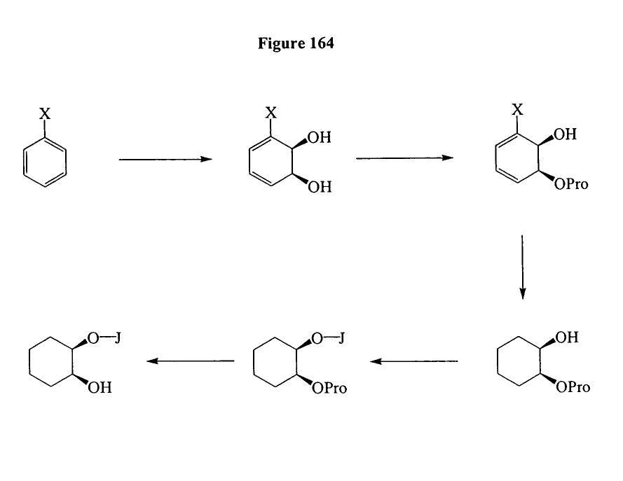

In another embodiment, the preparation of a stereoisomerically substantially pure compound of formula (84) may be carried out by a process as outlined in FIG. 164, comprising the steps of starting with compound of formula (49) and following a reaction sequence under suitable conditions analogous to the applicable portion that is described in FIG. 147, wherein all the formulae and symbols are as described above.

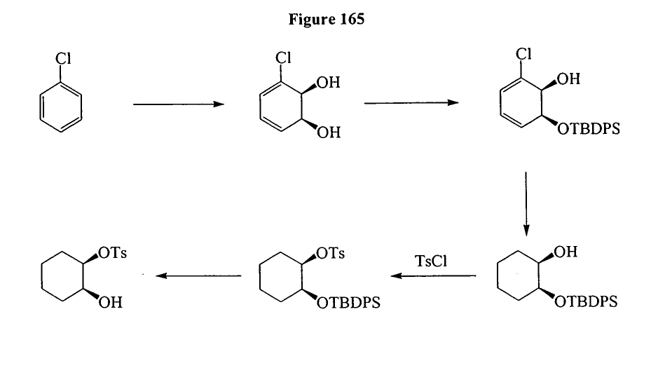

In another embodiment, the preparation of a stereoisomerically substantially pure compound of formula (62) may be carried out by a process as outlined in FIG. 165, comprising the steps of starting with compound of formula (58) and following a reaction sequence under suitable conditions analogous to the applicable portion that is described in FIG. 148, wherein all the formulae and symbols are as described above.

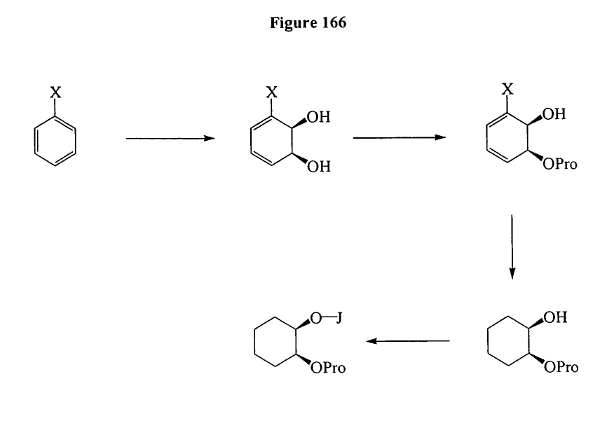

In another embodiment, the preparation of a stereoisomerically substantially pure compound of formula (99) may be carried out by a process as outlined in FIG. 166, comprising the steps of starting with compound of formula (49) and following a reaction sequence under suitable conditions analogous to the applicable portion that is described in FIG. 147, wherein all the formulae and symbols are as described above.

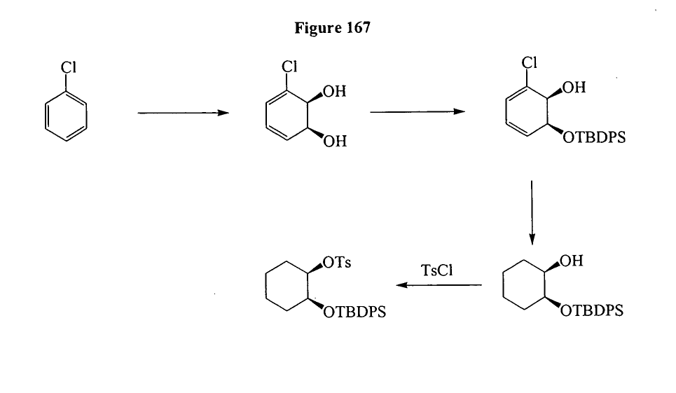

In another embodiment, the preparation of a stereoisomerically substantially pure compound of formula (100) may be carried out by a process as outlined in FIG. 167, comprising the steps of starting with compound of formula (58) and following a reaction sequence under suitable conditions analogous to the applicable portion that is described in FIG. 148, wherein all the formulae and symbols are as described above.

In another embodiment, the present invention provides a compound of formula (92), or a solvate or pharmaceutically acceptable salt thereof; wherein all the formulae and symbols are as described above.

In another embodiment, the present invention provides a compound of formula (99), or a solvate or pharmaceutically acceptable salt thereof; wherein all the formulae and symbols are as described above.

In another embodiment, the present invention provides a compound of formula (84), or a solvate or pharmaceutically acceptable salt thereof; wherein all the formulae and symbols are as described above.

In another embodiment, the present invention provides a compound of formula (54), or a solvate or pharmaceutically acceptable salt thereof; wherein all the formulae and symbols are as described above with the proviso that R₃, R₄ and R₅ cannot all be hydrogen.

In another embodiment, the present invention provides a compound of formula (74), or a solvate or pharmaceutically acceptable salt thereof; wherein all the formulae and symbols are as described above with the proviso that when R₃, R₄ and R₅ are all hydrogen then J is not a methanesulfonyl group.

In another embodiment, the present invention provides a compound of formula (96), or a solvate or pharmaceutically acceptable salt thereof; wherein all the formulae and symbols are as described above.

In another embodiment, the present invention provides a compound of formula (100), or a solvate or pharmaceutically acceptable salt thereof; wherein all the formulae and symbols are as described above.

In another embodiment, the present invention provides a compound of formula (62), or a solvate or pharmaceutically acceptable salt thereof; wherein all the formulae and symbols are as described above.

In another embodiment, the present invention provides a compound of formula (63), or a solvate or pharmaceutically acceptable salt thereof; wherein all the formulae and symbols are as described above.

In another embodiment, the present invention provides a compound of formula (78), or a solvate or pharmaceutically acceptable salt thereof; wherein all the formulae and symbols are as described above.

It is recognized that there may be one or more chiral centers in the compounds used within the scope of the present invention and thus such compounds will exist as various stereoisomeric forms. Applicants intend to include all the various stereoisomers within the scope of the invention. Though the compounds may be prepared as racemates and can conveniently be used as such, individual enantiomers also can be isolated or preferentially synthesized by known techniques if desired. Such racemates and individual enantiomers and mixtures thereof are intended to be included within the scope of the present invention. Pure enantiomeric forms if produced may be isolated by preparative chiral HPLC. The free base may be converted if desired, to the monohydrochloride salt by known methodologies, or alternatively, if desired, to other acid addition salts by reaction with other inorganic or organic acids. Acid addition salts can also be prepared metathetically by reacting one acid addition salt with an acid that is stronger than that of the anion of the initial salt.

The present invention also encompasses the pharmaceutically acceptable salts, esters, amides, complexes, chelates, solvates, crystalline or amorphous forms, metabolites, metabolic precursors or prodrugs of the compounds of the present invention. Pharmaceutically acceptable esters and amides can be prepared by reacting, respectively, a hydroxy or amino functional group with a pharmaceutically acceptable organic acid, such as identified below. A prodrug is a drug which has been chemically modified and may be biologically inactive at its site of action, but which is degraded or modified by one or more enzymatic or other in vivo processes to the parent bioactive form. Generally, a prodrug has a different pharmakokinetic profile than the parent drug such that, for example, it is more easily absorbed across the mucosal epithelium, it has better salt formation or solubility and/or it has better systemic stability (e.g., an increased plasma half-life).

Those skilled in the art recognize that chemical modifications of a parent drug to yield a prodrug include: (1) terminal ester or amide derivatives which are susceptible to being cleaved by esterases or lipases; (2) terminal peptides which may be recognized by specific or nonspecific proteases; or (3) a derivative that causes the prodrug to accumulate at a site of action through membrane selection, and combinations of the above techniques. Conventional procedures for the selection and preparation of prodrug derivatives are described in H. Bundgaard, Design of Prodrugs, (1985). Those skilled in the art are well-versed in the preparation of prodrugs and are well-aware of its meaning.

The present invention also encompasses the pharmaceutically acceptable complexes, chelates, metabolites, or metabolic precursors of the compounds of the present invention. Information about the meaning these terms and references to their preparation can be obtained by searching various databases, for example Chemical Abstracts and the U.S. Food and Drug Administration (FDA) website. Documents such as the followings are available from the FDA: Guidance for Industry, “In Vivo Drug Metabolism/Drug Interaction Studies—Study Design, Data Analysis, and Recommendations for Dosing and Labeling”, U.S. Department of Health and Human Services, Food and Drug Administration, Center for Drug Evaluation and Research (CDER), Center for Biologics Evaluation and Research (CBER), November 1999. Guidance for Industry, “In Vivo Drug Metabolism/Drug Interaction Studies in the DRUG DEVELOPMENT PROCESS: STUDIES IN VITRO”, U.S. Department of Health and Human Services, Food and Drug Administration, Center for Drug Evaluation and Research (CDER), Center for Biologics Evaluation and Research (CBER), April 1997.

The synthetic procedures described herein, especially when taken with the general knowledge in the art, provide sufficient guidance to those of ordinary skill in the art to perform the synthesis, isolation, and purification of the compounds of the present invention. Further, it is contemplated that the individual features of these embodiments and examples may be combined with the features of one or more other embodiments or examples.

As used herein, “treating arrhythmia” refers to therapy for arrhythmia. An effective amount of a composition of the present invention is used to treat arrhythmia in a warm-blooded animal, such as a human. Methods of administering effective amounts of antiarrhythmic agents are well known in the art and include the administration of an oral or parenteral dosage form. Such dosage forms include, but are not limited to, parenteral dosage form. Such dosage forms include, but are not limited to, parenteral solutions, tablets, capsules, sustained release implants, and transdermal delivery systems. Generally, oral or intravenous administration is preferred for some treatments. The dosage amount and frequency are selected to create an effective level of the agent without harmful effects. It will generally range from a dosage of from about 0.01 to about 100 mg/kg/day, and typically from about 0.1 to 10 mg/kg where administered orally or intravenously for antiarrhythmic effect or other therapeutic application.

In order to assess whether a compound has a desired pharmacological activity with the present invention, it may be subjected to a series of tests. The precise test to employ will depend on the physiological response of interest. The published literature contains numerous protocols for testing the efficacy of a potential therapeutic agent, and these protocols may be employed with the present compounds and compositions.

For example, in connection with treatment or prevention of arrhythmia, a series of four tests may be conducted. In the first of these tests, a compound of the present invention is given as increasing (doubling with each dose) intravenous infusion every 5 minutes to a conscious rat. The effects of the compound on blood pressure, heart rate and the ECG are measured continuously. Increasing doses are given until a severe adverse event occurs. The drug related adverse event is identified as being of respiratory, central nervous system or cardiovascular system origin. This test gives an indication as to whether the compound is modulating the activity of sodium channels and/or potassium channels, and in addition gives information about acute toxicity. The indices of sodium channel blockade are increasing P-R interval and QRS widening of the ECG. Potassium channel blockade results in Q-T interval prolongation of the ECG.

A second test involves administration of a compound as an infusion to pentobarbital anesthetized rats in which the left ventricle is subjected to electrical square wave stimulation performed according to a preset protocol described in further detail below. This protocol includes the determination of thresholds for induction of extrasystoles and ventricular fibrillation. In addition, effects on electrical refractoriness are assessed by a single extra beat technique. In addition effects on blood pressure, heart rate and the ECG are recorded. In this test, sodium channel blockers produce the ECG changes expected from the first test. In addition, sodium channel blockers also raise the thresholds for induction of extrasystoles and ventricular fibrillation. Potassium channel blockade is revealed by increasing refractoriness and widening of the Q-T intervals of the ECG.

A third test involves exposing isolated rat hearts to increasing concentrations of a compound. Ventricular pressures, heart rate, conduction velocity and ECG are recorded in the isolated heart in the presence of varying concentrations of the compound. The test provides evidence for direct toxic effects on the myocardium. Additionally, selectivity, potency and efficacy of action of a compound can be ascertained under conditions simulating ischemia. Concentrations found to be effective in this test are expected to be efficacious in the electrophysiological studies.

A fourth test is estimation of the antiarrhythmic activity of a compound against the arrhythmias induced by coronary artery occlusion in anaesthetized rats. It is expected that a good antiarrhythmic compound will have antiarrhythmic activity at doses which have minimal effects on either the ECG, blood pressure or heart rate under normal conditions.

All of the foregoing tests are performed using rat tissue. In order to ensure that a compound is not having effects which are only specific to rat tissue, further experiments are performed in dogs and primates. In order to assess possible sodium channel and potassium channel blocking action in vivo in dogs, a compound is tested for effects on the ECG, ventricular epicardial conduction velocity and responses to electrical stimulation. An anesthetized dog is subjected to an open chest procedure to expose the left ventricular epicardium. After the pericardium is removed from the heart a recording/stimulation electrode is sewn onto the epicardial surface of the left ventricle. Using this array, and suitable stimulation protocols, conduction velocity across the epicardium as well as responsiveness to electrical stimulation can be assessed. This information coupled with measurements of the ECG allows one to assess whether sodium and/or potassium channel blockade occurs. As in the first test in rats, a compound is given as a series of increasing bolus doses. At the same time possible toxic effects of a compound on the dog's cardiovascular system is assessed.

The effects of a compound on the ECG and responses to electrical stimulation are also assessed in intact, anesthetized monkeys (Macaca fascicularis). In this preparation, a blood pressure cannula and ECG electrodes are suitably placed in an anesthetized monkey. In addition, a stimulating electrode is placed onto the right atria and/or ventricle, together with monophasic action potential electrode. As in the tests described above, ECG and electrical stimulation response to a compound reveal the possible presence of sodium and/or potassium channel blockade. The monophasic action potential also reveals whether a compound widens the action potential, an action expected of a potassium channel blocker.

As another example, in connection with the mitigation or prevention of the sensation of pain, the following test may be performed. To determine the effects of a compound of the present invention on an animal's response to a sharp pain sensation, the effects of a slight prick from a 7.5 g weighted syringe fitted with a 23 G needle as applied to the shaved back of a guinea pig (Cavia porcellus) is assessed following subcutaneous administration of sufficient (50 μl, 10 mg/ml) solution in saline to raise a visible bleb on the skin. Each test is performed on the central area of the bleb and also on its periphery to check for diffusion of the test solution from the point of administration. If the test animal produces a flinch in response to the stimulus, this demonstrates the absence of blockade of pain sensation. Testing may be carried out at intervals for up to 8 hours or more post-administration. The sites of bleb formation are examined after 24 hours to check for skin abnormalities consequent to local administration of test substances or of the vehicle used for preparation of the test solutions.

The following examples are offered by way of illustration and not by way of limitation. In the Examples, and unless otherwise specified, starting materials were obtained from well-known commercial supply houses, e.g., Aldrich Chemical Company (Milwaukee, Wis.), and were of standard grade and purity. “Ether” and “ethyl ether” each refers to diethyl ether; “h.” refers to hours; “min.” refers to minutes; “GC” refers to gas chromatography; “v/v” refers to volume per volume; and ratios are weight ratios unless otherwise indicated.

General Experimental Procedures

Melting points were determined on a Fisher-Johns apparatus and are uncorrected. NMR spectra were acquired in the indicated solvent on a Brucker AC-200, Varian XL-300, Brucker AV-300 or AV-400. Mass spectra were recorded for EI on a Kratos MS50, for FAB/LSIMS on a Kratos Concept IIHQ and for ES on a Micromass (Waters) Quattro (I) MSMS, connected to a HP1090 Series 2 LC (Agilent), controlled by Masslynx version 3.3 software. Elemental analyses were performed on an Element Analyzer 1108 by D. & H. Malhow, University of Alberta, Edmonton, AB. Where analyses are indicated only by symbols of the elements, analytical results were within ±0.4% of the theoretical values. Whenever elemental analyses were not available, purity was determined by HPLC and capillary electrophoresis (CE). HPLC analyses were performed using a Gilson HPLC system (Gilson, Middleton, Wis.) with UV detection at 200 nm. A C₁₈ column with 150×4.6 mm, 5 μ particle size was used. The mobile phase was delivered isocratically or as a gradient at a flow rate of 1 mL/min and consisted of a combination of phosphate buffer (low or high pH) and acetonitrile. Samples were prepared at ˜100 μg/mL in mobile phase and 20 μL were injected into the HPLC. Purity was expressed in area%. CE analyses were performed using a P/ACE System MDQ (Beckman Coulter, Fullerton, Calif.). Uncoated silica capillaries with 60 (50 to detector) cm length and 75 μm internal diameter were used. The run buffer used was 100 mM sodium phosphate (pH 2.5). The separation voltage was either 23 or 25 kV (normal polarity) and the capillary cartridge temperature was maintained at 20° C. Samples (˜0.5 mg/mL in water) were injected by pressure at 0.5 psi for 6 seconds. Detection was by UV at 200 or 213 nm. Purity was expressed in area%. IR were recorded on a Perkin-Elmer 983 G spectrophotometer. Optical rotations were performed by F. Hoffman-La Roche Ltd (CH, Basel). Thin layer chromatography (TLC) was performed on E. Merck, TLC aluminum sheets 20×20 cm, Silica gel 60 F₂₅₄ plates. Flash chromatography⁴¹ was performed on E.M. Science silica gel 60 (70-230 mesh). Dry flash chromatography⁴² was performed with Sigma silica gel type H. Chromatotron chromatography (Harisson Research, USA) was performed on 4 mm plate with EM Science silica gel 60P F₂₅₄ with Gypsum or aluminum oxide 60P F₂₅₄ with Gypsum (type E). Preparative HPLC were performed on a Waters Delta Prep 4000 with a cartridge column (porasil, 10 μm, 125 Å, 40 mm×100 mm). GC analyses were performed on a Hewlett Packard HP 6890 equipped with 30 m×0.25 mm×0.25 μm capillary column HP-35 (crosslinked 35% PH ME siloxane) and a flame-ionization detector. High-boiling solvents (DMF, DMSO) were Sure/Seal™ from Aldrich, and tetrahydrofuran (THF) and ethylene glycol dimethyl ether (DME) were distilled from sodium-benzophenone ketyl. Organic extracts were dried with Na₂SO₄ unless otherwise noted. All moisture sensitive reactions were performed in dried glassware under a nitrogen or argon atmosphere.

Biological Activity Data

Assessment of Antiarrhythmic Efficacy

Antiarrhythmic efficacy may be assessed by investigating the effect of a compound on the incidence of cardiac arrhythmias in anesthetized rats subjected to coronary artery occlusion. Rats weighing 200-300 gms are subjected to preparative surgery and assigned to groups in a random block design. In each case, the animal is anesthetized with pentobarbital during surgical preparation. The left carotid artery is cannulated for measurement of mean arterial blood pressure and withdrawal of blood samples. The left jugular vein is also cannulated for injection of drugs. The thoracic cavity is opened and a polyethylene occluder loosely placed around the left anterior descending coronary artery. The thoracic cavity is then closed. An ECG is recorded by insertion of electrodes placed along the anatomical axis of the heart. In a random and double-blind manner, an infusion of vehicle or the compound to be tested is given about 15 min post-surgery. After 5 minutes infusion, the occluder is pulled so as to produce a coronary artery occlusion. ECG, arrhythmias, blood pressure, heart rate and mortality are monitored for 15 minutes after occlusion. Arrhythmias are recorded as ventricular tachycardia (VT) and ventricular fibrillation (VF) and scored according to Curtis, M. J. and Walker, M. J. A., Cardiovasc. Res. 22:656 (1988) (see Table 1).

TABLE 1

| Score | Description |
|---|---|
| 0 | 0-49 VPBs |
| 1 | 50-499 VPBs |
| 2 | >499 VPBs and/or 1 episode of spontaneously reverting VT or VF |
| 3 | >1 episode of VT or VF or both (>60s total combined duration) |
| 4 | VT or VF or both (60-119s total combined duration) |
| 5 | VT or VF or both (>119s total combined duration) |
| 6 | fatal VF starting at >15 min after occlusion |
| 7 | fatal VF starting at from 4 min and 14 min 59s after occlusion |
| 8 | fatal VF starting at from 1 min and 3 min 59s after occlusion |
| 9 | fatal VF starting <1 min after occlusion |

where: VPB=ventricular premature beats
VT=ventricular tachycardia
VF=ventricular fibrillation

Rats are excluded from the study if they did not exhibit pre-occlusion serum potassium concentrations within the range of 2.9-3.9 mM. Occlusion is associated with increases in R-wave height and “S-T” segment elevation; and an occluded zone (measured after death by cardiogreen dye perfusion) in the range of 25%-50% of total left-ventricular weight.

Results of the test compounds prepared by the method of the present invention may be expressed as values of a given infusion rate in micromol/kg/min. (ED₅₀AA) which will reduce the arrhythmia score in treated animals to 50% of that shown by animals treated only with the vehicle in which the test compound(s) is dissolved.

Measurement of Cardiovascular and Behavioral Effects

Preparative surgery is performed in Sprague Dawley rats weighing 200-300 gm and anaesthetized with 65 mg/kg (i.p.) pentobarbital. The femoral artery and vein are cannulated using polyethylene (PE)-10 tubing. Prior to surgery, this PE-10 tubing had been annealed to a wider gauge (PE-50) tubing for externalization. The cannulated PE-10/PE-50 tubing is passed through a trocar and exteriorised together with three (lead II) limb ECG leads (see below). The trocar is threaded under the skin of the back and out through a small incision at the mid-scapular region. A ground ECG electrode is inserted subcutaneously using a 20 gauge needle with the lead wire threaded through it. To place the other ECG electrodes, a small incision is made in the anterior chest region over the heart and ECG leads are inserted into the subcutaneous muscle layer in the region of the heart using a 20 guage needle. Other ECG leads are inserted into the subcutaneous muscle layer in the region near the base of the neck and shoulder (right side). The animal is returned to a clean recovery-cage with free access to food and water. The treatment and observational period for each animal commenced after a 24-hour recovery period.

A 15 minute-observational period is recorded followed by the intravenous infusion regime of the test compound at an initial dose of 2.0 μmol/kg/min (at 1 ml/hr). This rate is doubled every 5 minutes until one of the following effects is observed:
a) partial or complete convulsions
b) severe arrhythmias
c) bradycardia below 120 beats/minute
d) hypotension below 50mmHg
e) the dose exceeds 32 times the initial starting dose (i.e. 64 μmol/kg/min).

Blood pressure (BP), heart rate (HR) and ECG variables are continuously recorded while behavioral responses are also monitored and the total accumulative drug dose and drug infusion rate at which the response (such as convulsion, piloerection, ataxia, restlessness, compulsive chewing, lip-smacking, wet dog shake etc.) occurred are recorded.

Blood samples

Estimates of plasma concentrations of the test compound are determined by removing a 0.5 ml blood sample at the end of the experiment. Blood samples are centrifuged for 5 min at 4600×g and the plasma decanted. Brain tissue samples are also extracted and kept frozen (−20° C.) along with the plasma samples for chemical analysis.

Data Analysis

Electrocardiograph (ECG) parameters: PR, QRS, QT₁ (peak of T-wave), QT₂ (midpoint of T-wave deflection) and hemodynamic parameters: BP and HR are analyzed using the automated analysis function in LabView (National Instruments) with a customized autoanalysis software (Nortran Pharmaceuticals). The infused dose producing 25% from control (D₂₅) for all recorded ECG variables is determined.

Results of the tests can be expressed as D₂₅ (micromol/kg) which are the doses required to produce a 25% increase in the ECG parameter measured. The increases in P-R interval and QRS interval indicate cardiac sodium channel blockade while the increase in Q-T interval indicates cardiac potassium channel blockade.

Electrophysiological Test (In Vivo)

This experiment determines the potency of the test compound for its effects on haemodynamic and electrophysiological parameters under non-ischemic conditions.

Methods

Surgical Preparation

Male Sprague-Dawley rats weighing from 250-350 g are used. They are randomly selected from a single group and anesthetized with pentobarbital (65 mg/kg, ip.) with additional anesthetic given if necessary.

The trachea is cannulated and the rat is artificially ventilated at a stroke volume of 10 ml/kg, 60 strokes/minute. The right external jugular vein and the left carotid artery are cannulated for intravenous injections of compounds and blood pressure (BP) recording, respectively.

Needle electrodes are subcutaneously inserted along the suspected anatomical axis (right atrium to apex) of the heart for ECG measurement. The superior electrode is placed at the level of the right clavicle about 0.5 cm from the midline, while the inferior electrode is placed on the left side of the thorax, 0.5 cm from the midline and at the level of the ninth rib.

Two Teflon-coated silver electrodes are inserted through the chest wall using 27 G needles as guides and implanted in the epicardium of left ventricle (4-5 mm apart). Square pulse stimulation is provided by a stimulator controlled by a computer. In-house programmed software is used to determine the following: threshold current (iT) for induction of extra systoles, maximum following frequency (MFF), effective refractory period (ERP) and ventricular flutter threshold (VTt). Briefly, iT is measured as the minimal current (in μA) of a square wave stimulus required to capture and pace the heart at a frequency of 7.5 Hz and a pulse width of 0.5 msec; ERP is the minimum delay (in msec) for a second stimulus required to cause an extra systole with the heart entrained at a frequency of 7.5 Hz (1.5×iT and 0.2 msec pulse width), MFF is the maximum stimulation frequency (in Hz) at which the heart is unable to follow stimulation (1.5×iT and 0.2 msec pulse width); VTt is the minimum pulse current (in μA) to evoke a sustained episode of VT (0.2 msec pulse width and 50 Hz) (Howard, P. G. and Walker, M. J. A., Proc. West. Pharmacol. Soc. 33:123-127 (1990)).

Blood pressure (BP) and electrocardiographic (ECG) parameters are recorded and analyzed using LabView (National Instruments) with a customized autoanalysis software (Nortran Pharmaceuticals Inc.) to calculate mean BP (mmHg, ⅔ diastolic+⅓ systolic blood pressure), HR (bpm, 60/R-R interval); PR (msec, the interval from the beginning of the P-wave to the peak of the R-wave), QRS (msec, the interval from the beginning of the R-wave due to lack of Q wave in rat ECG, to the peak of the S-wave), QT (msec, the interval from the beginning of the R-wave to the peak of the T-wave).

Experimental Protocol

The initial infusion dose is chosen based on a previous toxicology study of the test compound in conscious rats. This is an infusion dose that did not produce a 10% change from pre-drug levels in haemodynamic or ECG parameters.

The animal is left to stabilize prior to the infusion treatment according to a predetermined random and blind table. The initial infusion treatment is started at a rate of 0.5 ml/hr/300 g (i.e., 0.5 μmol/kg/min). Each infusion dose is doubled (in rate) every 5 minutes. All experiments are terminated at 32 ml/hr/300 g (i.e., 32 μmol/kg/min). Electrical stimulation protocols are initiated during the last two minutes of each infusion level.

Data Analyses

Responses to test compounds are calculated as percent changes from pre-infusion values; this normalization is used to reduce individual variation. The mean values of BP and ECG parameters at immediately before the electrical stimulation period (i.e., 3 min post-infusion) are used to construct cumulative dose-response curves. Data points are fit using lines of best fit with minimum residual sum of squares (least squares; SlideWrite program; Advanced Graphics Software, Inc.). D₂₅'s (infused dose that produced 25% change from pre-infusion value) are interpolated from individual cumulative dose-response curves and used as indicators for determining the potency of compounds of the present invention.

Canine Vagal-AF Model

General Methods

Mongrel dogs of either sex weighing 15-49 kg are anesthetized with morphine (2 mg/kg im initially, followed by 0.5 mg/kg IV every 2 h) and α-chloralose (120 mg/kg IV followed by an infusion of 29.25 mg/kg/h; St.-Georges et al., 1997). Dogs are ventilated mechanically with room air supplemented with oxygen via an endotracheal tube at 20 to 25 breaths/minute with a tidal volume obtained from a nomogram. Arterial blood gases are measured and kept in the physiological range (SAO₂>90%, pH 7.30-7.45). Catheters are inserted into the femoral artery for blood pressure recording and blood gas measurement, and into both femoral veins for drug administration and venous sampling. Catheters are kept patent with heparinized 0.9% saline solution. Body temperature is maintained at 37-40° C. with a heating blanket.

The heart is exposed via a medial thoracotomy and a pericardial cradle is created. Three bipolar stainless steel, Teflon™-coated electrodes are inserted into the right atria for recording and stimulation, and one is inserted into the left atrial appendage for recording. A programmable stimulator (Digital Cardiovascular Instruments, Berkeley, Calif.) is used to stimulate the right atrium with 2 ms, twice diastolic threshold pulses. Two stainless steel, Teflon™-coated electrodes are inserted into the left ventricle, one for recording and the other for stimulation. A ventricular demand pacemaker (GBM 5880, Medtronics, Minneapolis, Minn.) is used to stimulate the ventricles at 90 beats/minute when (particular during vagal-AF) the ventricular rate became excessively slow. A P23 ID transducer, electrophysiological amplifier (Bloom Associates, Flying Hills, Pa.) and paper recorder (Astromed MT-95000, Toronto, ON, Canada) are used to record ECG leads II and III, atrial and ventricular electrograms, blood pressure and stimulation artefacts. The vagi are isolated in the neck, doubly-ligated and divided, and electrodes inserted in each nerve (see below). To block changes in β-adrenergic effects on the heart, nadolol is administered as an initial dose of 0.5 mg/kg iv, followed by 0.25 mg/kg IV every two hours.

Atrial Fibrillation Model

Drug effects to terminate sustained AF maintained during continuous vagal nerve stimulation are assessed. Unipolar hook electrodes (stainless steel insulated with Teflon™, coated except for the distal 1-2 cm) are inserted via a 21 gauge needle within and parallel to the shaft of each nerve. In most experiments, unipolar stimuli are applied with a stimulator (model DS-9F, Grass Instruments, Quincy, Mass.) set to deliver 0.1 ms square-wave pulses at 10 Hz and a voltage 60% of that required to produce asystole. In some experiments, bipolar stimulation is used. The voltage required to produce asystole ranged from 3-20 volts. Under control conditions, a short burst of rapid atrial pacing (10 Hz, four times diastolic threshold) is delivered to induce AF which is ordinarily sustained for more than 20 minutes. The vagal stimulation voltage is adjusted under control conditions, and then readjusted after each treatment to maintain the same bradycardic effect. AF is defined as rapid (>500 minute under control conditions), irregular atrial rhythm with varying electrogram morphology.

Measurement of Electrophysiological Variables and Vagal Response

Diastolic threshold current is determined at a basic cycle length of 300 ms by increasing the current 0.1 mA incrementally until stable capture is obtained. For subsequent protocols current is set to twice diastolic threshold. Atrial and ventricular ERP is measured with the extrastimulus method, over a range of S1S2 intervals at a basic cycle length of 300 ms. A premature extrastimulus S2 is introduced every 15 basic stimuli. The S1S2 interval is increased in 5 ms increments until capture occurred, with the longest S1S2 interval consistently failing to produce a propagated response defining ERP. Diastolic threshold and ERP are determined in duplicate and averaged to give a single value. These values are generally within 5 ms. The interval from the stimulus artefact and the peak of the local electrogram is measured as an index of conduction velocity. AF cycle length (AFCL) is measured during vagal-AF by counting the number of cycles (number of beats -1) over a 2-second interval at each of the atrial recording sites. The three AFCLs measurements are averaged to obtain an overall mean AFCL for each experimental condition.

The stimulus voltage-heart rate relationship for vagal nerve stimulation is determined under control conditions in most experiments. The vagal nerves are stimulated as described above with various voltages to determine the voltage which caused asystole (defined as a sinus pause greater than 3 seconds). The response to vagal nerve stimulation is confirmed under each experimental condition and the voltage adjusted to maintain the heart rate response to vagal nerve stimulation constant. In cases in which is not possible to produce asystole, vagal nerve stimulation is adjusted to a voltage which allowed two 20-minute episodes of vagal-AF to be maintained under control conditions (see below).

Experimental Protocols

One of the experimental groups studied is summarized in Table 3. Each dog received only one drug at doses indicated in Table 3. The first series of experiments are dose ranging studies, followed by blinded study in which 1-3 doses are given. All drugs are administered IV via an infusion pump, with drug solutions prepared freshly in plastic containers on the day of the experiment. Vagal stimulation parameters are defined under control conditions as described above, and maintenance of AF during 20 minutes of vagal nerve stimulation under control conditions is verified. After the termination of AF, the diastolic threshold and ERP of the atrium and ventricle are determined. Subsequently, these variables are reassessed in the atrium under vagal nerve stimulation. Electrophysiological testing usually took 15-20 minutes. The heart rate response to vagal nerve stimulation is confirmed and the vagal-AF/electrophysiological testing protocol is repeated. A pre-drug blood sample is obtained and vagal-AF reinstituted. Five minutes later, one of the treatments is administered at doses shown in Table 2. The total dose is infused over 5 minutes and a blood sample obtained immediately thereafter. No maintenance infusion is given. If AF terminated within 15 minutes, the electrophysiological measurements obtained under control conditions are repeated and a blood sample is obtained. If AF is not terminated by the first dose (within 15 minutes), a blood sample is obtained and vagal stimulation is discontinued to allow a return to sinus rhythm. The electrophysiological measurements are repeated and a third and final blood sample for this dose is obtained. AF is reinitiated and the vagal-AF/drug infusion/electrophysiological testing protocol is repeated until AF is terminated by the drug.

Statistical Analysis

Group data are expressed as the mean ±SEM. Statistical analysis is carried out for effective doses for AFCL, and ERP using a t-test with a Bonferroini correction for multiple comparisons. Drug effects on blood pressure, heart rate, diastolic threshold and ECG intervals are assessed at the median dose for termination of AF. Two tailed tests are used and a p<0.05 is taken to indicate statistical significance.

TABLE 2
Experimental Groups and Doses of Drugs

| | Dose | | Mean dose | Median dose |
|---|---|---|---|---|
| | range | Effective doses | required for | required for |
| | tested (μ | for terminating | termination of | termination of |
| Drug | mol/kg) | AF (μmol/kg) | AF (μmol/kg) | AF (μmol/kg) |
| Flecainide | 1.25-10 | 4-2.5;1-10 | 4 ± 2 | 2.5 |

A single drug was administered to each dog over the dose range specified until AF was terminated. The number of dogs in which AF was terminated at each dose is shown (number of dogs-dose, in μmol/kg). The mean ±SEM as well as the median dose required to terminate AF is shown. Each dog received only one drug.

Compounds prepared by the method of the present invention may be evaluated by this method. The effectiveness of flecainide as a control in the present study was comparable to that previously reported.

Canine Sterile Pericarditis Model

This model has been used to characterize the mechanisms of AF and atrial flutter (AFL). Waldo and colleagues have found that AF depends on reentry and that the site of termination is usually an area of slowed conduction. This canine model is prepared by dusting the exposed atria with talcum powder followed by “burst” pacing the atria over a period of days after recovery. AF is inducible two days after surgery, however, by the fourth day after surgical preparation; sustainable atrial flutter is the predominant inducible rhythm. The inducibility of AF at day 2 is somewhat variable, such that only 50% of dogs may have sustained AF (generally <60 minutes) for a requisite of 30 minutes. However, the sustainable atrial flutter that evolves by the fourth day is inducible in most preparations. Atrial flutter is more readily “mapped” for purposes of determining drug mechanisms. Inducibility of AF subsides after the fourth day post-surgery, similar to the AF that often develops following cardiac surgery that the sterile pericarditis model mimics. There may be an inflammatory component involved in the etiology of post-surgery AF that would provide a degree of selectivity to an ischaemia or acid selective drug. Similarly, while coronary artery bypass graft (CABG) surgery is performed to alleviate ventricular ischaemia, such patients may also be at risk for mild atrial ischaemia due to coronary artery disease (CAD). While atrial infarcts are rare, there has been an association from AV nodal artery stenosis and risk for AF following CABG surgery. Surgical disruption of the autonomic innervation of the atria may also play a role in AF following CABG.

Methods

Studies are carried out in a canine model of sterile percarditis to determine the potency and efficacy of compounds of the present invention in terminating atrial fibrillation/flutter. Atrial flutter or fibrillation was induced 2 to 4 days after creation of sterile pericarditis in adult mongrel dogs weighing 19 kg to 25 kg. In all instances, the atrial fibrillation or flutter lasted longer than 10 minutes.

Creation of the Sterile Pericarditis Atrial Fibrillation/Flutter Model

The canine sterile pericarditis model is created as previously described. At the time of surgery, a pair of stainless steel wire electrodes coated with FEP polymer except for the tip (O Flexon, Davis and Geck) are sutured on the right atrial appendage, Bachman's bundle and the posteroinferior left atrium close to the proximal portion of the coronary sinus. The distance from each electrode of each pair is approximately 5 mm. These wire electrodes are brought out through the chest wall and exteriorized posteriorly in the interscapular region for subsequent use. At the completion of surgery, the dogs are given antibiotics and analgesics and then are allowed to recover. Postoperative care included administration of antibiotics and analgesics.

In all dogs, beginning on postoperative day 2, induction of stable atrial fibrillation/flutter is attempted in the conscious, non-sedated state to confirm the inducibility and the stability of atrial fibrillation/flutter and to test the efficacy of the drugs. Atrial pacing is performed through the electrodes sutured during the initial surgery. On postoperative day 4, when stable atrial flutter is induced, the open-chest study is performed.

For the open-chest study, each dog is anesthetized with pentobarbital (30 mg/kg IV) and mechanically ventilated with 100% oxygen by use of a Boyle model 50 anesthesia machine (Harris-Lake, Inc.). The body temperature of each dog is kept within the normal physiological range throughout the study with a heating pad. With the dog anesthetized, but before the chest is opened, radiofrequency ablation of the His bundle is performed to create complete atrioventricular (AV) block by standard electrode catheter techniques. This is done to minimize the superimposition of atrial and ventricular complexes during subsequent recordings of unipolar atrial electrograms after induction of atrial flutter. After complete AV block is created, an effective ventricular rate is maintained by pacing of the ventricles at a rate of 60 to 80 beats per minute with a Medtronic 5375 Pulse Generator (Medtronic Inc.) to deliver stimuli via the electrodes sutured to the right ventricle during the initial surgery.

Determination of Stimulus Thresholds and Refractory Periods During Pacing

For the induction of AF/AFL, one of two previously described methods is used: (1) introduction of one or two premature atrial beats after a train of 8 paced atrial beats at a cycle length of 400 ms, 300 ms, 200 ms, or 150 ms, or (2) rapid atrial Pacing for Periods of 1 to 10 seconds at rates incrementally faster by 10 to 50 beats per minute than the spontaneous sinus rate until atrial flutter is induced or there is a loss of 1:1 atrial capture. Atrial pacing is performed from either the right atrial appendage electrodes or the posteroinferior left atrial electrodes. All pacing is performed using stimuli of twice threshold for each basic drive train with a modified Medtronic 5325 programmable, battery-poared stimulator with a pulse width of 1.8 ms.

After the induction of stable atrial fibrillation/flutter (lasting longer than 10 minutes), the atrial fibrillation/flutter cycle length is measured and the initial mapping and analysis are performed to determine the location of the atrial fibrillation/flutter reentrant circuit. Atrial flutter is defined as a rapid atrial rhythm (rate, >240 beats per minute) characterized by a constant beat-to-beat cycle length, polarity, morphology, and amplitude of the recorded bipolar electrograms.

Drug Efficacy Testing Protocol

1. Effective refractory periods (ERPs) are measured from three sites: right atrial appendage (RAA), posterior left atrium (PLA), and Bachman's Bundle (BB), at two basic cycle lengths 200 and 400 ms.

2. Pace induce A-Fib or AFL. This is attempted for one hour. If no arrhythmia is induced, no further study is done on that day.

3. If induced, AF must have been sustained for 10 minutes. Then a waiting period is allowed for spontaneous termination or 20 minutes, whichever came first.

4. AF is then reinduced and 5 minutes is allowed before starting drug infusion.

5. Drug is then infused in a bolus over 5 minutes.

6. If AF terminated with the first dose then a blood sample is taken and ERP measurements are repeated.

7. Five minutes is allowed for the drug to terminate. If there is no termination then the second dose is given over 5 minutes.

8. After termination and ERPs are measured, a second attempt to reinduce AF is tried for a period of ten minutes.

9. If reinduced and sustained for 10 minutes, a blood sample is taken and the study repeated from #3 above.

10. If no reinduction, then the study is over.

Compounds prepared by the method of the present invention may be evaluated by this method.

Assessment of Pain Blockage

CD-1 mice (20-30 g) are restrained in an appropriate holder. A tourniquet is placed at the base of the tail and a solution of the test compound (50 μl, 5 mg/ml) is injected into the lateral tail vein. The tourniquet is removed 10 min after the injection. Suitable dilutions of compound solution are used to obtain an ED₅₀ for pain blockade at various times after injection. Pain responses are assessed by pin prick at regular intervals up to 4 hours post injection and the duration of pain blockage is recorded for three animals for each test compound solution. Compounds prepared by the method of the present invention may be evaluated according to the method described.

In Vitro Assessment of Inhibition Activity of ION Channel Modulating Compounds on Different Cardiac Ionic Currents

Cell Culture:

The relevant cloned ion channels (e.g., cardiac hH1Na, Kv1.4, Kv1.5, Kv4.2, Kv2.1, HERG etc.) are studied by transient transfection into HEK cells using the mammalian expression vector pCDNA3. Transfections for each channel type are carried out separately to allow individual study of the ion channel of interest. Cells expressing channel protein are detected by cotransfecting cells with the vector pHook-1 (Invitrogen, San Diego, Calif., USA). This plasmid encoded the production of an antibody to the hapten phOX, which when expressed is displayed on the cell surface. Equal concentrations of individual channel and pHook DNA are incubated with 10× concentration of lipofectAce in Modified Eagle's Medium (MEM, Canadian Life Technologies) and incubated with parent HEK cells plated on 25 mm culture dishes. After 3-4 hours the solution is replaced with a standard culture medium plus 20% fetal bovine serum and 1% antimycotic. Transfected cells are maintained at 37 C in an air/5%CO2 incubator in 25 mm Petri dishes plated on glass coverslips for 24-48 hours to allow channel expression to occur. 20 min prior to experiments, cells are treated with beads coated with phOX. After 15 min, excess beads are ished off with cell culture medium and cells which had beads stuck to them are used for electrophysiological tests.

Solutions:

For whole-cell recording the control pipette filling solution contained (in mM): KCl, 130; EGTA, 5; MgCl2, 1; HEPES, 10; Na2ATP, 4; GTP, 0.1; and is adjusted to pH 7.2 with KOH. The control bath solution contained (in mM): NaCl, 135; KCI, 5; sodium acetate, 2.8; MgCl2, 1; HEPES, 10; CaCl2, 1; and is adjusted to pH 7.4 with NaOH. The test ion channel modulating compound is dissolved to 10 mM stock solutions in water and used at concentrations from 0.5 and 100 μM.

Electrophysiological Procedures:

Coverslips containing cells are removed from the incubator before experiments and placed in a superfusion chamber (volume 250 μl) containing the control bath solution at 22 C to 23 C. All recordings are made via the variations of the patch-clamp technique, using an Axopatch 200A amplifier (Axon Instruments, CA). Patch electrodes are pulled from thin-walled borosilicate glass (World Precision Instruments; FL) on a horizontal micropipette puller, fire-polished, and filled with appropriate solutions. Electrodes had resistances of 1.0-2.5 μohm when filled with control filling solution. Analog capacity compensation is used in all whole cell measurements. In some experiments, leak subtraction is applied to data. Membrane potentials have not been corrected for any junctional potentials that arose from the pipette and bath solution. Data are filtered at 5 to 10 kHz before digitization and stored on a microcomputer for later analysis using the pClamp6 software (Axon Instruments, Foster City, Calif.). Due to the high level of expression of channel cDNA's in HEK cells, there is no need for signal averaging. The average cell capacitance is quite small, and the absence of ionic current at negative membrane potentials allowed faithful leak subtraction of data.

Data Analysis:

The concentration-response curves for changes in peak and steady-state current produced by the test compound are computer-fitted to the Hill equation:
f=1−1/[1+(IC₅₀[D])^{n]} [1]
where f is the fractional current (f=Idrug/Icontrol) at drug concentration [D]; IC₅₀ is the concentration producing half-maximal inhibition and n is the Hill coefficient.

Compounds of the present invention may be evaluated by this method. The results show that compounds of the present invention tested have different degree of effectiveness in blocking various ion channels. Block is determined from the decrease in peak hH1 Na⁺ current, or in steady-state Kv1.5 and integrated Kv4.2 current in the presence of drug. To record Na⁺ current, cells are depolarized from the holding potential of −100 mV to a voltage of −30 mV for 10 ms to fully open and inactivate the channel. To record Kv1.5 and Kv4.2 current, cells are depolarized from the holding potential of −80 mV to a voltage of +60 mV for 200 ms to fully open the channel. Currents are recorded in the steady-state at a range of drug concentrations during stimulation every 4 s. Reduction in peak current (Na⁺ channel), steady-state current (Kv1.5 channel) or integrated current (Kv4.2) at the test potential of −30 mV (Na⁺channel) or +60 mV (Kv1.5 and Kv4.2 channel) is normalized to control current, then plotted against the concentration of test compound. Data are averaged from 4-6 cells. Solid lines are fit to the data using a Hill equation. The activity of compounds prepared by method of the present invention to modulate various ionic currents of interest may be similarly studied.

Assessment of Proarrhythmia (Torsade de Pointes) Risk of Ion Channel Modulating Compounds in Primates

Method

General Surgical Preparation:

All studies are carried out in male Macaca fascicularis weighing from 4 and 5.5 kg. Animals are fasted over night and pre-medicated with ketamine (10 mg/kg im). Both saphenous veins are cannulated and a saline drip instituted to keep the lines patent. Halothane anaesthesia (1.5% in oxygen) is administered via a face mask. Lidocaine spray (10% spray) is used to facilitate intubation. After achieving a sufficient depth of anaesthesia, animals are intubated with a 4 or 5 French endotrachial tube. After intubation halothane is administered via the endotracheal tube and the concentration is reduced to 0.75-1%. Artificial respiration is not used and all animals continue to breathe spontaneously throughout the experiment. Blood gas concentrations and blood pH are measured using a blood gas analyser (AVO OPTI I). The femoral artery is cannulated to record blood pressure.

Blood pressure and a modified lead II ECG are recorded using a MACLAB 4S recording system paired with a Macintosh PowerBook (2400c/180). A sampling rate of 1 kHz is used for both signals and all data is archived to a Jazz disc for subsequent analysis.

Vagal Nerve Stimulation:

Either of the vagi is isolated by blunt dissection and a pair of electrodes inserted into the nerve trunk. The proximal end of the nerve is crushed using a vascular clamp and the nerve is stimulated using square wave pulses at a frequency of 20 Hz with a 1 ms pulse width delivered from the MACLAB stimulator. The voltage (range 2-10V) is adjusted to give the desired bradycardic response. The target bradycardic response is a reduction in heart rate by half. In cases where a sufficient bradycardic response could not be obtained, 10 μg/kg neostigmine iv is administered. This dose of neostigmine is also given after administration of the test drug in cases where the test drug has vagolytic actions.

Test Compounds:

A near maximum tolerated bolus dose of the test compound, infused (iv) over 1 minute, is used to assess the risk of torsade de pointes caused by each test compound. The actual doses vary slightly depending on the animals' weight. Clofilium, 30 μmol/kg, is used as a positive comparison (control) for these studies. The expectation is that a high dose of drug would result in a high incidence of arrhythmias. The test compounds are dissolved in saline immediately before administration.

Experimental Protocol:

Each animal receives a single dose of a given drug iv. Before starting the experiment, two 30 second episodes of vagal nerve stimulation are recorded. A five minute rest period is allowed from episodes and before starting the experiment. The test solution is administered as an iv bolus at a rate of 5 ml/minute for 1 minute using an infusion pump (total volume 5 ml). ECG and blood pressure responses are monitored continuously for 60 minutes and the occurrence of arrhythmias is noted. The vagal nerve is stimulated for 30 seconds at the following times after injection of the drug: 30 seconds, 2, 5, 10, 15, 20, 25, 30 and 60 minutes.

Blood samples (1 ml total volume) are taken from each treated animal at the following times after drug administration: 30 seconds, 5, 10, 20, 30 and 60 minutes as well as 3, 6, 24 and 48 hours. Blood samples taken up to 60 minutes after drug administration are arterial while those taken after this time are venous. Samples are centrifuged, the plasma decanted and frozen. Samples are kept frozen before analysis of plasma concentration of the drug and potassium.

Statistics:

The effect of drugs on blood pressure, heart rate and ECG intervals are described as the mean±SEM for a group size of “n.”

Compounds of the present invention may be evaluated by this method.

Determination of CNS Toxicity

In order to assess the activity of ion channel compounds in vivo it is important to know the maximum tolerated dose. Here CNS toxicity was assessed by investigating the minimum dose of a compound which induces partial or complete convulsions in conscious rats. The procedure avoids using lethality as an end point as well as avoiding unnecessary suffering as the experiment is terminated if this appears likely. Should the drug precipitate a life threatening condition (e.g., severe hypotension or cardiac arrhythmias) the animals are sacrificed via an overdose of pentobarbital.

Rats weighing 200-250 g were anaesthetized with pentobarbital anesthetic and subjected to preparative surgery. The femoral artery was cannulated for measurement of blood pressure and withdrawal of blood samples. The femoral vein was cannulated for injection of drugs. ECG leads were inserted into the subcutaneous muscle layer in the region of the heart and in the region near the base of the neck and shoulder. All cannulae and ECG leads were exteriorized in the mid scalpular region. To alleviate post-operative pain narcotics and local anesthetics were used. Animals were returned to a recovery cage for at least 24 hours before commencing the experiment. Infusion of the compound was then commenced via the femoral vein cannula. The initial rate of infusion was set at 2.0 micromole/kg/min at a rate of 1 ml/hr. The infusion rate was doubled every minute until partial or complete convulsions were observed. The maximum infusion rate used was 64 micromole/kg/min. Rates were continuously monitored and end time an infusion rate noted.

All publications and patent applications mentioned in this specification are herein incorporated by reference to the same extent as if each individual publication or patent application was specifically and individually incorporated by reference.

From the foregoing it will be appreciated that, although specific embodiments of the invention have been described herein for purposes of illustration, various modifications may be made without deviating from the spirit and scope of the invention. Accordingly, the invention is not limited by the specific embodiments and examples contained in this patent.

### EXAMPLES

### Example 1

### Synthesis of (1S,2S)-3-Chloro-cyclohex-3-ene-1,2-diol (60)

Compound (59), the starting material for this reaction, was synthesized according to the procedure of Boyd et al (D. R. Boyd, N. D. Sharma, H. Dalton, D. A. Clarke, Chem. Commun., 1996, 45.) or purchased from Sigma-Aldrich. With compound (59) in hand, 5% Rh/Al₂O₃ (Lancaster, 3 g, 15 wt. %), THF (anhydrous, 200 ml) were charged to a hydrogenation bottle and saturated with hydrogen for about 120 minutes, using a Par hydrogenation equipment. Compound (59) (20 g, 0.13 mol) was added and hydrogenation continued at 20 psi. After about 1 hour, all starting material was consumed (by TLC analysis—10% MeOH/DCM, which indicated both product and some amount of over-reduced intermediate). There was a significant temperature increase during the reaction. After filtration through a celite plug to remove residual catalyst, the filtrate was concentrated to a light brownish solid. The crude product was recrystallized to give the desired compound (60) in 60-70% yield.

Compound 60: R_{ƒ} 0.63 (EtOAc). MP=111-112° C.; [α]_{D}=−158 (c1.1, MeOH); ¹H-NMR (400 MHz, CDCl₃) δ: 1.68-1.83 (m, 2H), 2.05-2.13 (m, 1H), 2.23-2.30 (m, 1H), 2.98 (s, 2H), 3.88-3.92 (overlap dt, J=4.0 Hz, J=3.9 Hz, J=9.7 Hz), 5.97 (t, J=3.74 Hz, J=8.1 Hz). ¹³C-NMR (100 MHz, CDCl₃) δ: 23.79, 24.98, 69.05, 70.58, 128.48, 131.12; ¹H-NMR (400 MHz, DMSO-d6) δ: 1.47-1.51 (m, 1H), 1.56-1.63 (m, 1H), 2.01-2.04 (m, 1H), 2.08-2.11 (m, 1H), 3.56-3.61 (m, 1H), 3.83 (dd, J=4.3 Hz, J=5.4 Hz), 4.61 (d, J=6.01), 5.09 (d, J=6.27), 5.86 (dd, J=1.65 Hz, J=4.83 Hz).

### Example 2

### Synthesis of (1S,2S)-Benzenesulfonic acid-3-chloro-2-hydroxy-cyclohex-3-enyl ester (61A)

To a solution of the (1S,2S)-3-chloro-cyclohex-3-ene-1,2-diol (60) in anhydrous CH₂Cl₂ at room temperature were added benzenesulfonyl chloride, Et₃N and catalytic amount of Bu₂SnO. The reaction mixture was stirred at room temperature under inert atmosphere until completion as monitored by TLC. The reaction was quenched with water, and the layers were separated. After using standard work-up and purification protocols, compound (61A) was obtained as a colorless oil.

Compound (61A): R_{ƒ} 0.47. ¹H-NMR (300 MHz, CDCl₃) δ: 1.64-1.71 (m, 1H), 1.99-2.09 (m, 2H), 2.17-2.26 (m, 2H). 2.64 (s, 1H), 4.23 (dd, J=1.0 Hz, J=4.0 Hz, 1H), 4.68-4.74 (overlap dt, J=3.6 Hz, J=3.6 Hz, J=10.7 Hz, 1H), 5.91 (dd, 1H, J=2.9 Hz, J=4.5 Hz), 7.54 (t, J=8.0 Hz, 2H), 7.62-7.66 (m, 1H), 7.92 (dd, J=1.0 Hz, J=8.0 Hz, 2H). ¹³C-NMR (75 MHz, CDCl₃) δ: 22.11, 23.68, 69.32, 79.70, 127.62, 128.19, 129.30, 129.71, 133.99, 136.48.

### Example 3

### Synthesis of (1S,2R)-Benzenesulfonic acid 2-hydroxy-cyclohexyl ester (62A)

Reduction and dehalogenation of compound 61A to form 62A were accomplished under standard hydrogenation conditions (Pd/C, 5-20% by weight and H₂ gas) in basic condition. After the reaction was deemed completed as monitored by TLC, the reaction mixture was filtered through a pad of Celite. The product (62A) was obtained as an oil after standard work-up and purification protocols.

Compound 62A: R_{f} 0.71. ¹H-NMR (300 MHz, CDCl₃) δ: 1.20-1.33 (m, 2H), 1.42-1.63 (m, 2H), 1.66-1.76 (m, 1H), 1.83-1.93 (m, 1H), 2.05 (bs, 1H), 3.79-3.823 (m, 1H), 4.61-4.66 (overlap dt, J=3.1 Hz, J=2.9 Hz, J=8.2 Hz, 1H), 7.50-7.56 (m, 2H), 7.60-7.66 (m, 1H), 7.90-7.94 (m, 2H). ¹³C-NMR (75 MHz, CDCl₃) δ: 20.69, 21.66, 27.71, 30.20, 68.94, 83.43, 127.58, 129.20, 133.70, 137.16.

### Example 4

### Synthesis of (1S,2R-cis)-Benzenesulfonic acid 2-[2(3,4-dimethoxy-phenyl)-ethoxyl-cyclohexyl ester (64A)

To a solution of (1S,2R)-benzenesulfonic acid 2-hydroxy-cyclohexyl ester (62A) in a suitable halohydrocarbon solvent (e.g., dichloromethane) was added a catalytic amount of a suitable Lewis acid (0.1-0.5 mole), followed by the addition of a solution of 2,2,2-trichloro-acetimidic acid 2-(3,4-dimethoxy-phenyl)ethyl ester (63) in a suitable halohydrocarbon solvent (e.g., dichloromethane). The reaction mixture was stirred under an inert atmosphere at a suitable temperature (e.g., around room temperature) until the consumption of 62A was considered complete as monitored by TLC. This was followed by standard work-up and purification protocols to provide 64A as an oil.

Compound 64A: R_{ƒ} 0.72. ¹H-NMR(400 MHz, CDCl₃) δ: 1.15-1.30 (m, 2H), 1.37-1.60 (m, 4H), 1.65-1.76 (m, 1H), 1.91-2.00 (m, 1H), 2.61-2.71 (m, 2H), 3.36-3.38 (m, 1H), 3.49 (t, J=7.0 Hz, 1H), 3.82 (s, 3H), 3.83 (s, 3H), 4.65-4.70 (m, 1H), 6.66-6.76 (m, 3H), 7.46-7.50 (m, 2H), 7.56-7.60 (m, 1H), 7.88-7.90 (dd, J=1.0 Hz, J=9.0 Hz, 2H).

### Example 5

### Synthesis of (1R,2R)-2-[(3R)-Hydroxypyrrolidinyl]-1-(3,4-dimethoxyphenethoxy)cyclohexane (66)

A round flask of appropriate size was charged with (1S,2R-cis)-benzenesulfonic acid 2-[2(3,4-dimethoxy-phenyl)-ethoxy]-cyclohexyl ester (64A) and 3-(R)-pyrrolidinol (65) in a suitable molar ratio, which generally may require an excess of 65. The reaction mixture was stirred at an elevated temperature (e.g., from about 40° C. to about 90° C. or higher) such that the reaction may proceed at a rate to allow completion within a reasonable time (e.g., about 2 h to about 48 h or longer). The reaction mixture was stirred under an inert atmosphere and monitored by TLC. This was followed by standard work-up and purification protocols to provide the title compound (66). This product exhibited a diastereomeric excess of greater than 98% (chiral CE).

Compound 66: R_{ƒ} 0.50. ¹H-NMR (CDCl₃, 300 MHz) δ: 1.15-1.37 (m, 4H), 1.51-2.05 (m, 6H), 2.38-2.52 (m, 2H), 2.60-2.68 (m, 1H), 2.76-2.81 (m, 3H), 2.90-2.97 (m, 2H), 3.28-3.34 (m, 1H), 3.51-3.59 (m, 1H), 3.69-3.77 (m, 1H), 3.82 (s, 3H), 3.84 (s, 3H), 4.18-4.21 (m, 1H), 6.71-6.78 (m, 3H). ¹³C-NMR (75 MHz, CDCl₃) δ: 22.98, 23.43, 27.22, 28.98, 34.11, 36.36, 48.46, 55.80, 63.54, 69.48, 70.97, 79.25, 111.16, 112.37, 120.72, 131.86, 147.38, 148.68.

### Example 6

### Synthesis of 2,2,2-Trichloro-acetimidic acid 2-(3,4-dimethoxy-phenyl)-ethyl ester (63)

In a typical procedure, the trichloroacetimidate 63 may be synthesized from the corresponding primary alcohol under basic condition using trichloroacetonitrile as the reagent. Generally, the reaction was completed after stirring at about room temperature for about 1 hour or longer. After using standard work-up protocols, the product may be recrystallized from an appropriate solvent system.

Accordingly, 3,4-dimethoxyphenethyl alcohol (DMPE) and trichloroacetonitrile were reacted together. The reaction was monitored and determined by HPLC analysis. Upon completion, the reaction mixture was subjected to standard work-up and purification protocols to provide the product 63.

### Example 7

### Isolation of (1S-cis)-3-chloro-3,5-cyclohexadiene-1,2-diol (59)

Compound 59 was obtained from Sigma-Aldrich as a frozen suspension in phosphate buffer. To recover the pure product from the suspension, the frozen suspension was thawed. To the suspension (25 mL) containing 5.0 g of the product was added saturated aqueous Na₂CO₃ solution (25 mL). The aqueous layer was extracted with EtOAc (3×25 mL), and the organic layers were combined and dried over anhydrous MgSO₄, filtered, and concentrated in vacuo to give 59 as a white solid (4.1 g, 82%).

Compound 59: R_{ƒ} 0.40. ¹H-NMR (400 MHz, DMSO-d₆) δ: 3.84 (t, 1H, J₁=J₂=6.5 Hz), 4.28-4.32 (m, 1H), 5.03 (d, 1H, J=6.8 Hz), 5.22 (d, 1H, J=6.5 Hz,), 5.73-5.82 (m, 2H, H-4), 6.11 (d, 1H, J=6.1 Hz). ¹³C-NMR (100 MHz, DMSO-d₆) δ 69.85, 70.96, 121.28, 122.66, 131.70, 135.39.

### Example 8

### Synthesis of 6-(tert-butyldiphenylsiloxy)-2-chloro-cyclohexa-2,4-dienol (95)

Imidazole (97 mg, 1.43 mmol) and tert-butylchlorodiphenylsilane (393 mg, 1.43 mmol) were added to a cooled (−20° C.) solution of (1S-cis)-3-chloro-3,5-cyclohexadiene-1,2-diol 59 (0.3 g, 1.36 mmol) in anhydrous CH₂Cl₂ (8 mL). After the solution had stirred at −20° C. for 18 h, the reaction was quenched by the addition of ice-cold water (10 mL). The organic layer was separated from the aqueous layer, which was further extracted with CH₂Cl₂ (2×10 mL). The organic layers were combined, washed with brine (10 mL), dried (anhydrous MgSO₄), and concentrated in vacuo to give a slurry (0.47 g). The crude product was purified by elution through a silica gel plug using a mixture of ethyl acetate:hexane to give 95 as a colorless syrup (0.41 g, 78%).

Compound 95: R_{ƒ} 0.40. ¹H-NMR (400 MHz, DMSO-d₆) δ: 1.02 (2, 9H), 3.71 (t, 1H, J₁=J₂=6.5 Hz), 4.42-4.45 (m, 1H), 5.44 (d, 1H, J=7.2 Hz), 5.67 (dd, 1H, J=12 Hz, J=2.4 Hz), 5.75-5.78 (m, 1H), 6.07 (d, 1H, J=5.7 Hz), 7.39-7.48 (m, 6H), 7.64-7.69 (m, 4H).

### Example 9

### Synthesis of (1S,2S)-cis-2-(tert-butyldiphenylsiloxy)cyclohexanol (96)

Sodium acetate (96 mg, 1.17 mmol) and Pd/C (10% by weight, 30 mg) were added to a solution of 6-(tert-butyldiphenylsiloxy)-2-chloro-cyclohexa-2,4-dienol (95) (0.30 g, 0.78 mmol) in ethanol. The reaction vessel charged with the resultant suspension was flushed twice with H₂ and the reaction mixture was stirred at room temperature under H₂ (charged balloon) with monitoring by TLC. Upon completion, the reaction mixture was filtered through a pad of Celite. The filtrate was concentrated in vacuo to give a solid residue, which was dissolved in CH₂Cl₂ and the resultant solution was washed twice with brine. The organic layer was dried (anhydrous MgSO₄) and concentrated in vacuo to give a colorless oil. The crude product was purified by flash column chromatography to give 96 as a colorless oil (0.19 g, 70%).

Compound 96: R_{ƒ} 0.65. ¹H-NMR (400 MHz, DMSO-d₆) δ: 1.02 (s, 9H), 1.12-1.25 (m, 3H), 1.31-1.35 (m, 1H), 1.46-1.57 (m, 3H), 1.66-1.74 (m, 1H), 3.51-3.52 (m, 1H), 3.74-3.76 (m, 1H), 4.26 (d, 1H, J=3.4 Hz), 7.35-7.43 (m, 6H), 7.62-7.65 (m, 2H), 7.69-7.71 (m, 2H). ¹H-NMR (400 MHz, CDCl₃) δ: 1.08 (s, 9H), 1.20-1.39 (m, 4H), 1.53-1.68 (m, 3H), 1.81-1.86 (m, 1H), 2.17 (s, 1H), 3.70-3.76 (m, 2H), 7.35-7.44 (m, 6H), 7.65-7.68 (m, 4). ¹³C-NMR (125 MHz, CDCl₃) δ: 19.25, 20.37, 22.65, 27.02, 29.72, 30.15, 70.43, 73.39, 127.57, 127.69, 129.68, 129.76, 135.70, 135.79.

### Example 10

### Synthesis of (1S-cis)-tert-butyl-{2-[2-(3,4-dimethoxy-phenyl)ethoxy]-cyclohexyloxy}diphenylsilane (97)

A two-necked round bottom flask equipped with a magnetic stir bar and an argon inlet was flushed with argon, and was charged with a solution of (1S,2S)-cis-2-(tert-butyldiphenylsiloxy)cyclohexanol 96 in anhydrous dichloromethane. To the cooled solution (about 0° C.) was added successively trimethylsilyl trifluoromethanesulfonate, and a solution of 2,2,2-trichloro-acetimidic acid 2-(3,4-dimethoxy-phenyl)ethyl ester 63 in CH₂Cl₂. The reaction mixture was stirred at around room temperature, and the progress of the reaction was monitored by TLC. Upon completion, the reaction mixture was quenched by the addition of water. The aqueous layer was extracted three times with CH₂Cl₂. The organic extracts were combined, washed successively with saturated NaHCO₃ and brine, dried (anhydrous MgSO₄), and concentrated in vacuo to give a pale yellow syrup. Purification of this crude material by flash preparatory TLC provided the product 97 as a light yellow oil.

Compound 97: R_{ƒ} 0.43. ¹H-NMR (300 MHz, CDCl₃) δ: 1.07 (s, 9H), 1.18-1.26 (m, 2H), 1.33-1.41 (m, 1H), 1.63-1.67 (m, 3H), 1.82-1.85 (m, 3H), 2.69-2.76 (m, 2H), 3.18-3.21 (m, 1H), 3.46-3.56 (m, 2H), 3.81 (s, 3H), 3.83 (s, 3H), 6.67-6.76 (m, 3H), 7.28-7.45 (m. 6H), 7.66-7.78 (m, 4H). ¹³C-NMR (75 MHz, CDCl₃) δ: 19.39, 21.49, 22.54, 26.53, 26.82, 27.03, 27.54, 31.13, 36.29, 55.73, 55.88, 70.18, 79.73, 111.08, 112.33, 120.77, 127.26, 127.39, 127.55, 127.68, 129.33, 129.44, 129.60, 132.13, 134.50, 134.69, 134.76, 135.19, 135.56, 135.91, 136.12, 147.26, 148.61.

### Example 11

### Synthesis of (1S-cis)-2-[2-(3,4-dimethoxyphenyl)ethoxy]cyclohexanol (98)

To a round bottom flask under argon atmosphere was charged (1S-cis)-tert-butyl-{2-[2-(3,4-dimethoxy-phenyl)ethoxy]cyclohexyloxy}diphenyl-silane 97 and tetrabutylammonium fluoride in THF. The reaction mixture was heated at about 50° C. to about 90° C. for about 7 h and was then quenched by the addition of ice-cold water. The aqueous layer was extracted with EtOAc (3×15 mL). The organic layers were combined, washed successively with diluted H₂SO₄ (10 mL, 2%) and brine (10 mL), dried (anhydrous MgSO₄), and concentrated in vacuo to give a light yellow oil. Purification of this crude oil by elution through a silica gel plug provided the product 98.

Compound 98: R_{ƒ} 0.46. ¹H-NMR (300 MHz, CDCl₃) δ: 1.16-1.31 (m, 2H), 1.40-1.61 (m, 4H), 1.62-1.80 (m, 3H), 2.79 (t, J=6.8 Hz, 2H), 3.32-3.37 (m, 1H), 3.52-3.60 (m, 1H), 3.67-3.77 (m, 2H), 3.81 (s, 3H), 3.83 (3H), 6.71-6.78 (m, 3H). ¹³C-NMR (75 MHz, CDCl₃) δ: 21.31, 21.79, 26.65, 30.31, 36.19, 55.73, 55.81, 68.66, 69.26, 78.51, 111.15, 112.19, 120.66, 131.69, 147.43, 148.72.

### Example 12

### Synthesis of (1S-cis)-4-nitro-benzenesulfonic acid 2-[2-(3,4-dimethoxy-phenyl)ethoxy]cyclohexyl ester (64A)

To a round bottom flask under nitrogen atmosphere was charged (1S-cis)-2-[2-(3,4-dimethoxyphenyl)ethoxy]cyclohexanol (98), anhydrous dichloromethane, and anhydrous pyridine. After the reaction mixture was cooled to about 0° C., a solution of 4-nitro-benzenesulfonyl chloride (NsCl) in anhydrous dichloromethane was added drop-wise. The reaction mixture was stirred at about 0° C. to about room temperature with monitoring by TLC. The reaction mixture was diluted with dichloromethane and aqueous H₂SO₄, and the aqueous layer was extracted with CH₂Cl₂ (2×). The organic layers were combined, washed successively with diluted aqueous H₂SO₄, and brine, dried (anhydrous MgSO₄), and concentrated in vacuo to give a yellow oil. Purification of this crude material by elution through a silica gel plug afforded the product 64A.

Compound 64A: R_{ƒ} 0.71. ¹H-NMR (400 MHz, CDCl₃) δ: 1.21-1.69 (m, 8H), 2.01-2.11 (m, 1H), 2.63 (t, 2H, J=6.9 Hz), 3.36-3.38 (m, 1H), 3.43-3.57 (m, 2H), 3.83 (s, 6H), 4.84-4.86 (m, 1H), 6.63-6.69 (m, 2H), 6.75 (d, 1H, J=8.1 Hz), 8.01-8.05 (m, 2H), 8.24-8.28 (m, 2H). ¹³C-NMR (100 MHz, CDCl₃) δ: 21.19, 21.57, 27.20, 29.00, 35.90, 55.76, 55.87, 69.93, 82.65, 111.07, 112.19, 120.63, 124.06, 128.92, 131.39, 143.45, 147.45, 148.68, 150.33.

### Example 13

### Synthesis of (R,R)-1-{2-[2-(3,4-dimethoxy-phenyl)-ethoxy]-cyclohexyl}-pyrrolidin-3-(R)-ol (66)

A round flask of appropriate size was charged with (1S-cis)-4-nitro-benzenesulfonic acid 2-[2-(3,4-dimethoxy-phenyl)-ethoxy]-cyclohexyl ester (64B) and 3-(R)-pyrrolidinol (65) in a suitable molar ratio, which generally may require an excess of 65. The reaction mixture was stirred at an elevated temperature (e.g., from about 40° C. to about 90° C. or higher) such that the reaction may proceed at a rate to allow completion within a reasonable time (e.g., about 2h to about 48h or longer). The reaction mixture was stirred under an inert atmosphere and monitored by TLC. This was followed by standard work-up and purification protocols to provide the title compound (66). This product exhibited a diastereomeric excess of greater than 98% (chiral CE).

Compound 66: R_{ƒ} 0.50. ¹H-NMR (CDCl₃, 300 MHz). δ: 1.21-1.37 (m, 3H), 1.61-1.75 (m, 3H), 1.86-2.08 (m, 2H), 2.43-2.66 (m, 4H), 2.75-2.88 (M, 4H), 2.98-3.05 (m, 2H), 3.31-3.38 (m, 1H), 3.52-3.59 (m, 1H), 3.71-3.79 (m, 1H), 3.93 9 (s, 3H), 3.85 (s, 3H), 4.19-4.23 (m, 1H), 6.71-6.79 (m, 3H). ¹³C-NMR (75 MHz, CDCl₃) δ: 23.01, 23.53, 27.44, 29.09, 34.09, 36.37, 48.92, 55.85, 55.93, 59.77, 63.79, 69.47, 70.96, 79.17, 111.19, 112.39, 120.75, 131.85, 147.45, 148.74.

### Example 14

### General Methods for the Preparation of Compound of Formula (57)

The present invention provides synthetic processes whereby compounds of formula (57) with trans-(1R,2R) configuration for the ether and amino functional groups may be prepared in stereoisomerically substantially pure form. Compounds of formulae (66), (67), (69) and (71) are some of the examples represented by formula (57). The present invention also provides synthetic processes whereby compounds of formulae (52), (53), and (55) may be synthesized in stereoisomerically substantially pure forms. Compounds (61) and (61 A) are examples of formula (52). Compounds (62) and (62A) are examples of formula (53). Compounds (64) and (64A) are examples of formula (55).

As outlined in FIG. 5, the preparation of a stereoisomerically substantially pure trans aminocyclohexyl ether compound of formula (57) may be carried out by following a process starting from a monohalobenzene (49), wherein X may be F, Cl, Br or I.

In a first step, compound (49) is transformed by well-established microbial oxidation to the cis-cyclohexandienediol (50) in stereoisomerically substantially pure form (T. Hudlicky et al., Aldrichimica Acta, 1999, 32, 35; and references cited therein). In a separate step, compound (50) may be selectively reduced under suitable conditions to compound (51) (e.g., H2-Rh/A1203; Boyd et al. JCS Chem. Commun. 1996, 45-46; Ham and Coker, J. Org. Chem. 1964, 29, 194-198; and references cited therein). In another separate step, the less hindered hydroxy group of formula (51) is selectively converted under suitable conditions into an “activated form” as represented by formula (52). An “activated form” as used herein means that the hydroxy group is converted into a good leaving group (—O-J) which on reaction with an appropriate nucleophile (e.g., HNR1R2) will result in a substitution product with substantial inversion of the stereochemical configuration of the activated hydroxy group. The leaving group (—O-J) may be but is not limited to an alkyl sulfonate such as a trifluoromethanesulfonate group (CF₃SO₃—) or a mesylate group (MsO—), an aryl sulfonate such as a benzenesulfonate group (PhSO₃—), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. In a typical reaction for the formation of an alkyl sulfonate (e.g., a mesylate) or an aryl sulfonate (e.g., a tosylate or a nosylate), compound (51) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (51) may be used to maximally convert the hydroxy group into the activated form. In a separate step, transformation of compound (52) to compound (53) may be effected by hydrogenation and hydrogenolysis in the presence of a catalyst under appropriate conditions. Palladium on activated carbon is one example of the catalysts. Hydrogenolysis of alkyl or alkenyl halide such as (52) may be conducted under basic conditions. The presence of a base such as sodium ethoxide, sodium bicarbonate, sodium acetate or calcium carbonate are some possible examples. The base may be added in one portion or incrementally during the course of the reaction.

In a separate step, alkylation of the free hydroxy group in compound (53) to form compound (55) is carried out under appropriate conditions with an alkylating reagent such as compound (54), where —O-Q represents a good leaving group which on reaction with a hydroxy function will result in the formation of an ether compound with retention of the stereochemical configuration of the hydroxy function. Haloacetimidate (e.g., trifluoroacetimidate or trichloroacetimidate) is one example for the —O-Q function. For some compound (54), it may be necessary to introduce appropriate protection groups prior to this step being performed. Suitable protecting groups are set forth in, for example, Greene, “Protective Groups in Organic Chemistry”, John Wiley & Sons, New York N.Y. (1991).

In a separate step, the resulted compound (55) is treated under suitable conditions with an amino compound of formula (56) to form compound (57) as the product. The reaction may be carried out with or without a solvent and at an appropriate temperature range that allows the formation of the product (57) at a suitable rate. An excess of the amino compound (56) may be used to maximally convert compound (55) to the product (57). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 5) generates the compound of formula (57) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

### Example 15

### Synthesis of Compound of Formula (15A), (16A), (17A), (18A), (19A), (20A), (21A), (22A), (23A), (24A), (25A), (26A), (27A), (28A), (29A), (30A), (31A), (32A), (33A), (34A), (35A), (36A), (37A), (38A), (39A), (40A), (41A), (42A), (43A), (44A), (45A), (46A), (47A), (48A)

The above compounds of formula (15A), (16A), (17A), (18A), (19A), (20A), (21A), (22A), (23A), (24A), (25A), (26A), (27A), (28A), (29A), (30A), (31A), (32A), (33A), (34A), (35A), (36A), (37A), (38A), (39A), (40A), (41A), (42A), (43A), (44A), (45A), (46A), (47A), (48A), as show in FIG. 4 may be prepared by similar methods described in Example 5 and Example 13 by reaction of the appropriate formula (55) with the appropriate formula (56). The respective formula (56) are shown in FIG. 4 corresponding to each aminocyclohexyl ether compound to be synthesized. Compound corresponding to formula (55) may be prepared from appropriate formula (53) and formula (54). Compound corresponding to formula (53) may be prepared according to methods similar to those described in Examples 1 to 3. Compound corresponding to formula (54) may be prepared from the appropriate corresponding alcohol shown in FIG. 4.

### Example 16

### General Methods for the Preparation of Compound of Formula (75)

As outlined in FIG. 45, the preparation of a stereoisomerically substantially pure trans aminocyclohexyl ether compound of formula (75) may be carried out by following a process starting from a monohalobenzene (49), wherein X may be F, Cl, Br or I.

In a first step, compound (49) is transformed by well-established microbial oxidation to the cis-cyclohexandienediol (50) in stereoisomerically substantially pure form (T. Hudlicky et al., Aldrichimica Acta, 1999, 32, 35; and references cited therein). In a separate step, compound (50) may be selectively reduced under suitable conditions to compound (51) (e.g., H2-Rh/A1203; Boyd et al. JCS Chem. Commun. 1996, 45-46; Ham and Coker, J. Org. Chem. 1964, 29, 194-198; and references cited therein). In another separate step, compound (51) is converted to compound (72) by reaction under appropriate conditions with an alkylating reagent such as compound (54), where —O-Q represents a good leaving group which on reaction with a hydroxy function will result in the formation of an ether compound with retention of the stereochemical configuration of the hydroxy function. Haloacetimidate (e.g., trifluoroacetimidate or trichloroacetimidate) is one example for the —O-Q function. For some compound (72), it may be necessary to introduce appropriate protection groups prior to this step being performed. Suitable protecting groups are set forth in, for example, Greene, “Protective Groups in Organic Chemistry”, John Wiley & Sons, New York N.Y. (1991).

In a separate step, transformation of compound (72) to compound (73) may be effected by hydrogenation and hydrogenolysis in the presence of a catalyst under appropriate conditions. Palladium on activated carbon is one example of the catalysts. Hydrogenolysis of alkyl or alkenyl halide such as (72) may be conducted under basic conditions. The presence of a base such as sodium ethoxide, sodium bicarbonate, sodium acetate or calcium carbonate is some possible examples. The base may be added in one portion or incrementally during the course of the reaction.

In another separate step, the hydroxy group of compound (73) is selectively converted under suitable conditions into an activated form as represented by compound (74). An “activated form” as used herein means that the hydroxy group is converted into a good leaving group (—O-J) which on reaction with an appropriate nucleophile (e.g., HNR1R2) will result in a substitution product with substantial inversion of the stereochemical configuration of the activated hydroxy group. The leaving group (—O-J) may be but is not limited to an alkyl sulfonate such as a trifluoromethanesulfonate group (CF3SO3-) or a mesylate group (MsO—), an aryl sulfonate such as a benzenesulfonate group (PhSO3-), a mono- or poly-substituted benzenesulfonate group, a mono- or poly-halobenzenesulfonate group, a 2-bromobenzenesulfonate group, a 2,6-dichlorobenzenesulfonate group, a pentafluorobenzenesulfonate group, a 2,6-dimethylbenzenesulfonate group, a tosylate group (TsO—) or a nosylate (NsO—), or other equivalent good leaving groups. The hydroxy group may also be converted into other suitable leaving groups according to procedures well known in the art. In a typical reaction for the formation of an alkyl sulfonate (e.g., a mesylate) or an aryl sulfonate (e.g., a tosylate or a nosylate), compound (73) is treated with a hydroxy activating reagent such as an alkyl sulfonyl halide (e.g., mesyl chloride (MsCl)) or an aryl sulfonyl halide (e.g., tosyl chloride (TsCl) or nosyl chloride (NsCl)) in the presence of a base, such as pyridine or triethylamine. The reaction is generally satisfactorily conducted at about 0° C., but may be adjusted as required to maximize the yields of the desired product. An excess of the hydroxy activating reagent (e.g., mesyl chloride, tosyl chloride or nosyl chloride), relative to compound (73) may be used to maximally convert the hydroxy group into the activated form.

In a separate step, the resulted compound (74) is treated under suitable conditions with an amino compound of formula (56) to form compound (75) as the product. The reaction may be carried out with or without a solvent and at an appropriate temperature range that allows the formation of the product (75) at a suitable rate. An excess of the amino compound (56) may be used to maximally convert compound (74) to the product (75). The reaction may be performed in the presence of a base that can facilitate the formation of the product. Generally the base is non-nucleophilic in chemical reactivity. When the reaction has proceeded to substantial completion, the product is recovered from the reaction mixture by conventional organic chemistry techniques, and is purified accordingly. Protective groups may be removed at the appropriate stage of the reaction sequence. Suitable methods are set forth in, for example, Greene, “Protective Groups in Organic Chemistry”, John Wiley & Sons, New York N.Y. (1991).

The reaction sequence described above (FIG. 45) generates the compound of formula (75) as the free base. The free base may be converted, if desired, to the monohydrochloride salt by known methodologies, or alternatively, if desired, to other acid addition salts by reaction with an inorganic or organic acid under appropriate conditions. Acid addition salts can also be prepared metathetically by reaction of one acid addition salt with an acid that is stronger than that giving rise to the initial salt.

### Example 17

### Synthesis of Compound of Formula (15B), (16B), (17B), (18B), (19B), (20B), (21B), (22B), (23B), (24B), (25B), (26B), (27B), (28B), (29B), (30B), (31B), (32B), (33B), (34B), (35B), (36B), (37B), (38B), (39B), (40B), (41B), (42B), (43B), (44B), (45B), (46B), (47B), (48B)

The above compounds of formula (15B), (16B), (17B), (18B), (19B), (20B), (21B), (22B), (23B), (24B), (25B), (26B), (27B), (28B), (29B), (30B), (31B), (32B), (33B), (34B), (35B), (36B), (37B), (38B), (39B), (40B), (41B), (42B), (43B), (44B), (45B) (46B), (47B), (48B) as show in FIG. 4 may be prepared by similar methods described in Example 16 by reaction of the appropriate formula (74) with the appropriate formula (56). The respective formula (56) is shown in FIG. 4 corresponding to each aminocyclohexyl ether compound to be synthesized. Compound corresponding to formula (74) may be prepared from appropriate formula (73) with the appropriate activating reagent as described in Example 16 above. Compound corresponding to formula (73) may be prepared from appropriate formula (72) by hydrogenation and hydrogenolysis reduction as described in Example 16 above. Compound corresponding to formula (72) may be prepared according to methods similar to those described in Examples 16 by reaction of formula (60) with the appropriate compound corresponding to formula (54). Compound corresponding to formula (54) may be prepared from the appropriate corresponding alcohol shown in FIG. 4.

1. A method of stereoselectively making an aminocyclohexyl ether comprising
reacting 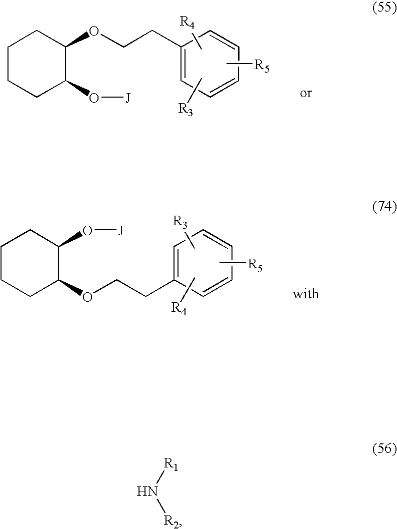
to form the aminocyclohexyl ether having the formula 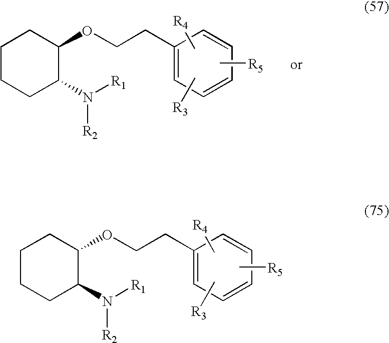
respectively,
wherein independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, and C₇-C₁₂aralkyl; or R₁ and R₂, when taken together with the nitrogen atom to which they are directly attached in formula (57) or (75), form a ring denoted by formula (I): 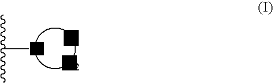
wherein the ring of formula (I) is formed from the nitrogen as shown as well as three to nine additional ring atoms independently carbon, nitrogen, oxygen, or sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆, R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen; and
wherein O-J is a leaving group.

2. The method defined in claim 1, wherein before said reacting step, the method further comprises
alkylating 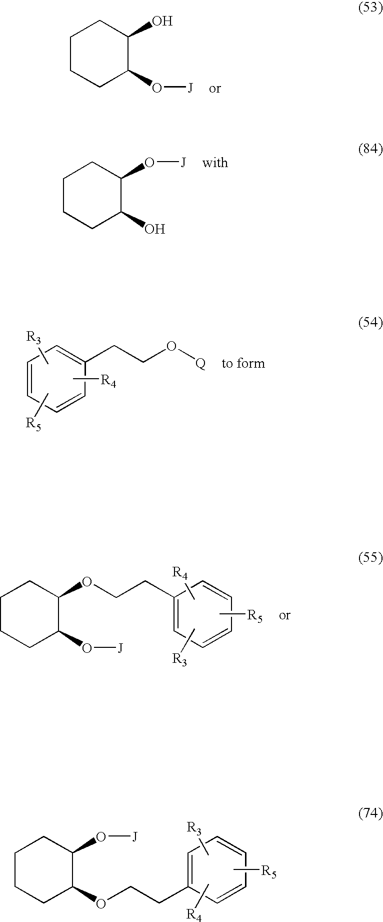
respectively;
wherein O-J is an alkyl sulfonate or an aryl sulfonate; and
wherein O-Q is a leaving group that reacts with —OH in formula (53) or (84) to form said ether of formula (55) or (74), such that the stereochemical configuration of the hydroxyl group is retained in the ether; and
optionally protecting 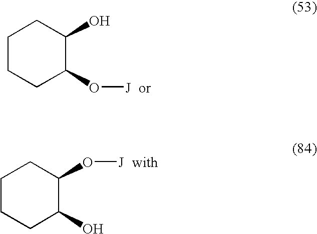
before said alkylating step.

3. The method defined in claim 2
wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy, and wherein R₃, R₄ and R₅ are independently selected from the group consisting of hydrogen, hydroxy and C₁-C₆alkoxy, with the proviso that R₃, R₄ and R₅ cannot all be hydrogen; and
wherein O-J is selected from an alkyl sulfonate or an aryl sulfonate.

4. The method defined in claim 3,
wherein 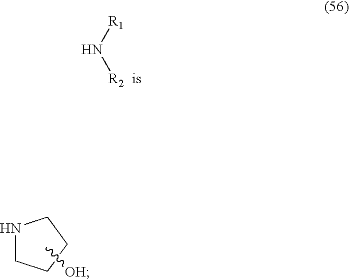
and
wherein at least one of R₃, R₄ and R₅ is C₁-C₆alkoxy; and
wherein O-J is a mesylate, a benzenesulfonate, a mono- or poly-alkylbenzenesulfonate, a mono- or poly-halobenzenesulfonate, tosylate or nosylate.

5. The method defined in claim 4,
wherein 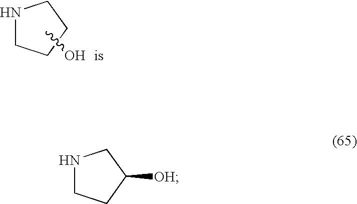
and
wherein O-J is a mesylate, a benzenesulfonate, a tosylate, 2-bromobenzenesulfonate, a 2,6-dichlorobenzenesulfonate or a nosylate; and
wherein 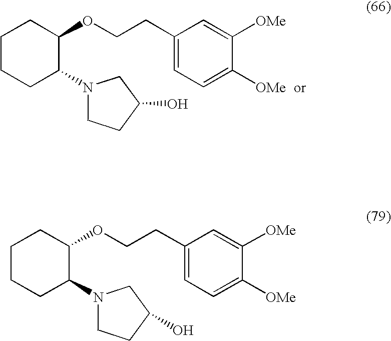
is formed.

6. The method defined in claim 5 wherein 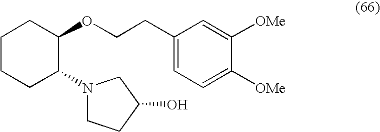
is formed.

7. The method defined in claim 2,
wherein O-J is a mesylate, a benzenesulfonate, a tosylate, a 2-bromobenzenesulfonate, a 2,6-dichlorobenzenesulfonate or a nosylate; and
wherein at least one of R₃, R₄ and R₅ is C₁-C₆alkoxy; and wherein O-Q is trichloroacetimidate.

8. The method defined in claim 7

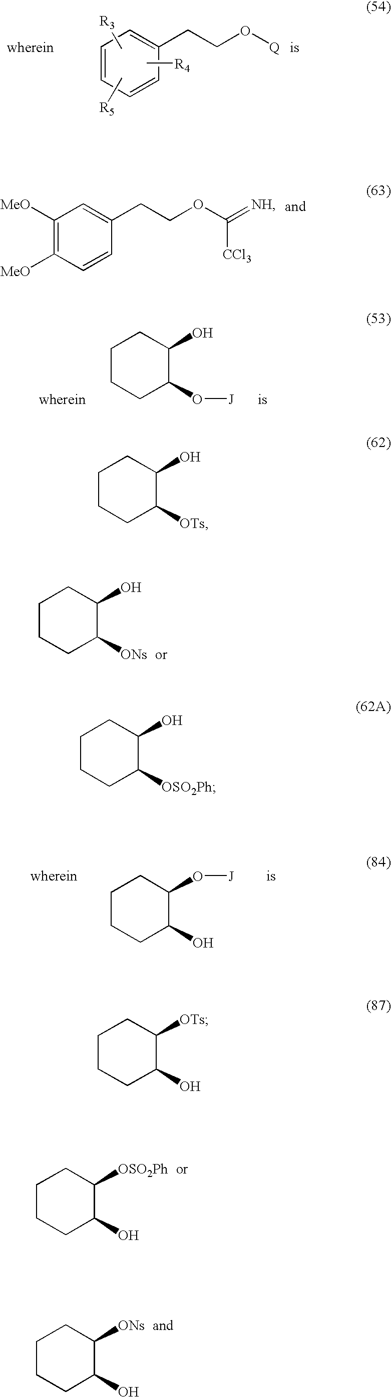

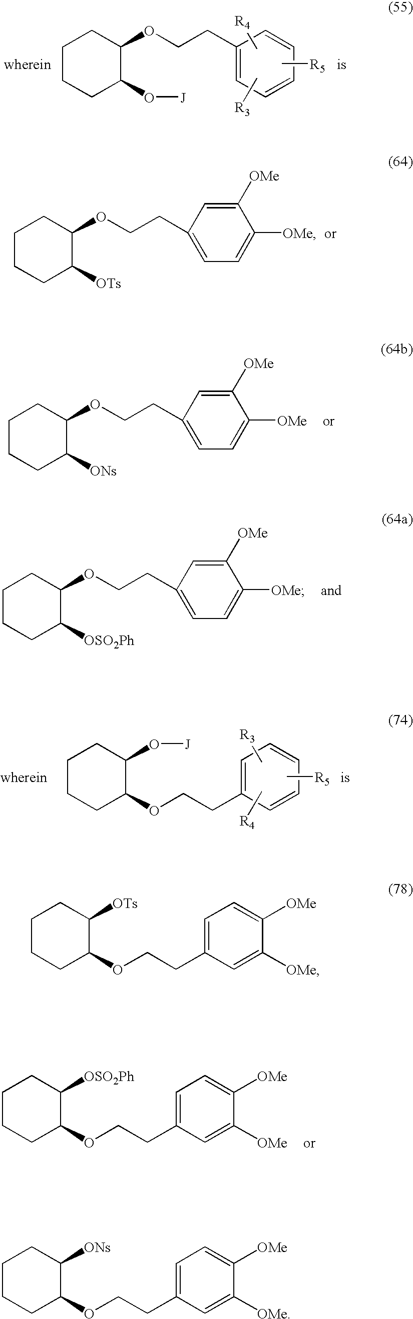

9. The method defined in claim 1,
wherein before said reacting step, the method further comprises
activating 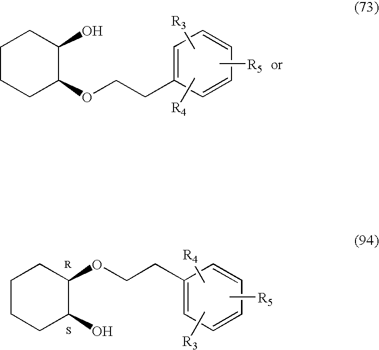
with a hydroxy activating reagent to form 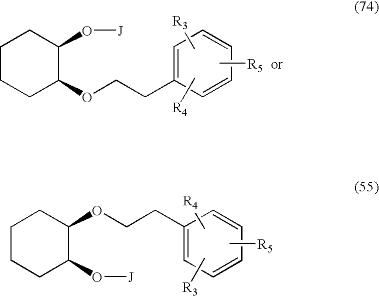
respectively.

10. The method defined in claim 9,
wherein at least one of R₃, R₄ and R₅ is C₁-C₆alkoxy; and
wherein the hydroxy activating reagent is an alkyl sulfonyl halide or an aryl sulfonyl halide.

11. The method defined in claim 10,
wherein the hydroxy activating reagent is tosyl halide, benzenesulfonyl halide or nosyl halide; and

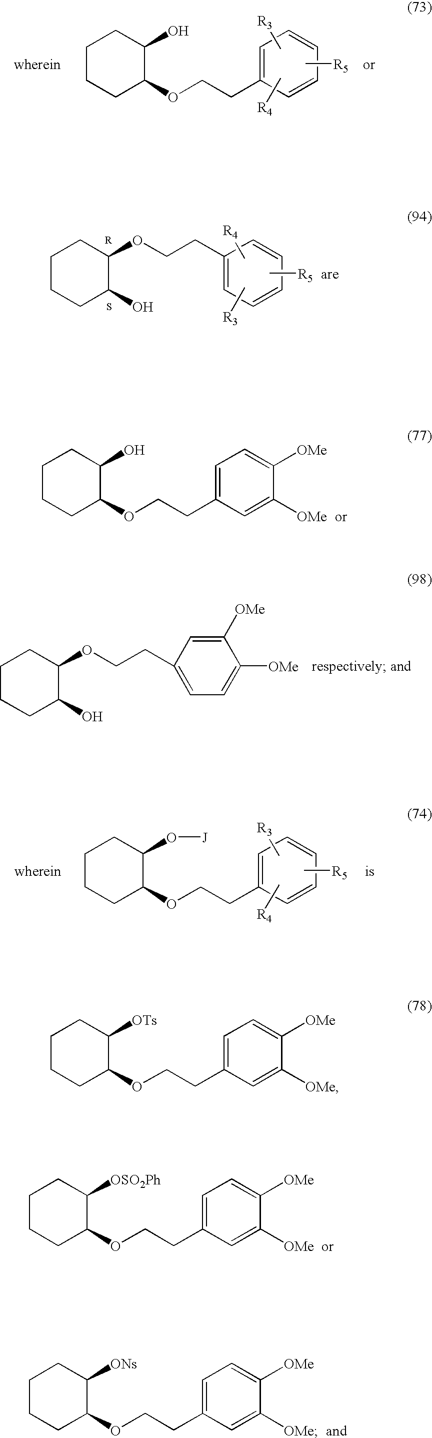

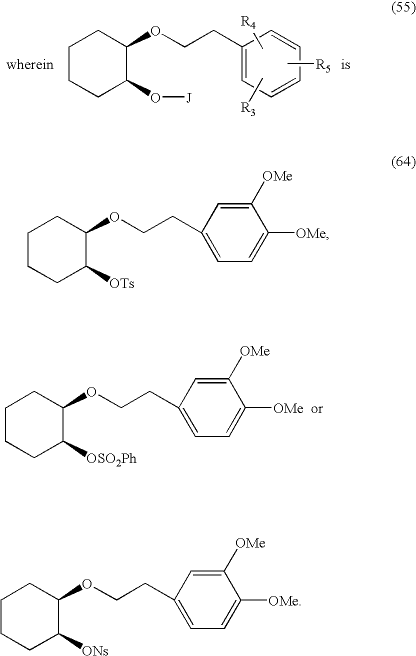

12. The method defined in claim 9,
wherein before said activating step, the method further comprises
hydrogenating and hydrogenolyzing 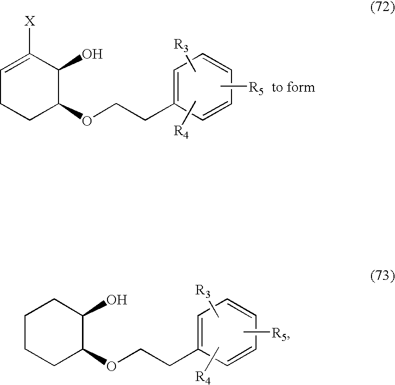
wherein X is a halide.

13. The method defined in claim 12, wherein

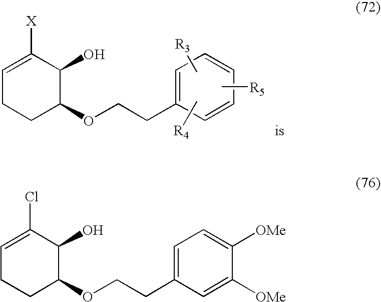

14. The method defined in claim 12, further comprising before said hydrogenating and hydrogenolyzing step,
alkylating

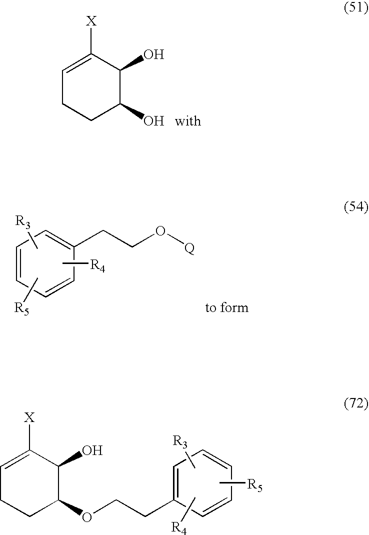

15. The method defined in claim 9,
wherein before said activating step, the method further comprises
deprotecting 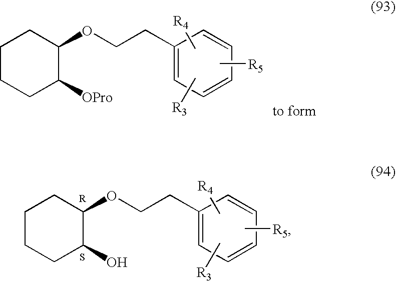
wherein Pro is a protecting group.

16. The method defined in claim 15
wherein

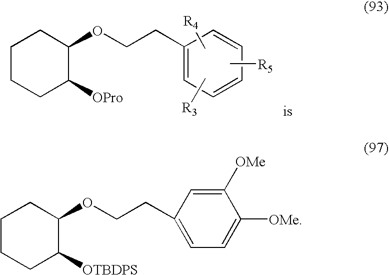

17. The method defined in claim 15
wherein before said deprotecting step, the method further comprises
alkylating

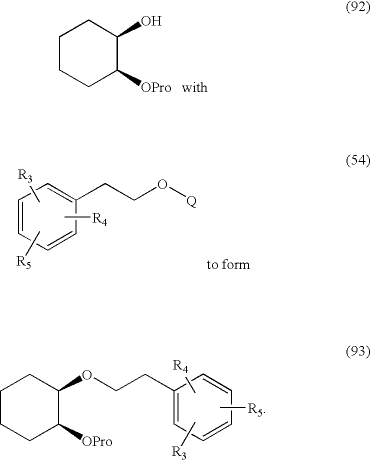

18. The method defined in claim 17

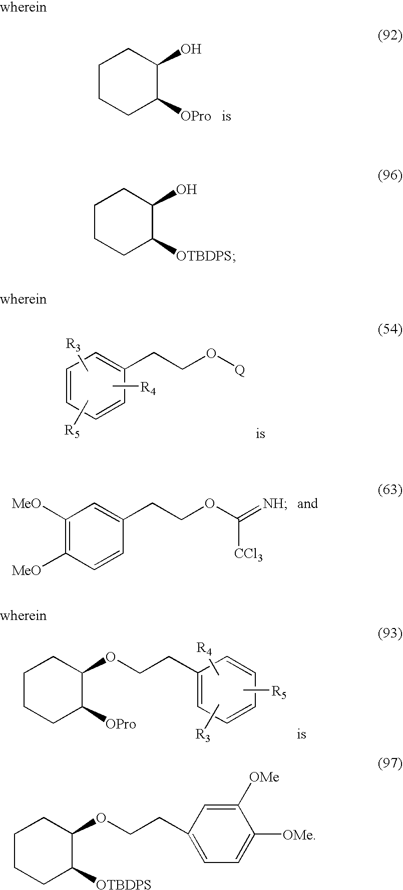

19. The method as defined in claim 17, further comprising before said alkylating step, hydrogenating and hydrogenolyzing

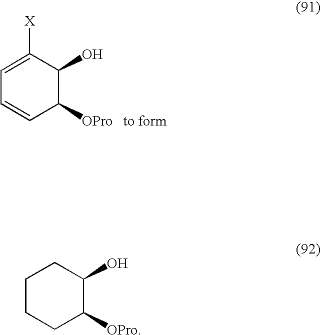

20. The method defined in claim 2, further comprising before the alkylating step hydrogenating and hydrogenolyzing 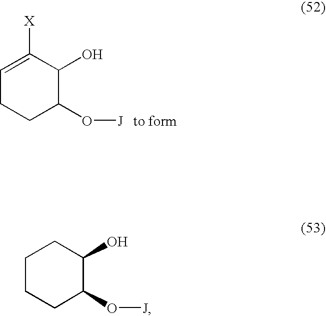
wherein X is a halide.

21. The method defined in claim 20, wherein

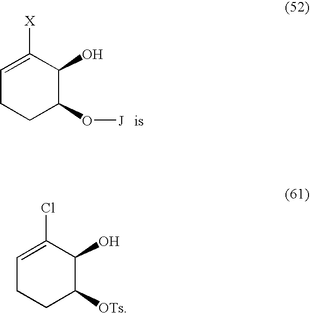

22. The method defined in claim 20, further comprising before said hydrogenating and hydrogenolyzing step, activating 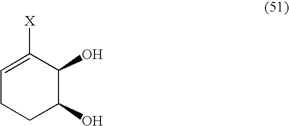
with a hydroxy activating reagent to form

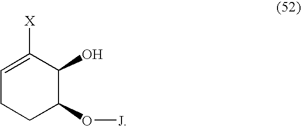

23. The method defined in claim 2, further comprising before said alkylating step deprotecting 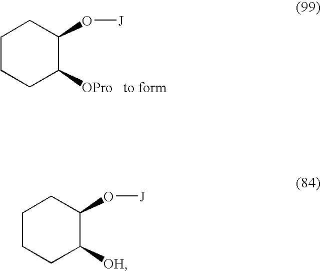
wherein Pro is a protecting group.

24. The method defined in claim 23, further comprising before said deprotecting step, activating 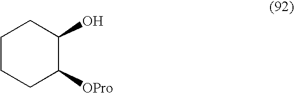
with a hydroxy activating reagent to form

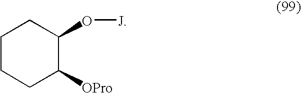

25. The method defined in claim 24, further comprising before said activating step, hydrogenating and hydrogenolyzing

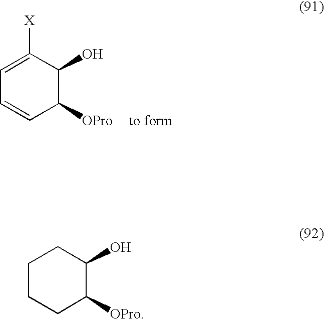

26. The method defined in claim 24,
wherein the hydroxy activating reagent is tosyl halide, benzenesulfonyl halide or nosyl halide;
wherein 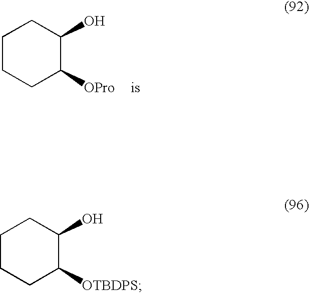
and
wherein

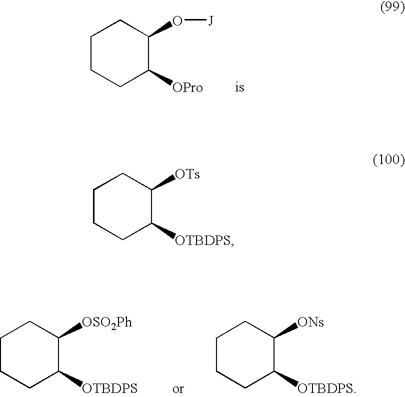

27. The method defined in claim 1, wherein 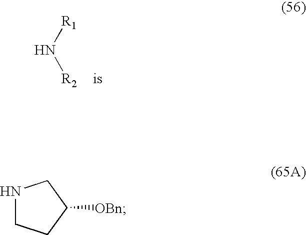
and
wherein 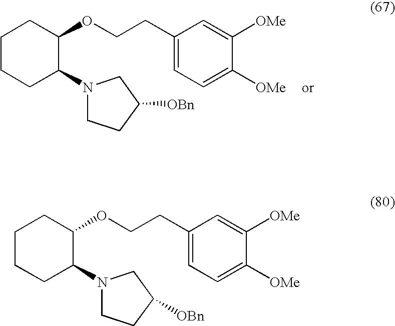
is formed.

28. The method defined in claim 2, further comprising before said alkylating step, removing a functional group G or G₁ from 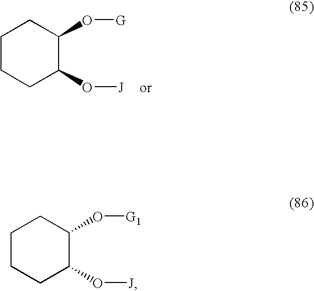
respectively, to form 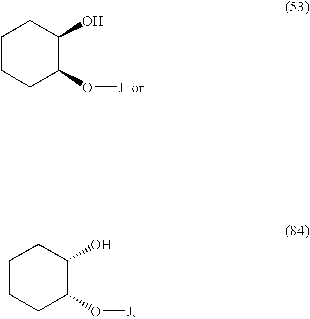
respectively.

29. The method defined in claim 2, further comprising, before said alkylating step separating a racemic mixture of

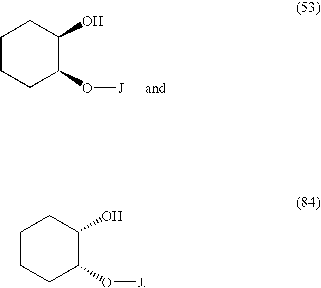

30. The method defined in claim 29,
wherein said separation step further comprises functionalizing one or both of 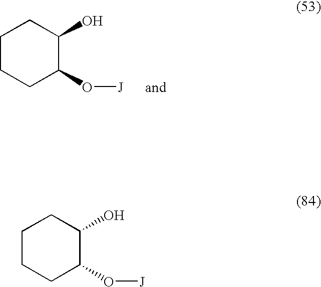
such that the compounds are capable of resolution;
performing resolution to separate the compounds; and
optionally removing the functional group on said one or both functionalized compounds.

31. The method defined in claim 29
wherein before said separating step the method further comprises
activating 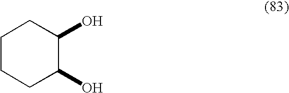
with a hydroxy activating reagent to form
the racemic mixture of

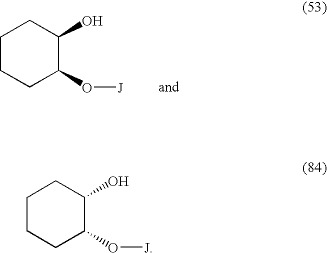

32. The method defined in claim 30
wherein 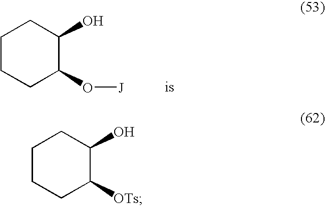
wherein 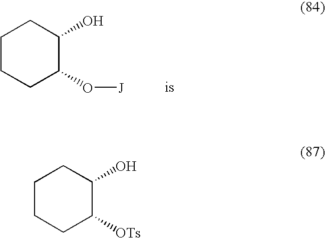
and is enzymatically functionalized with 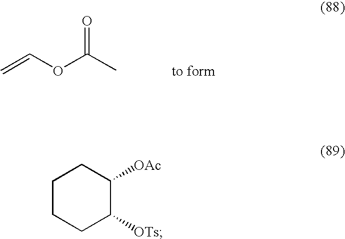
performing resolution to separate

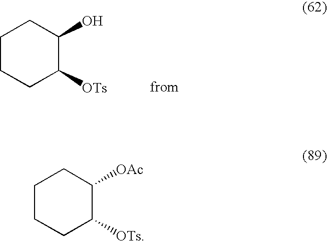

33. The method defined in claim 30
wherein 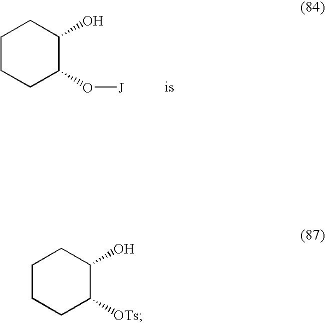
and
wherein 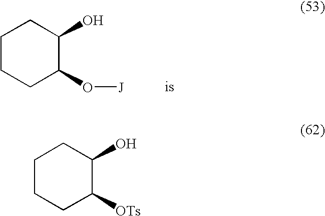
and is functionalized with 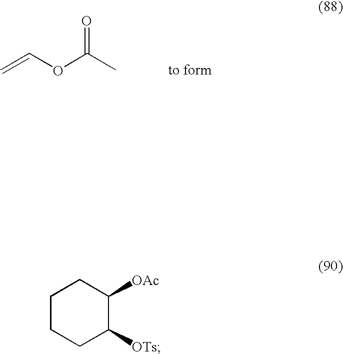
further comprising
performing resolution to separate 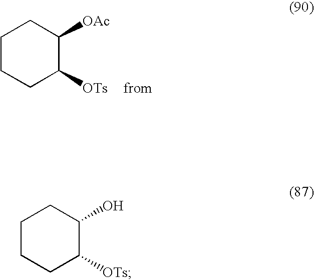
and
removing the functional group from

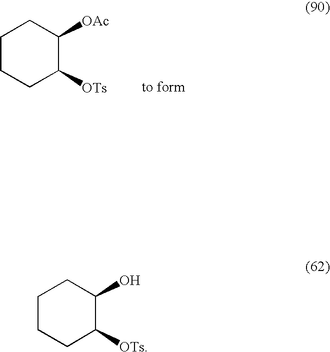

34. The method defined in claim 29 further comprising before said separating step,
activating 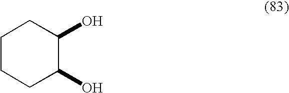
with a hydroxy activating reagent to form the racemic mixture.

35. A method of stereoselectively making an aminocyclohexyl ether comprising
alkylating 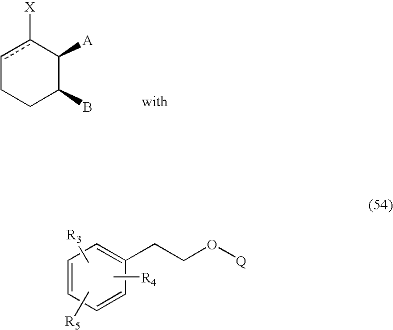
to form a reaction product; and
optionally hydrogenating and hydrogenolyzing 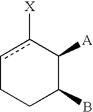
or the reaction product to reduce optional double bond and remove halide if present;
reacting the reaction product of the alkylating step with 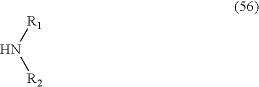
to form 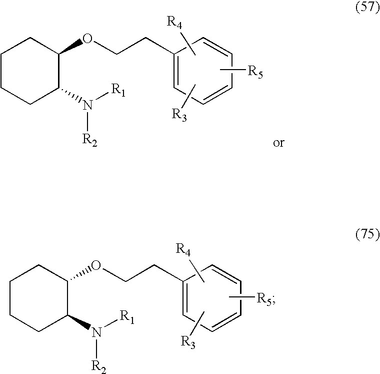
wherein - - - is an optional double bond;
wherein X is H or halide;
wherein A is OH, or a leaving group;
wherein B is OH, a leaving group, or a protecting group;
wherein only one of A and B may be OH;
wherein only one of A and B may be a leaving group;
wherein —O-Q is a leaving group;
wherein independently at each occurrence, R₁ and R₂ are independently hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or
R₁ and R₂, when taken together with the nitrogen atom to which they are directly attached in formula (9), form a ring denoted by formula (I): 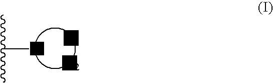
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two oxygen and/or sulfur heteroatoms; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆, R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen.

36. The method as defined in claim 35
wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy, and wherein R₃, R₄ and R₅ are independently selected from the group consisting of hydrogen, hydroxy and C₁-C₆alkoxy, with the proviso that R₃, R₄ and R₅ cannot all be hydrogen; and
wherein O-J is an alkyl sulfonate or an aryl sulfonate.

37. The method as defined in claim 36,
wherein 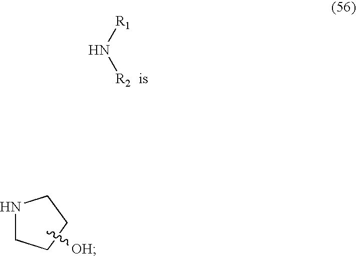
and
wherein at least one of R₃, R₄ and R₅ is C₁-C₆alkoxy; and
wherein O-J is a mesylate, a benzenesulfonate, a mono- or poly-alkylbenzenesulfonate, a mono- or poly-halobenzenesulfonate, tosylate or nosylate.

38. The method as defined in claim 37,
wherein 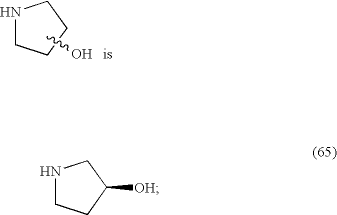
and
wherein O-J is a mesylate, a benzenesulfonate, a tosylate, 2-bromobenzenesulfonate, a 2,6-dichlorobenzenesulfonate or a nosylate; and
wherein 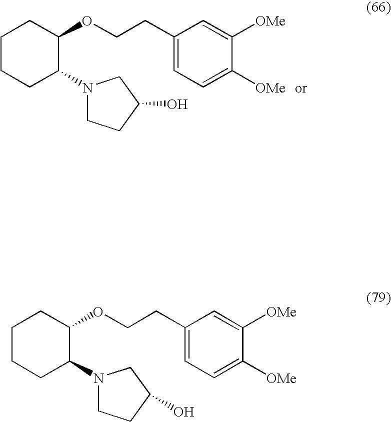
is formed.

39. The method as defined in claim 35 wherein 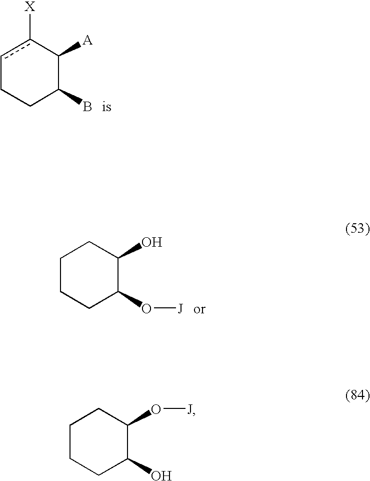
and the alkylating step further comprises
alkylating 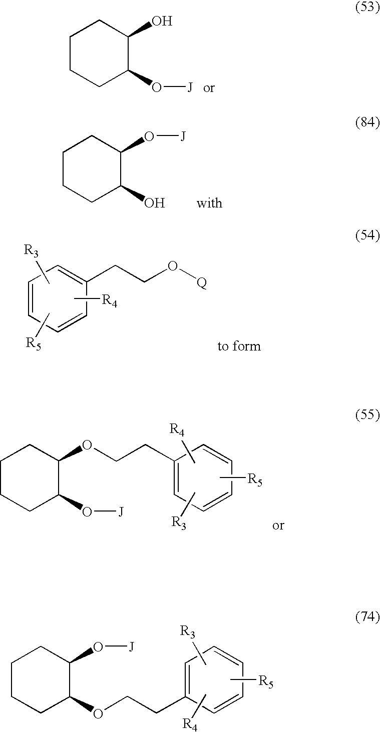
respectively;
wherein O-J is an alkyl sulfonate or an aryl sulfonate; and
wherein O-Q is a leaving group that reacts with —OH in formula (53) or (84) to form said ether of formula (55) or (74), such that the stereochemical configuration of the hydroxyl group is retained in the ether; and
optionally protecting 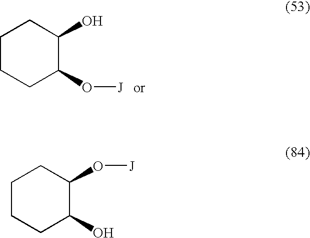
before said alkylating step.

40. The method as defined in claim 35,
wherein 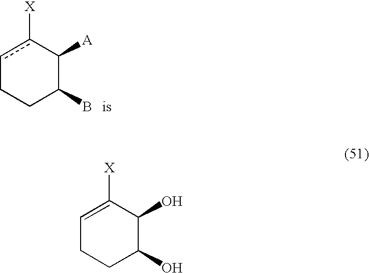
and wherein the alkylating step further comprises
alkylating 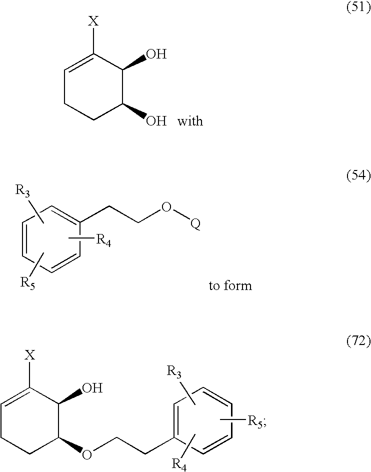
wherein the method further comprises hydrogenating and hydrogenolyzing 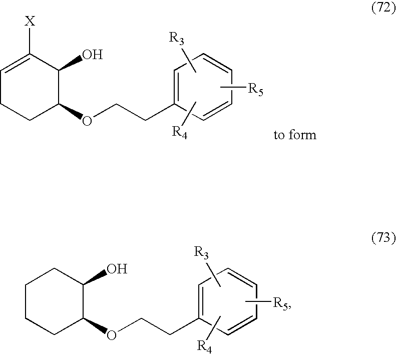
wherein X is a halide; and
activating 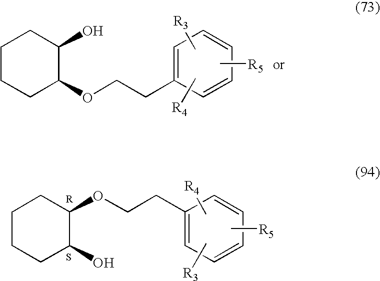
with a hydroxy activating reagent to form 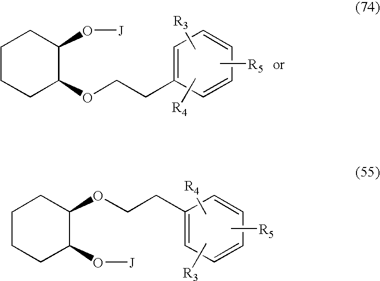
respectively.

41. The method as defined in claim 35, wherein 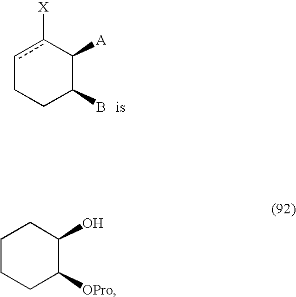
further comprising before said alkylating step, hydrogenating and hydrogenolyzing 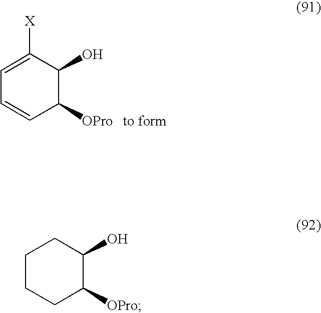
wherein the method further comprises
alkylating 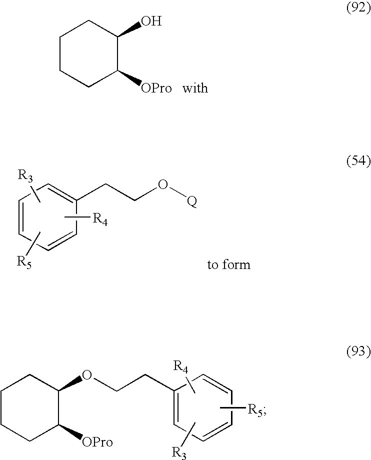
deprotecting 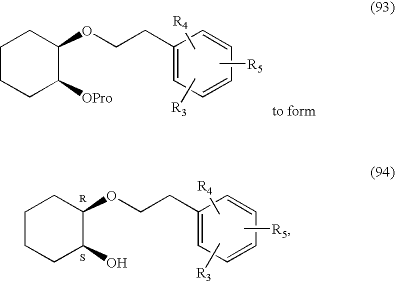
wherein Pro is a protecting group; and
activating 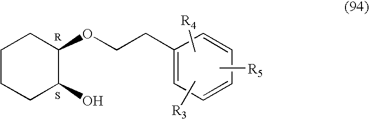
with a hydroxy activating reagent to form

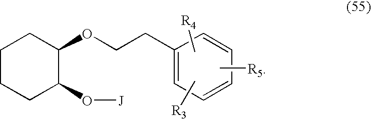

42. The method as defined in claim 39, further comprising before the alkylating step hydrogenating and hydrogenolyzing 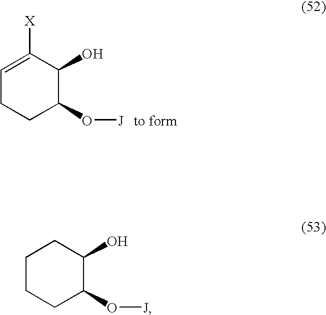
wherein X is a halide.

43. The method as defined in claim 42, further comprising before said hydrogenating and hydrogenolyzing step,
activating 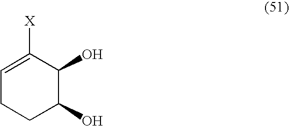
with a hydroxy activating reagent to form

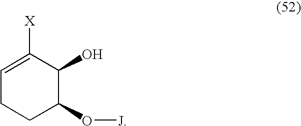

44. The method as defined in claim 39, further comprising before the alkylating step hydrogenating and hydrogenolyzing 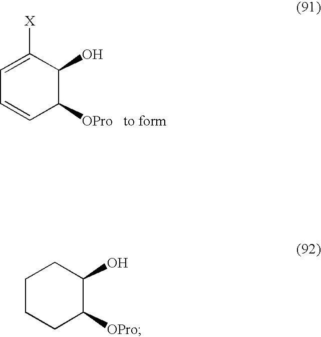
activating 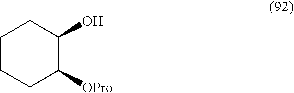
with a hydroxy activating reagent to form 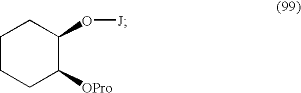
and deprotecting 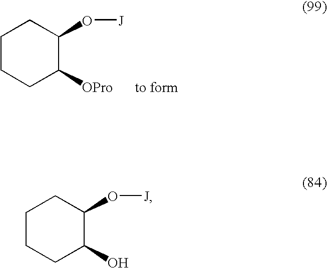
wherein Pro is a protecting group.

45. The method as defined in claim 39, further comprising, before the alkylating step, removing a functional group G or G₁ from 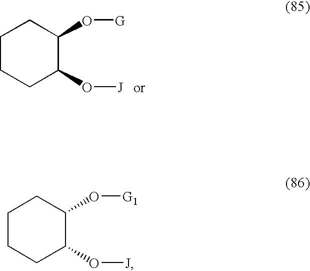
respectively, to form 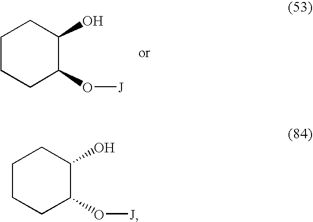
respectively.

46. The method as defined in claim 39 further comprising, before said alkylating step,
separating a racemic mixture of

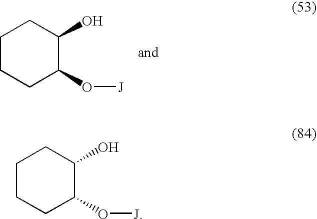

47. The method as defined in claim 46 wherein said separation step further comprises
functionalizing one or both of 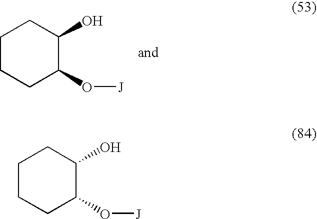
such that the compounds are capable of resolution;
performing resolution to separate the compounds; and
optionally removing the functional group on said one or both functionalized compounds.

48. The method as defined in claim 46 wherein before said separating step the method further comprises
activating 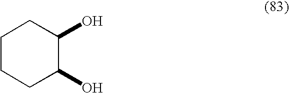
with a hydroxy activating reagent to form the racemic mixture of

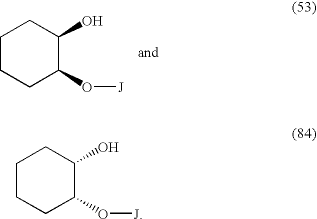

49. A method comprising
alkylating 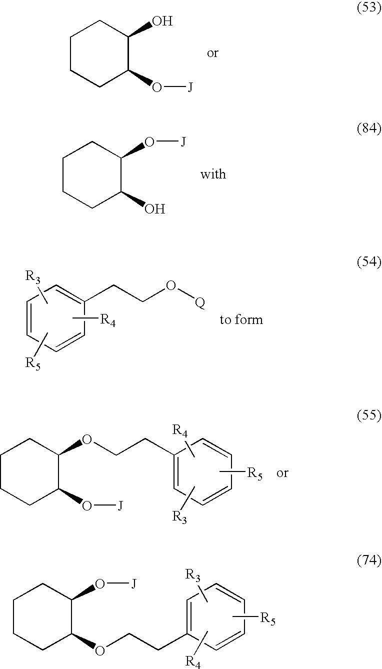
respectively;
optionally protecting 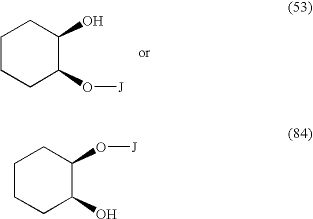
before said reacting step;
wherein O-Q is a leaving group that reacts with —OH in formula (53) or (84) to form said ether of formula (55) or (74), such that the stereochemical configuration of the the hydroxyl group is retained in the ether;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-J is a leaving group.

50. A method comprising
activating 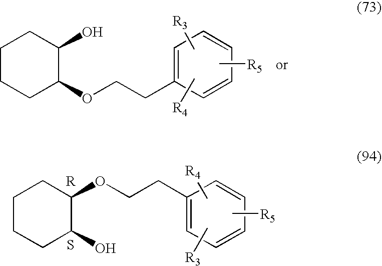
with a hydroxy activating reagent to form 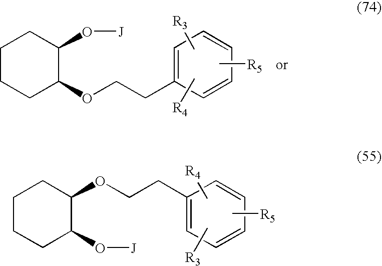
respectively;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-J is a leaving group.

51. A method comprising
hydrogenating and hydrogenolyzing 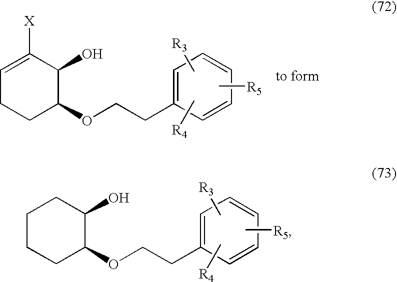
wherein X is a halide;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen.

52. A method comprising
alkylating 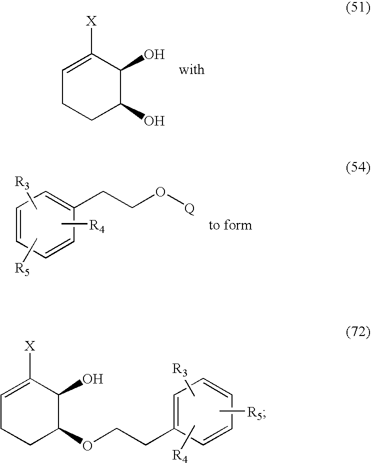
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen;
wherein X is a halide; and
wherein O-Q is a leaving group that reacts with —OH to form said ether, such that the stereochemical configuration of the hydroxyl group is retained in the ether.

53. A method comprising
alkylating 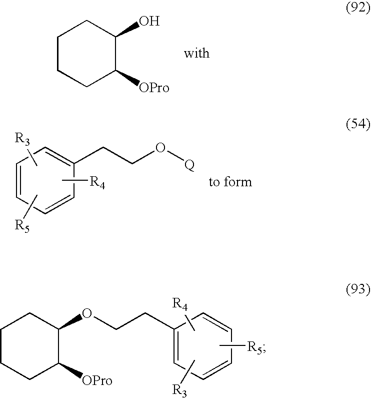
wherein Pro is a protecting group;
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl, or C₁-C₆alkyl with the proviso that R₃, R₄ and R₅ cannot all be hydrogen; and
wherein O-Q is a leaving group that reacts with —OH to form said ether, such that the stereochemical configuration of the hydroxyl group is retained in the ether.

54. A method comprising
hydrogenating and hydrogenolyzing 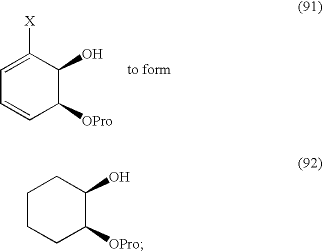
wherein Pro is a protecting group; and
wherein X is a halide.

55. A method comprising
hydrogenating and hydrogenolyzing 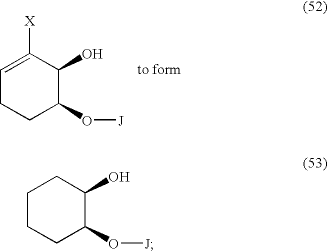
wherein X is a halide; and
wherein O-J is a leaving group.

56. A method comprising
activating 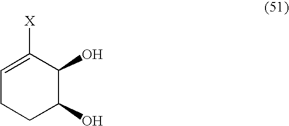
with a hydroxy activating reagent to form 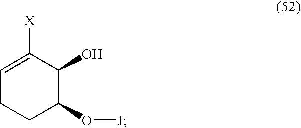
wherein X is a halide; and
wherein O-J is a leaving group.

57. A method comprising
activating 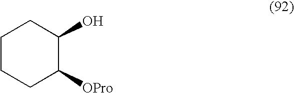
with a hydroxy activating reagent to form 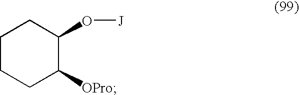
wherein Pro is a protecting group; and
wherein O-J is a leaving group.

58. A method comprising
hydrogenating and hydrogenolyzing 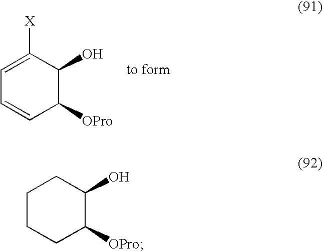
wherein X is a halide; and
wherein Pro is a protecting group.

59. A method comprising
removing a functional group G or G₁ from 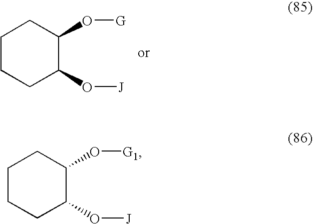
respectively, to form 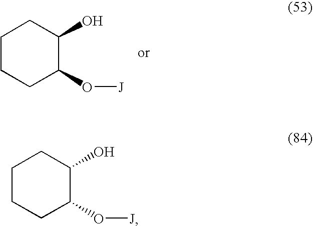
respectively;
wherein O-J is a leaving group.

60. A method comprising separating a racemic mixture of

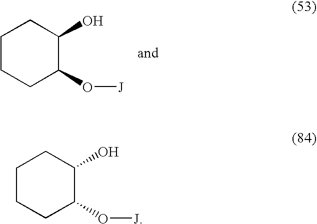

61. The method defined in claim 57 wherein said separation step further comprises
functionalizing one or both of 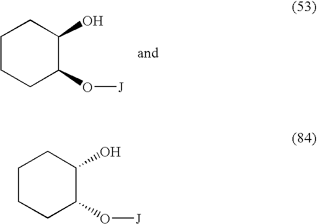
such that the compounds are capable of resolution;
performing resolution to separate the compounds; and
optionally removing the functional group on said one or both functionalized compounds.

62. A method comprising
activating 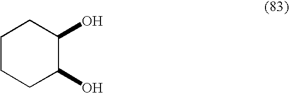
with a hydroxy activating reagent to form the racemic mixture of 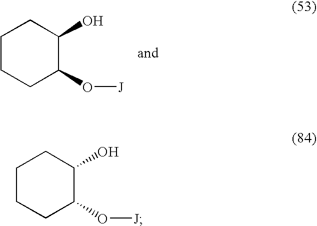
wherein O-J is a leaving group.

63. A method for stereoselectively making an aminocyclohexyl ether of formula (57): 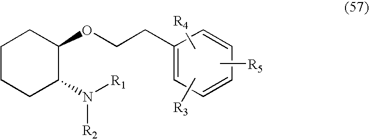
wherein independently at each occurrence, R₁ and R₂ are selected from hydrogen, C₁-C₈alkyl, C₃-C₈alkoxyalkyl, C₁-C₈hydroxyalkyl, and C₇-C₁₂aralkyl; or
R₁ and R₂ are selected from C₃-C₈alkoxyalkyl, C₁-C₈hydroxyalkyl, and C₇-C₁₂aralkyl; or R₁, and R₂, when taken together with the nitrogen atom to which they are directly attached in formula (57), form a ring denoted by formula (I): 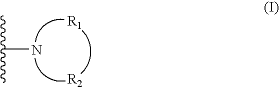
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two heteroatoms selected from the group consisting of carbon, nitrogen, oxygen, and sulfur; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen, comprising:
(a) reacting 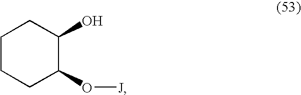
wherein O-J is a leaving group, with 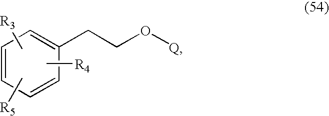
wherein R₃, R₄ and R₅ are as defined above and O-Q is a leaving group that reacts with the hydroxy group (—OH) in formula (53) to form an ether of formula (55), 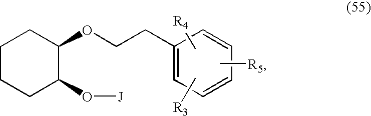
such that the stereochemical configuration of the hydroxy group is retained in the ether;
(b) optionally protecting compound of formula (53) before the first reaction; and
(c) reacting the ether of formula (55) with 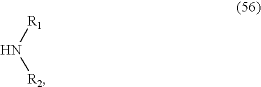
wherein R₁ and R₂ are as defined above, to form the aminocyclohexyl ether of formula (57).

64. A method of claim 63, further comprising before said first reaction (a), hydrogenating and hydrogenolyzing 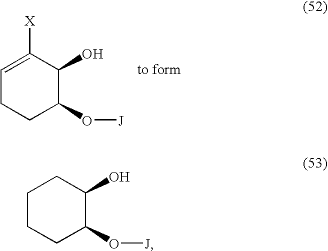
wherein X is a halide.

65. A method of claim 64, further comprising before said hydrogenating and hydrogenolyzing reaction, activating 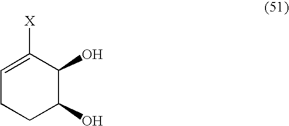
with a hydroxy activating reagent to form

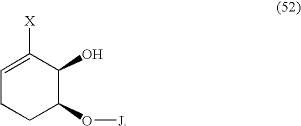

66. A method of claim 63, further comprising before said first reaction (a), separating a racemic mixture of 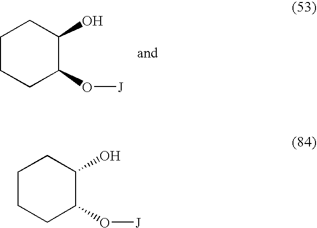
to obtain (53), wherein said separation step further comprises optionally functionalizing one or both of 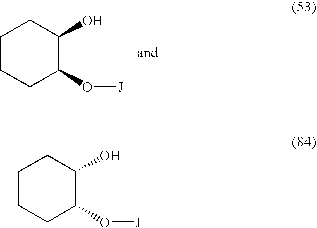
such that the compounds are amenable to resolution;
performing resolution to separate the compounds; and
optionally removing the functional group on said one or both functionalized compounds.

67. A method of claim 66, wherein said separation step comprises enzymatic resolution, crystallization and/or chromatographic resolution.

68. A method of claim 66, wherein said resolution is lipase mediated.

69. A method of claim 63, further comprising before said first reaction, removing a functional group G from

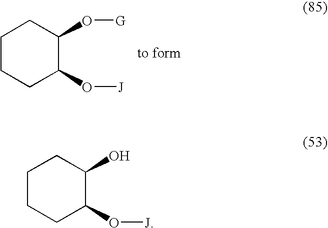

70. The method of any one of claims 63, 64, 65, 66, 67, and 68, wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy;
wherein R₃, R₄ and R₅ are independently hydrogen, hydroxy or C₁-C₆alkoxy, with the proviso that R₃, R₄ and R₅ cannot all be hydrogen;
wherein O-J is an alkyl sulfonate or an aryl sulfonate;
wherein O-Q is an imidate ester, an O-carbonate, a S-carbonate, an O-sulfonyl derivative, or a phosphate derivative; and
wherein, if present, X is Cl.

71. The method of any one of claims 63, 64, 65, 66, 67, and 68,
wherein 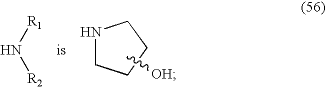
wherein at least one of R₃, R₄ and R₅ is C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate; and
wherein, if present, X is Cl.

72. The method of any one of claims 63, 64, 65, 66, 67, and 68,
wherein 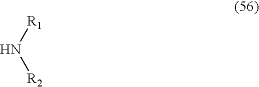
is 3R-pyrrolidinol (65) or 3S-pyrrolidinol (65A);
wherein R₃ is hydrogen, and R₄ and R₅ are C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate; and
wherein, if present, X is Cl.

73. The method of any one of claims 63, 64, 65, 66, 67, and 68, 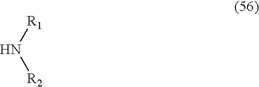
wherein is 3R-pyrrolidinol (65);
wherein R₃ is hydrogen, R₄ is methoxy at C3 of the phenyl group and R₅ is methoxy at C4 of the phenyl group;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate or pentafluorobenzimidate; and
wherein, if present, X is Cl,
such that the aminocyclohexyl ether of formula (57)

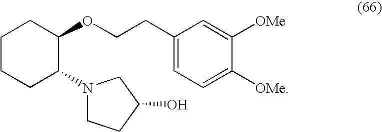

74. A method for stereoselectively making an aminocyclohexyl ether of formula (75): 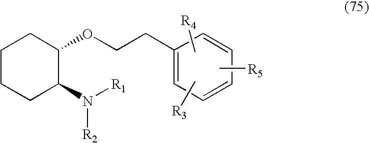
wherein independently at each occurrence, R₁ and R₂ are hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or R₁ and R₂, when taken together with the nitrogen atom to which they are directly attached in formula (75), form a ring denoted by formula (I): 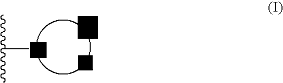
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two heteroatoms selected from the group consisting of oxygen and sulfur; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆, R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen, comprising:
(a) reacting 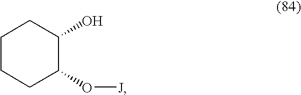
wherein O-J is a leaving group,
with 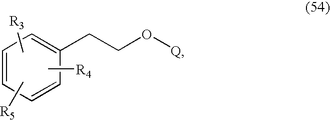
wherein R₃, R₄ and R₅ are as defined above and O-Q is a leaving group that reacts with the hydroxy group (—OH) in formula (84) to form an ether of formula (74), 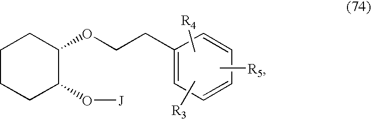
such that the stereochemical configuration of the hydroxy group is retained in the ether;
(b) optionally protecting compound of formula (84) before the first reaction; and
(c) reacting the ether of formula (74) with 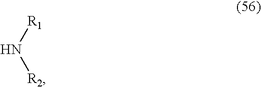
wherein R₁ and R₂ are as defined above, to form the aminocyclohexyl ether of formula (75).

75. A method of claim 74, further comprising before said first reaction (a), deprotecting 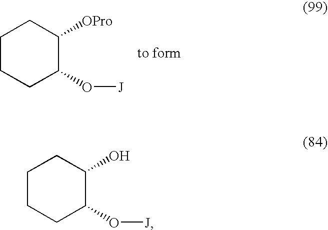
wherein Pro is a protecting group.

76. A method of claim 75, further comprising before said deprotecting reaction, activating 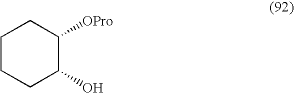
with a hydroxy activating reagent to form 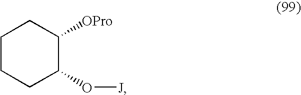
and optionally further comprising before said activating reaction, hydrogenating and hydrogenolyzing 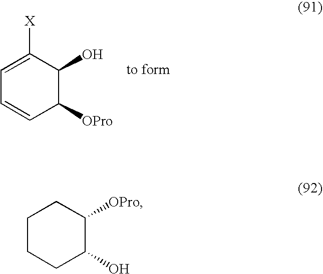
wherein X is a halide.

77. A method of claim 74, further comprising before said first reaction (a), separating a racemic mixture of 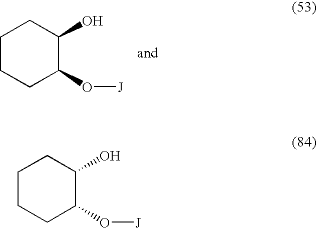
to obtain (84), wherein said separation step further comprises optionally functionalizing one or both of 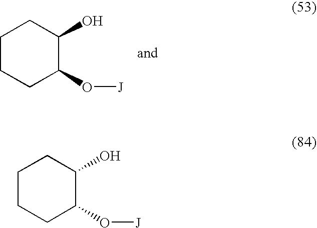
such that the compounds are amenable to resolution;
performing resolution to separate the compounds; and
optionally removing the functional group on said one or both functionalized compounds.

78. A method of claim 77, wherein said separation step comprises enzymatic resolution, crystallization and/or chromatographic resolution.

79. A method of claim 77, wherein said resolution is lipase mediated.

80. A method of claim 74, further comprising before said first reaction (a), removing a functional group G₁ from

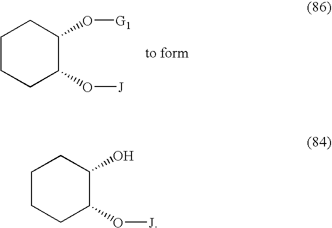

81. The method of any one of claims 74, 75, 76, 77, 78 and 79,
wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy;
wherein R₃, R₄ and R₅ are independently hydrogen, hydroxy or C₁-C₆alkoxy, with the proviso that R₃, R₄ and R₅ cannot all be hydrogen;
wherein O-J is an alkyl sulfonate or an aryl sulfonate;
wherein O-Q is selected from an imidate ester, an O-carbonate, a S-carbonate, an O-sulfonyl derivative, and a phosphate derivative;
wherein, if present, Pro is TBDPS; and
wherein, if present, X is Cl.

82. The method of any one of claims 74, 75, 76, 77, 78 and 79,
wherein 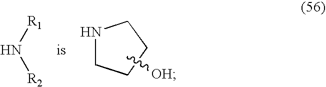
wherein at least one of R₃, R₄ and R₅ is C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, Pro is TBDPS; and
wherein, if present, X is Cl.

83. The method of any one of claims 74, 75, 76, 77, 78 and 79,
wherein 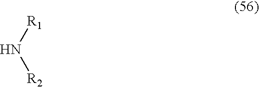
is 3R-pyrrolidinol (65) or 3S-pyrrolidinol (65A);
wherein R₃ is hydrogen, and R₄ and R₅ are C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, Pro is TBDPS; and
wherein, if present, X is Cl.

84. The method of any one of claims 74, 75, 76, 77, 78 and 79,
wherein 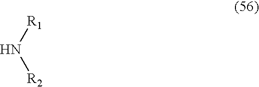
is 3R-pyrrolidinol (65)
wherein R₃ is hydrogen, R₄ is methoxy at C3 of the phenyl group and R₅ is methoxy at C4 of the phenyl group;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate or pentafluorobenzimidate;
wherein, if present, Pro is TBDPS; and
wherein, if present, X is Cl,
such that the aminocyclohexyl ether of formula (79) is

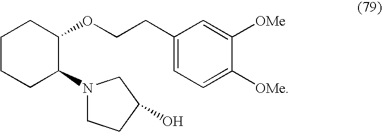

85. A method for stereoselectively making an aminocyclohexyl ether of formula (75): 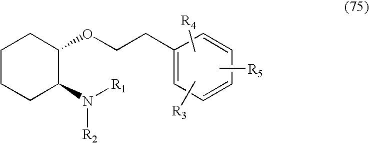
wherein independently at each occurrence, R₁ and R₂ are hydrogen, C₁-C₈alkyl, C₃-C₈alkoxyalkyl, C₁-C₈hydroxyalkyl, or C₇-C₁₂aralkyl; or
R₁ and R₂ are independently C₃-C₈alkoxyalkyl, C₁-C₈hydroxyalkyl, or C₇-C₁₂aralkyl; or R₁ and R₂, when taken together with the nitrogen atom to which they are directly attached in formula (75), form a ring denoted by formula (I): 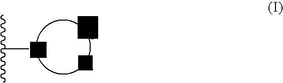
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two heteroatoms selected from the group consisting of oxygen and sulfur; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3-yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆, R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen, comprising:
(a) reacting 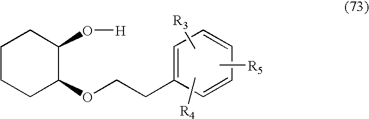
with a hydroxy activating reagent to form 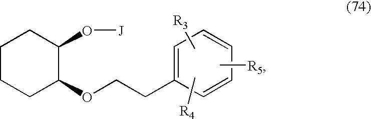
wherein O-J is a leaving group, R₃, R₄ and R₅ are as defined above; and
(b) reacting the product of the first reaction, compound of formula (74) with 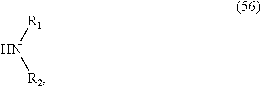
wherein R₁ and R₂ are as defined above, to form the aminocyclohexyl ether of formula (75).

86. A method of claim 85, further comprising before said first reaction (a),
hydrogenating and hydrogenolyzing 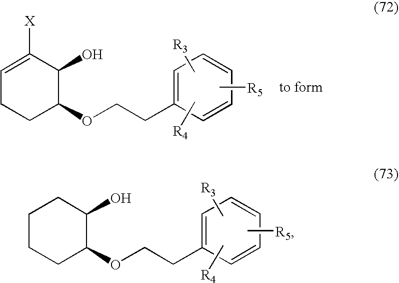
wherein X is a halide.

87. A method of claim 86, further comprising before said hydrogenating and hydrogenolyzing reaction, reacting 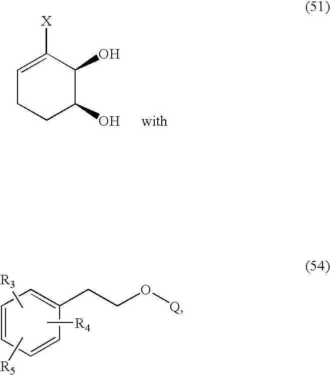
wherein O-Q is a leaving group that reacts preferentially with one of the hydroxy groups (—OH) in formula (51) to form an ether of formula (72), such that the stereochemical configuration of said hydroxy group is retained in the ether (72).

88. The method of any one of claims 85, 86 and 87, wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy;
wherein R₃, R₄ and R₅ are independently hydrogen, hydroxy or C₁-C₆alkoxy, with the proviso that R₃, R₄ and R₅ cannot all be hydrogen;
wherein O-J is an alkyl sulfonate or an aryl sulfonate;
wherein O-Q is selected from an imidate ester, an O-carbonate, a S-carbonate, an O-sulfonyl derivative, and a phosphate derivative; and
wherein, if present, X is Cl.

89. The method of claims 85, 86 and 87,
wherein 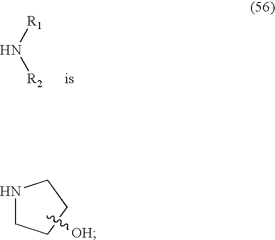
wherein at least one of R₃, R₄ and R₅ is C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, X is Cl.

90. The method of any one of claims 85, 86 and 87,
wherein 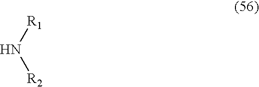
is 3R-pyrrolidinol (65) or 3S-pyrrolidinol (65A);
wherein R₃ is hydrogen, and R₄ and R₅ are C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, X is Cl.

91. The method of any one of claims 85, 86 and 87,
wherein 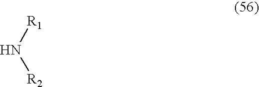
is 3R-pyrrolidinol (65);
wherein R₃ is hydrogen, R₄ is methoxy at C3 of the phenyl group and R₅ is methoxy at C4 of the phenyl group;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate or pentafluorobenzimidate; and
wherein, if present, X is Cl,
such that the aminocyclohexyl ether of formula (75) is

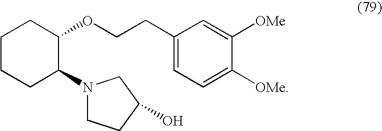

92. A method for stereoselectively making an aminocyclohexyl ether of formula (57): 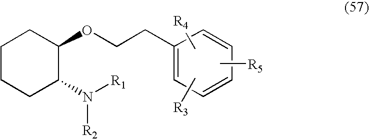
wherein independently at each occurrence, R₁ and R₂ are hydrogen, C₁-C₈alkyl, C₃-C₈alkoxyalkyl, C₁-C₈hydroxyalkyl, or C₇-C₁₂aralkyl; or
R₁ and R₂ are C₃-C₈alkoxyalkyl, C₁-C₈hydroxyalkyl, or C₇-C₁₂aralkyl; or R₁ and R₂, when taken together with the nitrogen atom to which they are directly attached in formula (57), form a ring denoted by formula (I): 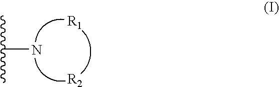
wherein the ring of formula (I) is formed from the nitrogen as shown as well as three to nine additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, C₁-C₃hydroxyalkyl, oxo, C₂-C₄acyl, C₁-C₃alkyl, C₂-C₄alkylcarboxy, C₁-C₃alkoxy, and C₁-C₂₀alkanoyloxy, or may be substituted to form a spiro five- or six-membered heterocyclic ring containing one or two heteroatoms selected from the group consisting of oxygen and sulfur; or any two adjacent additional carbon ring atoms may be fused to a C₃-C₈carbocyclic ring, and any one or more of the additional nitrogen ring atoms may be substituted with substituents selected from the group consisting of hydrogen, C₁-C₆alkyl, C₂-C₄acyl, C₂-C₄hydroxyalkyl and C₃-C₈alkoxyalkyl; or
R₁ and R₂, when taken together with the nitrogen atom to which they are directly attached in formula (I), may form a bicyclic ring system selected from the group consisting of 3-azabicyclo[3.2.2]nonan-3-yl, 2-azabicyclo[2.2.2]octan-2-yl, 3-azabicyclo[3.1.0]hexan-3yl, and 3-azabicyclo[3.2.0]heptan-3-yl; and
wherein R₃, R₄ and R₅ are independently bromine, chlorine, fluorine, carboxy, hydrogen, hydroxy, hydroxymethyl, methanesulfonamido, nitro, cyano, sulfamyl, trifluoromethyl, C₂-C₇alkanoyloxy, C₁-C₆alkyl, C₁-C₆alkoxy, C₂-C₇alkoxycarbonyl, C₁-C₆thioalkyl, aryl or N(R₆,R₇) where R₆ and R₇ are independently hydrogen, acetyl, methanesulfonyl or C₁-C₆alkyl; or R₃, R₄ and R₅ are independently hydrogen, hydroxy or C₁-C₆alkoxy; with the proviso that R₃, R₄ and R₅ cannot all be hydrogen, comprising:
(a) hydrogenating and hydrogenolyzing 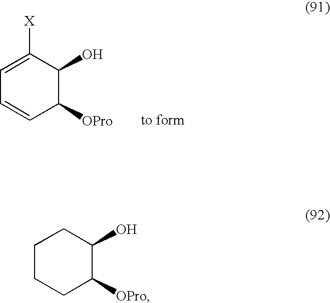
wherein Pro is a protecting group, X is a halide;
(b) alkylating 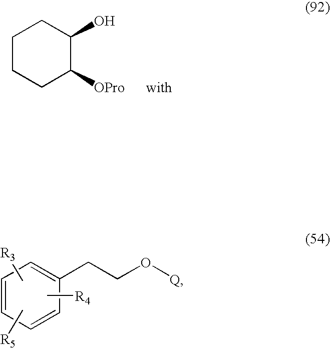
wherein R₃, R₄ and R₅ are as defined above and O-Q is a leaving group that reacts with the hydroxy group (—OH) in formula (92) to form an ether of formula (93) 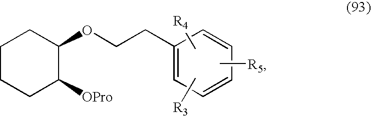
such that the stereochemical configuration of the hydroxy group is retained in the ether;
(c) deprotecting 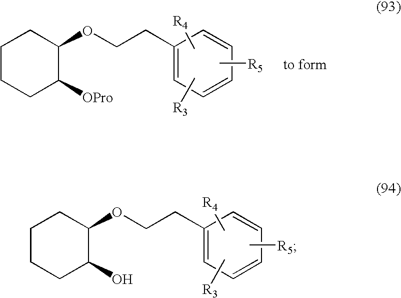
(d) activating 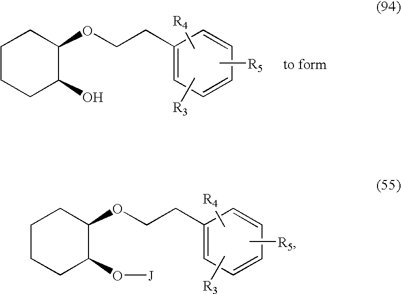
wherein O-J is a leaving group; and
(e) reacting 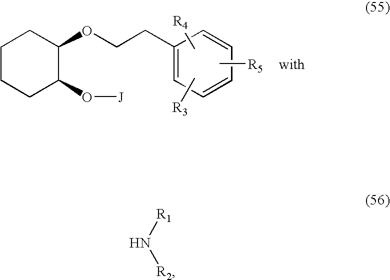
wherein R₁ and R₂ are as defined above, to form the aminocyclohexyl ether of formula (57).

93. A method of claim 92, further comprising before said first reaction (a), protecting one of the hydroxyl groups in formula (50)

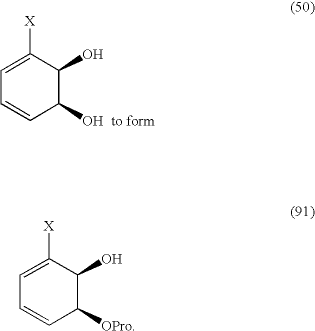

94. The method of any one of claims 92 and 93,
wherein the ring of formula (I) is formed from the nitrogen as shown as well as four to six additional ring atoms independently selected from the group consisting of carbon, nitrogen, oxygen, and sulfur; where any two adjacent ring atoms may be joined together by single or double bonds, and where any one or more of the additional carbon ring atoms may be substituted with one or two substituents selected from the group consisting of hydrogen, hydroxy, oxo, C₁-C₃alkyl, and C₁-C₃alkoxy, and
wherein R₃, R₄ and R₅ are independently hydrogen, hydroxy or C₁-C₆alkoxy, with the proviso that R₃, R₄ and R₅ cannot all be hydrogen;
wherein O-J is selected from an alkyl sulfonate or an aryl sulfonate;
wherein O-Q is selected from an imidate ester, an O-carbonate, a S-carbonate, an O-sulfonyl derivative, and a phosphate derivative;
wherein, if present, Pro is TBDPS; and
wherein, if present, X is Cl.

95. The method of any one of claims 92 and 93,
wherein 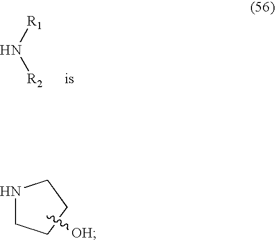
wherein at least one of R₃, R₄ and R₅ is C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, Pro is TBDPS; and
wherein, if present, X is Cl.

96. The method of any one of claims 92 and 93,
wherein 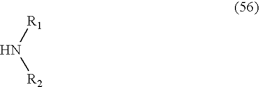
is 3R-pyrrolidinol (65) or 3S-pyrrolidinol (65A);
wherein R₃ is hydrogen, and R₄ and R₅ are C₁-C₆alkoxy;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate, a pentafluorobenzimidate, an imidazole carbonate derivative, an imidazolethiocarbonate, an O-sulfonyl derivative, a diphenyl phosphate, a diphenylphosphineimidate, or a phosphoroamidate;
wherein, if present, Pro is TBDPS; and
wherein, if present, X is Cl.

97. The method of any one of claims 92 and 93
wherein 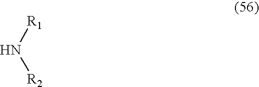
is 3R-pyrrolidinol (65);
wherein R₃ is hydrogen, R₄ is methoxy at C3 of the phenyl group and R₅ is methoxy at C4 of the phenyl group;
wherein O-J is a mesylate, a benzenesulfonate, a mono- or poly-alkylbenzenesulfonate, a mono- or poly-halobenzenesulfonate, a tosylate or a nosylate;
wherein O-Q is a trihaloacetimidate or pentafluorobenzimidate;
wherein, if present, Pro is TBDPS; and
wherein, if present, X is Cl,
such that the aminocyclohexyl ether of formula (57) is [chemical structure]
